(19) United States
(12) Patent Application Publication
(10) Pub. No.: US 2011/0014669 A1
(43) Pub. Date: Jan. 20, 2011

(54) Production of 1,4 Butanediol in a Microorganism

(75) Inventors: Kevin T. Madden, Arlington, MA (US); David M. Young, Newtonville, MA (US); Stanley Bower, La Jolla, CA (US); Chi-Li Liu, Decatur, IL (US)

Correspondence Address: FISH & RICHARDSON P.C. (BO), P.O. BOX 1022, MINNEAPOLIS, MN, 55440-1022, US

(73) Assignees: Microbia, Inc.; Tate & Lyle Ingredients Americas, Inc.

(21) Appl. No.: 12/692,943

(22) Filed: Jan. 25, 2010

Related U.S. Application Data
(60) Provisional application No. 61/146,979, filed on Jan. 23, 2009.

(51) Int. Cl. C12P 7/18; C12N 1/00; C12N 1/21

(52) U.S. Cl. 435/158; 435/243; 435/252.32

(57) ABSTRACT

A biological method for the conversion of L-glutamate to 1,4-butanediol that involves a decarboxylation step and avoids production of 4-hydroxybutyrate as an intermediate is described. The method includes: (a) conversion of L-glutamate to L-glutamate 5-phosphate; (b) conversion of L-glutamate 5-phosphate to L-glutamate 5-semialdehyde; (c) conversion of L-glutamate 5-semialdehyde to 5-hydroxy-L-norvaline; (d) conversion of 5-hydroxy-L-norvaline to 5-hydroxy-2-oxopentanoate; (e) conversion of 5-hydroxy-2-oxopentanoate to 4-hydroxybutanal; and (f) the conversion of 4-hydroxybutanal to 1,4-butanediol.

This application claims the benefit of priority of U.S. Ser. No. 61/146,979, filed Jan. 23, 2009, the entire content of which is hereby incorporated by reference.

### BACKGROUND

Approximately 1.4 million metric tons of 1,4-butanediol (BDO) are produced annually from fossil fuel-based chemical building blocks such as butane, butadiene, acetylene and formaldehyde. BDO is a chemical intermediate used in the manufacture of a variety of polymers, solvents and fine chemicals. Representative chemicals that can be derived from BDO include gamma-butyrolactone (GBL), pyrrolidones and tetrahydrofuran (THF). BDO and chemicals made through further processing of BDO include: thermoplastics, spandex fibers, cements, inks and cleaning agents.

A need exists for developing alternative strategies for producing the aforementioned chemical building blocks through processes that substitute renewable for fossil fuel-based feedstocks. Biological production routes present the further possible advantages of reduced energy utilization, lower CO₂ emissions and decreased capital expenditures.

The fermentative production of four-carbon dicarboxylic acids, such as succinate, has been explored as a means to provide a bio-based feedstock for the production of BDO and related chemicals. In these approaches hydrogenation technologies are employed to further process succinic acid to BDO. Despite the high, demonstrated efficiency of these fermentation processes (e.g., yields in excess of 1.5 moles succinic acid per mole of dextrose), their economic competitiveness has been challenged by costs associated with complex media components, purification of the dicarboxylic acid, waste disposal, and the integration of biological and hydrogenation processes.

More recently, multiple groups have demonstrated the potential for directly producing BDO through fermentation using bacteria that were constructed using metabolic engineering approaches (see, for example, WO 2008/115840). Natural isolates have never been shown to produce BDO, although a wide variety of organisms are capable of catabolizing this chemical. In principle, BDO produced through fermentation can be purified using methods such as micro- and nano-filtration or liquid-liquid extraction of the broth with a water immiscible solvent and subsequent distillation to isolate the BDO. This process simplicity, relative to the integrated fermentation and chemical modification process described above, offers the potential for a dramatic reduction in capital expenditures as well as utility and fixed costs. The theoretical efficiency (e.g., moles of BDO per mole of substrate) of a direct BDO process is much lower than that attained via fermentation to a dicarboxylic acid due to the increased metabolic energy and reducing equivalents required in the biosynthesis of BDO. Therefore, maximal fermentation yield and productivity are essential for the attainment of an economical BDO manufacturing process through direct fermentation.

The theoretical efficiency of producing BDO from dextrose is approximately one mole BDO per mole dextrose; the precise value will vary depending on specific pathway of choice and other assumptions. Many of the proposed metabolic engineering strategies for BDO production entail construction of a pathway in which carbon flows through the 4-hydroxybutyrate (4HB) intermediate. As examples, succinate, [symbol] −ketoglutarate and glutamate are key central metabolites that have been identified as substrates to be utilized for further biosynthesis to BDO via 4HB. Regardless of the elected pathway and production organism, metabolic engineering strategies are likely to involve manipulations that employ both native and heterologous genes required to produce both 4HB and BDO. In principle, non-4HB pathways to produce BDO can be identified, and these pathways may be advantageous in that the engineered strains are likely to be subject to different metabolic and physiological constraints.

### SUMMARY

The present disclosure provides a recombinant microorganism having a novel BDO biosynthetic pathway. Activities required for this novel BDO biosynthetic pathway can include, but are not limited to: a glutamate-5-kinase, a glutamate-5-semialdehyde dehydrogenase (glutamyl phosphate reductase), an oxidoreductase activity, a transaminase (aminotransferase), a keto-acid decarboxylase, and a second oxidoreductase activity. Construction of this pathway enables the biosynthesis of BDO from z,33 − ketoglutarate and glutamate. Additional modifications can be performed to optimize flux to these central metabolites, to reduce drain of these central metabolites and pathway intermediates to competing pathways, and to overcome other limitations that interfere with the efficient production of BDO. Preferred microorganisms to be modified to enable the biological production of BDO include bacteria that possess a high intrinsic ability to produce glutamate.

Described herein is a recombinant microbial cell comprising at least two (three, four, five or six) nucleic acid molecules selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol,
wherein the cell produces at least one of 5-hydroxy-L-norvaline, 5-hydroxy-2-oxopentanoate, 4-hydroxybutanal and 1,4-butanediol.

Also described is a recombinant microbial cell comprising at least two (three or four) nucleic acid molecules selected from:
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
(c) a nucleic acid molecule encoding a polypeptide that catalyzes the conversion of 5-hydroxy-2-oxopentanoate to 4-hydroxybutanal;
and
(d) a nucleic acid molecule encoding a polypeptide that catalyzes the conversion of 4-hydroxybutanal to 1,4-butanediol,
wherein the cell produces at least one of 5-hydroxy-L-norvaline, 5-hydroxy-2-oxopentanoate, 4-hydroxybutanal and 1,4-butanediol.

Described herein is a recombinant microbial cell comprising a nucleic acid molecule encoding a polypeptide that catalyzes the conversion of 5-hydroxy-2-oxopentanoate to 4-hydroxybutanal, wherein the cell produces 4-hydroxybutanal. In some cases the recombinant microbial cell further comprises at least one (two, three four or five nucleic acid molecule selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
and
(e) a nucleic acid molecule encoding a polypeptide that catalyzes the conversion of 4-hydroxybutanal to 1,4-butanediol.

In some cases the microbial cell comprises at least one (two, or three) nucleic acid molecule selected from the group consisting of:
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
and
(c) a nucleic acid molecule encoding a polypeptide that catalyzes the conversion of 4-hydroxybutanal to 1,4-butanediol.

Described herein is a recombinant microbial cell comprising one or both of:
(a) a nucleic acid molecule encoding a polypeptide that catalyzes the conversion of L-glutamate 5-semialdehyde to 5-hydroxy-L-norvaline; and
(b) a nucleic acid molecule encoding a polypeptide that catalyzes the conversion of 5-hydroxy-2-oxopentanoate to 4-hydroxybutanal,
wherein the cell produces at least one of 5-hydroxy-L-norvaline, 5-hydroxy-2-oxopentanoate, 4-hydroxybutanal and 1,4-butanediol.

In some cases: the cell comprises both: (a) a nucleic acid molecule encoding a polypeptide that catalyzes the conversion of L-glutamate 5-semialdehyde to 5-hydroxy-L-norvaline; and (b) a nucleic acid molecule encoding a polypeptide that catalyzes the conversion of 5-hydroxy-2-oxopentanoate to 4-hydroxybutanal; the cell further comprises one or both of: (c) a nucleic acid molecule encoding a polypeptide that catalyzes the conversion of 5-hydroxy-L-norvaline to 5-hydroxy-2-oxopentanoate; and (d) a nucleic acid molecule encoding a polypeptide that catalyzes the conversion of 4-hydroxybutanal to 1,4-butanediol; the cell further comprises both: (c) a nucleic acid molecule encoding a polypeptide that catalyzes the conversion of 5-hydroxy-L-norvaline to 5-hydroxy-2-oxopentanoate; and (d) a nucleic acid molecule encoding a polypeptide that catalyzes the conversion of 4-hydroxybutanal to 1,4-butanediol; the cell further comprising one or both of: (e) a heterologous nucleic acid molecule encoding a polypeptide that catalyzes the conversion of L-glutamate to L-glutamate 5-phosphate; and (f) a heterologous nucleic acid molecule encoding a polypeptide that catalyzes the conversion of L-glutamate 5-phosphate to L-glutamate 5-semialdehyde; the cell further comprises both of: (e) a heterologous nucleic acid molecule encoding a polypeptide that catalyzes the conversion of L-glutamate to L-glutamate 5-phosphate; and (f) a heterologous nucleic acid molecule encoding a polypeptide that catalyzes the conversion of L-glutamate 5-phosphate to L-glutamate 5-semialdehyde.

In some cases: the cell produces 5-hydroxy-L-norvaline, the cell produces 5-hydroxy-2-oxopentanoate; the cell produces 4-hydroxybutanal; the cell produces 1,4-butanediol; the cell produces 5-hydroxy-L-norvaline, 5-hydroxy-2-oxopentanoate, 4-hydroxybutanal and 1,4-butanediol.

In some cases, the recombinant microbial cell comprises at least one of:
(a) a nucleic acid molecule encoding a polypeptide that catalyzes the conversion of L-glutamate to L-glutamate 5-phosphate, wherein expression of the nucleic acid molecule is under the control of an inducible promoter;
(b) a nucleic acid molecule encoding a polypeptide that catalyzes the conversion of L-glutamate 5-phosphate to L-glutamate 5-semialdehyde, wherein expression of the nucleic acid molecule is under the control of an inducible promoter;
(c) a nucleic acid molecule encoding a polypeptide that catalyzes the conversion of L-glutamate 5-semialdehyde to 5-hydroxy-L-norvaline, wherein expression of the nucleic acid molecule is under the control of an inducible promoter;
(d) a nucleic acid molecule encoding a polypeptide that catalyzes the conversion of 5-hydroxy-L-norvaline to 5-hydroxy-2-oxopentanoate, wherein expression of the nucleic acid molecule is under the control of an inducible promoter;
(e) a nucleic acid molecule encoding a polypeptide that catalyzes the conversion of 5-hydroxy-2-oxopentanoate to 4-hydroxybutanal, wherein expression of the nucleic acid molecule is under the control of an inducible promoter;
and
(f) a nucleic acid molecule encoding a polypeptide that catalyzes the conversion of 4-hydroxybutanal to 1,4-butanediol, wherein expression of the nucleic acid molecule is under the control of an inducible promoter.

In some cases recombinant microbial cell comprises an endogenous nucleic acid molecule encoding a polypeptide that catalyzes the conversion of L-glutamate to L-glutamate 5-phosphate, wherein expression of the nucleic acid molecule is under the control of an inducible promoter.

In some cases recombinant microbial cell comprises an endogenous nucleic acid molecule encoding a polypeptide that catalyzes the conversion of L-glutamate 5-phosphate to L-glutamate 5-semialdehyde, wherein expression of the nucleic acid molecule is under the control of an inducible promoter.

Also described is: a recombinant microbial comprising:
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
and
(c) a nucleic acid molecule encoding a polypeptide that catalyzes the conversion of 4-hydroxybutanal to 1,4-butanediol,
wherein the cell has been modified to reduce the expression or activity of an endogenous polypeptide that catalyzes the conversion of pyrroline 5-carboxylate to proline.
In some cases: the polypeptide that catalyzes the conversion of pyrroline 5-carboxylate to proline is pyrroline 5-carboxylate reductase; the cell does not comprise a nucleic acid molecule encoding active pyrroline 5-carboxylate reductase; the microbial cell further comprises a nucleic acid molecule encoding a polypeptide that catalyzes the conversion of L-glutamate to L-glutamate 5-phosphate, wherein expression of the nucleic acid molecule is under the control of an inducible promoter; the cell further comprises a nucleic acid molecule encoding a polypeptide that catalyzes the conversion of L-glutamate 5-phosphate to L-glutamate 5-semialdehyde.

In some cases the microbial cells described herein comprise one or both of:
(a) a nucleic acid molecule encoding a polypeptide that catalyzes the conversion of L-glutamate to L-glutamate 5-phosphate, wherein expression of the nucleic acid molecule is under the control of an inducible promoter; and
(b) a nucleic acid molecule encoding a polypeptide that catalyzes the conversion of L-glutamate 5-phosphate to L-glutamate 5-semialdehyde, wherein expression of the nucleic acid molecule is under the control of an inducible promoter.

In various embodiments: the recombinant microbial cell comprises a nucleic acid molecule encoding a polypeptide that catalyzes the conversion of 4-hydroxybutanal to 1,4-butanediol; the cell has been modified to reduce the expression or activity of a polypeptide that catalyzes the conversion of pyrroline 5-carboxylate to proline; the polypeptide that catalyzes the conversion of pyrroline 5-carboxylate to proline is pyrroline 5-carboxylate reductase; the cell does not comprise a nucleic acid molecule encoding active pyrroline 5-carboxylate reductase; the polypeptide that catalyzes the conversion of 5-hydroxy-2-oxopentanoate to 4-hydroxybutanal is at least 80% identical to any of SEQ ID NOs:36-43, 59-70 or any of the sequences represented by the Genbank Accession numbers in FIG. 17.

In some cases: (a) the polypeptide that catalyzes the conversion of L-glutamate to L-glutamate 5-phosphate is at least 80% identical to any of SEQ ID NOs:1-9 or any of the sequences represented by the Genbank Accession numbers in FIG. 13; (b) the polypeptide that catalyzes the conversion of L-glutamate 5-phosphate to L-glutamate 5-semialdehyde is at least 80% identical to any of SEQ ID NOs:10-15 or any of the sequences represented by the Genbank Accession numbers in FIG. 14; (c) the polypeptide that catalyzes the conversion of L-glutamate 5-semialdehyde to 5-hydroxy-L-norvaline is at least 80% identical to any of SEQ ID NOs:16-27 or any of the sequences represented by the Genbank Accession numbers in FIG. 15;
(d) the polypeptide that catalyzes the conversion of 5-hydroxy-L-norvaline to 5-hydroxy-2-oxopentanoate is at least 80% identical to any of SEQ ID NOs:28-35, 58 or any of the sequences represented by the Genbank Accession numbers in FIG. 16; (e) the polypeptide catalyzes the conversion of 5-hydroxy-2-oxopentanoate to 4-hydroxybutanal is at least 80% identical to any of SEQ ID NOs:36-43, 59-70 or any of the sequences represented by the Genbank Accession numbers in FIG. 17; and (f) the polypeptide that catalyzes the conversion of 4-hydroxybutanal to 1,4-butanediol is at least 80% identical to any of SEQ ID NOs:44-50 or any of the sequences represented by the Genbank Accession numbers in FIG. 18.

In some cases: (a) the polypeptide that catalyzes the conversion of L-glutamate 5-semialdehyde to 5-hydroxy-L-norvaline is at least 80% identical to any of SEQ ID NOs:16-27 or any of the sequences represented by the Genbank Accession numbers in FIG. 15; (b) the polypeptide that catalyzes the conversion of 5-hydroxy-L-norvaline to 5-hydroxy-2-oxopentanoate is at least 80% identical to any of SEQ ID NOs:28-35, 58 or any of the sequences represented by the Genbank Accession numbers in FIG. 16; (c) the polypeptide catalyzes the conversion of 5-hydroxy-2-oxopentanoate to 4-hydroxybutanal is at least 80% identical to any of SEQ ID NOs:36-43, 59-70 or any of the sequences represented by the Genbank Accession numbers in FIG. 17; and (d) the polypeptide that catalyzes the conversion of 4-hydroxybutanal to 1,4-butanediol is at least 80% identical to any of SEQ ID NOs:44-50 or any of the sequences represented by the Genbank Accession numbers in FIG. 18.

In some cases: the polypeptide that catalyzes the conversion of L-glutamate to L-glutamate 5-phosphate is a variant having one or more amino acid changes that confer reduced inhibition compared to an otherwise identical polypeptide lacking the one or more amino acid changes; and the polypeptide that catalyzes the conversion of L-glutamate 5-semialdehyde to 5-hydroxy-L-norvaline is a variant having one or more amino acid changes that confer reduced inhibition compared to an otherwise identical polypeptide lacking the one or more amino acid changes.

In some cases: the microbial cell is a bacterium; the bacterium is a coryneform bacterium or a bacterium of the genus Arthrobacter, Bacillus, Escherichia, Pseudomonas or Rhodococcus; the bacterium is a Corynebacterium glutamicum, Corynebacterium acetoglutamicum, Corynebacterium melassecola, Corynebacterium thermoaminogenes, Brevibacterium lactofermentum, Brevibacterium lactis, Brevibacterium ketoglutamicum, Brevibacterium saccharolyticum or Brevibacterium flavum bacterium; the bacterium is a Corynebacterium glutamicum bacterium; the recombinant microbial cell is produced by genetic modification of a cell selected from the group consisting of: ATCC 13032, ATCC 21157, ATCC 21158, ATCC 21159, ATCC 21355, NRRL B-11423, NRRL B-11294, ATCC 10798, ATCC 25208, ATCC 25377 and ATCC 25378; at least one of the polypeptides is heterologous to the cell; at least two of the polypeptides are heterologous to the cell; at least three of the polypeptides are heterologous to the cell; at least four of the polypeptides are heterologous to the cell; and at least five of the polypeptides are heterologous to the cell.

In some cases the nucleic acid molecule is an isolated nucleic acid molecule.

In some cases: the polypeptide that catalyzes the conversion of L-glutamate to L-glutamate 5-phosphate is a glutamate 5-kinase; the polypeptide that catalyzes the conversion of L-glutamate to L-glutamate 5-phosphate comprises an amino acid sequence that is at least 80% identical to any of SEQ ID NOs:1-9 or any of the sequences represented by the Genbank Accession numbers in FIG. 13; the polypeptide that catalyzes the conversion of L-glutamate 5-phosphate to L-glutamate 5-semialdehyde is a glutamate-5-semialdehyde dehydrogenase; the polypeptide that catalyzes the conversion of L-glutamate 5-phosphate to L-glutamate 5-semialdehyde comprises an amino acid sequence that is at least 80% identical to any of SEQ ID NOs:10-15 or any of the sequences represented by the Genbank Accession numbers in FIG. 14; the polypeptide that catalyzes the conversion of L-glutamate 5-semialdehyde to 5-hydroxy-L-norvaline is a long chain alcohol NAD+ oxidoreductase, L-iditol 2-dehydrogenase, or a zinc-containing alcohol dehydrogenase; the polypeptide that catalyzes the conversion of L-glutamate 5-semialdehyde to 5-hydroxy-L-norvaline comprises an amino acid sequence that is at least 80% identical to any of SEQ ID NOs:16-27 or any of the sequences represented by the Genbank Accession numbers in FIG. 15; the polypeptide that catalyzes the conversion of 5-hydroxy-L-norvaline to 5-hydroxy-2-oxopentanoate is a branched-chain-amino-acid transaminase; the polypeptide that catalyzes the conversion of 5-hydroxy-L-norvaline to 5-hydroxy-2-oxopentanoate uses alpha-ketoglutarate as an amino acceptor; the polypeptide that catalyzes the conversion of 5-hydroxy-L-norvaline to 5-hydroxy-2-oxopentanoate comprises an amino acid sequence that is at least 80% identical to any of SEQ ID NOs:28-35, 58 or any of the sequences represented by the Genbank Accession numbers in FIG. 16; the polypeptide that catalyzes the conversion of 5-hydroxy-2-oxopentanoate to 4-hydroxybutanal is a decarboxylase.

Also described is a recombinant microbial host wherein the polypeptide that catalyzes the conversion of 5-hydroxy-2-oxopentanoate to 4-hydroxybutanal is a branched chain alpha-keto decarboxylase, a pyruvate decarboxylase or a benzoylformate decarboxylase; the polypeptide that catalyzes the conversion of 5-hydroxy-2-oxopentanoate to 4-hydroxybutanal comprises an amino acid sequence that is at least 80% identical to any of SEQ ID NOs:36-43, 59-70 or any of the sequences represented by the Genbank Accession numbers in FIG. 17; the polypeptide that catalyzes the conversion of 4-hydroxybutanal to 1,4-butanediol is a 4-hydroxybutyrate dehydrogenase, a 1,3 propanediol dehydrogenase, cinnamyl-alcohol dehydrogenase, or an alcohol dehydrogenase; the polypeptide that catalyzes the conversion of 4-hydroxybutanal to 1,4-butanediol comprises an amino acid sequence that is at least 80% identical to any of SEQ ID NOs:44-50 or any of the sequences represented by the Genbank Accession numbers in FIG. 18.

In some cases the recombinant microbial cell comprises a polypeptide that catalyzes the conversion of L-glutamate 5-semialdehyde to 5-hydroxy-L-norvaline and is at least 80%, 85%, 90%, 95%, 98% or 100% identical to SEQ ID NO:18.

In some cases the recombinant microbial cell comprises a polypeptide that catalyzes the conversion of 5-hydroxy-L-norvaline to 5-hydroxy-2-oxopentanoate and is at least 80%, 85%, 90%, 95%, 98% or 100% identical to SEQ ID NO:58.

In some cases the recombinant microbial cell comprises a polypeptide that catalyzes the conversion of 5-hydroxy-2-oxopentanoate to 4-hydroxybutanal and is at least 80%, 85%, 90%, 95%, 98% or 100% identical to SEQ ID NO:36.

In some cases the recombinant microbial cell comprises a polypeptide that catalyzes the conversion of 4-hydroxybutanal to 1,4-butanediol is at least 80%, 85%, 90%, 95%, 98% or 100% identical to SEQ ID NO:47.

In some cases the polypeptide has 1, 2, 3, 4, 5, 6, 7, 8, 9, 10 or fewer amino acid changes compared to a polypeptide sequence disclosed herein. In some cases the amino acid changes are conservative changes within the following groups: polar (S, T, N, and Q); positively charged (R, H and K); negatively charged (D and E); and hydrophobic (A, I, L, M, F, W, Y and V).

In some cases: the recombinant microbial host has a genetic modification that decreases the activity or expression of one or more enzymes selected from
(a) an enzyme that catalyzes the conversion of alpha-ketoglutarate to isocitrate;
(b) an enzyme that catalyzes the conversion of alpha-ketoglutarate to succinyl-CoA;
(c) an enzyme that catalyzes the conversion of L-glutamate to alpha-ketoglutarate;
(d) an enzyme that catalyzes the conversion of L-glutamate to L-glutamine;
(e) an enzyme that catalyzes the conversion of L-glutamate to L-1-pyrroline 5-carboxylate;
(f) an enzyme that catalyzes the conversion of L-proline to L-1-pyrroline 5-carboxylate;
and
(g) an enzyme that catalyzes the conversion of L-1-pyrroline 5-carboxylate to L-proline.

In some cases: the recombinant microbial host has a genetic modification that increases the activity or expression of one or more enzymes selected from:

(a) an enzyme that catalyzes the conversion of alpha-ketoglutarate to isocitrate;
(b) an enzyme that catalyzes the conversion of alpha-ketoglutarate to succinyl CoA;
and
(c) an enzyme that catalyzes the conversion of L-glutamate to alpha-ketoglutarate.

In some cases: the recombinant microbial host further comprises a nucleic acid molecule encoding one or more polypeptides selected from the group consisting of:

(a) succinyl-CoA synthetase;
(b) CoA-independent succinic semialdehyde dehydrogenase;
(c) α-ketoglutarate dehydrogenase complex;
(d) 4-aminobutyrate aminotransferase;
(e) glutamate decarboxylase;
(f) CoA-dependent succinic semialdehyde dehydrogenase;
(g) 4-hydroxybutyrate dehydrogenase;
(h) α-ketoglutarate decarboxylase;
(i) 4-hydroxybutyryl-CoA transferase;
(j) butyrate kinase;
(k) phosphotransbutyrylase; and
(l) alcohol/aldehyde dehydrogenase.

In some cases: the recombinant microbial host further comprises nucleic acid molecules encoding at least two polypeptides selected from the group consisting of:

(a) succinyl-CoA synthetase;
(b) CoA-independent succinic semialdehyde dehydrogenase;
(c) α-ketoglutarate dehydrogenase complex;
(d) 4-aminobutyrate aminotransferase;
(e) glutamate decarboxylase;
(f) CoA-dependent succinic semialdehyde dehydrogenase;
(g) 4-hydroxybutyrate dehydrogenase;
(h) α-ketoglutarate decarboxylase;
(i) 4-hydroxybutyryl-CoA transferase;
(j) butyrate kinase;
(k) phosphotransbutyrylase; and
(l) alcohol/aldehyde dehydrogenase.

In some cases the recombinant microbial cell does not produce 4-hydroxybutyrate.

In various cases the polypeptide encoded by the nucleic acid molecule is operably linked to expression control sequences that permit the expression of the polypeptide.

In various cases the cell produces at least one of 5-hydroxy-L-norvaline, 5-hydroxy-2-oxopentanoate, 4-hydroxybutanal and 1,4-butanediol, e.g., when cultured on an appropriate carbon source such as glucose or

Also disclosed is a method for the production of 1,4-butanediol comprising: providing a recombinant microbial cell as described herein; and culturing the host cell under conditions whereby 1,4-butanediol is produced. The method can also include isolating the produced 1,4-butanediol.

Also disclosed is a method for the production gamma-butyrolactone comprising: providing a recombinant microbial cell as described herein; and culturing the host cell under conditions whereby 1,4-butanediol is produced; and oxidizing the 1,4-butanediol to produce gamma-butyrolactone.

Also disclosed is a method for preparing tetrahydrofuran (THF) comprising: providing a recombinant microbial cell as described herein; culturing the host cell under conditions whereby 1,4-butanediol is produced; isolating the produced 1,4-butanediol; and hydrogenolysis of the produced 1,4-butanediol to produce THF.

Also disclosed is a method for preparing pyrrolidone or N-methyl-pyrrolidone comprising: providing a recombinant microbial cell as described herein; culturing the host cell under conditions whereby 1,4-butanediol is produced; isolating the produced 1,4-butanediol; and further processing the produced 1,4-butanediol to produce pyrrolidone or N-methyl-pyrrolidone.

Also described is: a polypeptide comprising the amino acid sequence of SEQ ID NO: 36 having an amino acid change selected from the group consisting of: (a) Gln changed to Lys, Arg or His at position 362; and (b) Phe changed to Trp or Tyr at position 382; a branched-chain alpha-keto acid decarboxylase (e.g., Lactotococcus lactis branched-chain alpha-keto acid decarboxylase (KdcA)) having an amino acid change selected from the group consisting of: (a) Gln changed to Lys, Arg or His at position 362; and (b) Phe changed to Trp or Tyr at position 382; a polypeptide comprising the amino acid sequence of SEQ ID NO: 36 having (a) Gln changed to Lys, Arg or His at position 362; and (b) Phe changed to Trp or Tyr at position 382; a branched-chain alpha-keto acid decarboxylase (e.g., Lactotococcus lactis branched-chain alpha-keto acid decarboxylase (KdcA)) having: (a) Gln changed to Lys, Arg or His at position 362; and Phe changed to Trp or Tyr at position 382. In some cases the Gln at position 362 is changed to Lys. In some cases the Phe at position 382 is changed to Trp. Also described is a branched-chain alpha-keto acid decarboxylase polypeptide having one or two amino acid changes selected from the group consisting of: (a) Gln changed to Lys, Arg or His at the position corresponding to position 362 in SEQ ID NO: 36; and (b) Phe changed to Trp or Tyr at the position corresponding to position 382 in SEQ ID NO: 36. Also described is a polypeptide comprising the amino acid sequence of SEQ ID NO: 36 having an amino acid change selected from the group consisting of: (a) Gln changed to Lys, Arg or His at position 362; and (b) Phe changed to Ala, Ile, Leu, Met, Val, Trp or Tyr at position 382; a branched-chain alpha-keto acid decarboxylase (e.g., Lactotococcus lactis branched-chain alpha-keto acid decarboxylase (KdcA)) having an amino acid change selected from the group consisting of: (a) Gln changed to Lys, Arg or His at position 362; and (b) Phe changed to Ala, Ile, Leu, Met, Val, Trp or Tyr at position 382; a polypeptide comprising the amino acid sequence of SEQ ID NO: 36 having (a) Gln changed to Lys, Arg or His at position 362; and (b) Phe changed to Ala, Ile, Leu, Met, Val, Trp or Tyr at position 382; a branched-chain alpha-keto acid decarboxylase (e.g., Lactotococcus lactis branched-chain alpha-keto acid decarboxylase (KdcA)) having: (a) Gln changed to Lys, Arg or His at position 362; and Phe changed to Ala, Ile, Leu, Met, Val, Trp or Tyr at position 382. In some cases the Gln at position 362 is changed to Lys. Also described is a branched-chain alpha-keto acid decarboxylase polypeptide having one or two amino acid changes selected from the group consisting of: (a) Gln changed to Lys, Arg or His at the position corresponding to position 362 in SEQ ID NO: 36; and (b) Phe changed to Ala, Ile, Leu, Met, Val, Trp or Tyr at the position corresponding to position 382 in SEQ ID NO: 36. Also disclosed are isolated nucleic acid molecules encoding such polypeptides, vectors (including expression vectors comprising such nucleic acid molecules and cells (including microbial cells) comprising such nucleic acid molecules or vectors. These nucleic acid molecules are useful in embodiments entailing the use of a nucleic acid molecule encoding a polypeptide that catalyzes the conversion of 5-hydroxy-2-oxopentanoate to 4-hydroxybutanal.

### DEFINITIONS

Corresponding: An amino acid that is “corresponding” to an amino acid in a reference sequence occupies a site that is homologous to the site in the reference sequence. Corresponding amino acids can be identified by alignment of related sequences. Amino acid sequences can be compared to protein sequences available in public databases using algorithms such as BLAST, FASTA, ClustalW, which are well known to those skilled in the art.

Expression: The term “expression” refers to the production of a gene product (i.e., RNA or protein). For example, “expression” includes transcription of a gene to produce a corresponding mRNA, and/or translation of such an mRNA to produce the corresponding peptide, polypeptide, or protein.

Functionally linked: The phrase “functionally linked” or “operably linked” refers to a promoter or promoter region and a coding or structural sequence in such an orientation and distance that transcription of the coding or structural sequence may be directed by the promoter or promoter region.

Functionally transformed: As used herein, the term “functionally transformed” refers to a host cell that has been caused to express one or more polypeptides as described herein, such that the expressed polypeptide is functional and is active at a level higher than is observed with an otherwise identical cell (i.e., a parental cell) that has not been so transformed. In many embodiments, functional transformation involves introduction of a nucleic acid encoding the polypeptide(s) such that the polypeptide(s) is/are produced in an active form and/or appropriate location. Alternatively or additionally, in some embodiments, functional transformation involves introduction of a nucleic acid that regulates expression of such an encoding nucleic acid. Functional transformation may comprise introduction of one or more nucleic acid sequences by, for example, mating, transduction, conjugation or transformation of naturally-, chemically-, or electro-competent cells.

Gene: The term “gene”, as used herein, generally refers to a nucleic acid encoding a polypeptide, optionally including certain regulatory elements that may affect expression of one or more gene products (i.e., RNA or protein). A gene may be in chromosomal DNA, plasmid DNA, cDNA, synthetic DNA, or other DNA that encodes a peptide, polypeptide, protein, or RNA molecule, and may include regions flanking the coding sequence involved in the regulation of expression.

Heterologous: The term “heterologous”, means from a source organism other than the host cell. For example, “heterologous” as used herein refers to genetic material or a polypeptide(s) that does not naturally occur in the species in which it is present and/or being expressed. It will be understood that, in general, when heterologous genetic material or a polypeptide is selected for introduction into and/or expression by a host cell, the particular source organism from which the heterologous genetic material or polypeptide may be selected is not critical to the practice of the present disclosure. Relevant considerations may include, for example, how closely related the potential source and host organisms are in evolution, or how related the source organism is with other source organisms from which sequences of other relevant polypeptides have been selected. Where a plurality of different heterologous polypeptides and/or nucleic acids are to be introduced into and/or expressed by a host cell, different polypeptides or nucleic acids may be from different source organisms, or from the same source organism. To give but one example, in some cases, individual polypeptides may represent individual subunits of a complex protein activity and/or may be required to work in concert with other polypeptides in order to achieve the goals of the present disclosure. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acids whose sequences encode the polypeptide that have been adjusted to accommodate codon preferences of the host cell and/or to link the encoding sequences with regulatory elements active in the host cell.

Homologous: The term “homologous”, as used herein, means from the same source organism as the host cell. For example, as used here to refer to genetic material or to polypeptides, the term “homologous” refers to genetic material or a polypeptide(s) that naturally occurs in the organism in which it is present and/or being expressed, although optionally at different activity levels and/or in different amounts.

Host cell: As used herein, the “host cell” is a cell that is manipulated according to the present disclosure to produce BDO as described herein. A “modified host cell”, as used herein, is any host cell which has been modified, engineered, or manipulated in accordance with the present disclosure as compared with a parental cell. In some embodiments, the parental cell is a naturally occurring parental cell. Typically, the host cell is a microbial cell such as a bacterial, fungal cell or a yeast cell.

Hybridization: “Hybridization” refers to the ability of a strand of nucleic acid to join with a complementary strand via base pairing. Hybridization occurs when complementary sequences in the two nucleic acid strands bind to one another.

Isolated: The term “isolated”, as used herein, means that the isolated entity has been separated from at least one component with which it was previously associated. When most other components have been removed, the isolated entity is “purified” or “concentrated”. Isolation and/or purification and/or concentration may be performed using any techniques known in the art including, for example, fractionation, extraction, precipitation, or other separation.

Modified: The term “modified”, as used herein, refers to a host cell that has been modified to increase or otherwise improve the production of BDO or a BDO intermediate, as compared with an otherwise identical host organism that has not been so modified. In principle, such “modification” in accordance with the present disclosure may comprise any chemical, physiological, genetic, or other modification that appropriately alters production of BDO in a host organism as compared with such production in an otherwise identical cell not subject to the same modification. In most embodiments, however, the modification will comprise a genetic modification. For example, a genetic modification can entail: the addition of all or a portion of gene that is not naturally present in the host cell, the addition of all or a portion of a gene that is already present in the host cell, the deletion of all or a portion of a gene that is naturally in the host cell, an alteration (e.g., a sequence change in) of a gene that is naturally present in the host cell (e.g., a sequence change that increases expression, a sequence change that decreases expression, a sequence change that increases enzymatic, transport or other activity of a polypeptide, a sequence change that decreases enzymatic, transport or other activity of a polypeptide) and combinations thereof. In some cases, a modification comprises at least one chemical, physiological, genetic, or other modification; in other cases, a modification comprises more than one chemical, physiological, genetic, or other modification. In certain aspects where more than one modification is utilized, such modifications can comprise any combination of chemical, physiological, genetic, or other modifications (e.g., one or more genetic, chemical and/or physiological modification(s)).

Promoter: As is known in the art, the term “promoter” or “promoter region” refers to a DNA sequence, usually found upstream (5′) to a coding sequence, that controls expression of the coding sequence by controlling production of messenger RNA (mRNA) by providing the recognition site for RNA polymerase and/or other factors necessary for start of transcription at the correct site.

Recombinant: A “recombinant” host cell, as that term is used herein, is a host cell that has been genetically modified. For example, a “recombinant cell” can be a cell that contains a nucleic acid sequence not naturally occurring in the cell, or an additional copy or copies of an endogenous nucleic acid sequence, wherein the nucleic acid sequence is introduced into the cell or an ancestor thereof by human action. A recombinant cell includes, but is not limited to: a cell which has been genetically modified by deletion of all or a portion of a gene, a cell that has had a mutation introduced into a gene, a cell that has had a nucleic acid sequence inserted, for example, to add or disrupt a functional gene or modify its regulation, and a cell that has a gene that has been modified by both removing and adding a nucleic acid sequence. A “recombinant vector” or “recombinant DNA or RNA construct” refers to any nucleic acid molecule generated by the hand of man. For example, a recombinant construct may be a vector such as a plasmid, cosmid, virus, autonomously replicating sequence, phage, or linear or circular single-stranded or double-stranded DNA or RNA molecule. A recombinant nucleic acid may be derived from any source and/or capable of genomic integration or autonomous replication where it includes two or more sequences that have been linked together by the hand of man. Recombinant constructs may, for example, be capable of introducing a 5′ regulatory sequence or promoter region and a DNA sequence for a selected gene product into a cell in such a manner that the DNA sequence is transcribed into a functional mRNA, which may or may not be translated and therefore expressed.

Reduced inhibition. A polypeptide with “reduced inhibition” includes a polypeptide that is less inhibited by the presence of an inhibitory factor as compared to a wild-type form of the polypeptide or a polypeptide that is less inhibited by the presence of an inhibitory factor as compared to the corresponding endogenous polypeptide expressed in the organism into which the polypeptide has been introduced. In certain cases, the inhibitory factor is an allosteric inhibitor. In certain cases, the inhibitory factor may be a product or an intermediate of a BDO biosynthetic pathway, e.g., a product produced by the polypeptide that is inhibited or a product that inhibits an earlier step in the pathway. This type of inhibition is commonly referred to as feedback inhibition, and reduced inhibition includes reduced feedback inhibition. For example, a wild-type glutamate kinase from E. coli may have 10-fold less activity in the presence of a given concentration of proline, or proline plus ADP, respectively. A glutamate kinase with reduced inhibition may have, for example, 5-fold less, 2-fold less, or wild-type levels of activity in the presence of the same concentration of proline or proline plus ADP. Once a variant enzyme having one or more amino acid changes which result in reduced inhibition has been identified, it can be used as a model by those skilled in the art to create variants of a heterologous enzyme by making the same or a similar amino acid change(s) at the corresponding position(s) in the heterologous enzyme. The amino acid change in the heterologous enzyme need not be identical to the change in the model enzyme. For example, if the amino acid change in the model enzyme changes a basic amino acid to a neutral amino acid, e.g., Ala, the change in the heterologous enzyme can change a basic amino acid to a different neutral amino acid, e.g., Gly.

Selectable: The term “selectable” is used to refer to a marker whose expression confers a phenotype facilitating identification, and specifically facilitating survival or death, of cells containing the marker. A selectable marker can be a nucleotide sequence that confers antibiotic resistance in a host. These selectable markers include ampicillin, cefazolin, augmentin, cefoxitin, ceftazidime, ceftiofur, cephalothin, enrofloxicin, kanamycin, spectinomycin, streptomycin, tetracycline, ticarcillin, tilmicosin, or chloramphenicol resistance genes. Additional selectable markers include genes that can complement nutritional auxotrophies present in a particular host strain (e.g., leucine, tryptophan, or homoserine auxotrophies). Alternatively, strategies exist to identify cells that do or do not contain a particular marker. An example of a negative selection marker is the sacB gene, encoding the levansucrase gene from Bacillus subtilis. Modified cells that contain an active sacB gene can be identified by a lack of growth on medium containing sucrose. The sacB gene product acts to polymerize sucrose to form levan that is toxic to many bacteria including C. glutamicum. (see Jager, W., et al. J. Bacteriol. 174:5462-5465, 1992).

Sequence Identity: As used herein, the term “sequence identity” refers to a comparison between two sequences (e.g., two nucleic acid sequences or two amino acid sequences) and assessment of the degree to which they contain the same residue at the same position. As is known to those of ordinary skill in the art, an assessment of sequence identity includes an assessment of which positions in different sequences should be considered to be corresponding positions; adjustment for gaps, etc. is permitted. Furthermore, an assessment of residue identity can include an assessment of degree of identity such that consideration can be given to positions in which the identical residue (e.g., nucleotide or amino acid) is not observed, but a residue sharing one or more structural, chemical, or functional features is found. Identity can be determined by sequence alignment. The percent identity between the two sequences is a function of the number of identical positions shared by the sequences, taking into account the number of gaps, and the length of each gap, which needs to be introduced for optimal alignment of the two sequences. Any of a variety of algorithms or approaches may be utilized to calculate sequence identity. For example, in some embodiments, the Needleman and Wunsch (1970) J. Mol. Biol. 48:444-453 algorithm can be utilized. This algorithm has been incorporated into the GAP program in the GCG software package (available at http://www.gcg.com). In some such embodiments, the Needleman and Wunsch algorithm is employed using either a Blosum 62 matrix or a PAM250 matrix, and a gap weight of 16, 14, 12, 10, 8, 6, or 4 and a length weight of 1, 2, 3, 4, 5, or 6. In some embodiments, sequence alignment is performed using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A default set of parameters (and the one that can be used if the practitioner is uncertain about what parameters should be applied to determine if a molecule is within a sequence identity or homology limitation of the disclosure) are a Blosum 62 scoring matrix with a gap penalty of 12, a gap extend penalty of 4, and a frameshift gap penalty of 5. In some embodiments, a sequence alignment is performed using the algorithm of Meyers and Miller ((1989) CABIOS, 4:11-17). This algorithm has been incorporated into the ALIGN program (version 2.0). In some such embodiments, this algorithm is employed using a PAM 120 weight residue table, a gap length penalty of 12 and a gap penalty of 4. In some embodiments, a sequence alignment is performed using the ClustalW program. In some such embodiments, default values, namely: DNA Gap Open Penalty=15.0, DNA Gap Extension Penalty=6.66, DNA Matrix=Identity, Protein Gap Open Penalty=10.0, Protein Gap Extension Penalty=0.2, and Protein matrix=Gonnet, are employed. Identity can be calculated according to the procedure described by the ClustalW documentation. A pairwise score is calculated for every pair of sequences that are to be aligned. These scores are presented in a table in the results. Pairwise scores are calculated as the number of identities in the best alignment divided by the number of residues compared (gap positions are excluded). Both of these scores are initially calculated as percent identity scores and are converted to distances by dividing by 100 and subtracting from 1.0 to give number of differences per site. In certain embodiments, the length of a sequence aligned for comparison purposes is at least 30%, at least 40%, at least 50%, at least 60%, at least 70%, at least 80%, at least 90%, at least 95% or 100% of the length of the reference sequence. The comparison of sequences and determination of percent identity between two sequences can be accomplished using a mathematical algorithm. In certain cases, useful enzymes (polypeptides) have at least 50%, 60%, 70%, 75%, 80%, 85%, 90%, 95%, or 98% identity to a selected enzyme (polypeptide) and retain the ability to carry out the same enzymatic reaction as the selected enzyme (polypeptide).

Transformation: The term “transformation”, as used herein, typically refers to a process of introducing a nucleic acid molecule into a host cell. Transformation typically achieves a genetic modification of the cell. The introduced nucleic acid may integrate into a chromosome of a cell, or may replicate autonomously. A cell that has undergone transformation, or a descendant of such a cell, is “transformed” and is a “recombinant” cell. Recombinant cells are modified cells as described herein. If the nucleic acid that is introduced into the cell comprises a coding region encoding a desired protein, and the desired protein is produced in the transformed microorganism and is substantially functional, such a transformed microorganism is “functionally transformed.” Cells herein may be transformed with, for example, one or more of a vector, a plasmid or a linear piece (e.g., a linear piece of DNA created by linearizing a circular vector) of DNA to become functionally transformed.

Yield: The term “yield”, as used herein, refers to the amount of a desired product (e.g., BDO or BDO intermediate) produced (molar or weight/volume) divided by the amount of carbon source (e.g., dextrose) consumed (molar or weight/volume) multiplied by 100.

Vector: The term “vector” as used herein refers to a DNA or RNA molecule (such as a plasmid, linear piece of DNA, cosmid, bacteriophage, yeast artificial chromosome, or virus, among others) that carries nucleic acid sequences into a host cell. The vector or a portion of it can be permanently or transiently inserted into the genome of the host cell.

### FIGURES

FIG. 1A depicts a biological pathway for the conversion of L-glutamate to 1,4-butanediol. There are six primary conversions in the pathway: (a) conversion of L-glutamate to L-glutamate 5-phosphate; (b) conversion of L-glutamate 5-phosphate to L-glutamate 5-semialdehyde; (c) conversion of L-glutamate 5-semialdehyde to 5-hydroxy-L-norvaline; (d) conversion of 5-hydroxy-L-norvaline to 5-hydroxy-2-oxopentanoate; (e) conversion of 5-hydroxy-2-oxopentanoate to 4-hydroxybutanal; and (f) the conversion of 4-hydroxybutanal to 1,4-butanediol. These conversions are labeled A1-A6 respectively. In addition to the primary conversions, FIG. 1A also depicts seven other conversion steps which may be useful to either perform and/or inhibit when converting L-glutamate to 1,4-butanediol. These conversions are labeled B1-B7.

FIG. 1B is a table providing information about polypeptides useful in the production BDO. DNA sequences are not provided for each protein, but any suitable degenerate sequences, including codon optimized sequences, can be used to encode the disclosed polypeptides.

FIG. 1C provides SEQ ID NO: 52, SEQ ID NO: 57, SEQ ID NO: 69 and SEQ ID NO: 70 from the table in FIG. 1B.

FIG. 2 is a restriction map of PCR amplicon containing the wild type version of the Renibacterium salmoninarum gene encoding glutamate 5-kinase. The fragment contains the gene, preceded by an upstream C. glutamicum RBS sequence, flanked by a number of restriction sites that can be used in the cloning and subcloning of the gene in E. coli and C. glutamicum. Several of the restriction sites are specific for blunt-cutter enzymes which are used to clone the genes into polycistronic operons.

FIG. 3 is a restriction map of synthesized DNA fragment containing the Renibacterium salmoninarum gene for glutamate-5-semialdehyde dehydrogenase. The nucleotide sequence of the gene is nearly identical to that of the wild type allele except for 17 silent nucleotide substitutions which were introduced in order to remove several restriction sites which may be required for downstream cloning procedures. The fragment contains the gene, preceded by an upstream C. glutamicum ribosomal binding sequence (RBS), flanked by a number of restriction sites that may be used in the cloning and subcloning of the gene in E. coli and/or C glutamicum. Several of the restriction sites are specific for blunt-cutter enzymes which can be used to clone the gene into polycistronic operons.

FIG. 4 is a restriction map of synthesized DNA fragment containing a codon optimized nosE allele encoding a zinc-containing alcohol dehydrogenase from Nostoc sp. strain GSV224. The fragment contains the gene, preceded by an upstream C. glutamicum RBS sequence, flanked by a number of restriction sites that may be used in the cloning and subcloning of the gene in E. coli and C. glutamicum. Several of the restriction sites are specific for blunt-cutter enzymes which can be used to clone the genes into polycistronic operons.

FIG. 5 is a restriction map of synthesized DNA fragment containing a codon optimized YALI0D01265g allele encoding a branch-chained aminotransferase from Yarrowia lipolytica. The fragment contains the gene, preceded by an upstream C. glutamicum RBS sequence, flanked by a number of restriction sites that are used in the cloning and subcloning of the gene in E. coli and C. glutamicum. Several of the restriction sites are specific for blunt-cutter enzymes which can be used to clone the genes into polycistronic operons.

FIG. 6 is a restriction map of synthesized DNA fragment containing a codon optimized kdcA allele encoding a branch-chained α-keto acid decarboxylase from Lactococcus lactis NIZO B 1157. The fragment contains the gene, preceded by an upstream C. glutamicum RBS sequence, flanked by a number of restriction sites that can be used in the cloning and subcloning of the gene in E. coli and C. glutamicum. Several of the restriction sites are specific for blunt-cutter enzymes which can be used to clone the genes into polycistronic operons.

FIG. 7 is a restriction map of synthesized DNA fragment containing a codon optimized ADH6 allele encoding an alcohol dehydrogenase from Saccharomyces cerevisiae. The fragment contains the gene, preceded by an upstream C. glutamicum RBS sequence, flanked by a number of restriction sites that can be used in the cloning and subcloning of the gene in E. coli and C. glutamicum. Several of the restriction sites are specific for blunt-cutters enzyme which can be used to clone the genes into polycistronic operons.

FIG. 8 is a restriction map of E. coli cloning vector, pUC18.

FIG. 9A is a restriction map of C. glutamicum deletion/insertion vector MB3965.

FIG. 9B is a graphic depiction of the galK locus of C. glutamicum ATCC 13032.

FIG. 9C is a restriction map of plasmid MB5718.

FIG. 9D is a restriction map of plasmid MB5628.

FIG. 9E is a restriction map of plasmid MB5733.

FIG. 9F is a restriction map of plasmid MB5735.

FIG. 9G is a graphic depiction showing the deletion of the proC locus of C. glutamicum ATCC 13032.

FIG. 9H is a restriction map of plasmid MB5712.

FIG. 9I is a restriction map of plasmid MB5713.

FIG. 10A is a restriction map of C. glutamicum episomal vector MB4124.

FIG. 10B is a graph showing in vitro KdcA enzyme activity.

FIG. 11A is a cloning strategy which can be used to construct multi-gene operons.

FIG. 11B is a restriction map of plasmid MB5782.

FIGS. 12A and 12B are schematics depicting growth results of various microbial strains in three different concentrations of BDO.

FIGS. 13-21 are tables disclosing a list of some of the candidate genes that may be applicable to the 1,4-butanediol (BDO) biosynthetic pathway described herein. FIGS. 13-21 are referenced throughout the description. Each reference and information designated by each of the Genbank Accession numbers are hereby incorporated by reference in their entirety. The order of Genbank Accession numbers, genes, polypeptides and sequences presented in the tables is not indicative of their relative importance and/or suitability to any of the embodiments disclosed herein.

FIG. 22 is a schematic of an expected typical chromatogram analyzing BDO and BDO intermediates.

FIG. 23A is a graph showing BDO production of certain C. glutamicum strains.

FIG. 23B is a graph showing BDO production of C. glutamicum strain ME124 replicates.

FIG. 23C is a graph showing the bioconversion of extracellular P5C to BDO by C. glutamicum strain ME124.

FIG. 24 is a graph showing enzymatic activity of KdcA_{L1}.

FIG. 25 is a graph showing enzymatic activity of NosE_{Np}.

### DETAILED DESCRIPTION

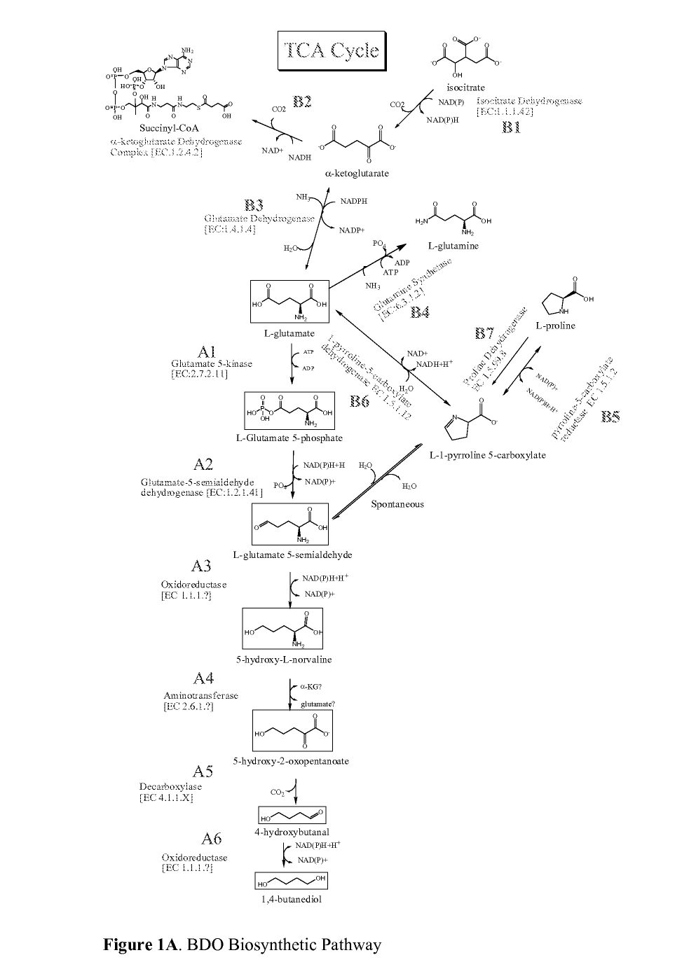

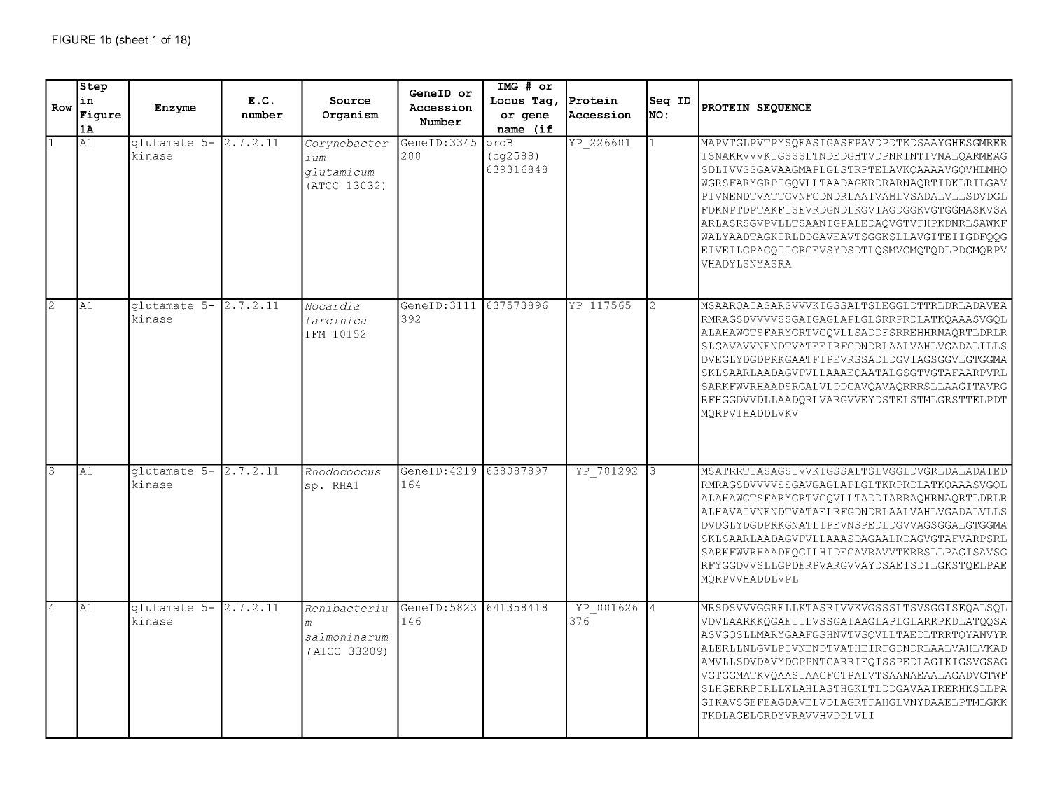

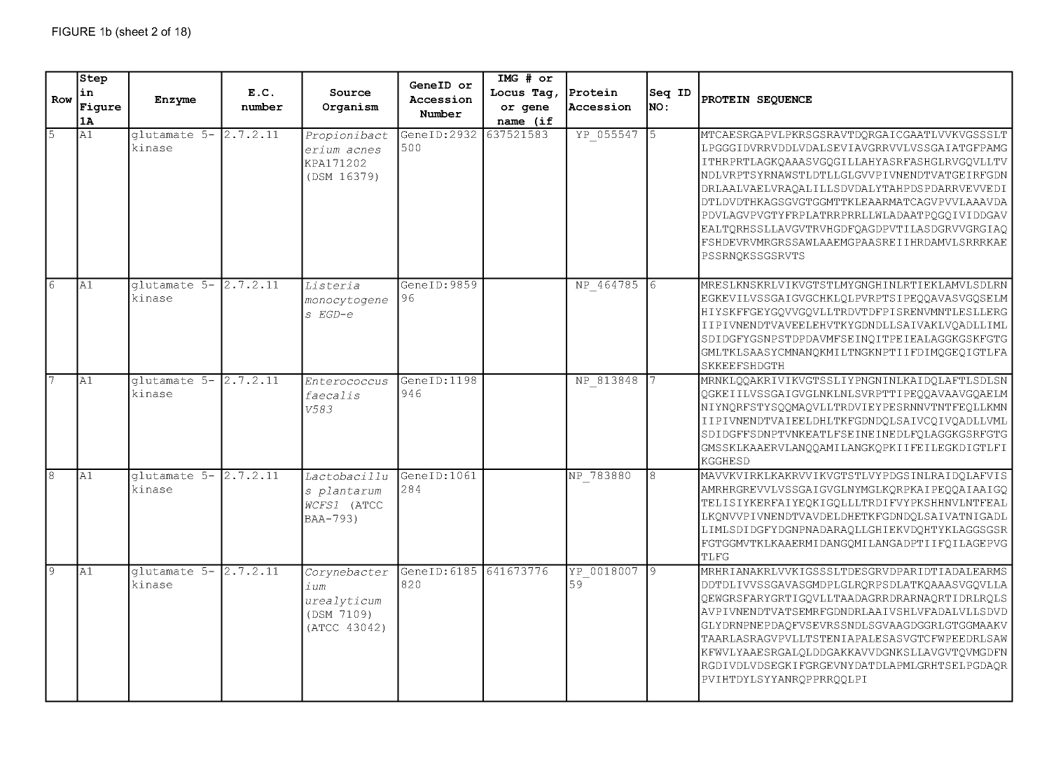

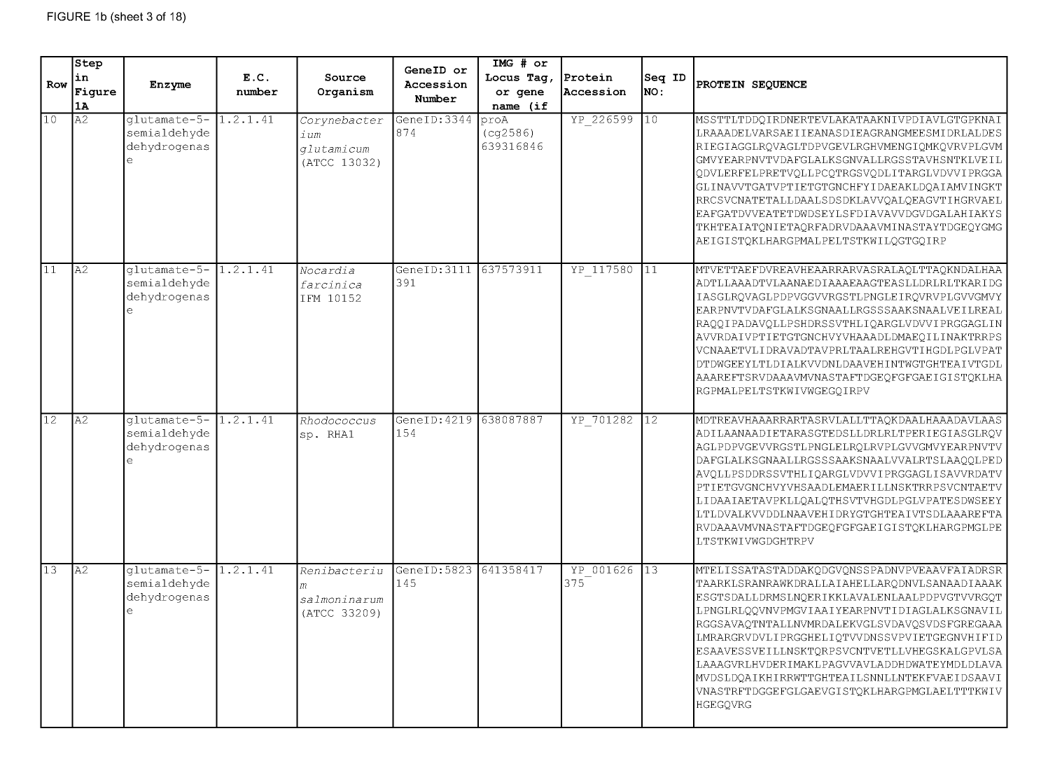

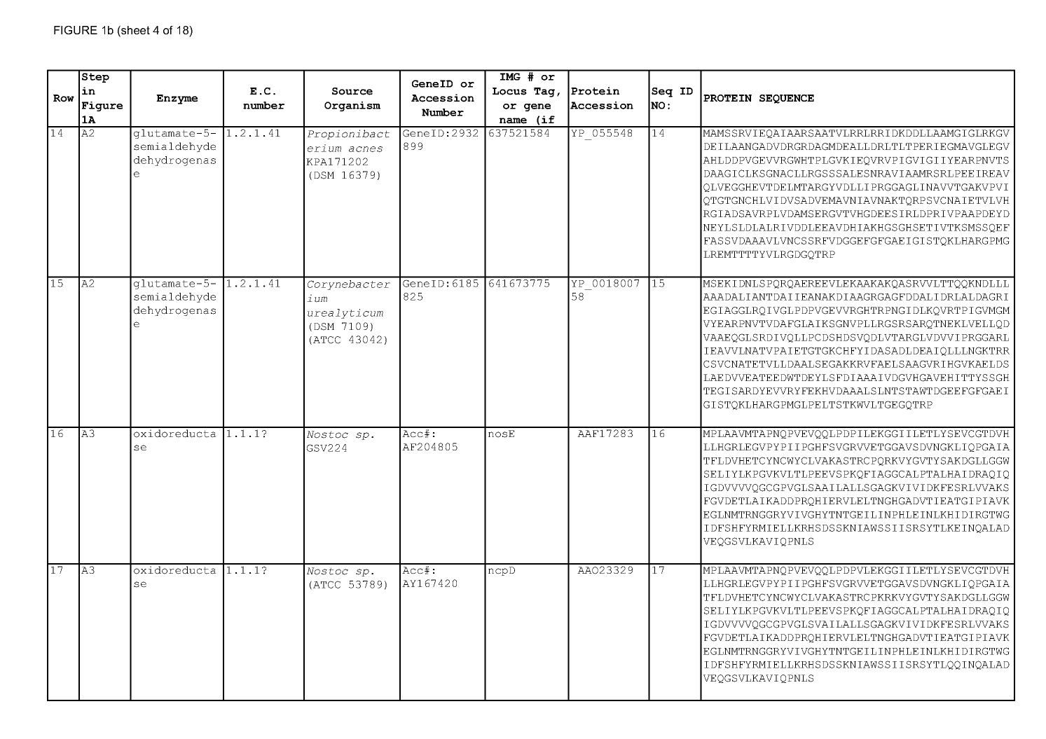

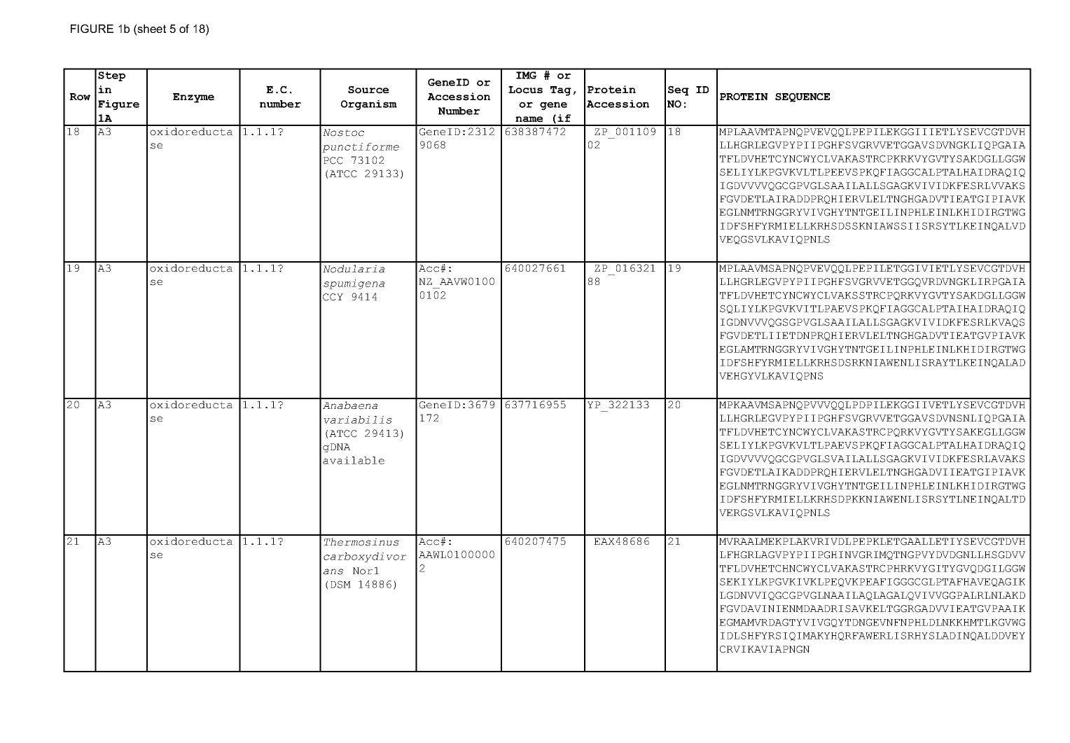

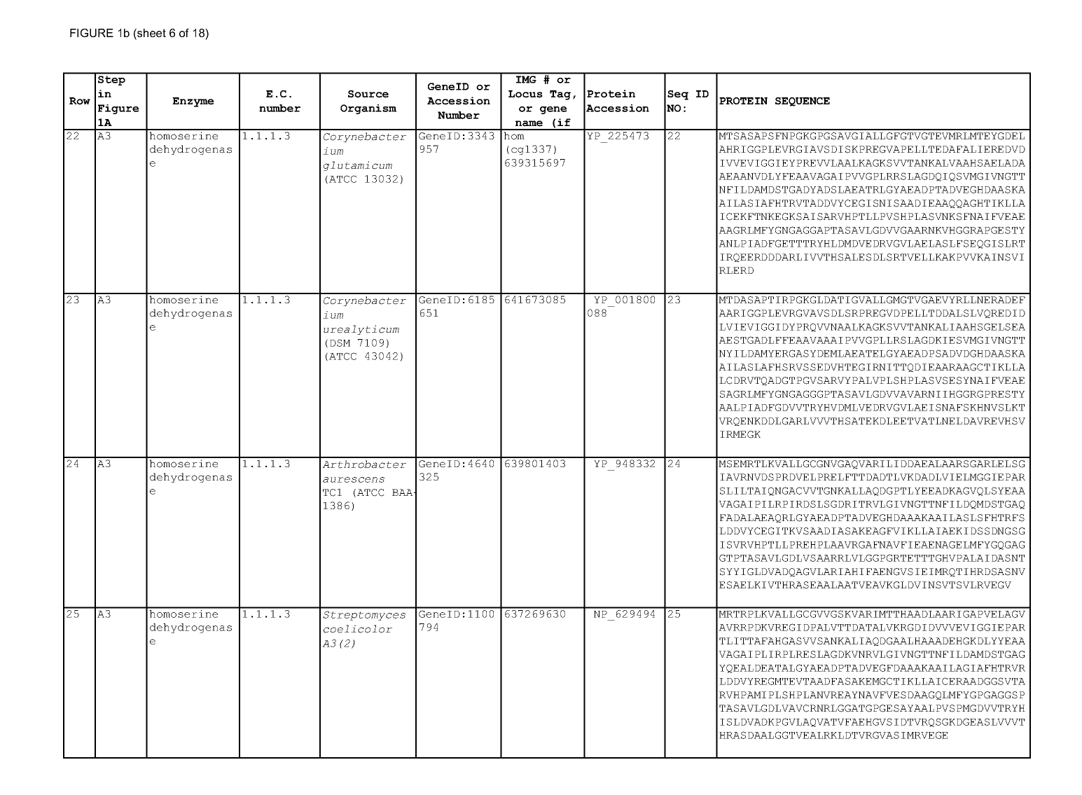

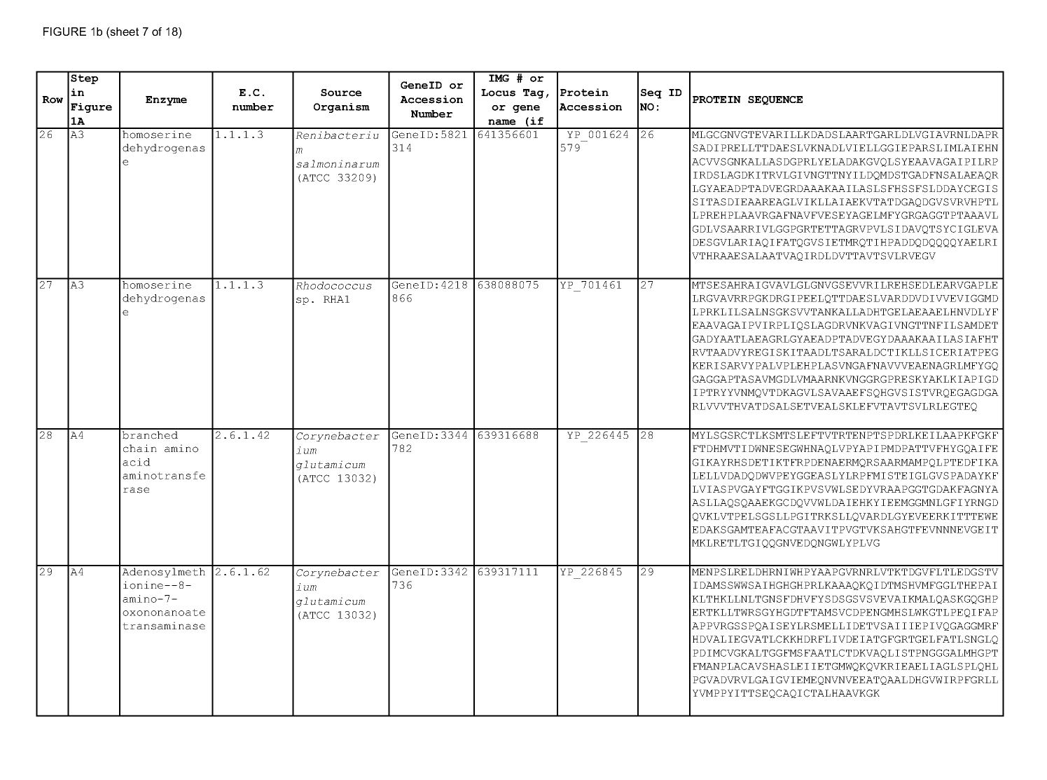

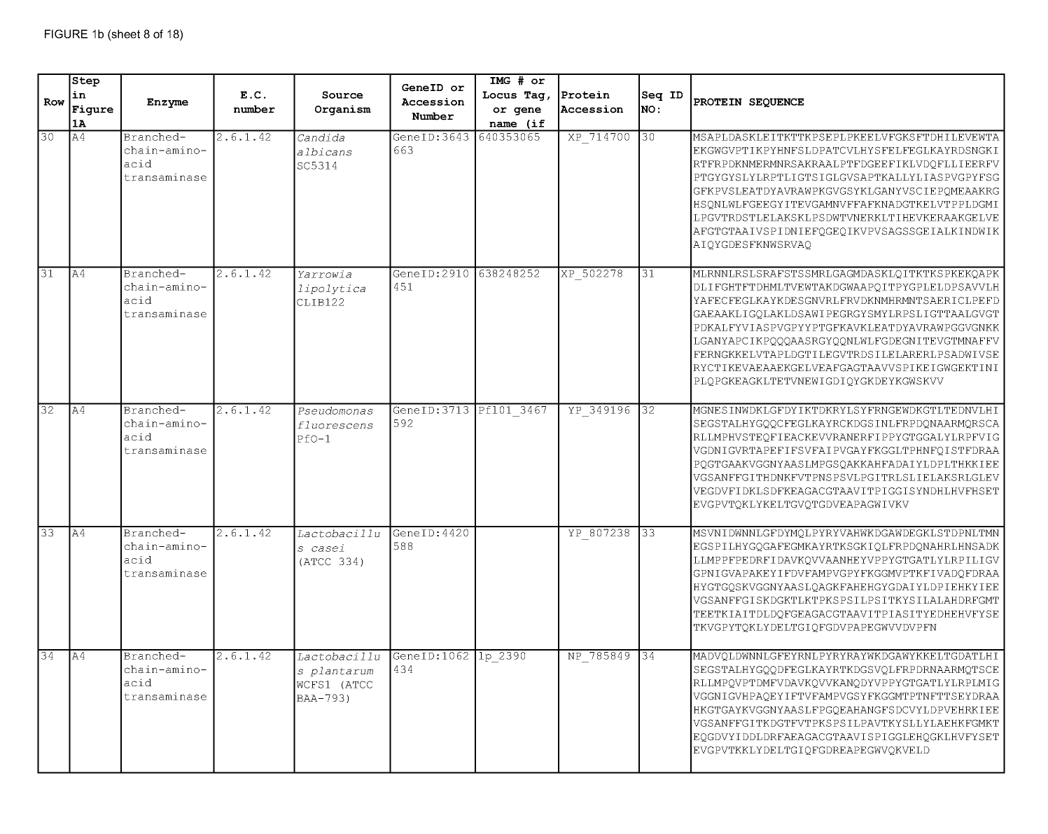

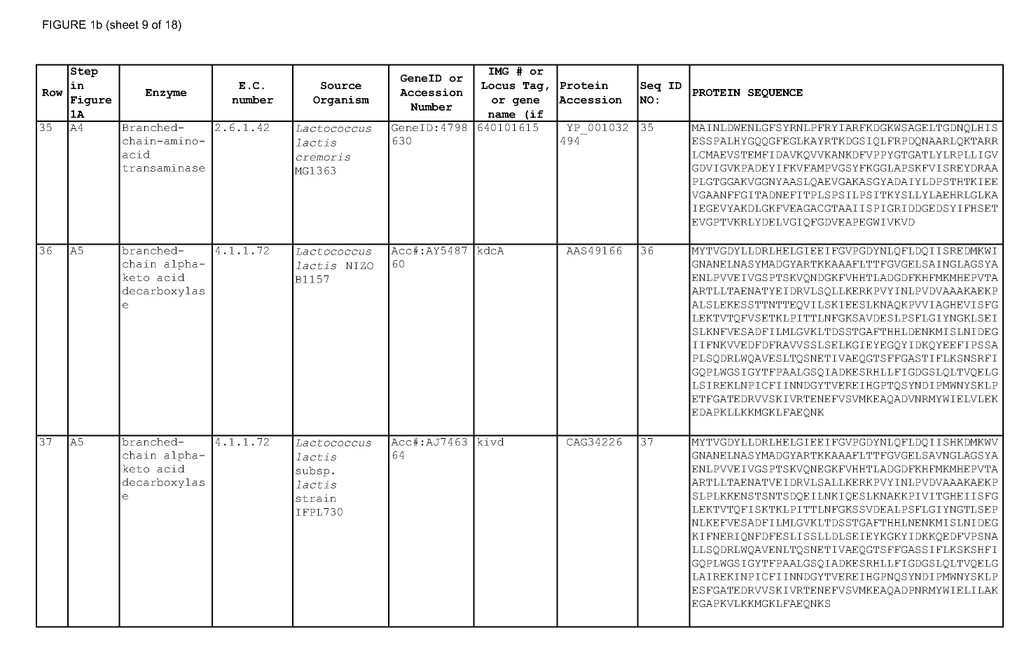

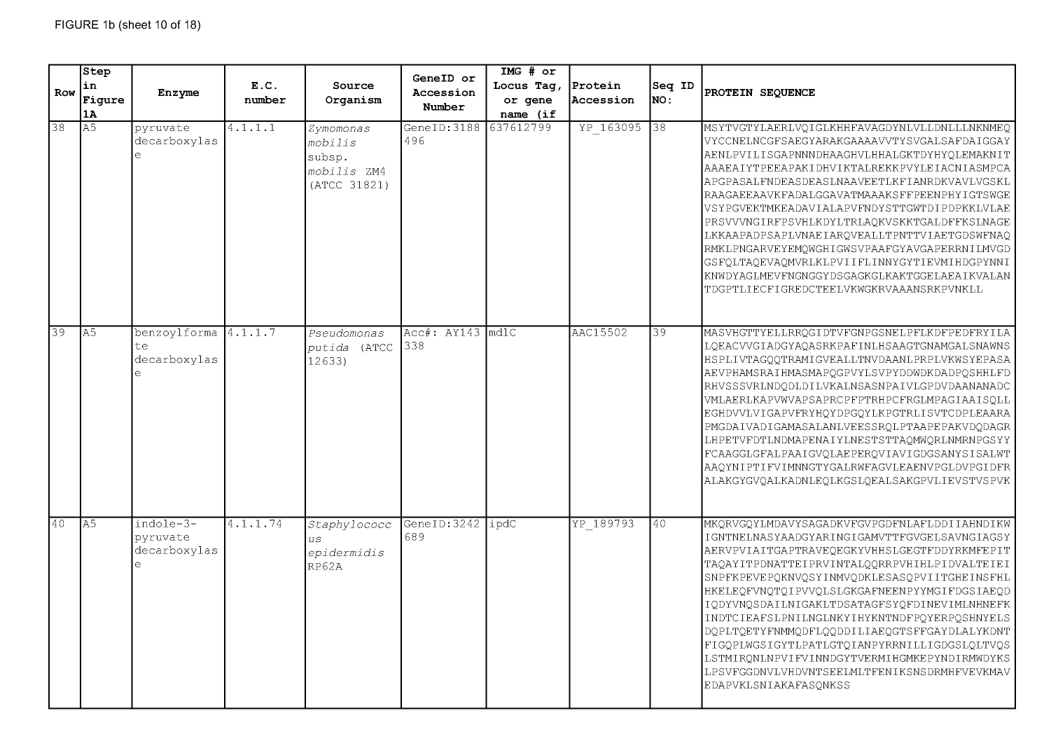

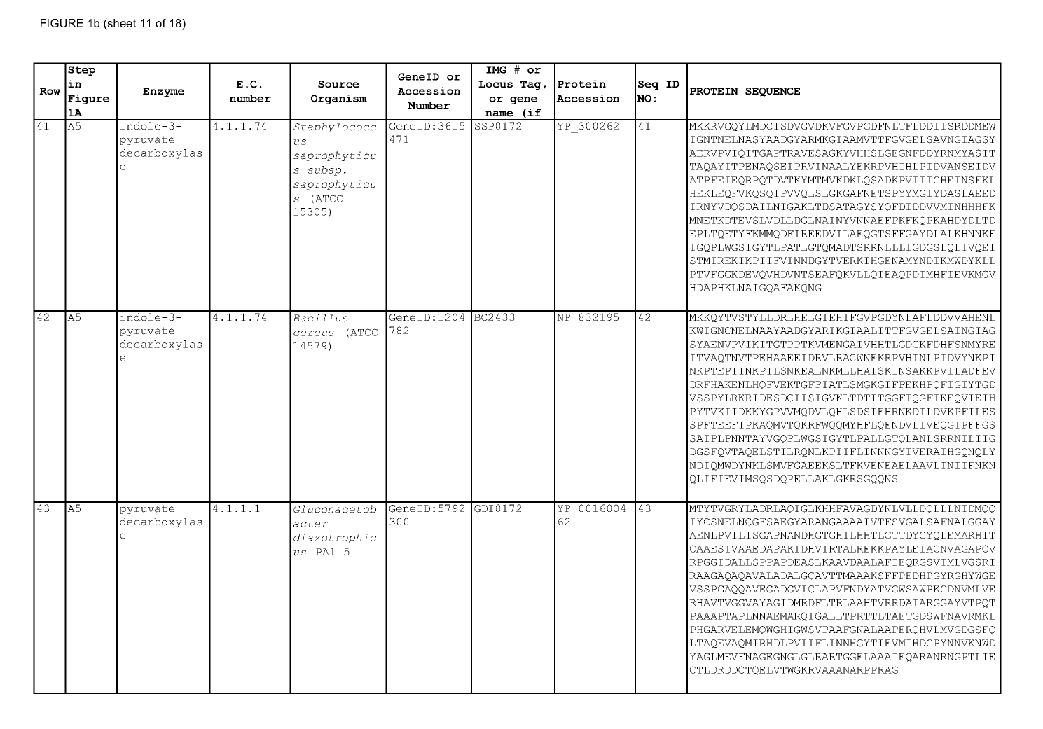

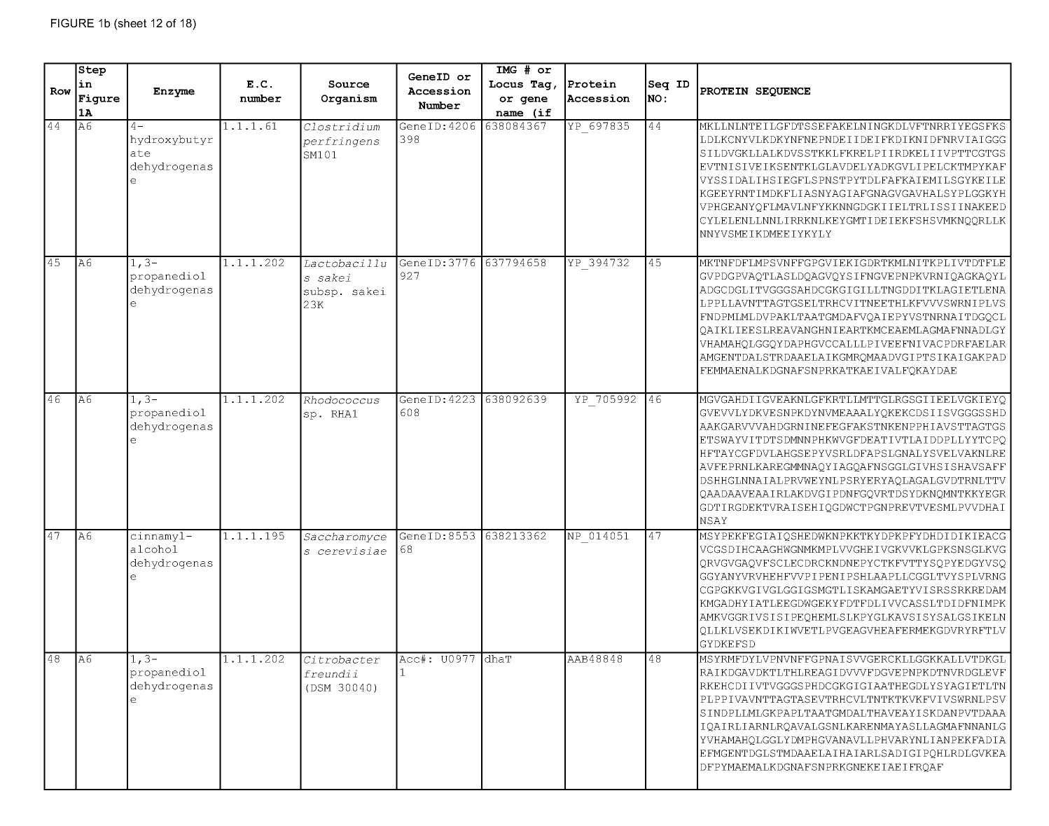

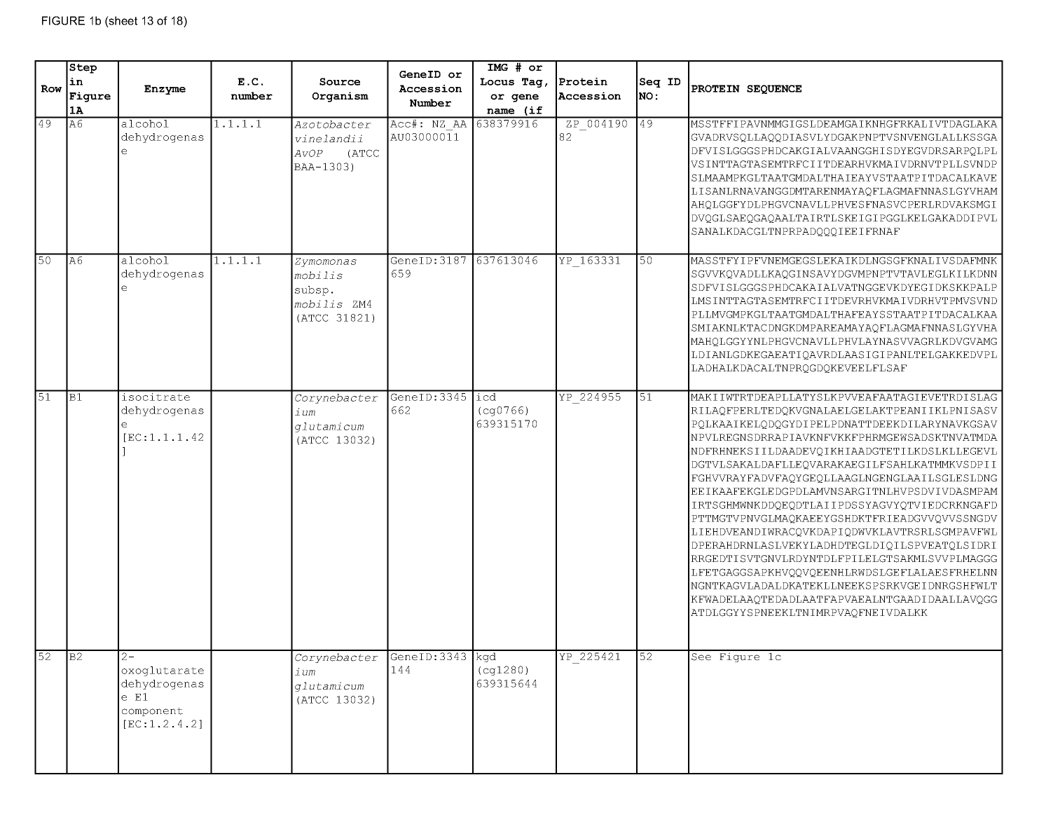

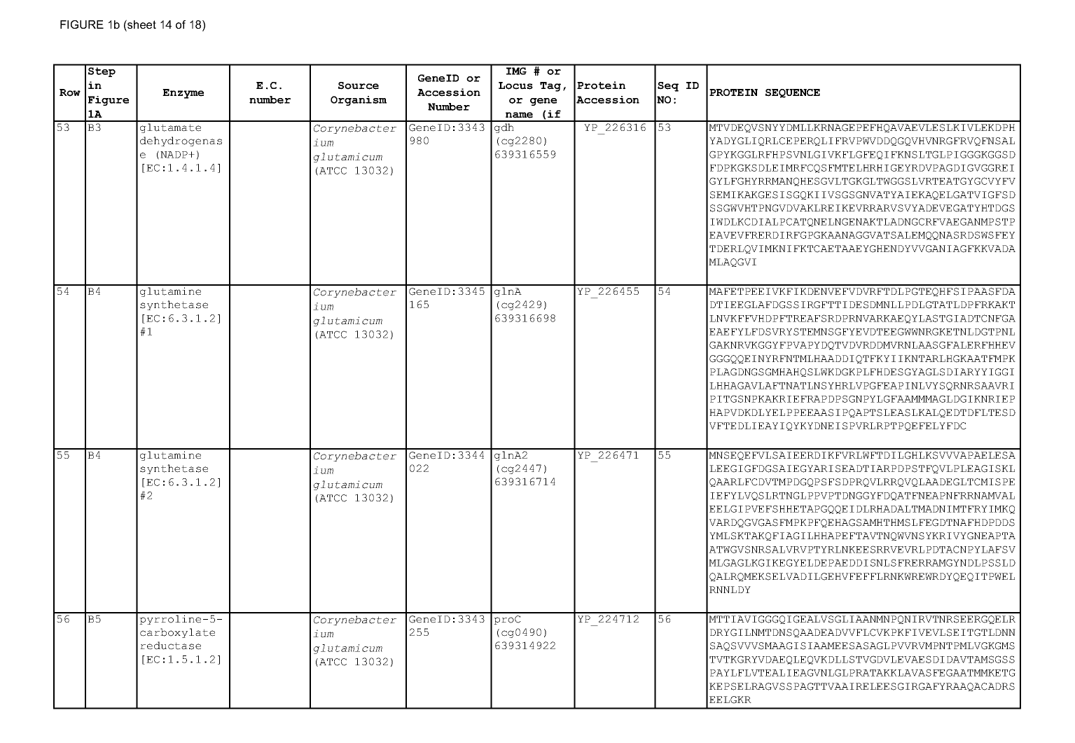

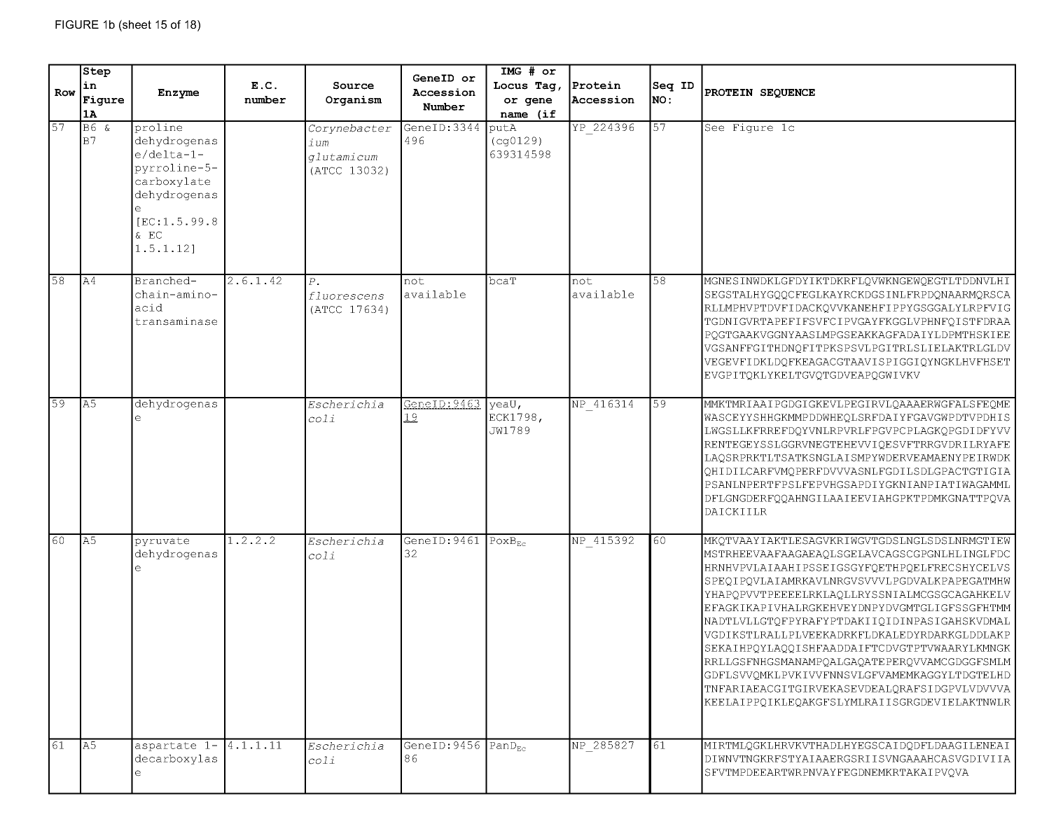

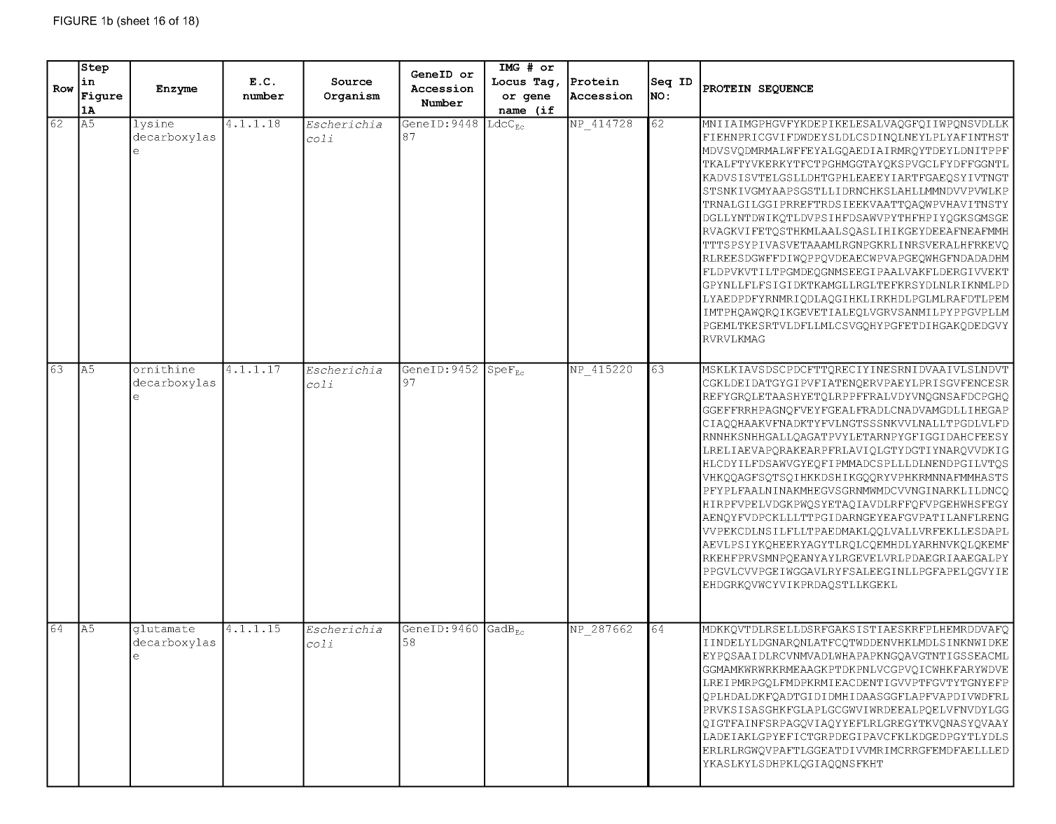

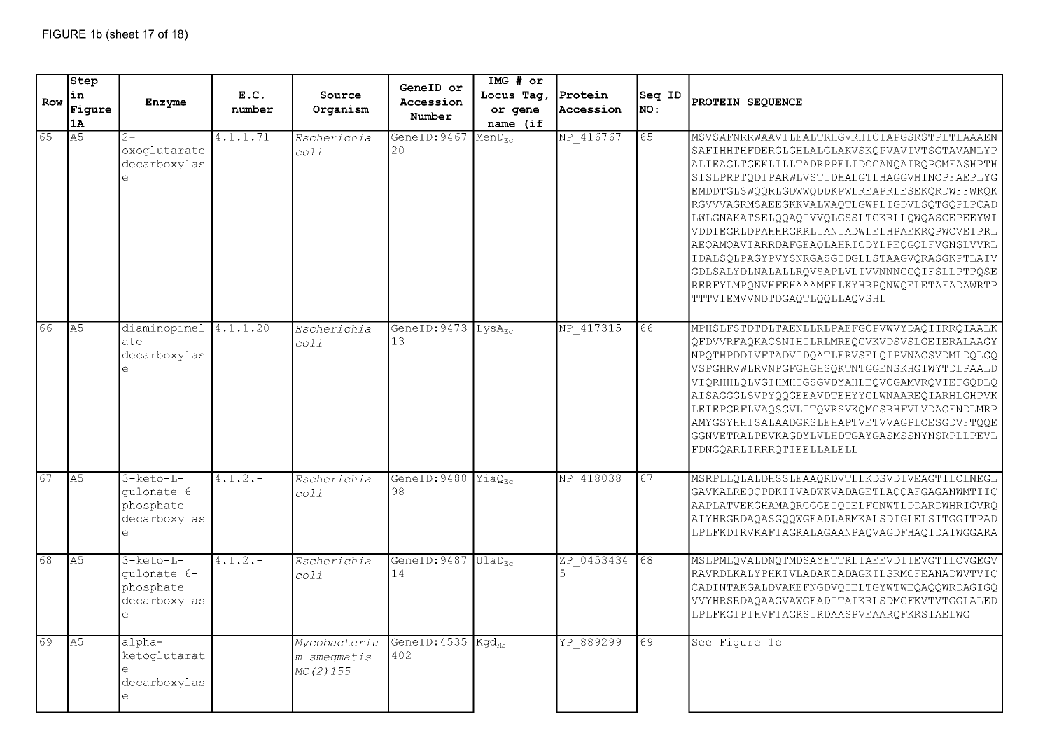

A biological method for the conversion of L-glutamate to 1,4-butanediol is described herein. Among other things, the pathway involves a decarboxylation step and does not involve production of 4-hydroxybutyrate as an intermediate. There are six primary conversions in the pathway: (a) conversion of L-glutamate to L-glutamate 5-phosphate; (b) conversion of L-glutamate 5-phosphate to L-glutamate 5-semialdehyde; (c) conversion of L-glutamate 5-semialdehyde to 5-hydroxy-L-norvaline; (d) conversion of 5-hydroxy-L-norvaline to 5-hydroxy-2-oxopentanoate; (e) conversion of 5-hydroxy-2-oxopentanoate to 4-hydroxybutanal; and (f) the conversion of 4-hydroxybutanal to 1,4-butanediol. These conversions are labeled A1-A6 respectively in FIG. 1A and can be catalyzed by enzymes described in greater detail below.

Described herein are cells that have been genetically modified so that they can carry out all of these conversions. In some cases the genetic modification entails providing the cell with one or more nucleic acid molecules that collectively encode six enzymes, each of which catalyzes of the conversions A1-A6. In some cases, the unmodified cell can already carry out one or more of the conversions and it is not necessary to modify the cell by providing the cell with nucleic acid molecules encoding all six enzymes. In some cases it can be useful to increase the expression or activity of an enzyme already expressed by the unmodified cell. Since the pathway outlined above begins with glutamate, the cell selected for modification desirably produces a relatively high level of glutamate, produces a high level of a product derived from glutamate, and/or one of the intermediates in such pathways. In certain cases, the pathway can begin with proline. Of course, while the cells can be genetically modified to increase (or provide) expression or activity of enzymes catalyzing some or all of conversions A1-A6, the cells can be modified in other ways as well. For example, the cells can be modified to increase production of glutamate and/or proline. Methods for increasing proline production are described in U.S. Pat. No. 3,650,899 and U.S. Pat. No. 4,444,885.

In certain cases, depending on the availability of certain intermediates in the pathway, only conversions A3-A6 (steps (c)-(f), above) are utilized to produce the end product. In some cases it may be desirable to produce one or more intermediates in addition to or instead of the end product, and in such circumstances only a subset of the conversions are utilized.

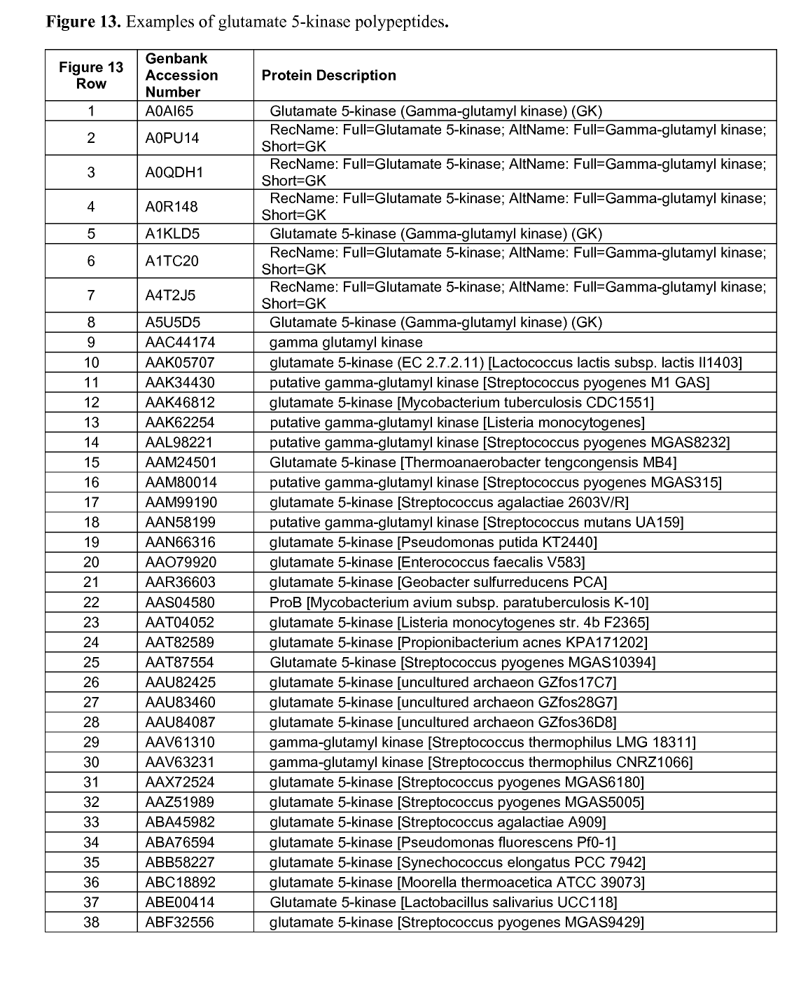

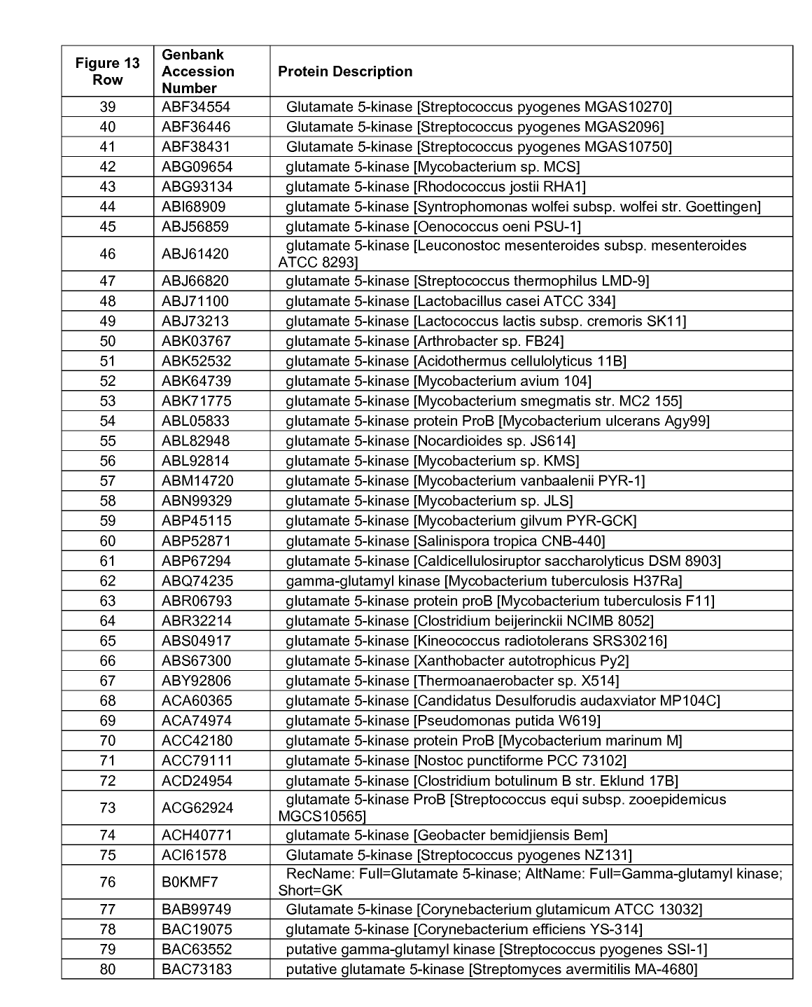

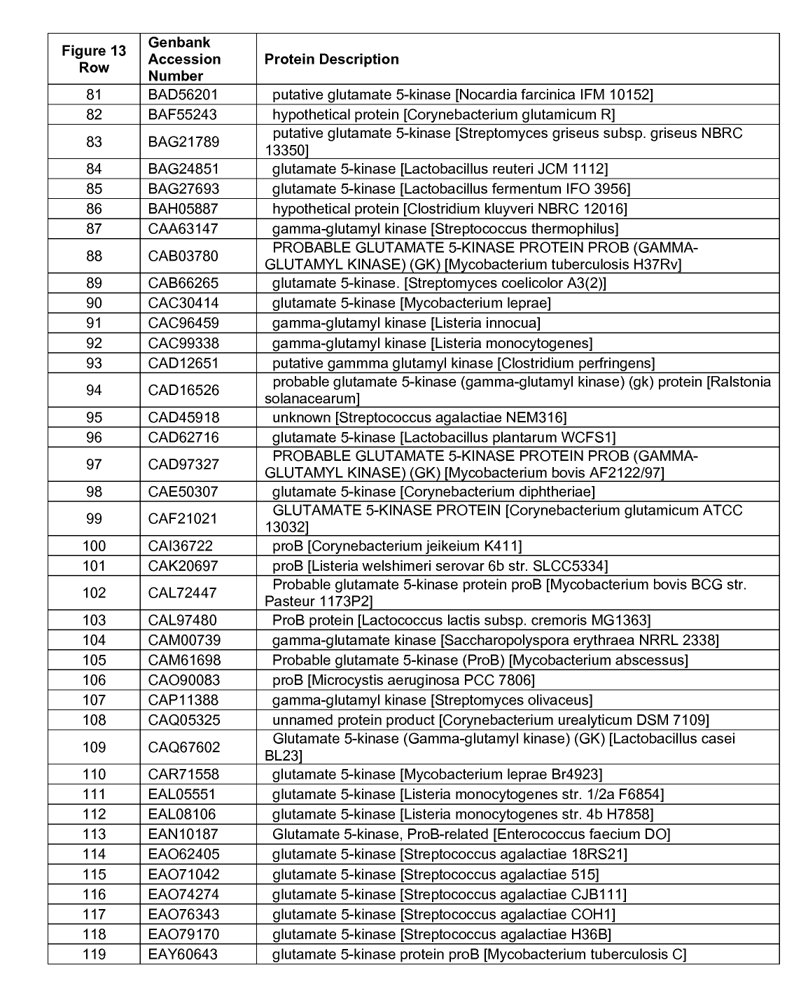

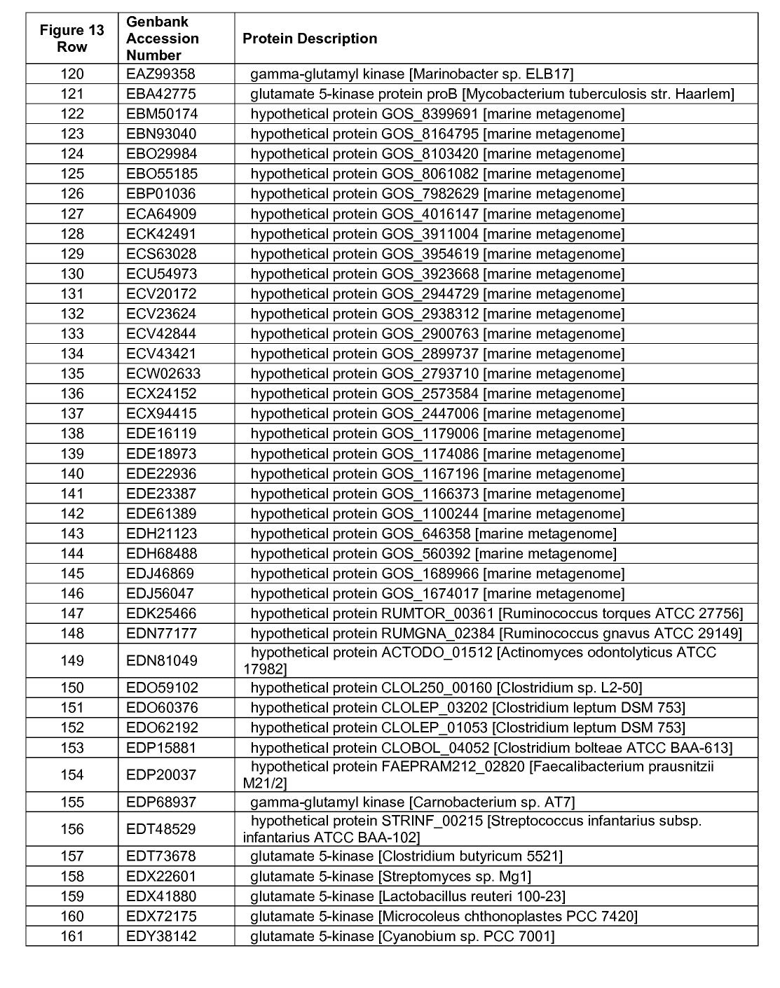

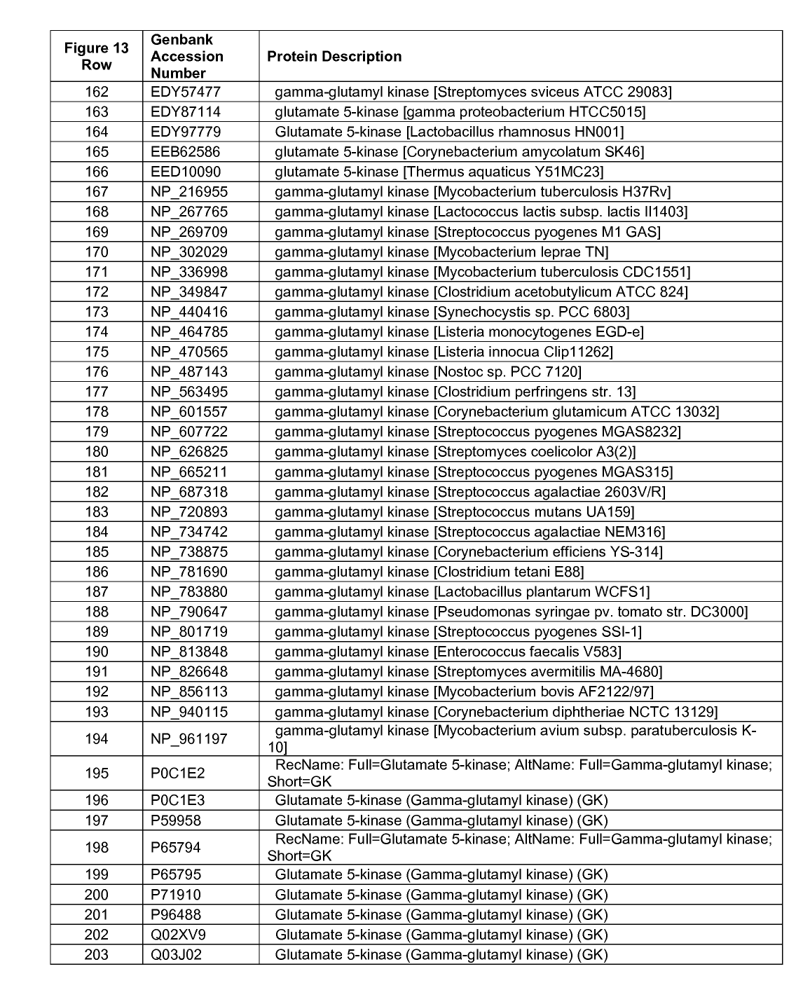

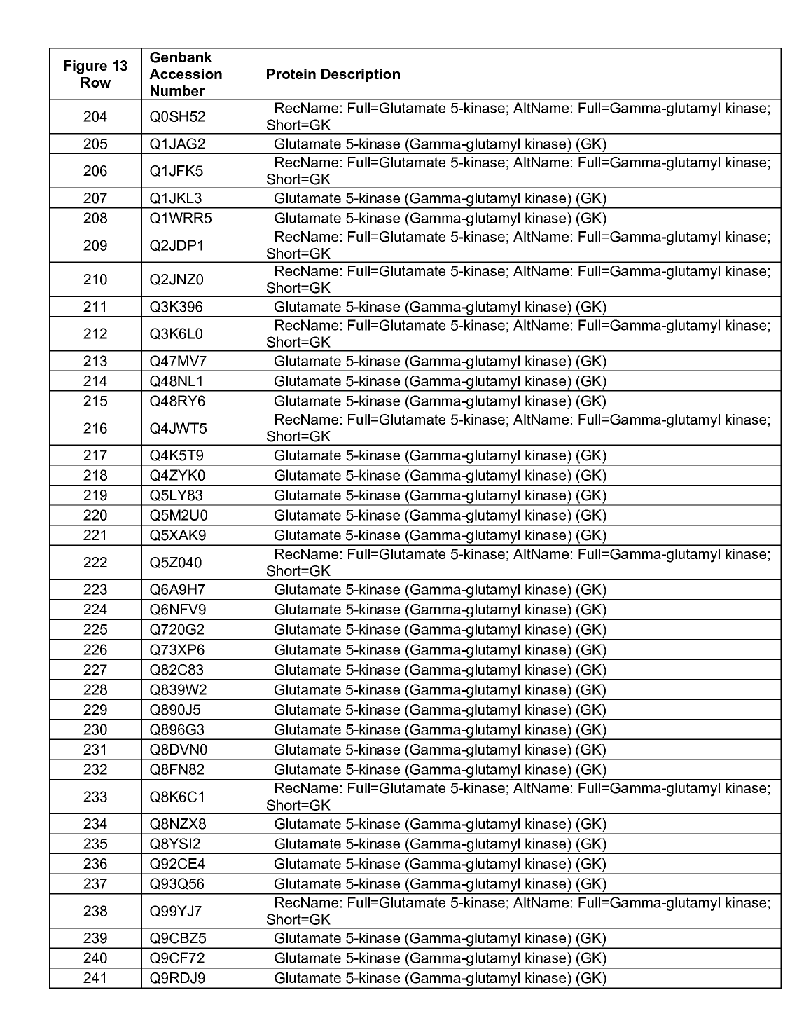

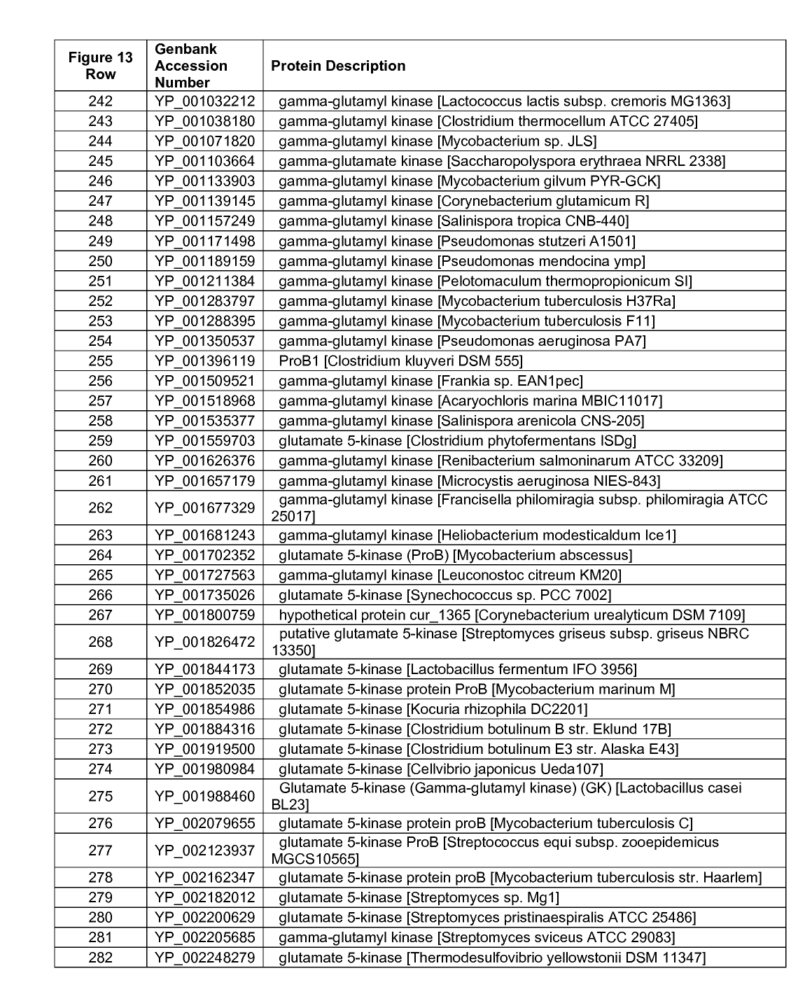

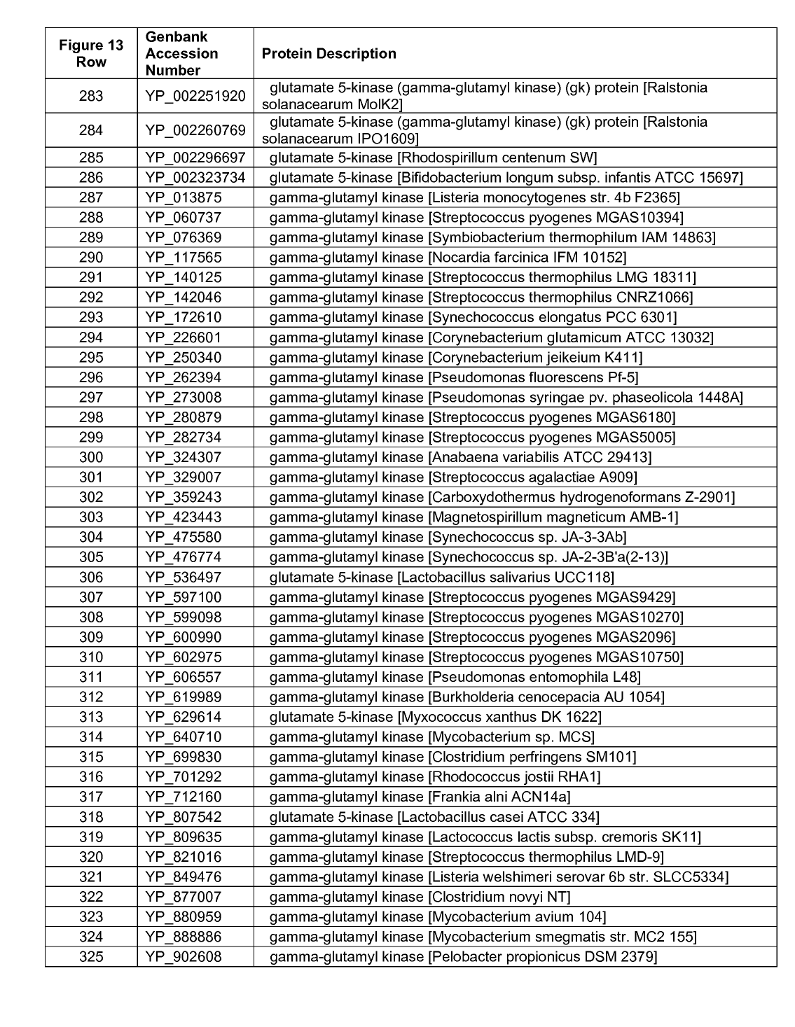

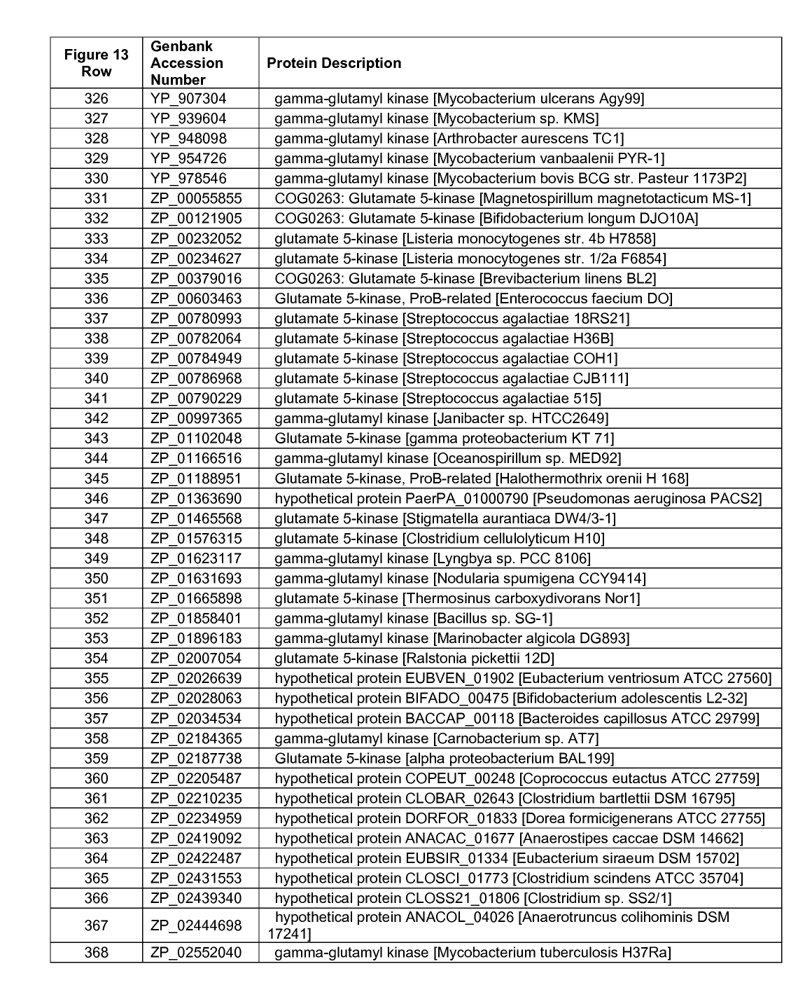

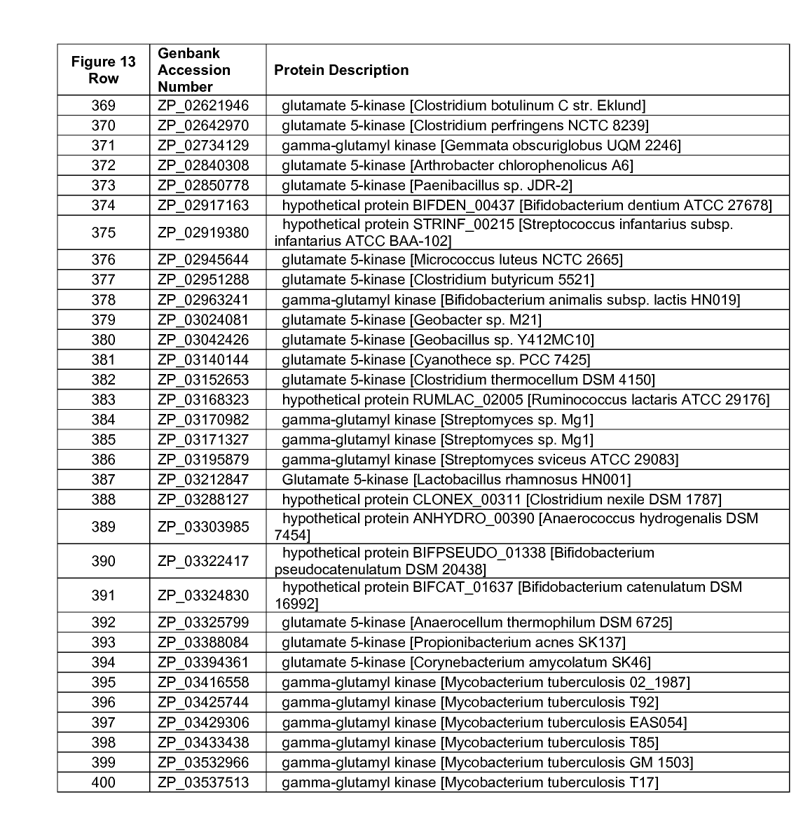

Glutamate 5-kinase [EC 2.7.2.11] is an example of an enzyme capable of catalyzing conversion A1 in FIG. 1A. This enzyme catalyzes the ATP-dependent conversion of L-glutamate to L-glutamate 5-phosphate. Examples of glutamate 5-kinases are represented by the Genbank GI numbers in FIG. 1B and the corresponding amino acid sequences (SEQ ID NOs:1-9) as well as the polypeptides represented by the Genbank Accession numbers in FIG. 13. Some glutamate 5-kinases are inhibited by proline and/or ADP. In certain cases it would be advantageous to use a glutamate 5-kinase protein that exhibits reduced inhibition to proline and/or ADP. Examples of such reduced inhibited glutamate 5-kinase alleles which may be useful herein are disclosed in, for example, Krishna and Leisinger (1979) Biochemical Journal 181(1):215-22; Rushlow et al. (1985) Gene 39(1):109-12 (for example, the E428A allele); Wadano et al. (1986) Biochemistry & Molecular Biology 83B (4):757-61; and Csonka et al. (1988) Gene 64(2):199-205 (for example, the D107N allele). One skilled in the art can generate reduced inhibition variants of other glutamate 5-kinases by first aligning the amino acid sequence of an identified reduced inhibition glutamate 5-kinase variant (e.g., one of the variants noted above) with the amino acid sequence of a glutamate 5-kinase that is inhibition sensitive. Next, the amino acid modifications in the reduced inhibition glutamate 5-kinase variant are used to identify the position and type (e.g., Gln to Pro) of potential amino acid modifications in the inhibition sensitive glutamate 5-kinase. The amino acid change(s) can be introduced and the newly created variant can be tested to determine whether it exhibits reduced inhibition. In selecting a reduced inhibition glutamate 5-kinase variant to guide the creation of additional variants, it can be useful to choose a variant that has a relatively high degree of sequence homology to the glutamate 5-kinase that is to be modified. Of course, similar methods can be used to create reduced inhibition variants of other enzymes. Certain polypeptides capable of catalyzing conversion A1 in FIG. 1A have an amino acid sequence that is at least about 75%, 85%, 90%, 95% or 100% identical to that of any of SEQ ID NOs:1-9 or the polypeptides represented by the Genbank Accession numbers in FIG. 13.

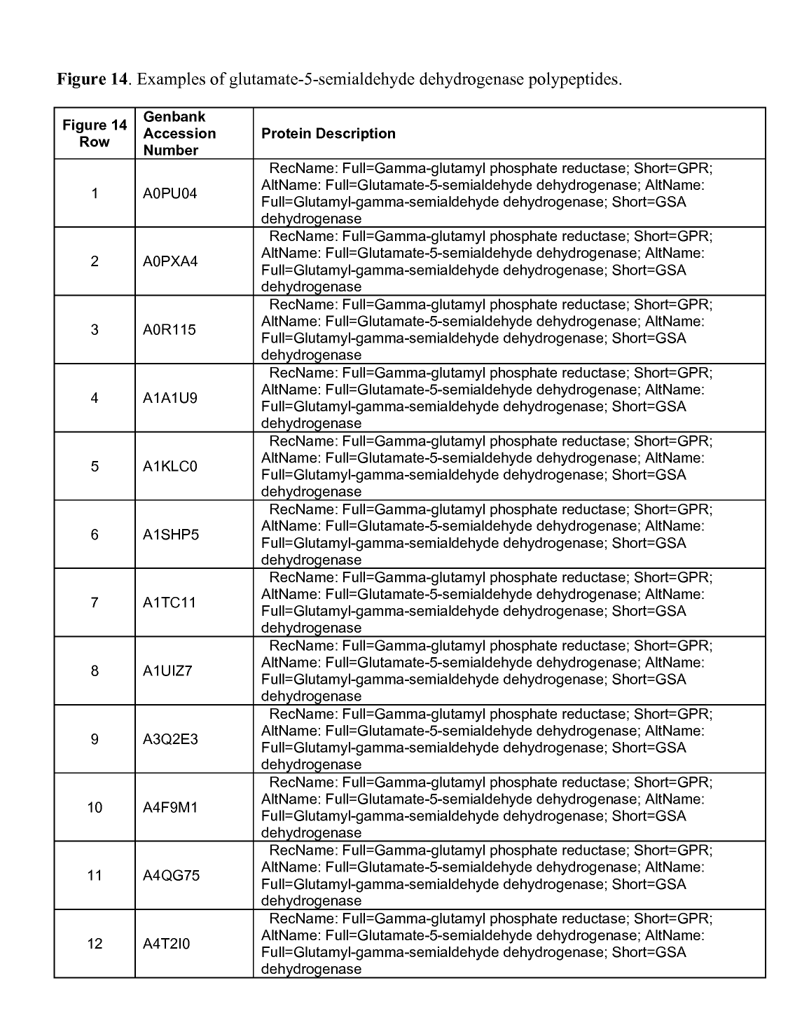

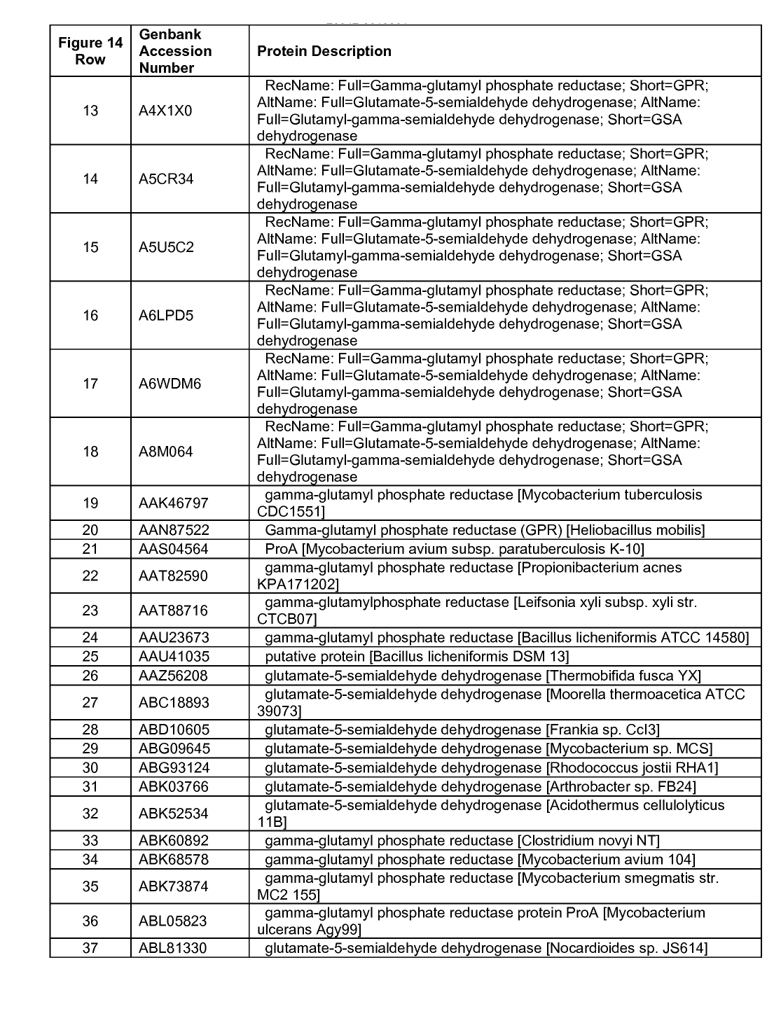

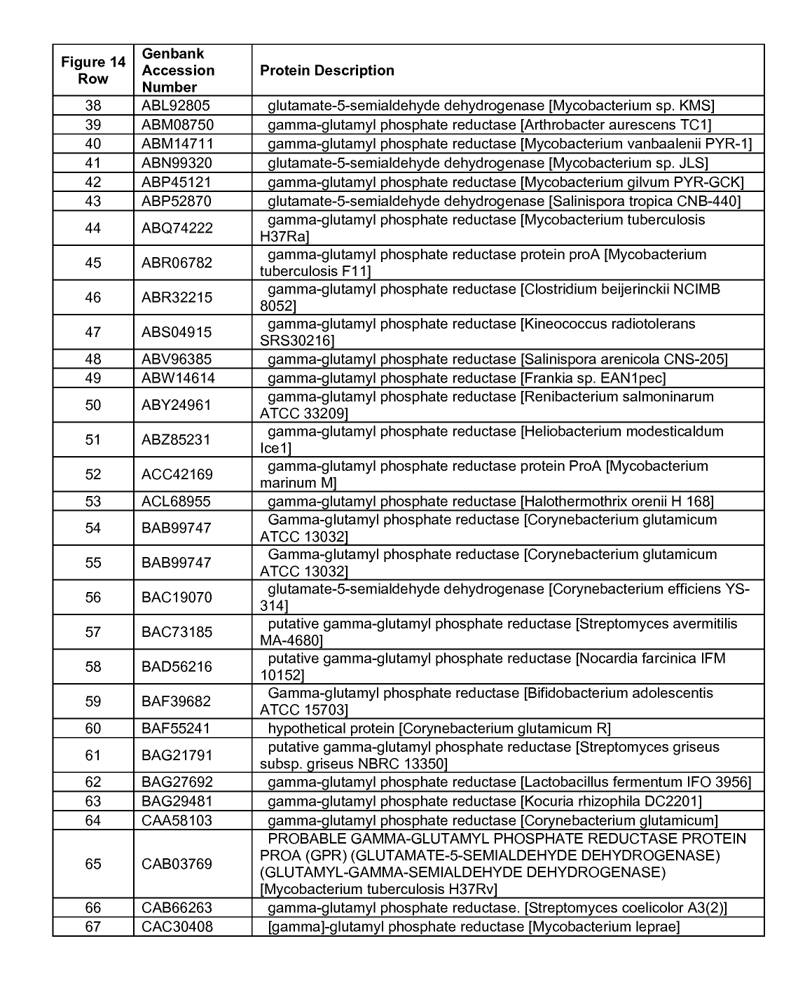

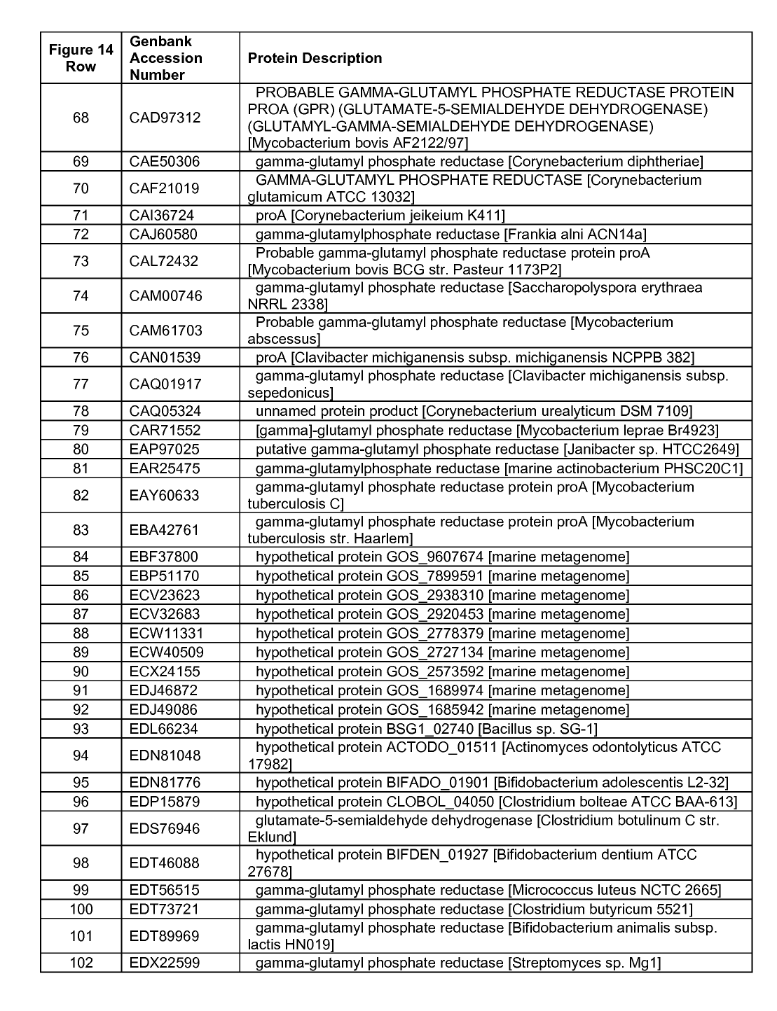

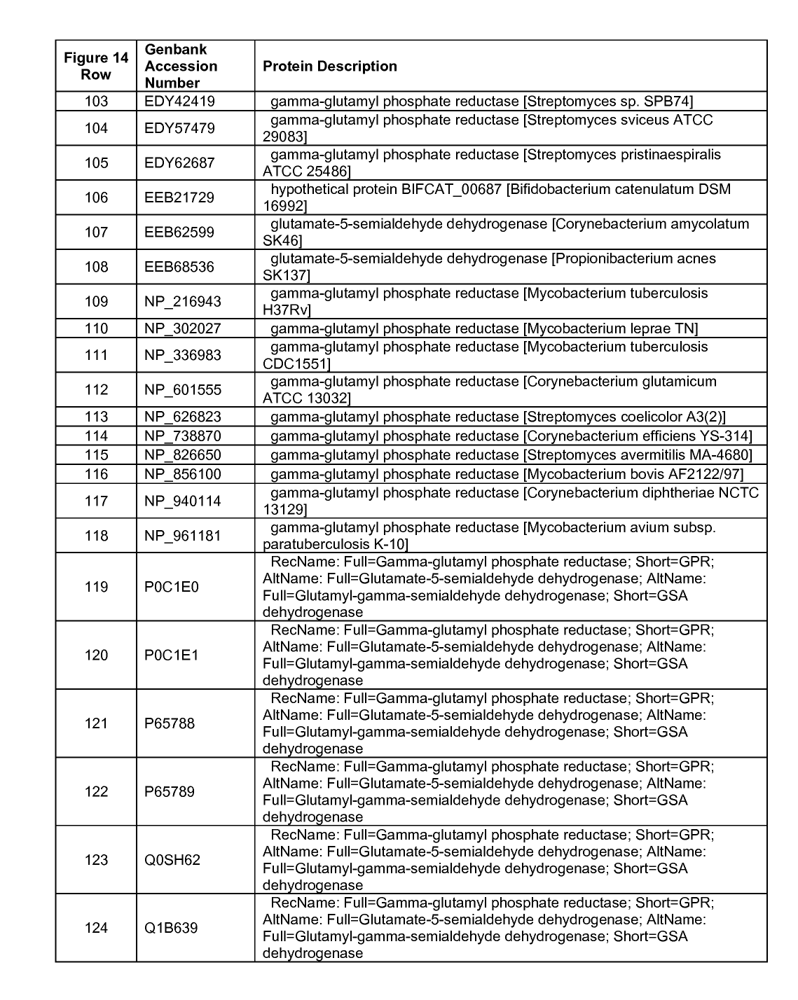

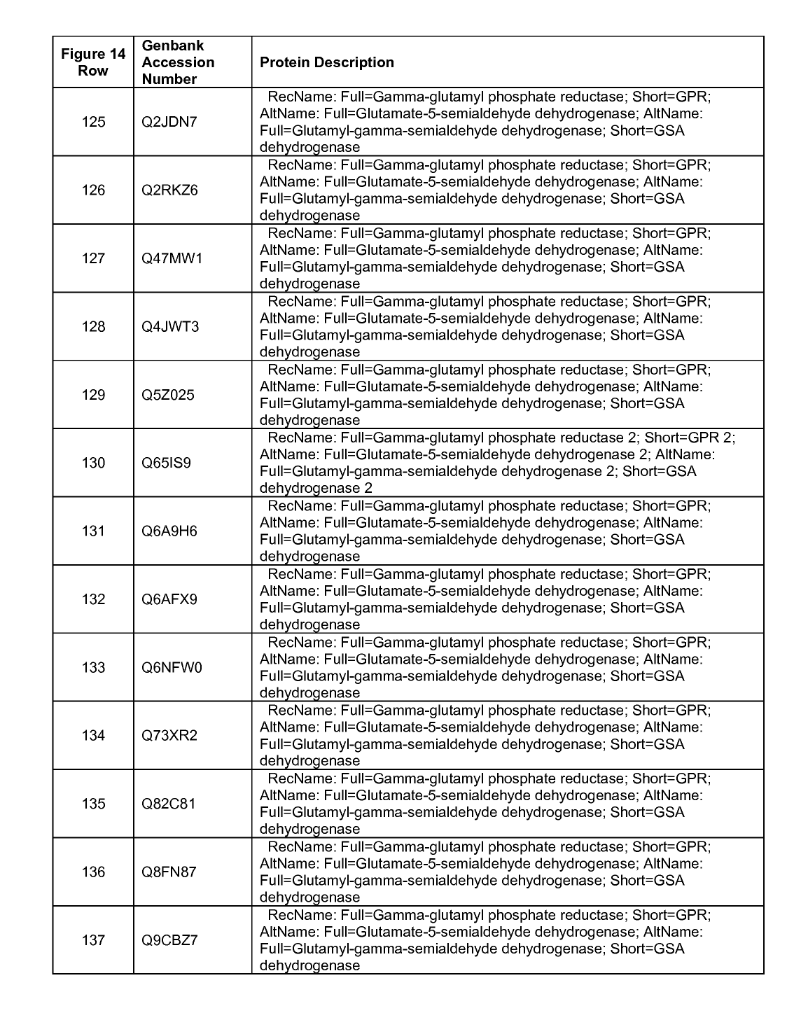

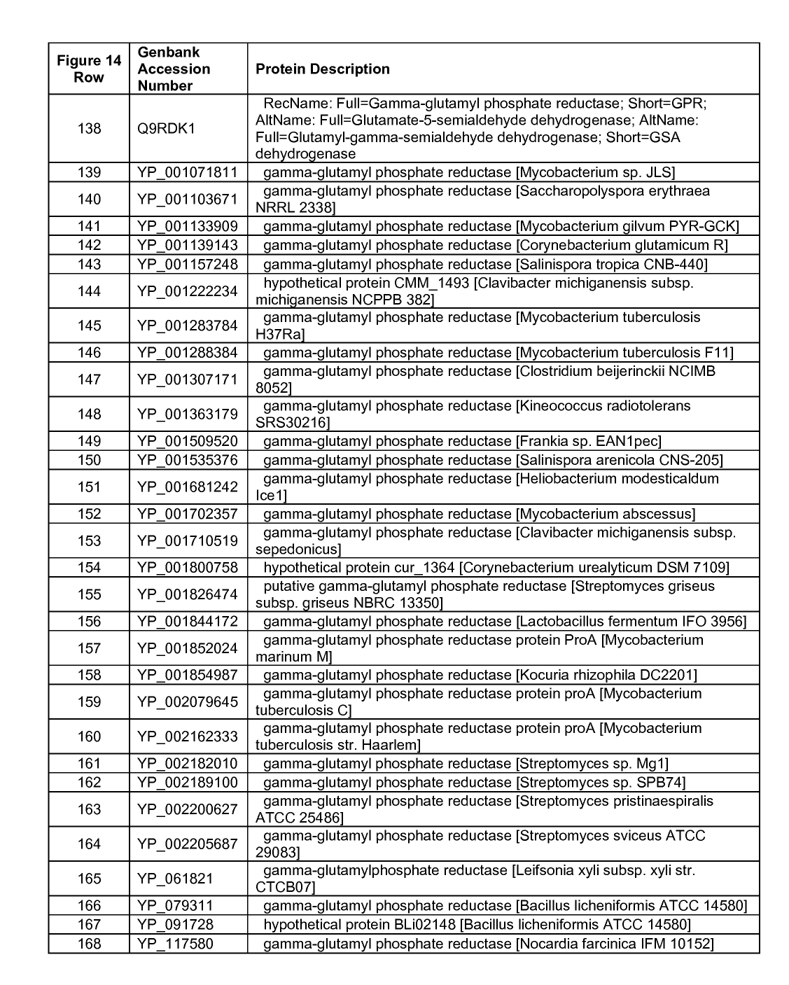

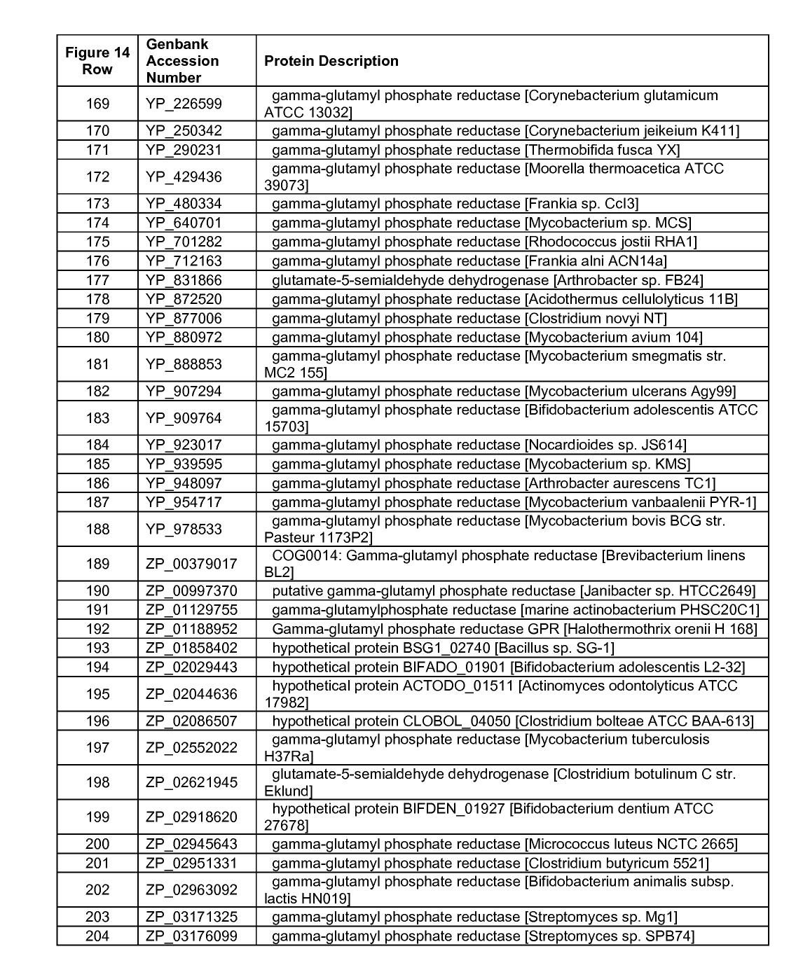

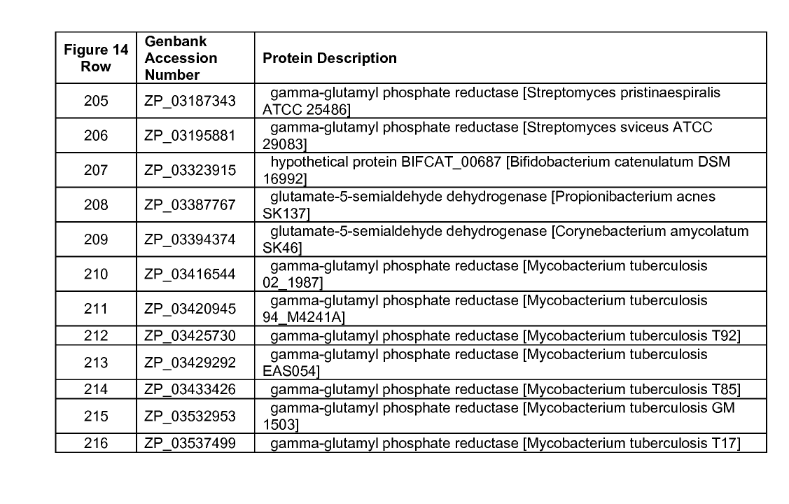

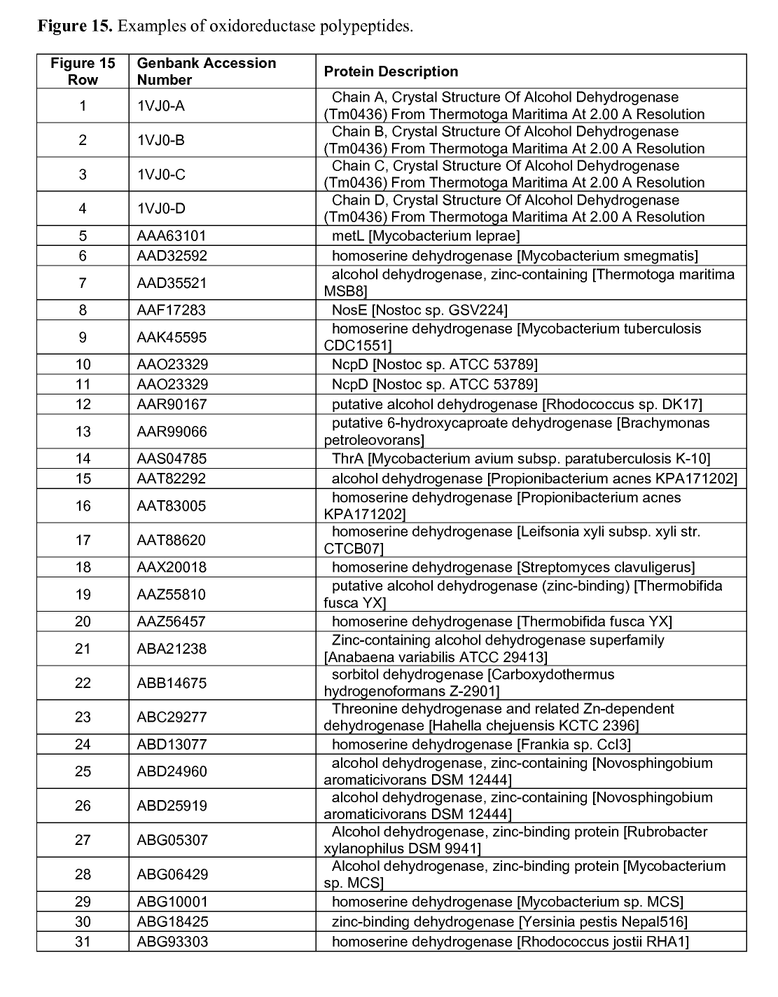

Glutamate-5-semialdehyde dehydrogenase [EC:1.2.1.41] is an example of an enzyme capable of catalyzing conversion A2 in FIG. 1A. Examples of enzymes that catalyze the NADP/NADPH dependent conversion of L-glutamate 5-phosphate to L-glutamate 5-semialdehyde are glutamate-5-semialdehyde dehydrogenases [EC:1.2.1.41] represented by the Genbank GI numbers in FIG. 1B and the corresponding amino acid sequences (SEQ ID NOs:10-15) as well as the polypeptides represented by the Genbank Accession numbers in FIG. 14. Certain polypeptides capable of catalyzing conversion A2 in FIG. 1A have an amino acid sequence that is at least about 75%, 85%, 90%, 95% or 100% identical to that of any of SEQ ID NOs:10-15 or the polypeptides represented by the Genbank Accession numbers in FIG. 14.

In certain cases, the enzymes which catalyze conversions A1 and A2 function together in a multi-subunit protein complex. This may be advantageous because it allows the A2 conversion to occur by preventing cyclization of the A1 product, L-glutamate 5-phosphate, to (S)-5-oxopyrrolidine-2-carboxylic acid. In certain cases, the A1 and A2 conversions are catalyzed by a single bifunctional polypeptide. For example, NCBI-GeneID: 824727 represents the Arabidopsis thaliana (thale cress) delta 1-pyrroline-5-carboxylate synthase 2 which encodes both activities.

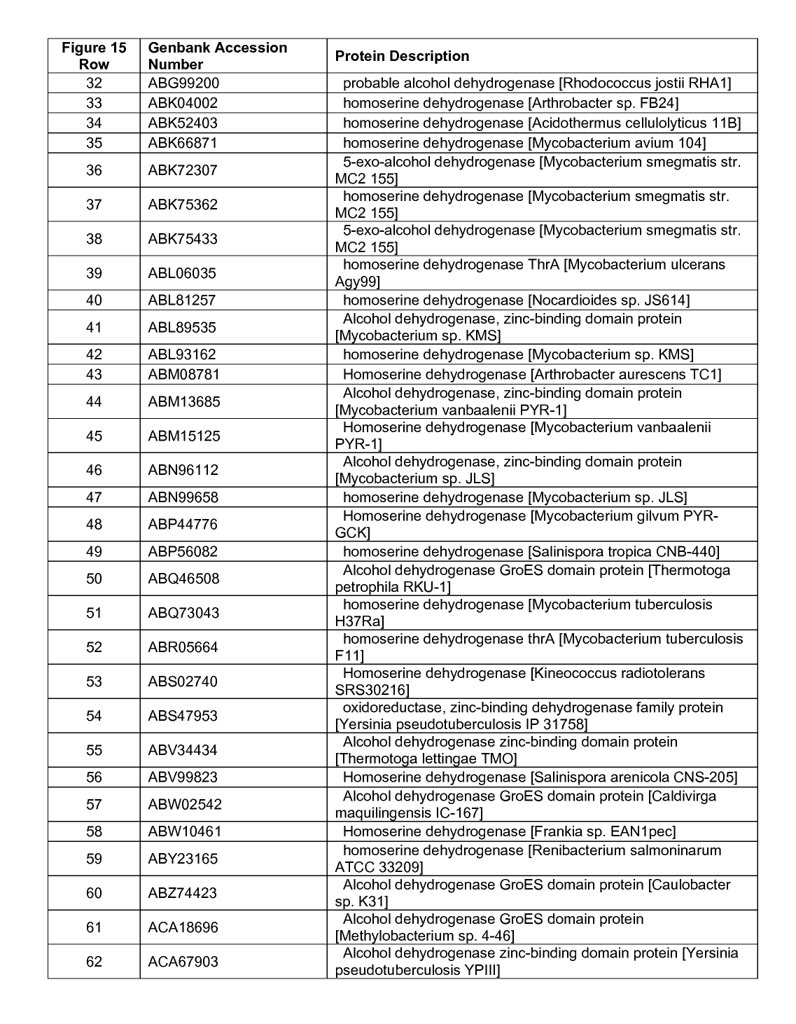

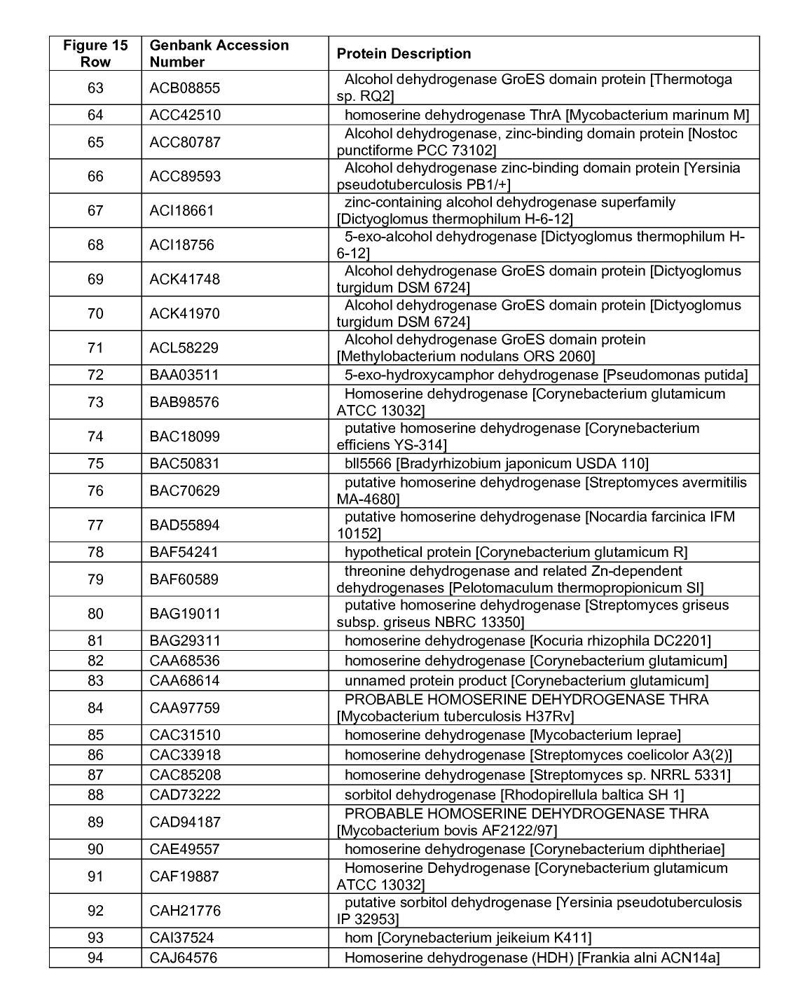

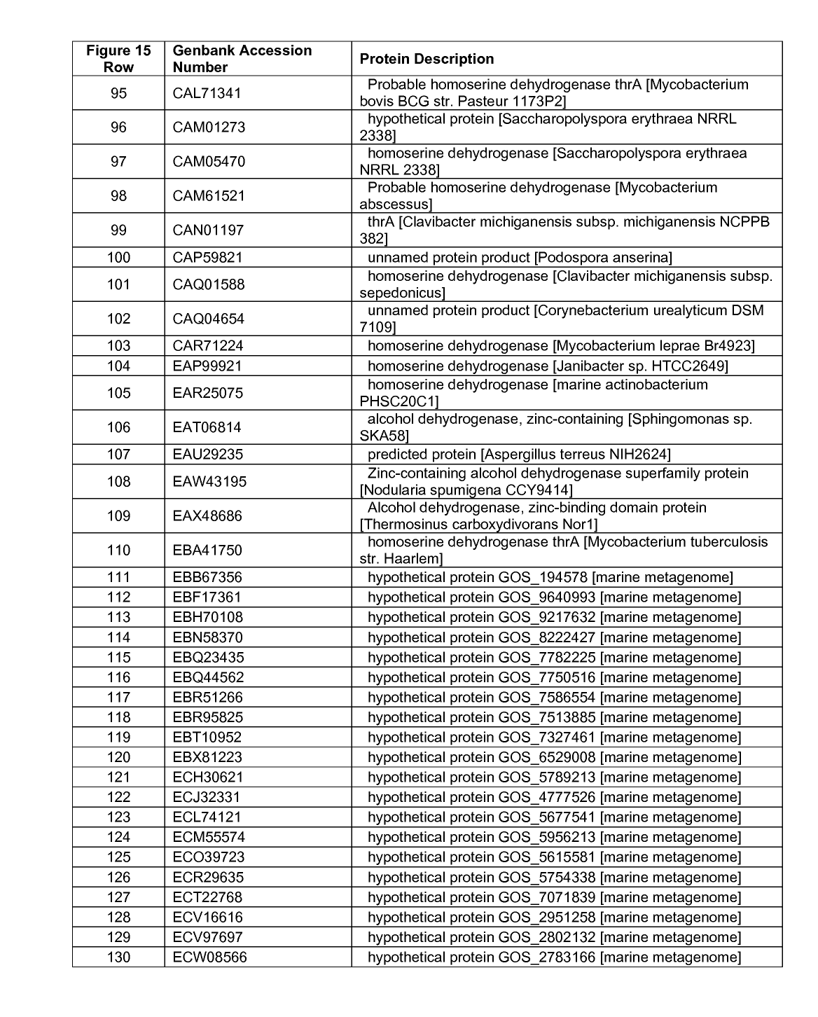

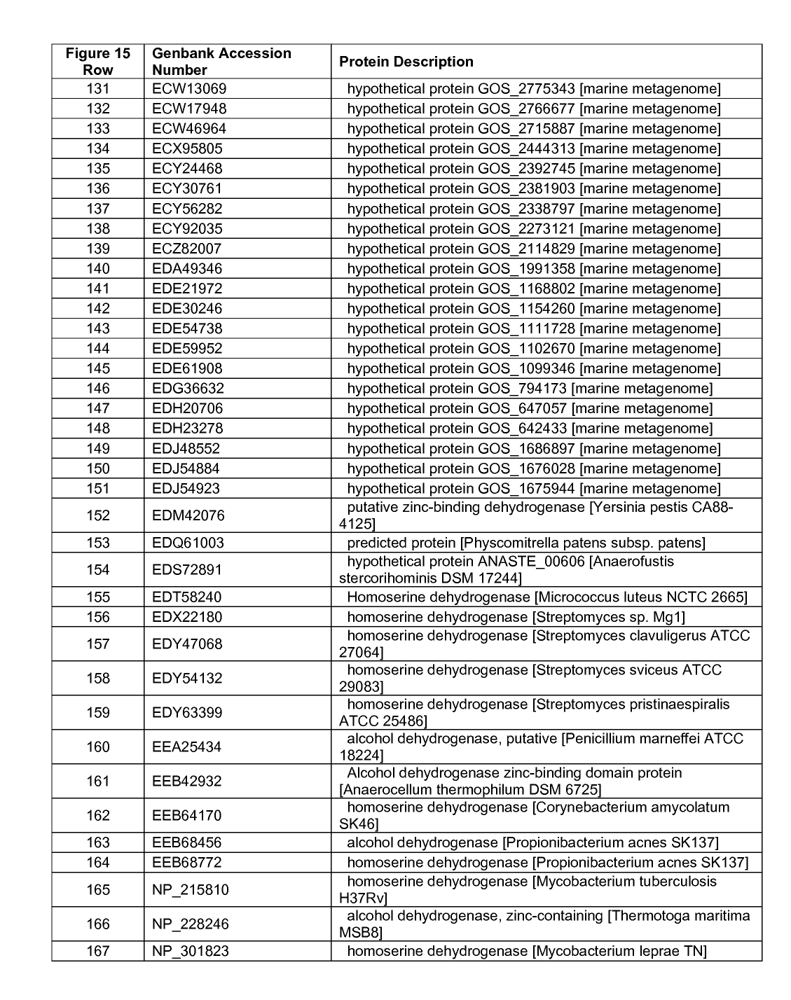

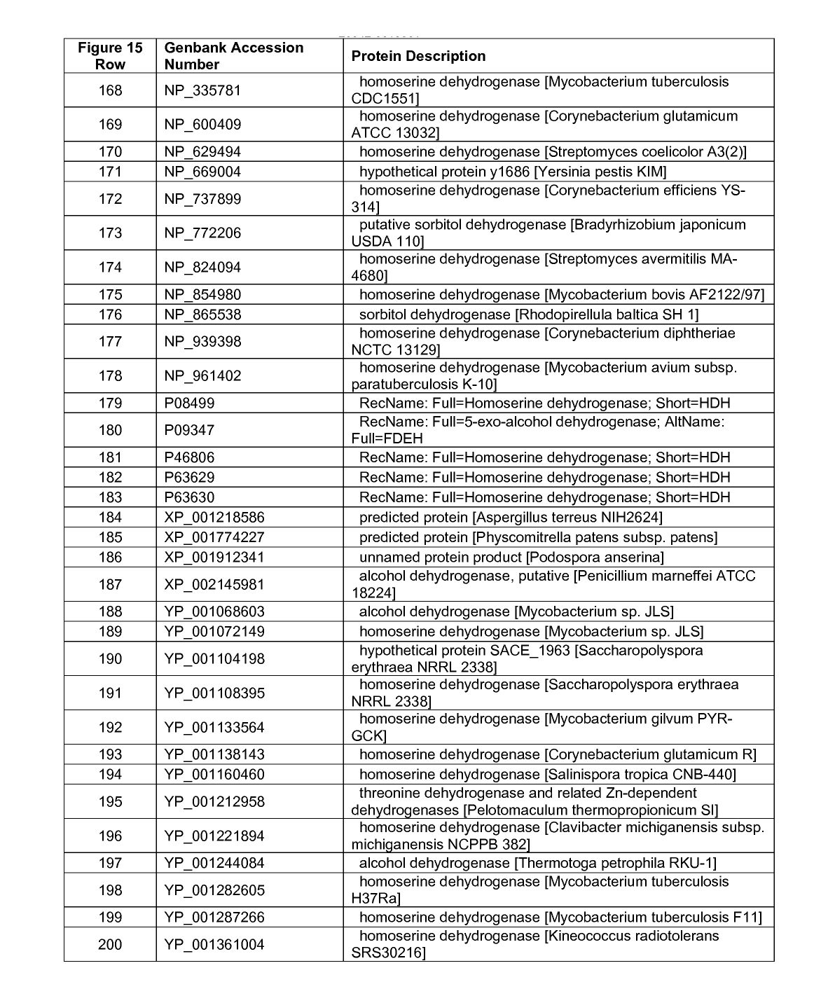

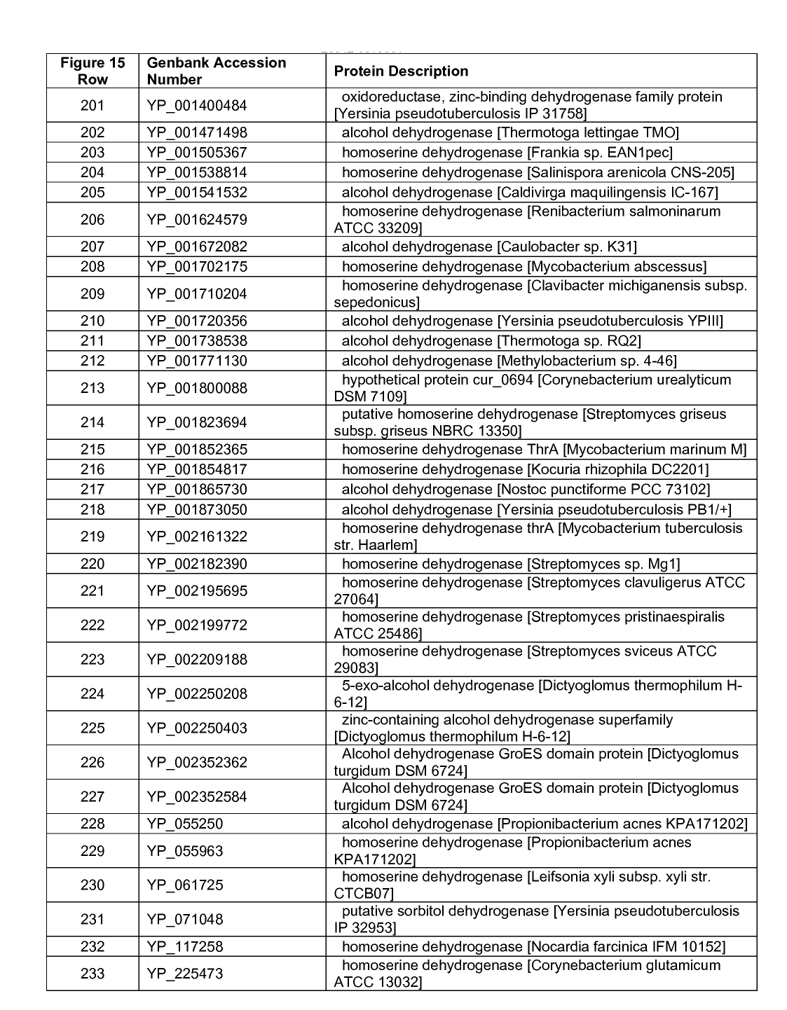

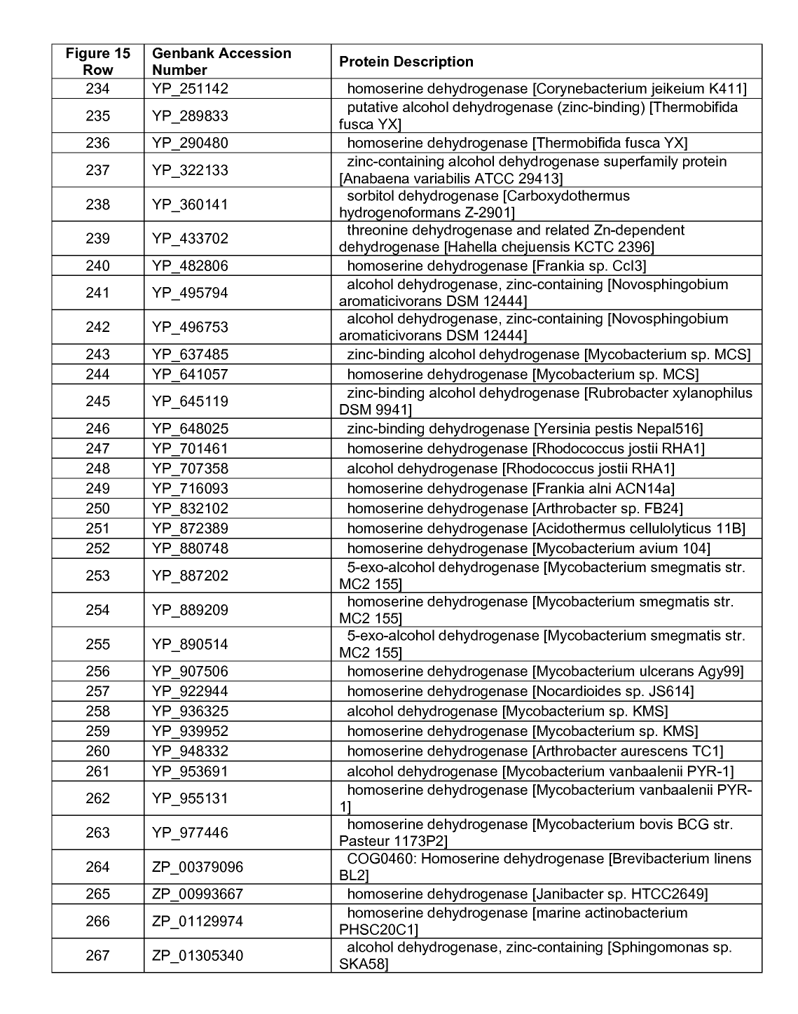

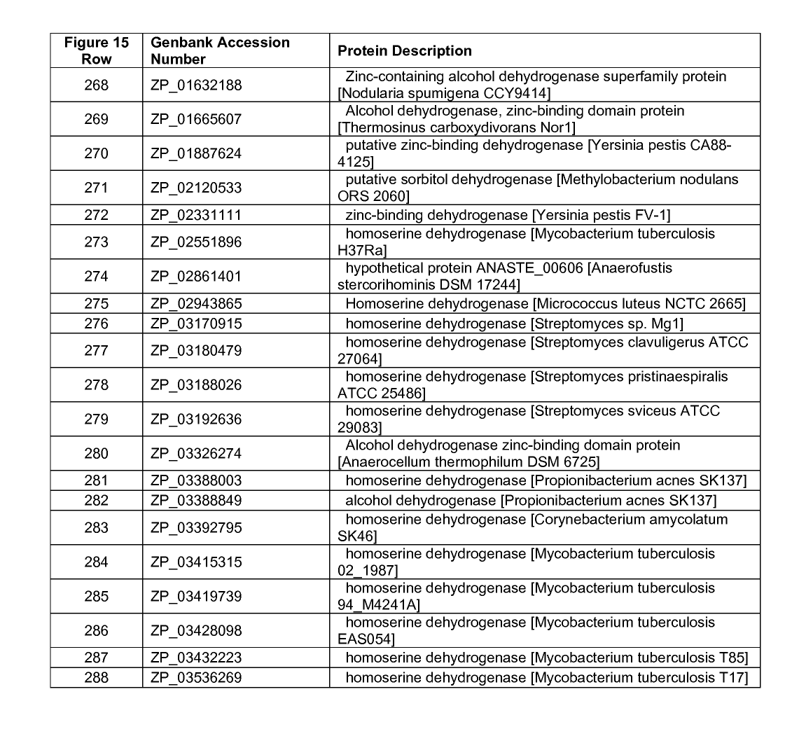

Oxidoreductases [EC 1.1.1.X] are enzymes that carry out oxidation/reduction reactions on alcohols or aldehydes using NAD/NADH or NADP/NADPH as a cofactor. Certain of these enzymes are capable of catalyzing conversion A3 in FIG. 1A. Examples of enzymes that may catalyze the NAD(P)/NAD(P)H dependent conversion of L-glutamate 5-semialdehyde to 5-hydroxy-L-norvaline are certain oxidoreductases [EC 1.1.1.X], including certain homoserine dehydrogenases [EC 1.1.1.3] represented by the Genbank GI numbers in FIG. 1B and the corresponding amino acid sequences (SEQ ID NOs:16-27) as well as the polypeptides represented by the Genbank Accession numbers in FIG. 15. Certain polypeptides capable of catalyzing conversion A3 in FIG. 1A have an amino acid sequence that is at least about 75%, 85%, 90%, 95% or 100% identical to that of any of SEQ ID NOs:16-27 or the polypeptides represented by the Genbank Accession numbers in FIG. 15. In certain cases where the oxidoreductase is a wild-type homoserine dehydrogenase negatively regulated by threonine or another structurally related inhibitor, an allele with reduced inhibition may be employed. For example, the following homoserine dehydrogenase reduced inhibited alleles may be useful in the present disclosure: C. glutamicum G378E, C. urealyticum G378E, A. aurescens G365E, R. salmoninarum G356E, Rhodococcus sp. G368E, S. coelicolor G362E, or, if another homoserine dehydrogenase source species is used, one having the glycine to glutamic acid change at the corresponding amino acid.

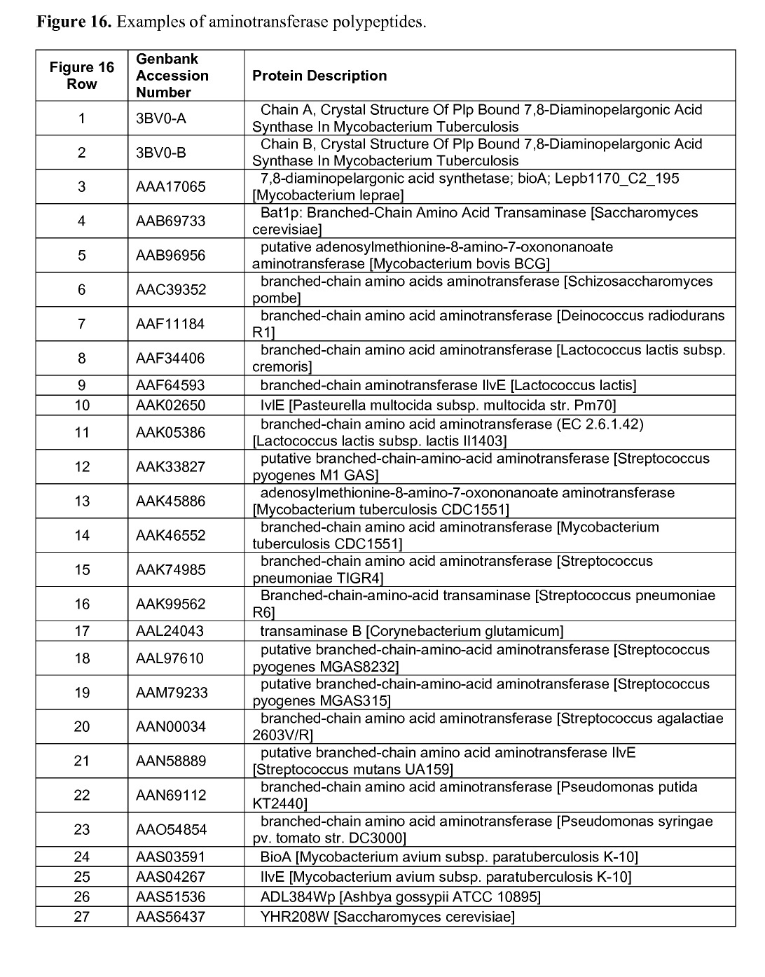

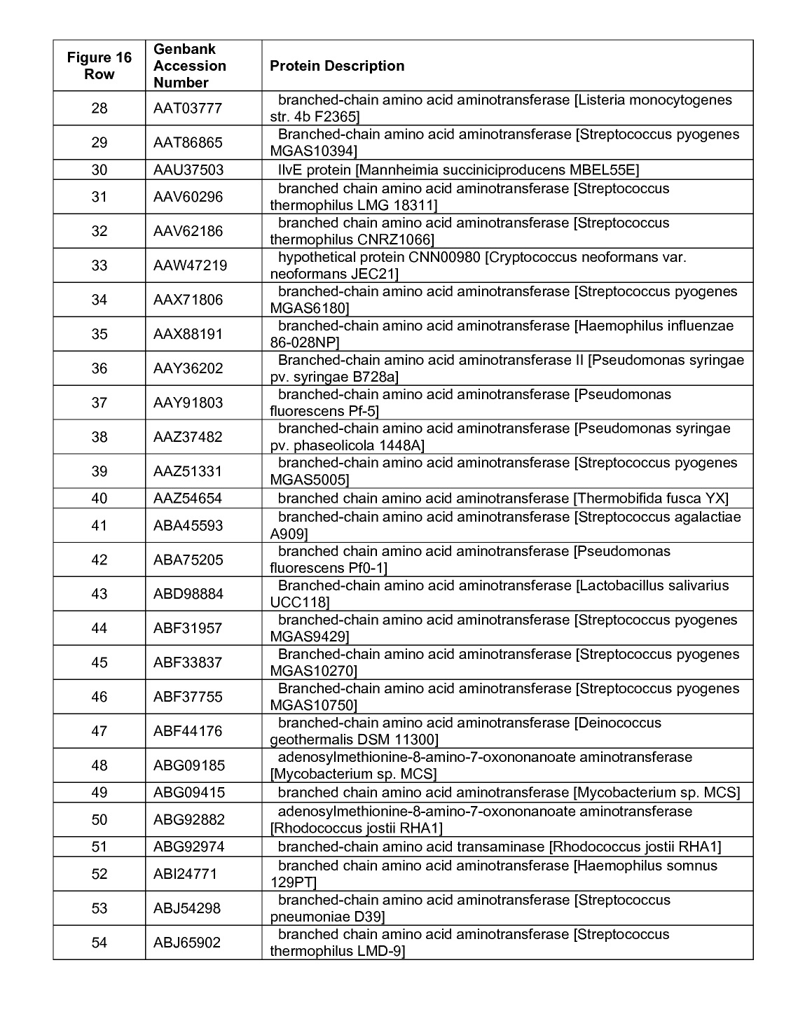

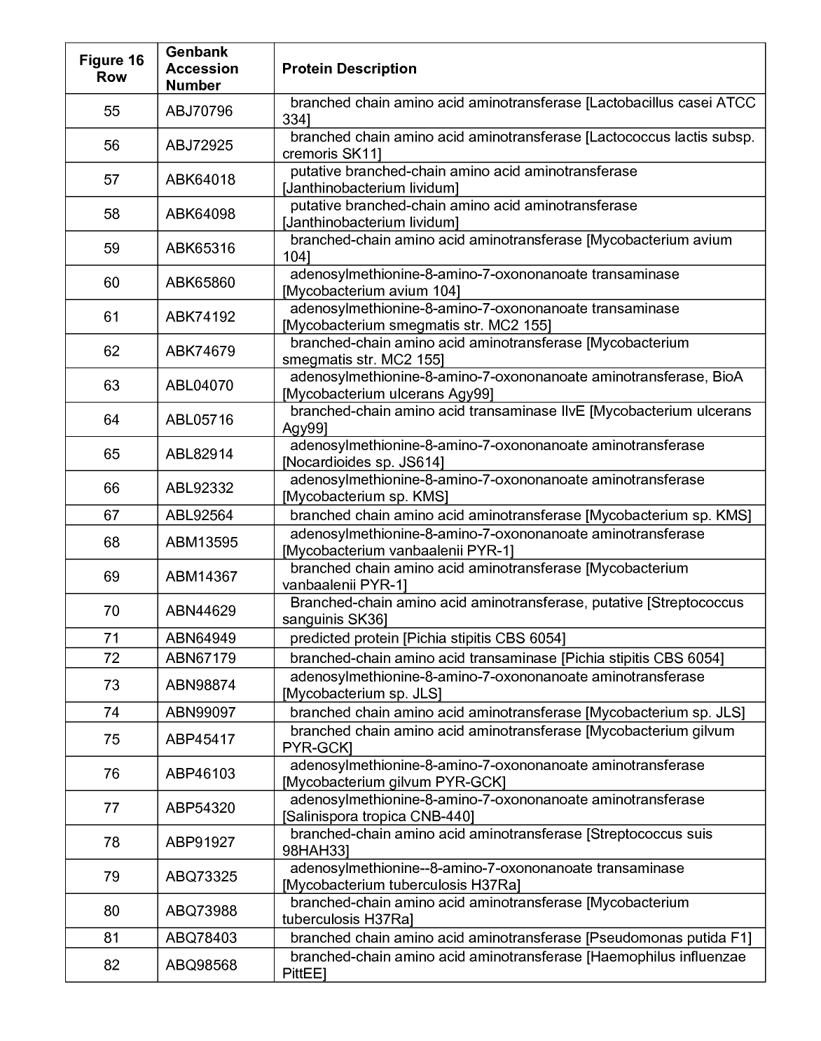

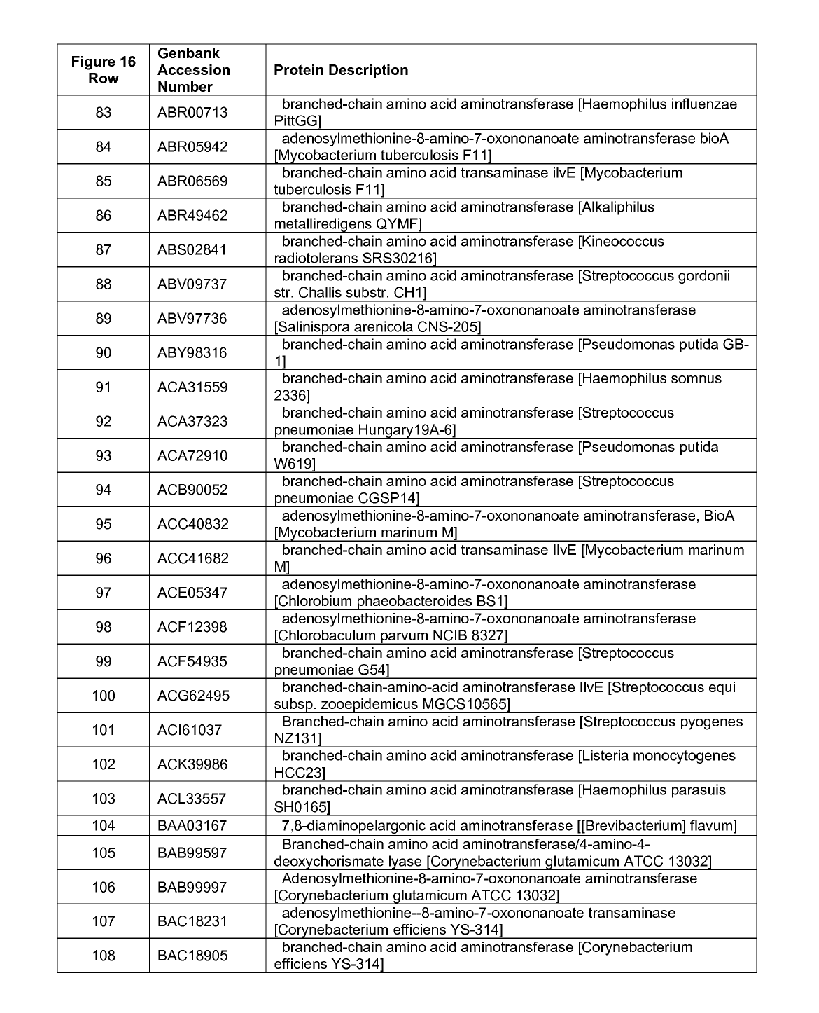

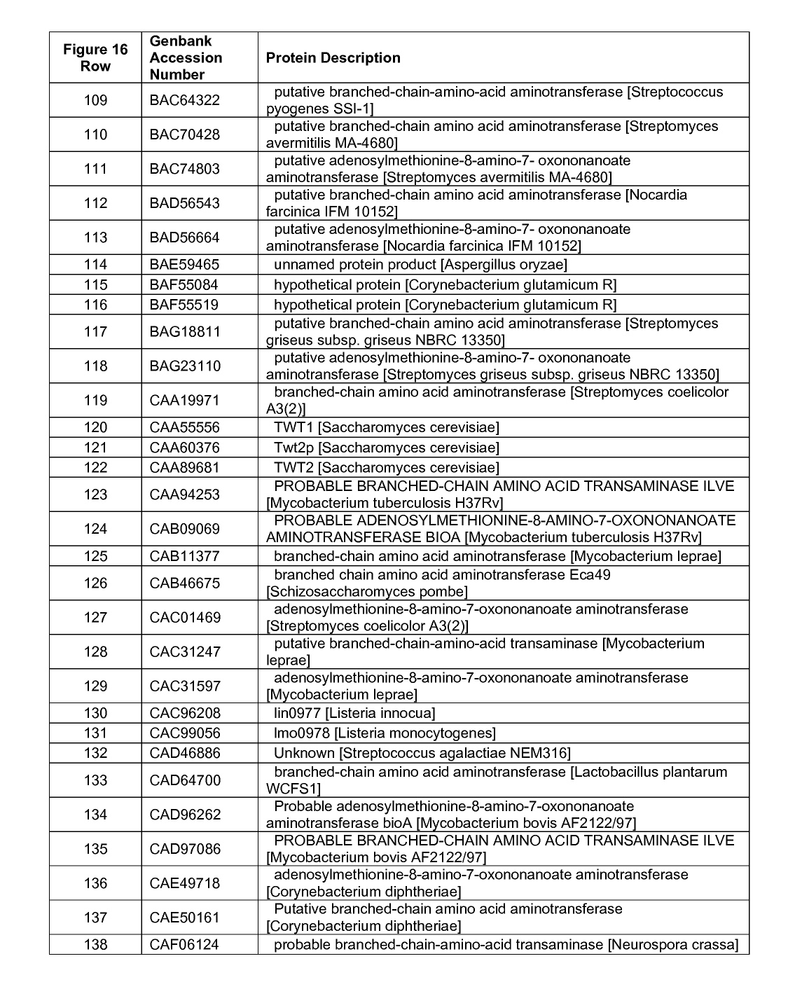

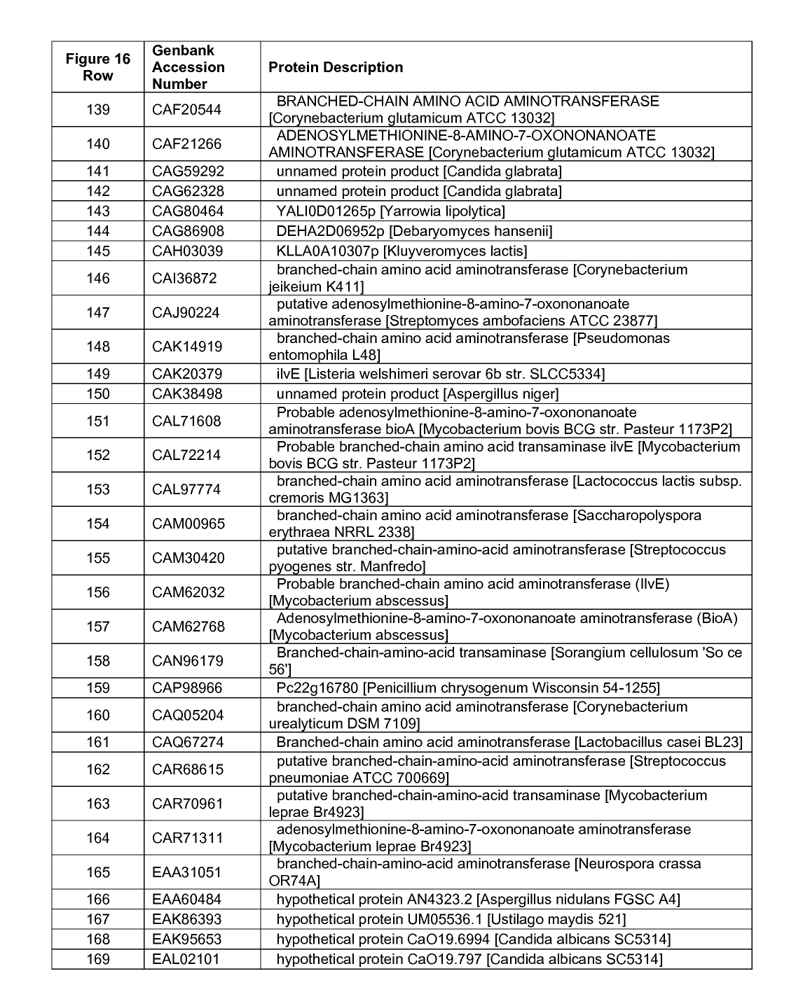

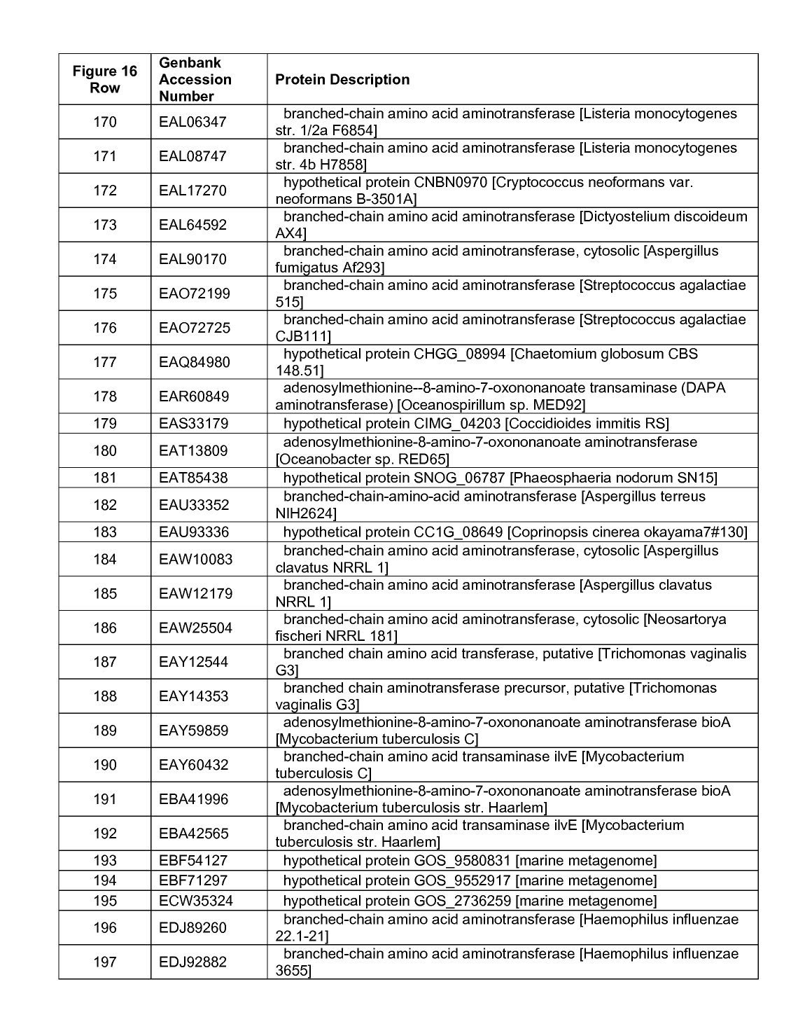

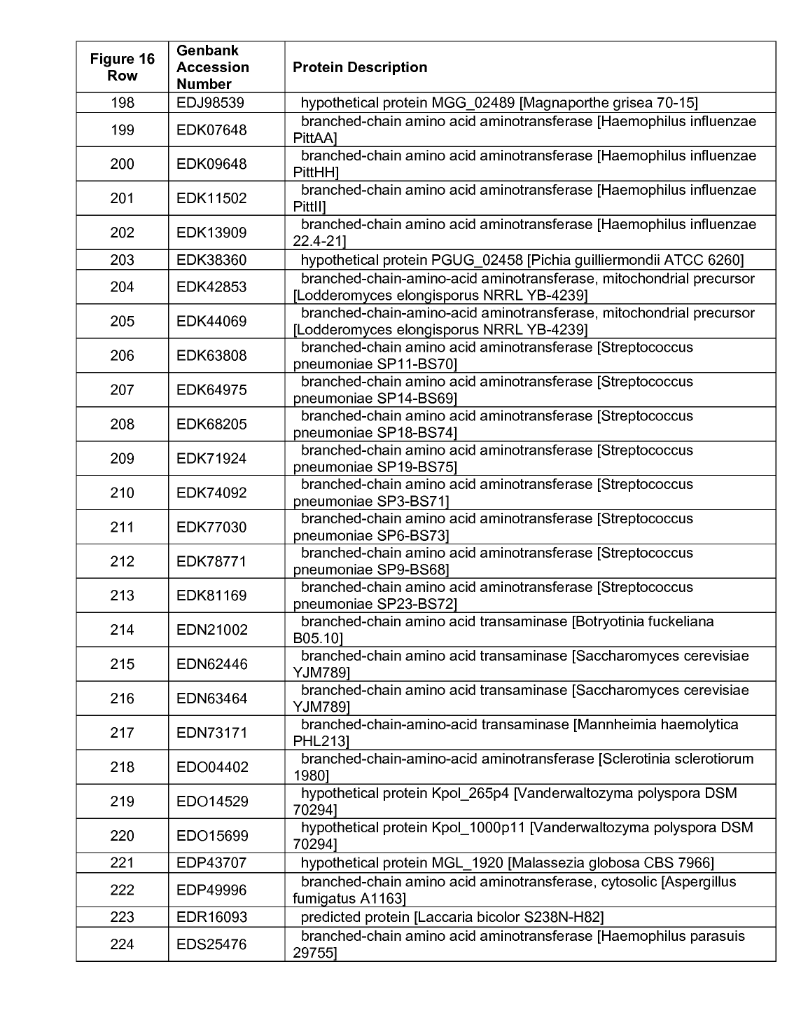

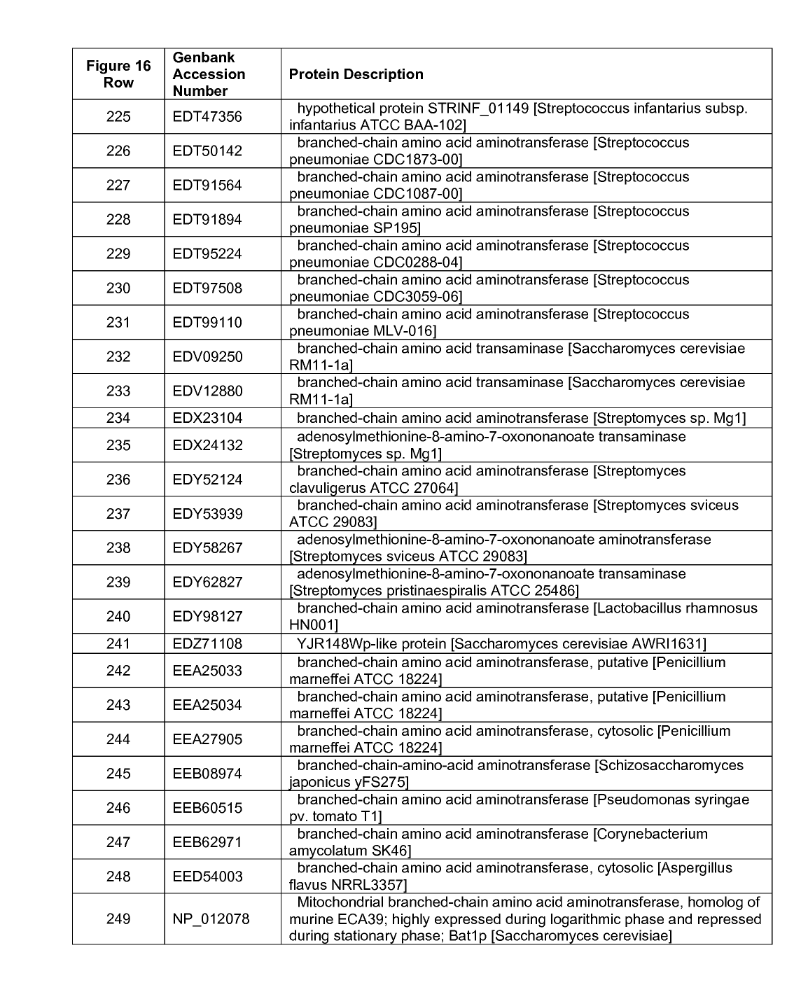

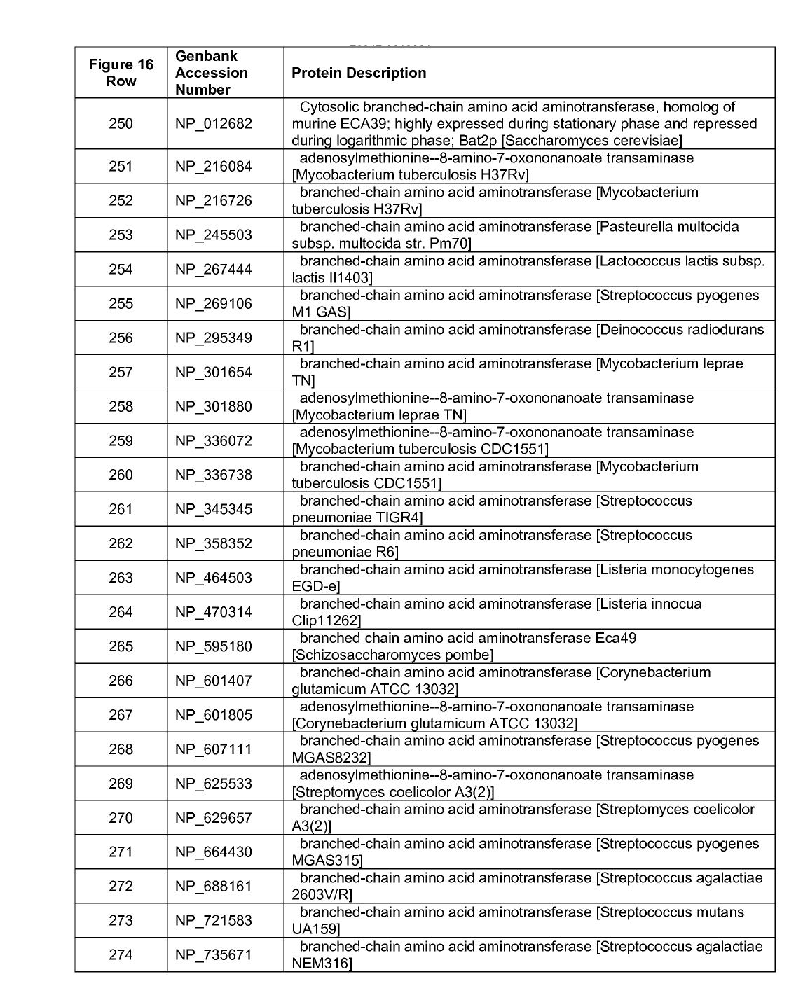

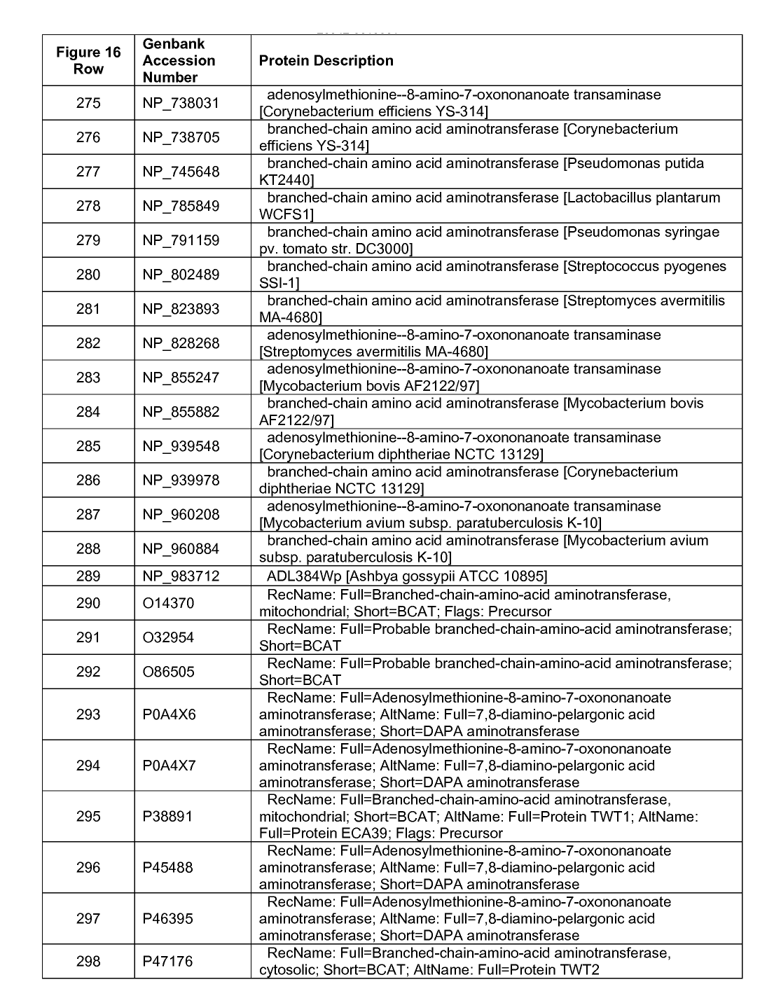

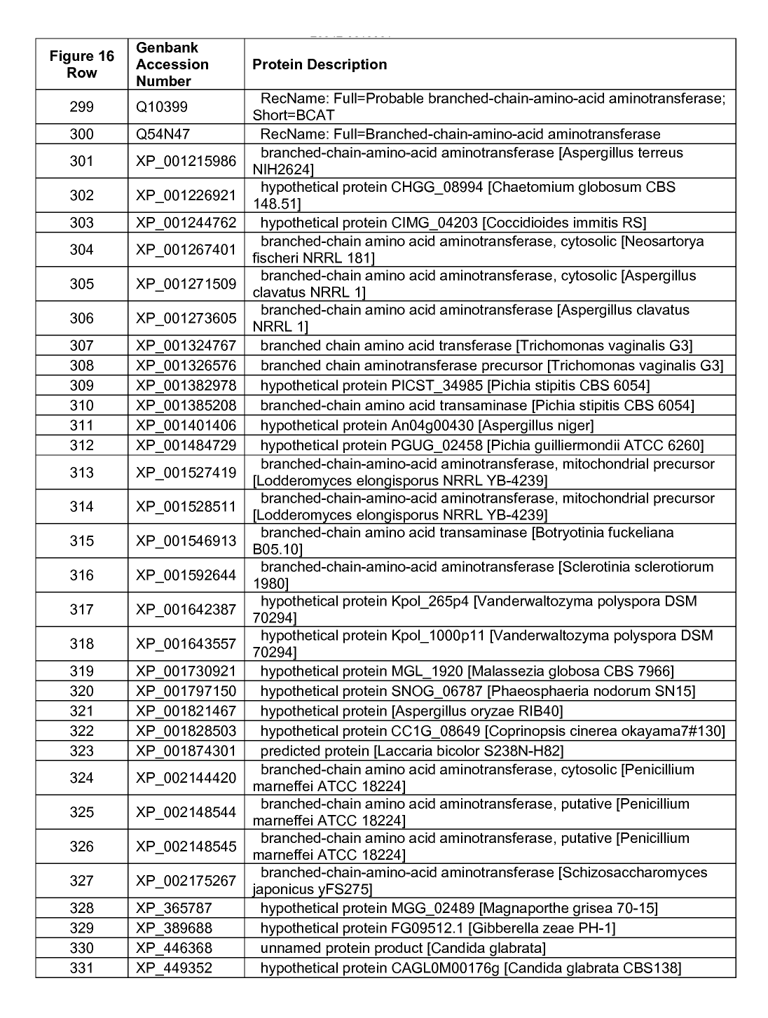

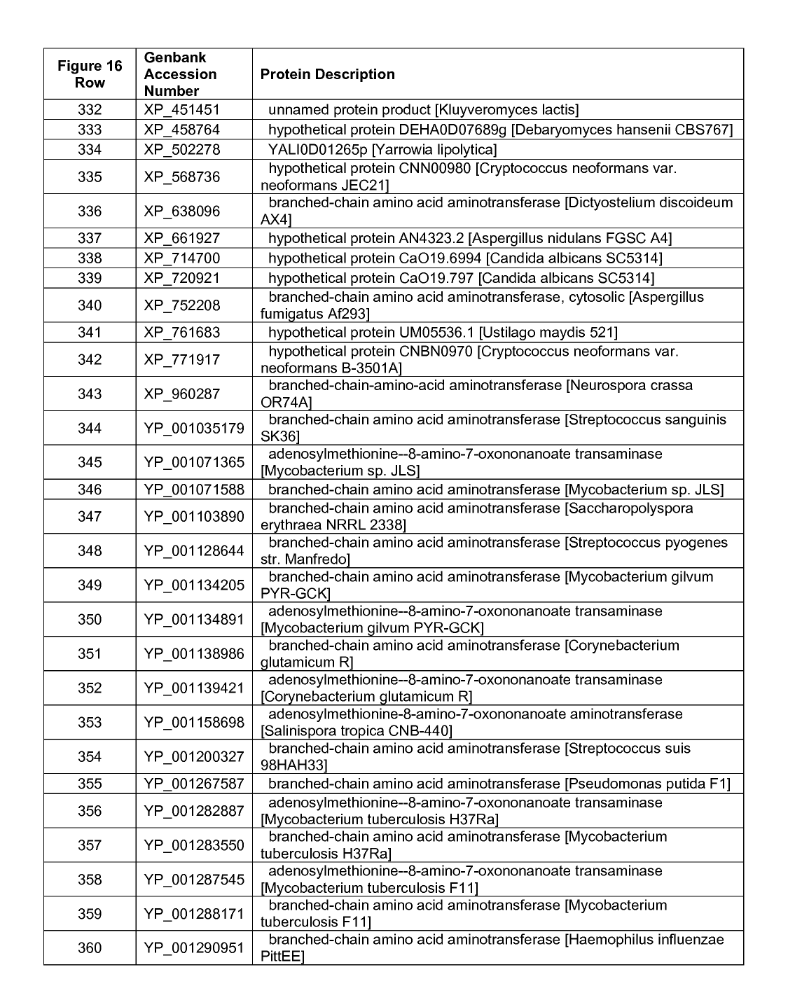

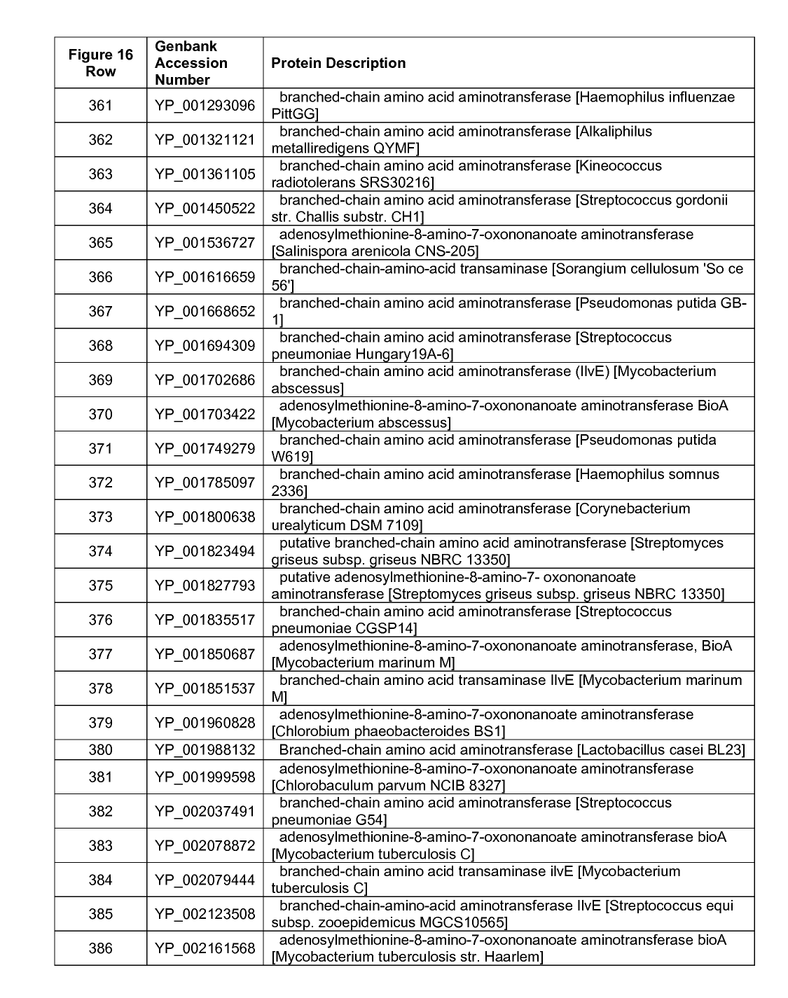

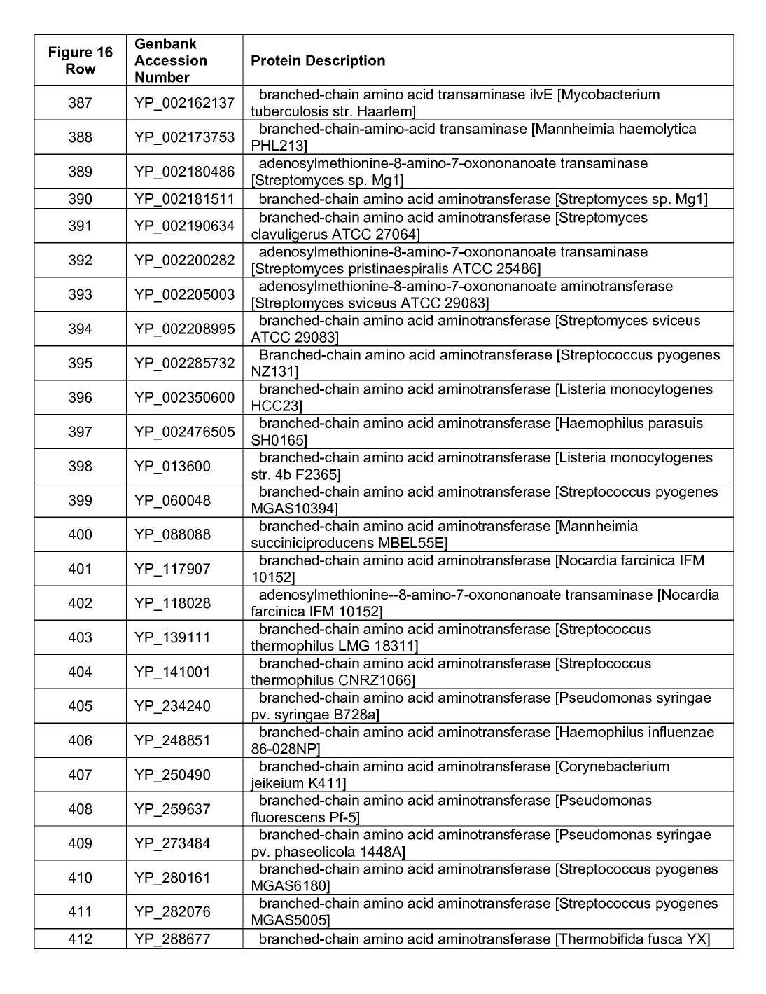

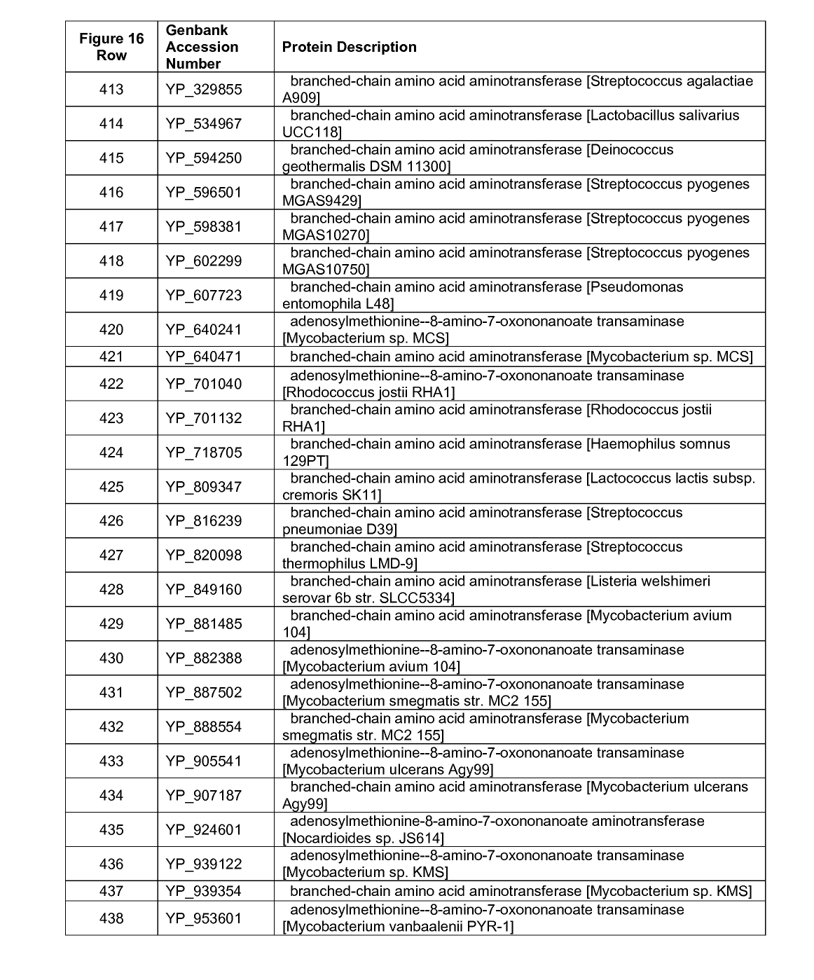

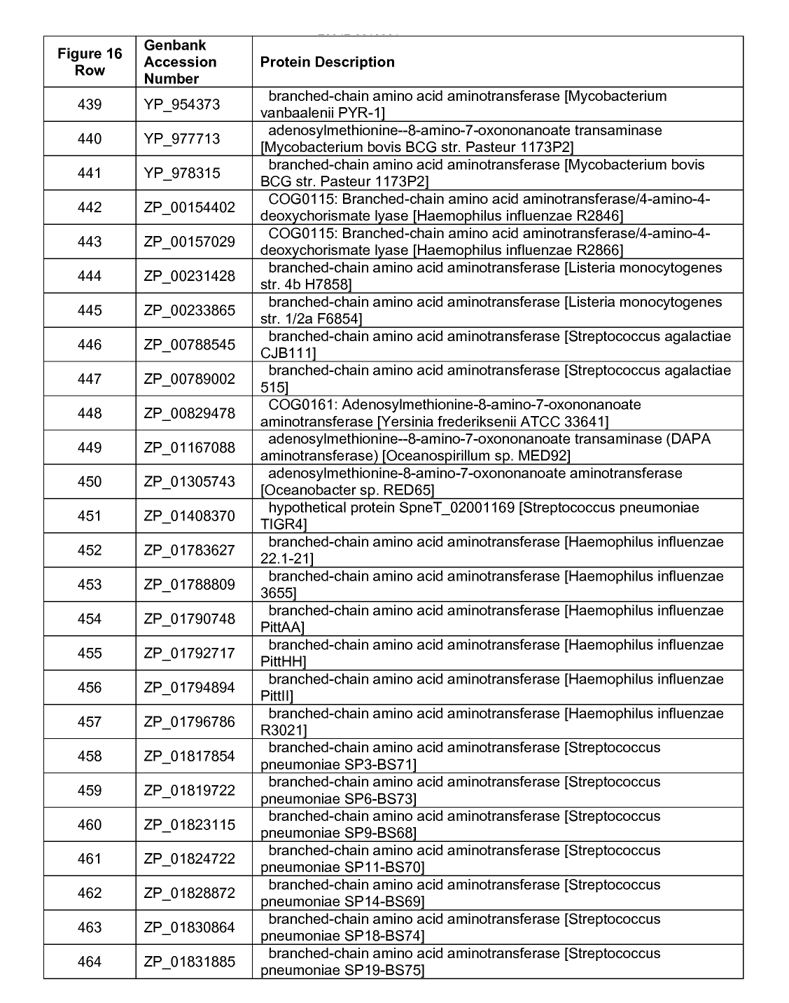

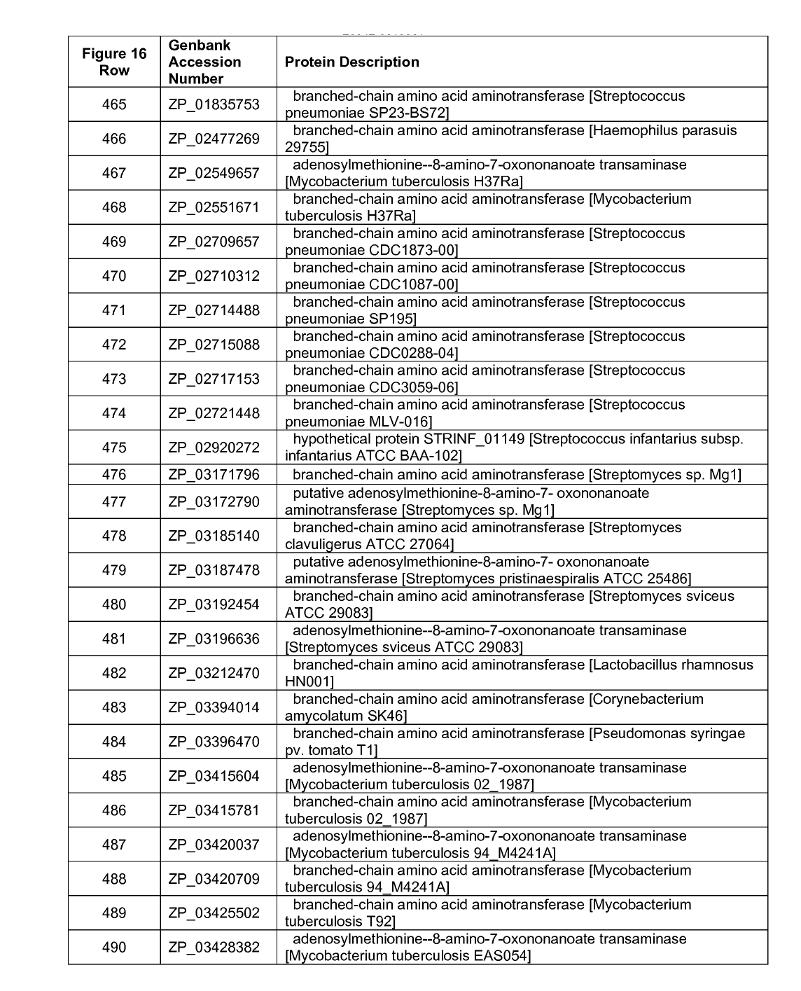

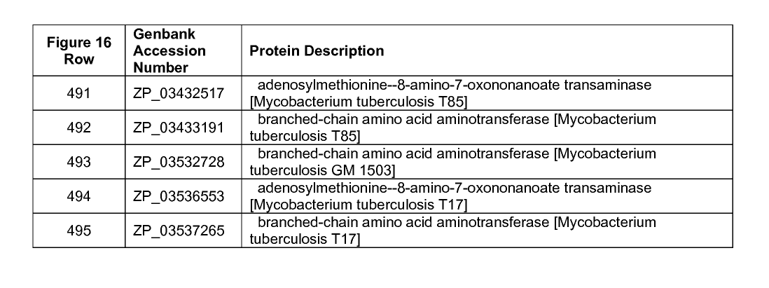

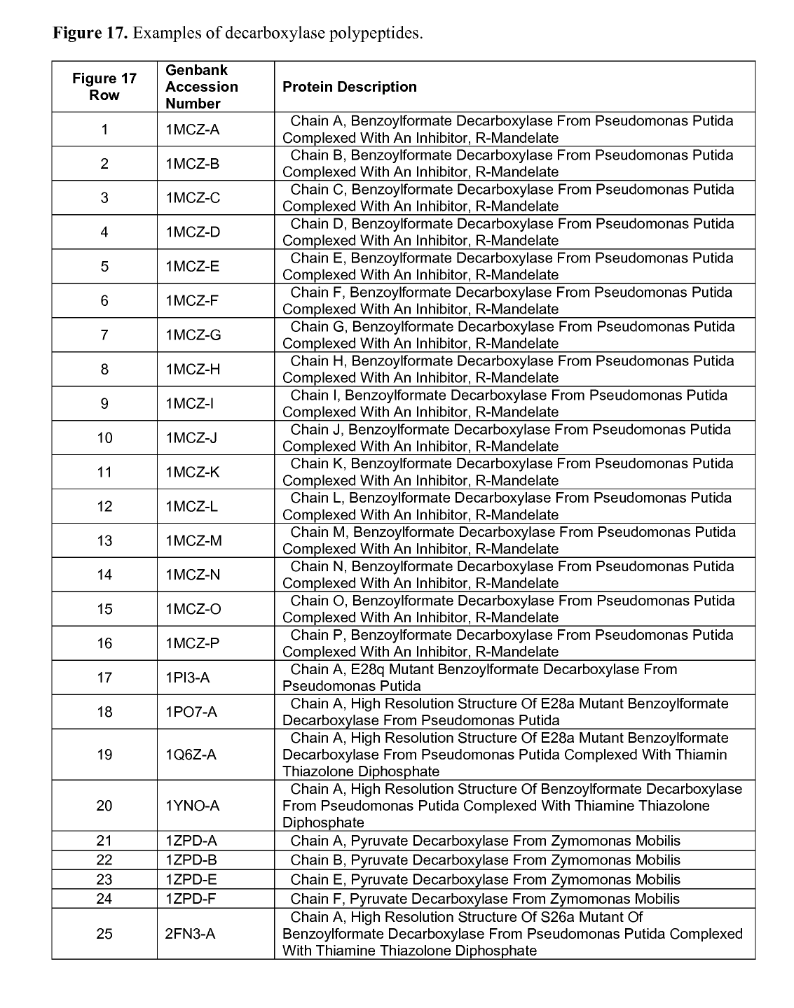

Aminotransferases [EC 2.6.1.X] are enzymes that transfer nitrogenous groups. Certain of these enzymes are capable of catalyzing conversion A4 in FIG. 1A. An aminotransferase useful in the present disclosure may use [symbol] −ketoglutarate as an amino acceptor. Examples of enzymes that may catalyze the conversion of 5-hydroxy-L-norvaline to 5-hydroxy-2-oxopentanoate include certain branched-chain-amino-acid aminotransferase/transaminases [EC 2.6.1.42] and certain adenosylmethionine-8-amino-7-oxononanoate transaminases [EC 2.6.1.62] represented by the Genbank GI numbers in FIG. 1B and the corresponding amino acid sequences (SEQ ID NOs:28-35 and 58) as well as the polypeptides represented by the Genbank Accession numbers in FIG. 16. Certain polypeptides capable of catalyzing conversion A4 in FIG. 1A have an amino acid sequence that is at least about 75%, 85%, 90%, 95% or 100% identical to that of any of SEQ ID NOs:28-35 and 58 or the polypeptides represented by the Genbank Accession numbers in FIG. 16.

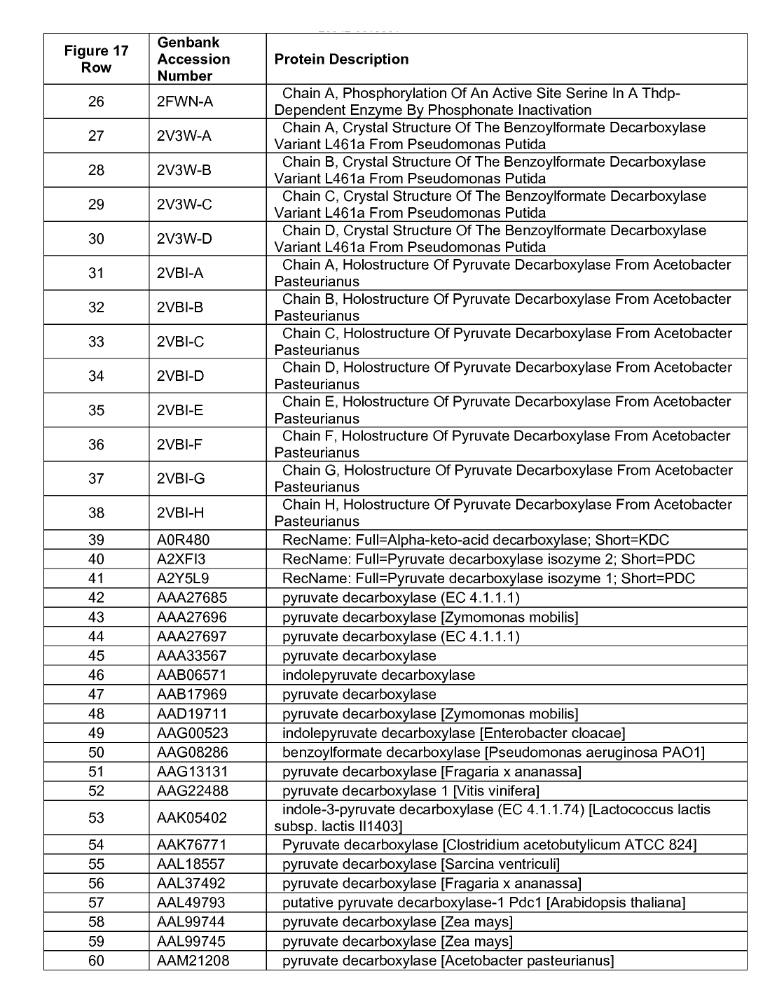

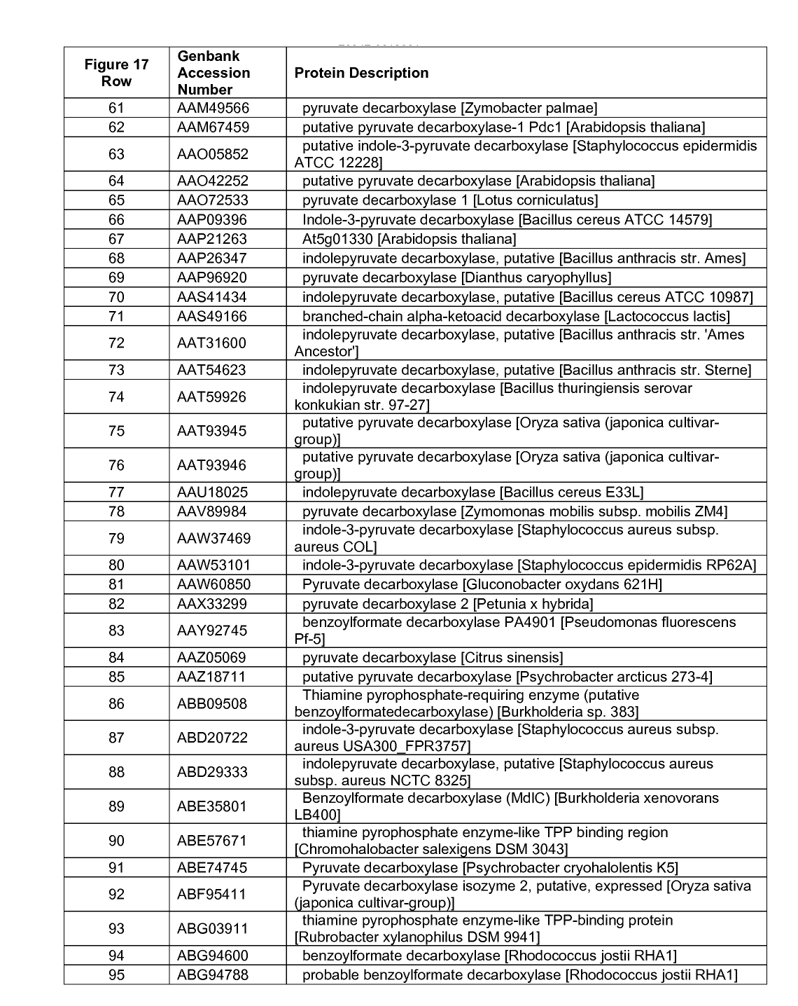

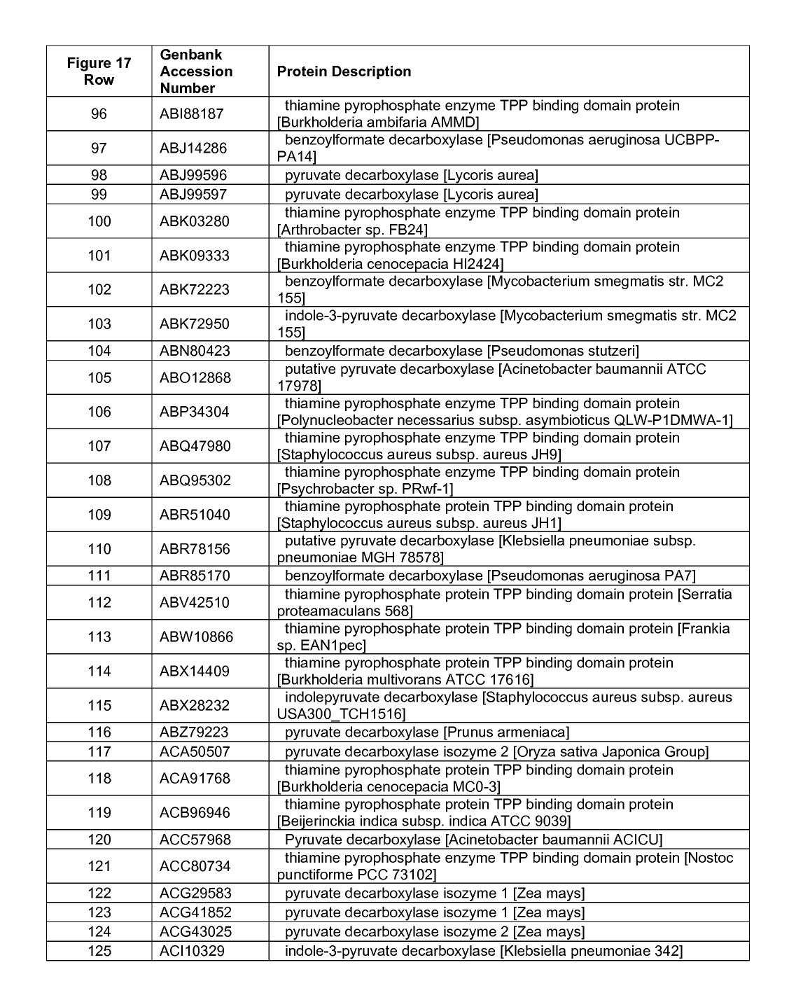

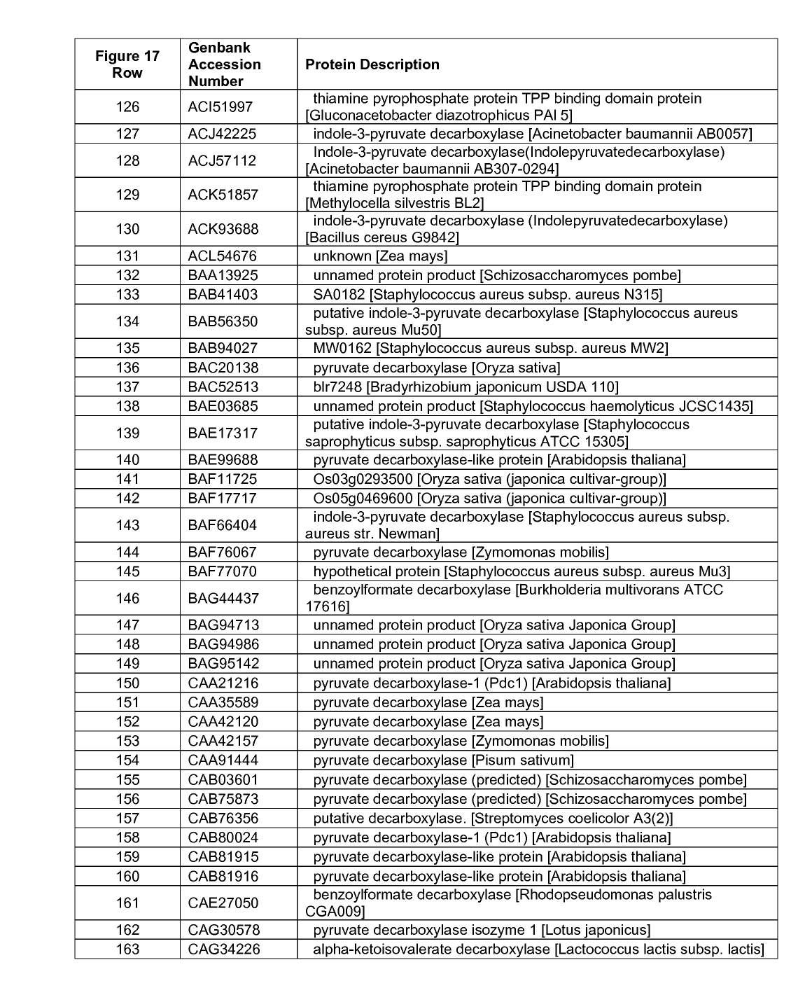

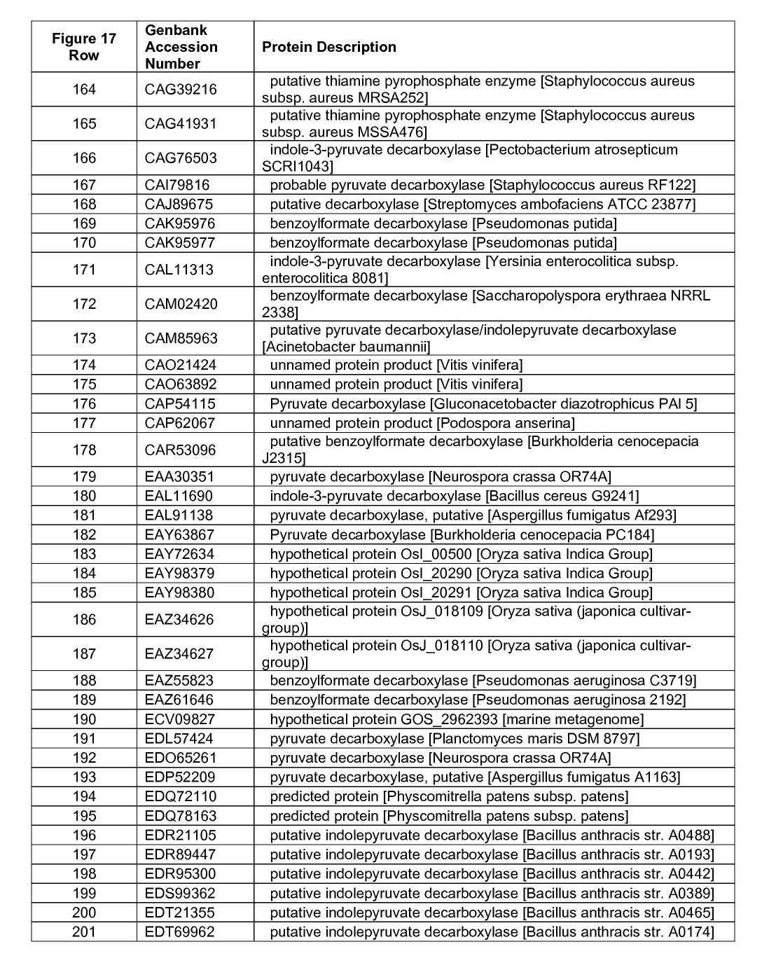

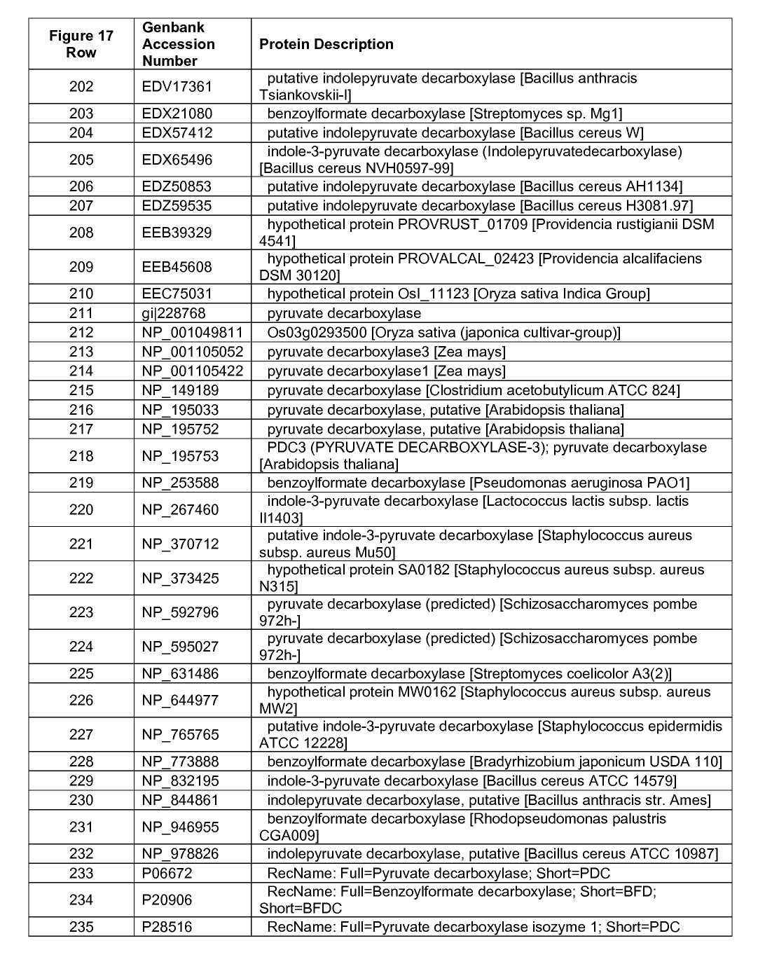

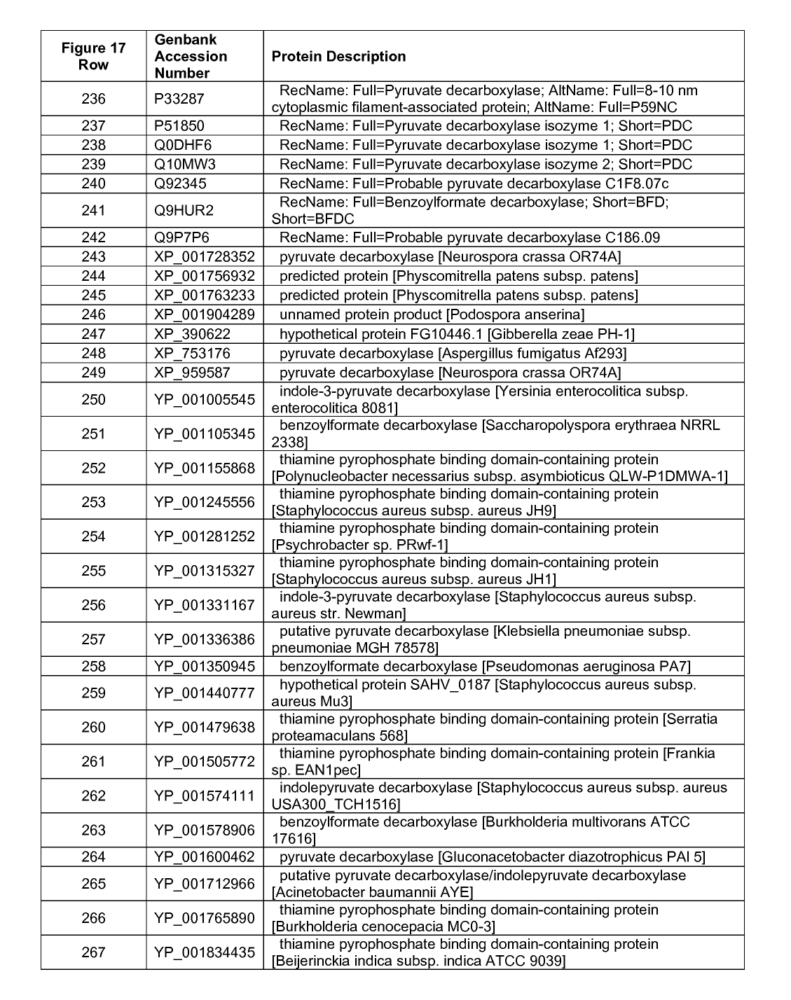

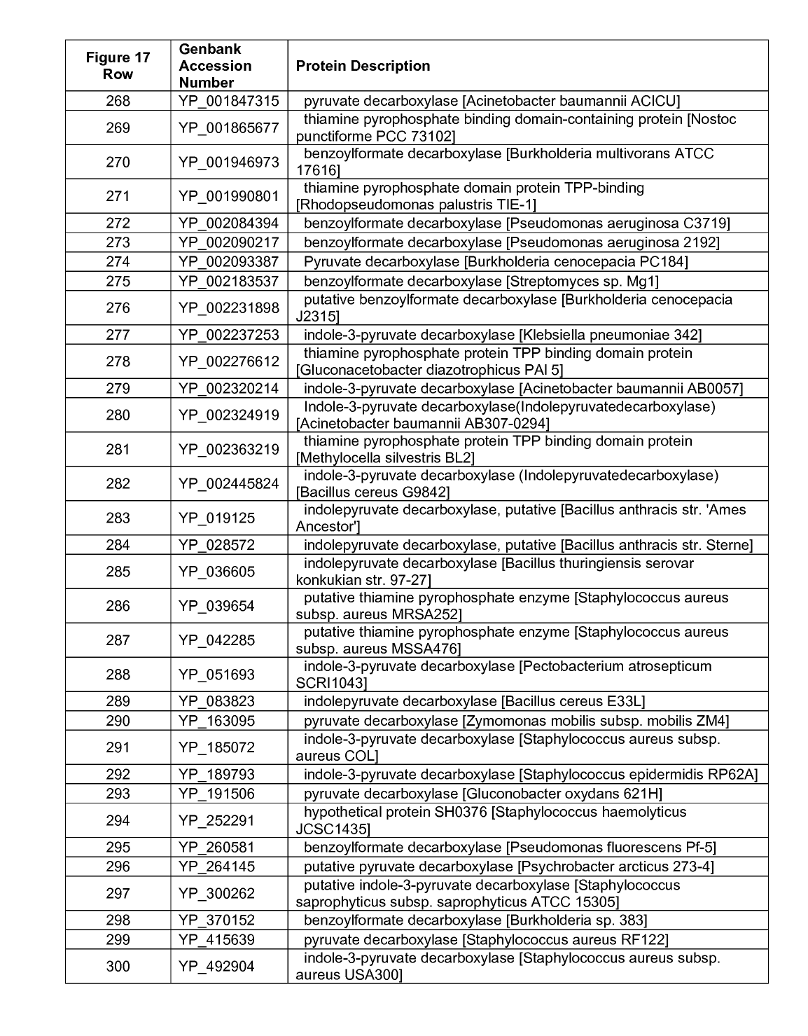

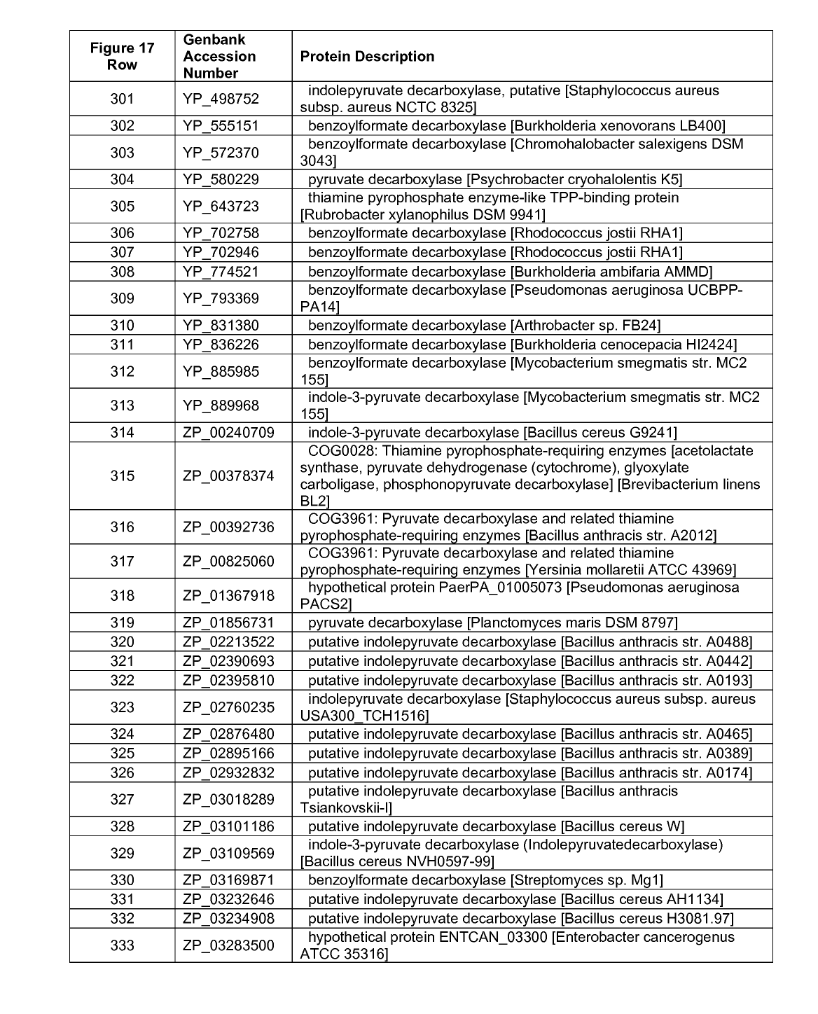

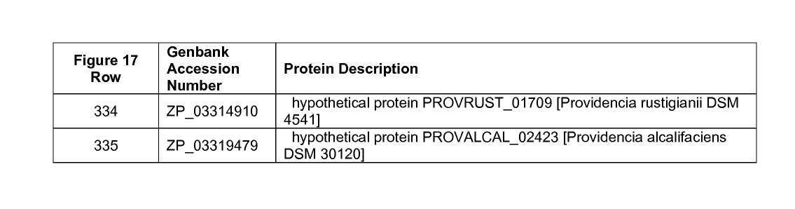

Decarboxylases [EC 4.1.1.X] are enzymes that remove carboxyl groups. Certain of these enzymes are capable of catalyzing conversion AS in FIG. 1A. Examples of enzymes that may catalyze the conversion of 5-hydroxy-2-oxopentanoate to 4-hydroxybutanal include certain branched chain alpha-keto decarboxylases [EC 4.1.1.72], certain pyruvate decarboxylases [EC 4.1.1.1], certain benzoylformate dehydrogenases [EC:4.1.1.7] and certain indole-3-pyruvate decarboxylases [EC 4.1.1.74] represented by the Genbank GI numbers in FIG. 1B and the corresponding amino acid sequences (SEQ ID NOs:36-43 and 59-70) as well as the polypeptides represented by the Genbank Accession numbers in FIG. 17. Certain polypeptides capable of catalyzing conversion AS in FIG. 1A have an amino acid sequence that is at least about 75%, 85%, 90%, 95% or 100% identical to that of any of SEQ ID NOs:36-43, 59-70 or the polypeptides represented by the Genbank Accession numbers in FIG. 17. In certain cases, the decarboxylase is not membrane bound.

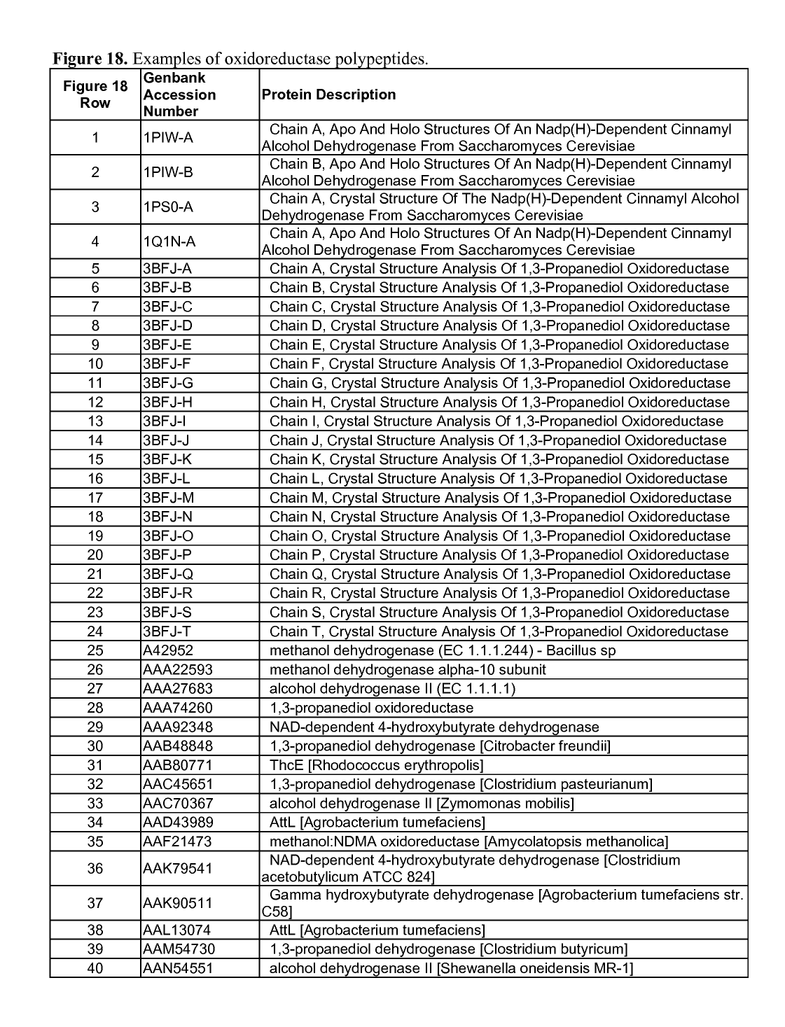

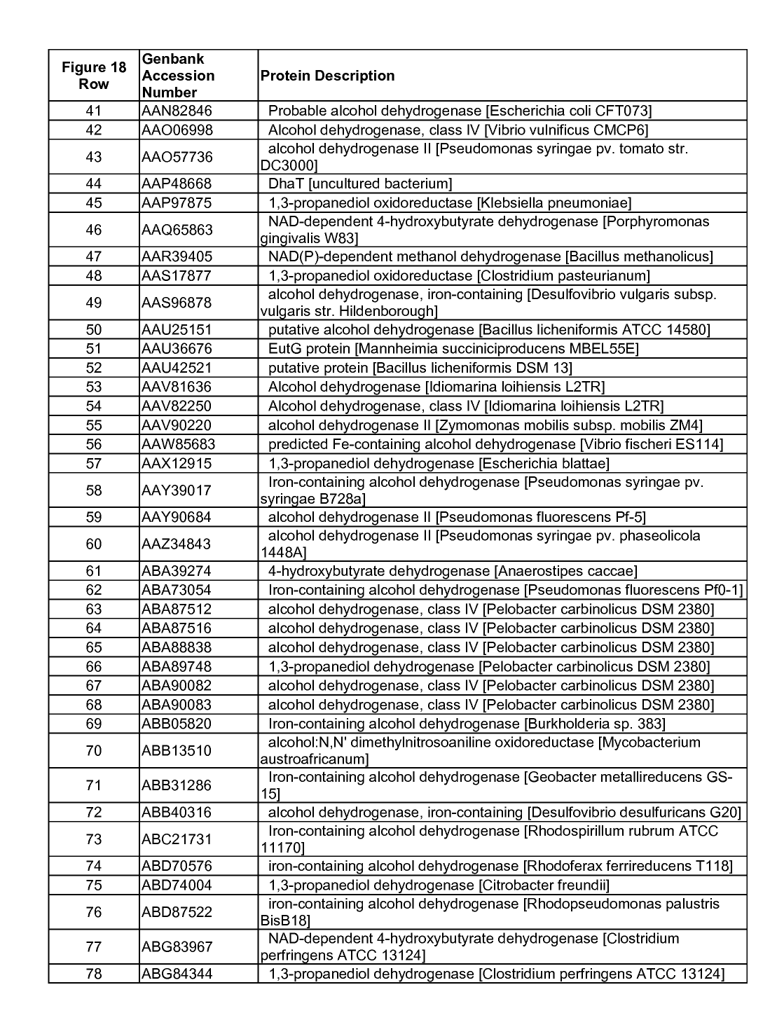

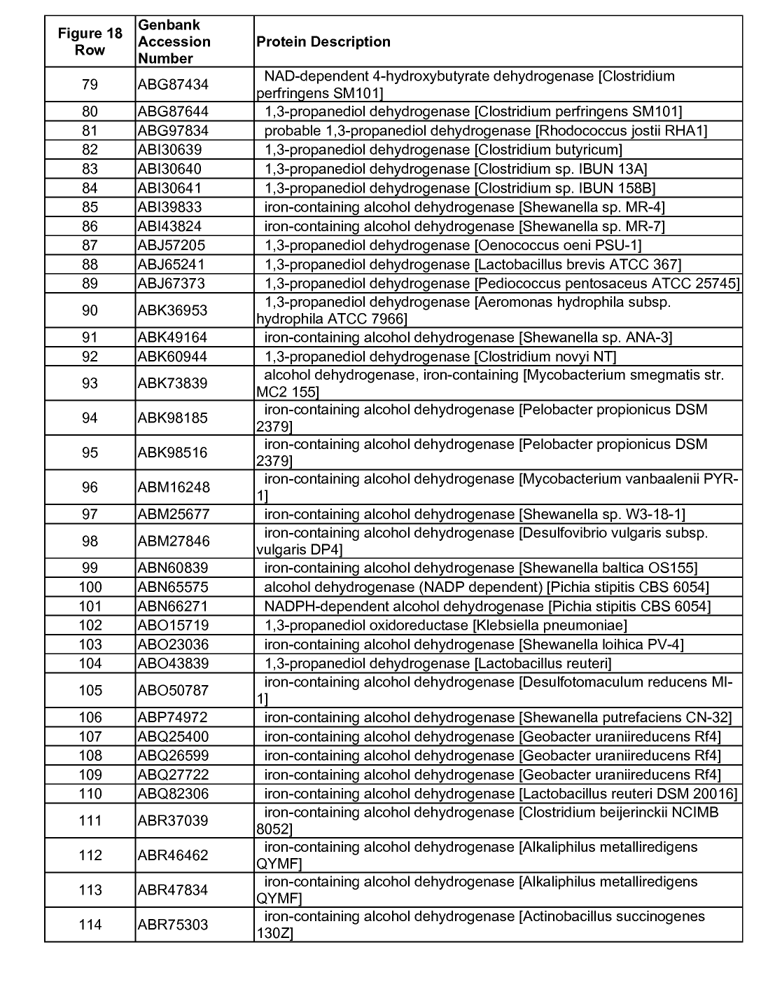

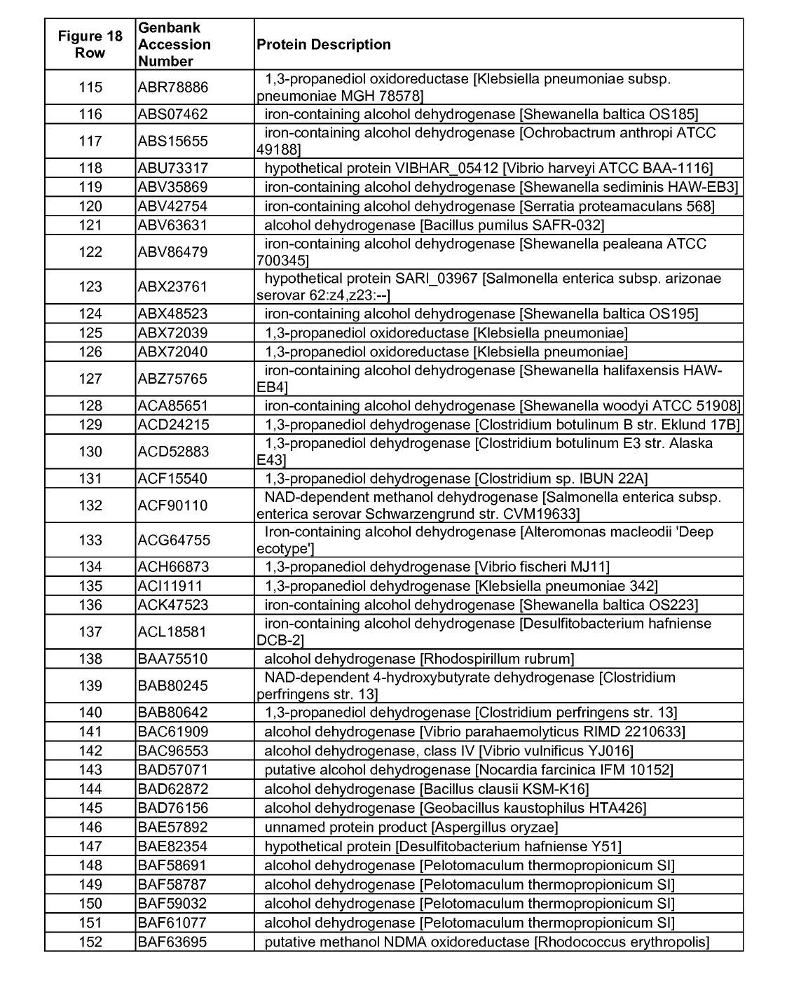

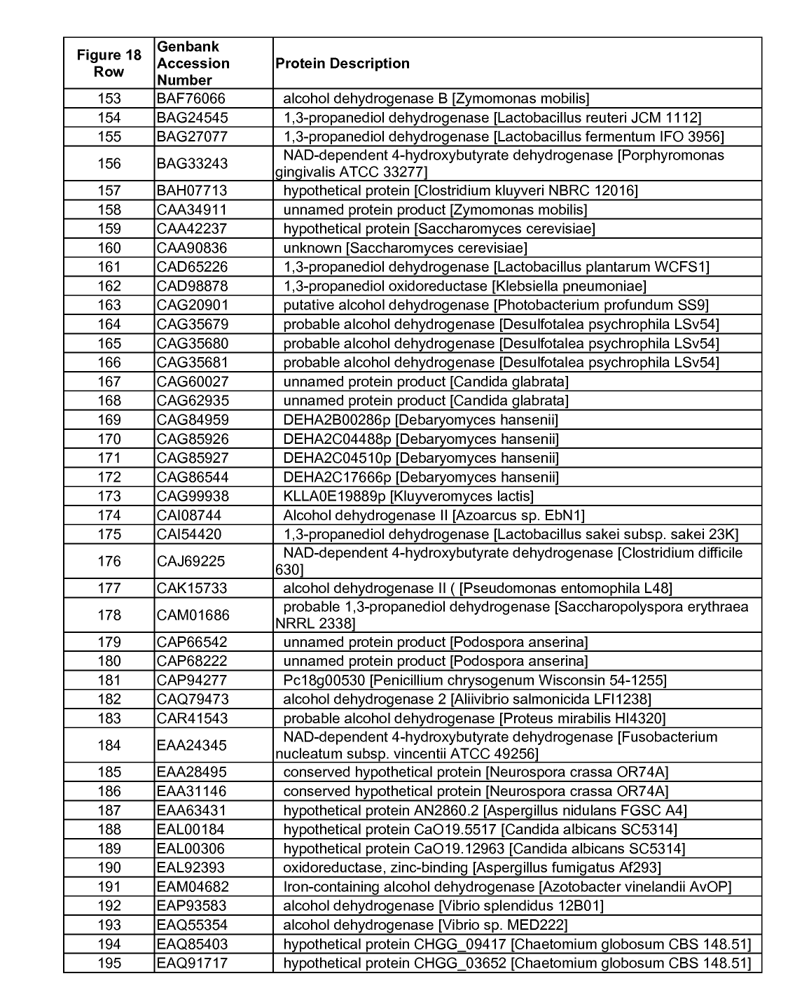

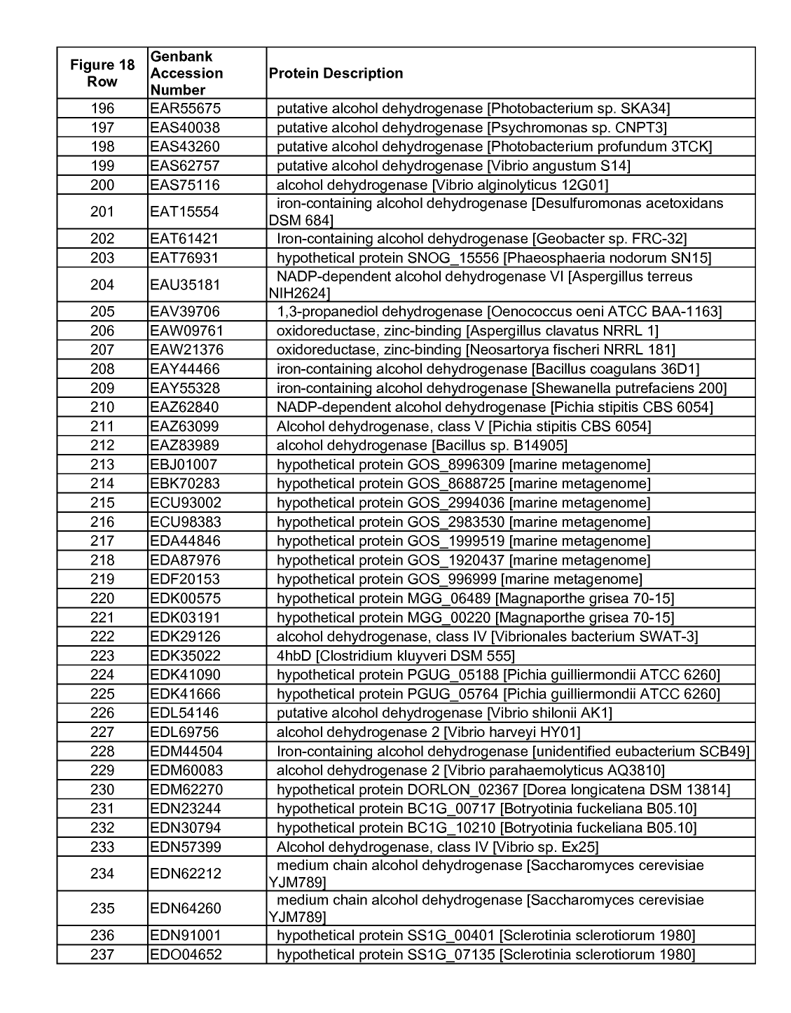

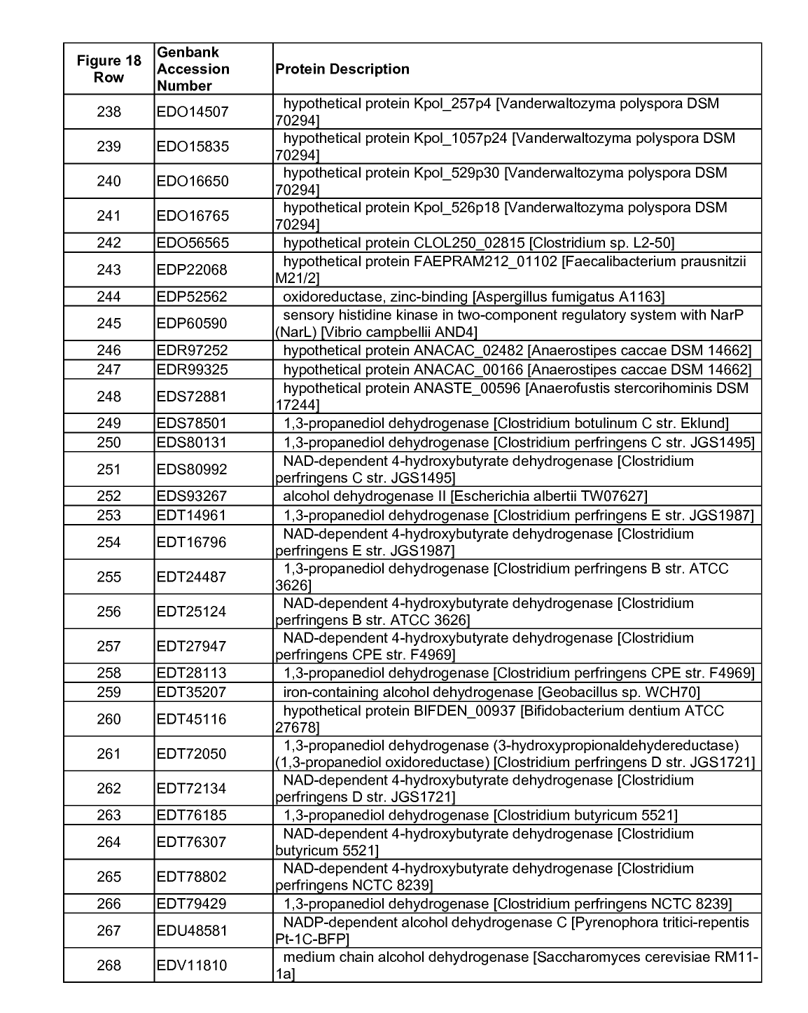

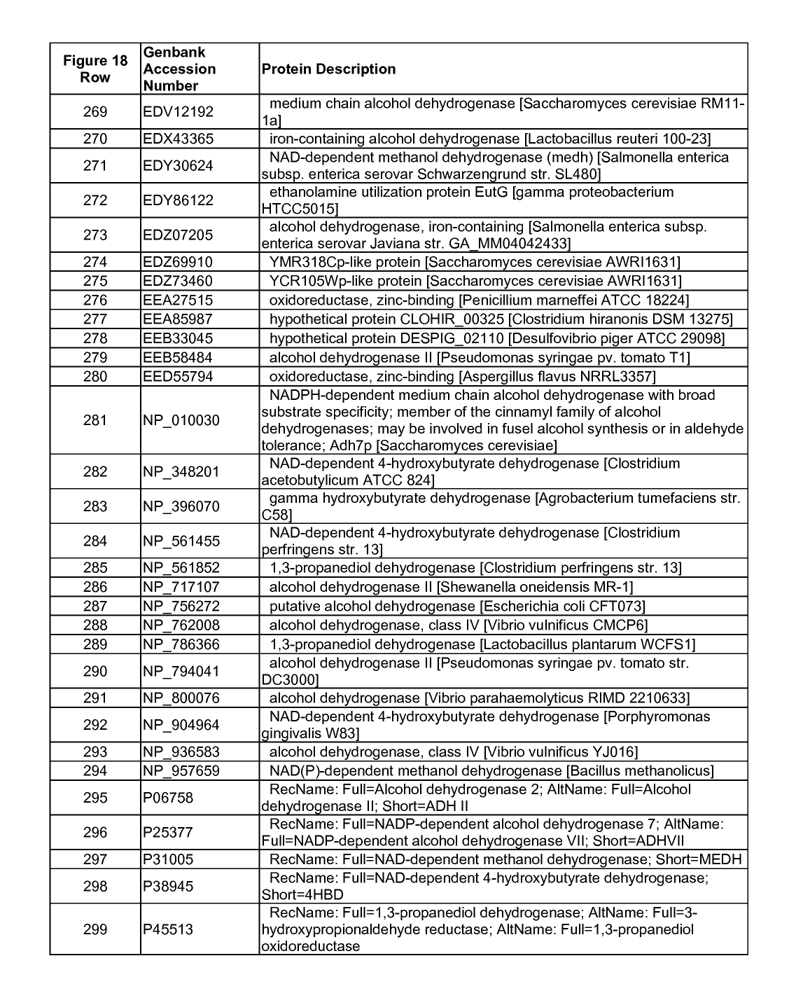

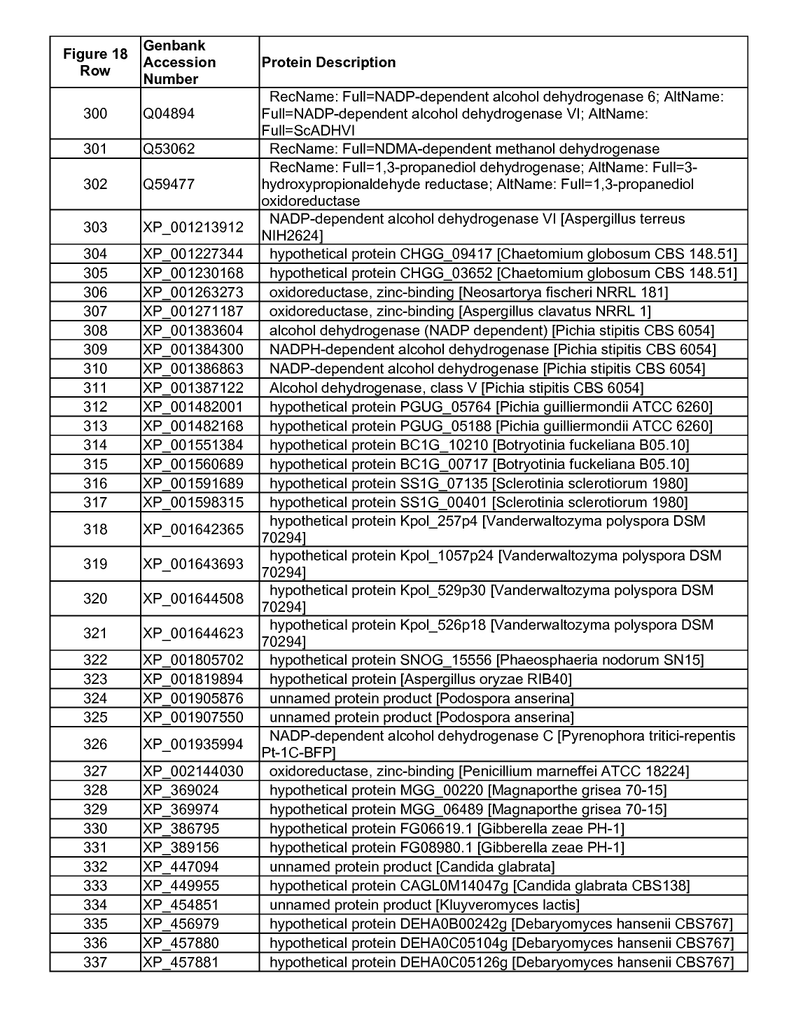

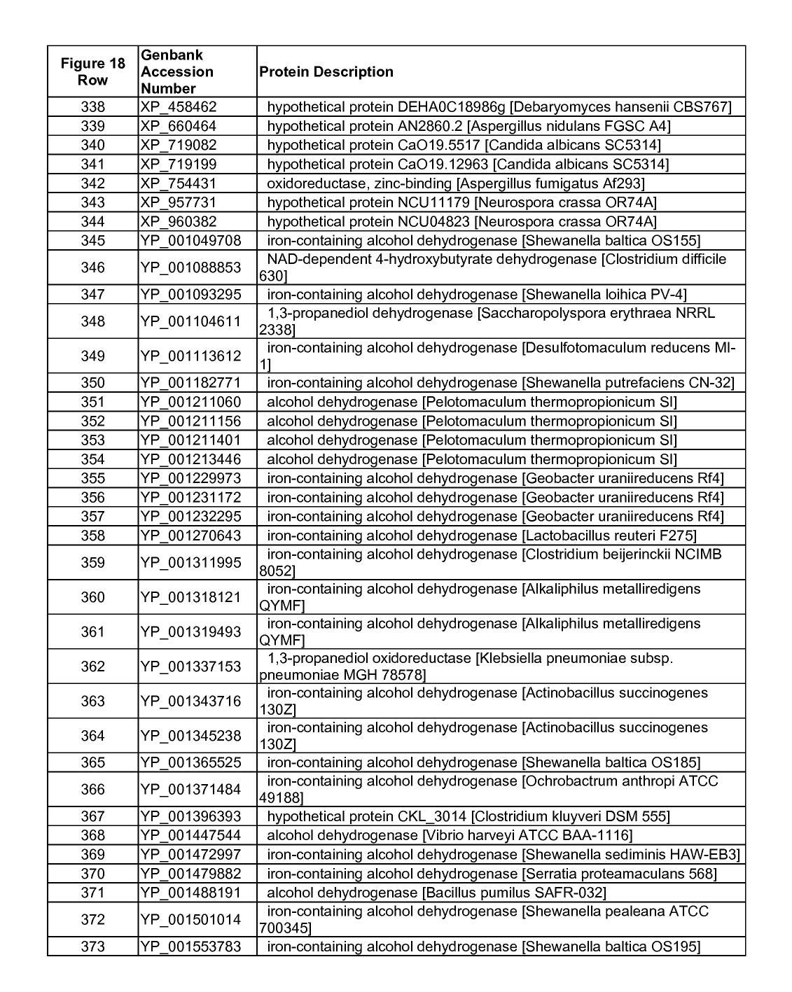

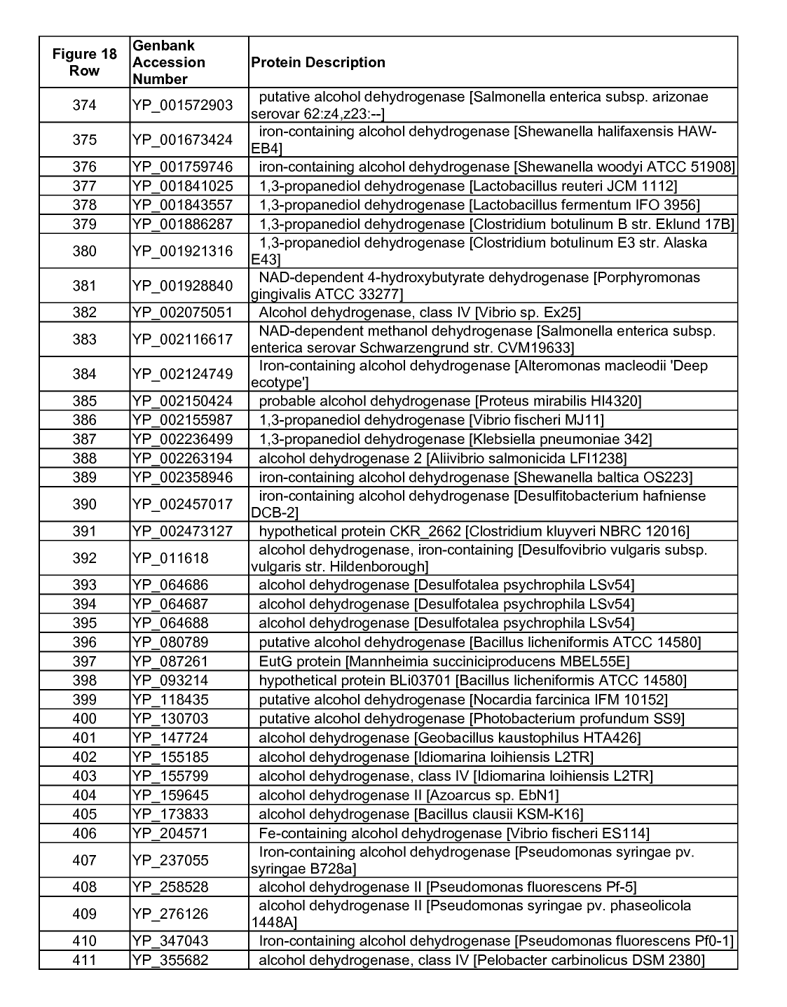

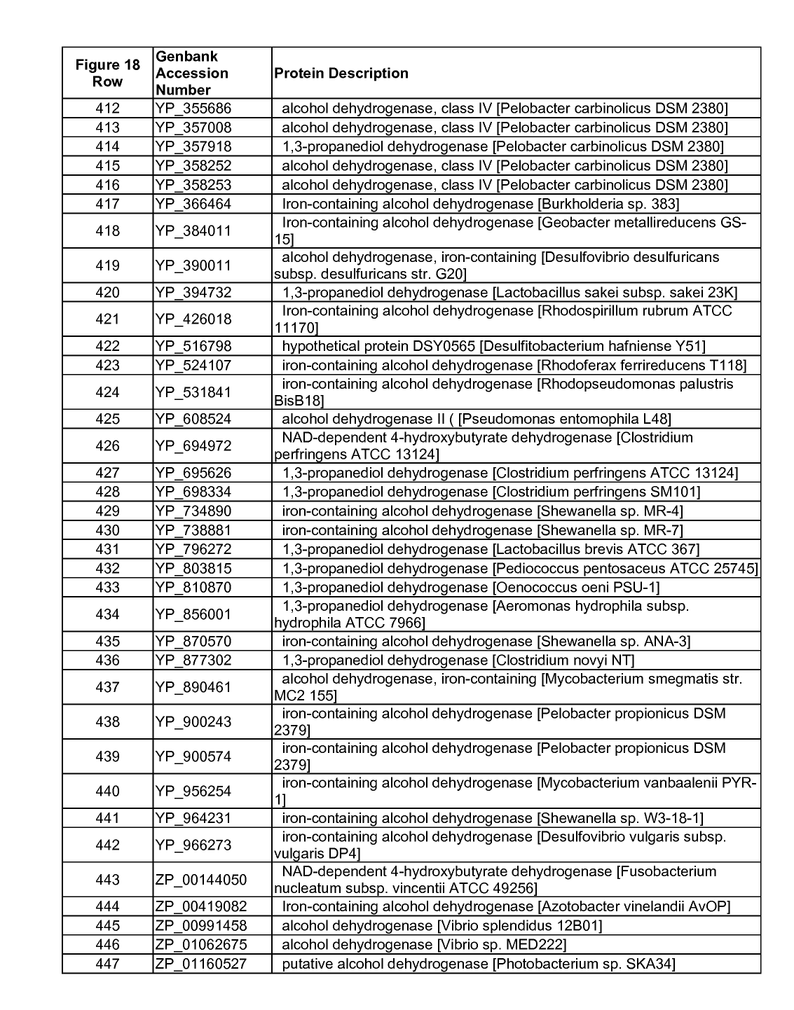

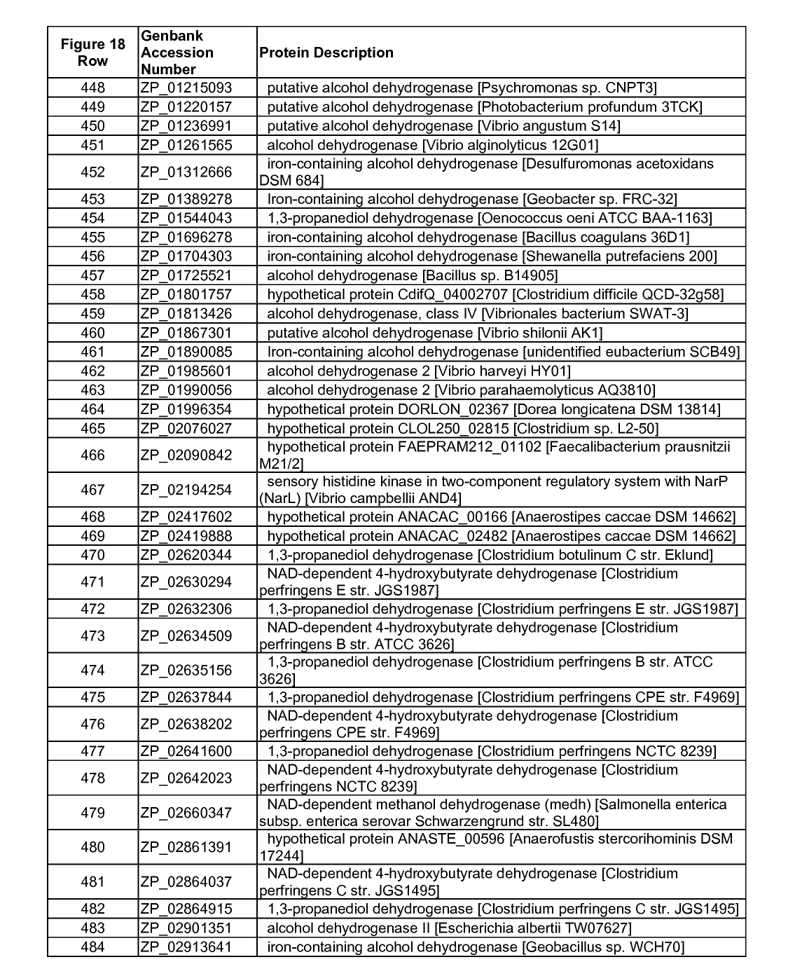

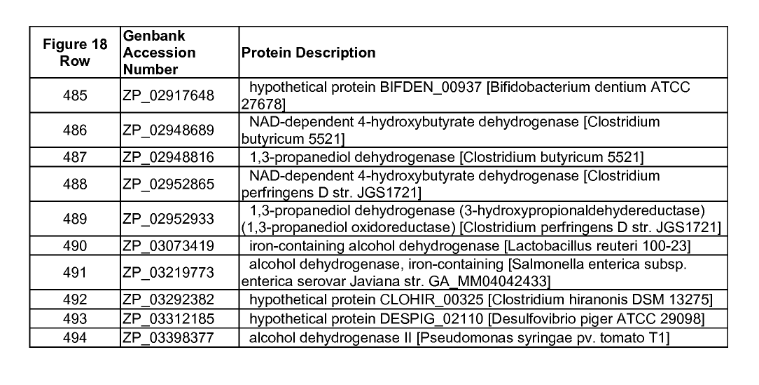

Oxidoreductases [EC 1.1.1.X] are enzymes that carry out oxidation/reduction reactions on alcohols or aldehydes using NAD/NADH or NADP/NADPH as a cofactor. Certain of these enzymes are capable of catalyzing conversion A6 in FIG. 1A. Examples of enzymes that may catalyze the conversion of 4-hydroxybutanal to butane-1,4-diol include certain 4-hydroxybutyrate dehydrogenases [EC 1.1.1.61], certain 1,3 propanediol dehydrogenases [EC 1.1.1.202], certain cinnamyl-alcohol dehydrogenases [EC:1.1.1.195], and certain alcohol dehydrogenases [EC 1.1.1.1] represented by the Genbank GI numbers in FIG. 1B and the corresponding amino acid sequences (SEQ ID NOs:44-50) as well as the polypeptides represented by the Genbank Accession numbers in FIG. 18. In some cases a useful oxidoreductase has an amino acid sequence that is at least about 75%, 85%, 90%, 95% or 100% identical to that of any of SEQ ID NOs:44-50 or the polypeptides represented by the Genbank Accession numbers in FIG. 18.

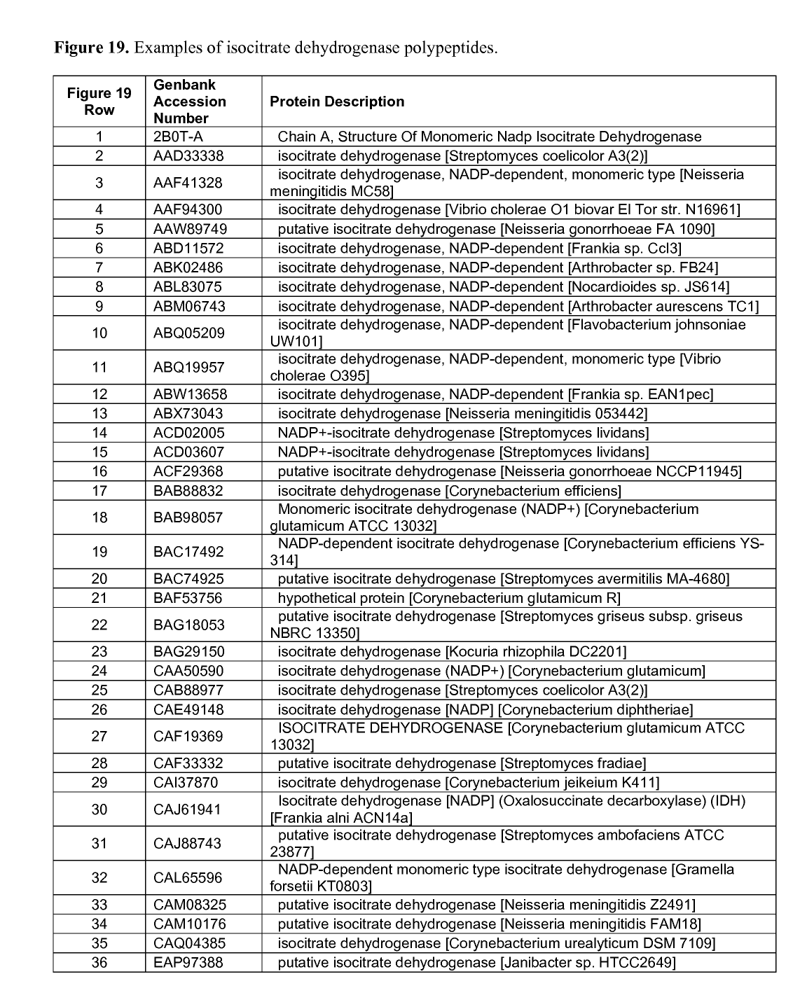

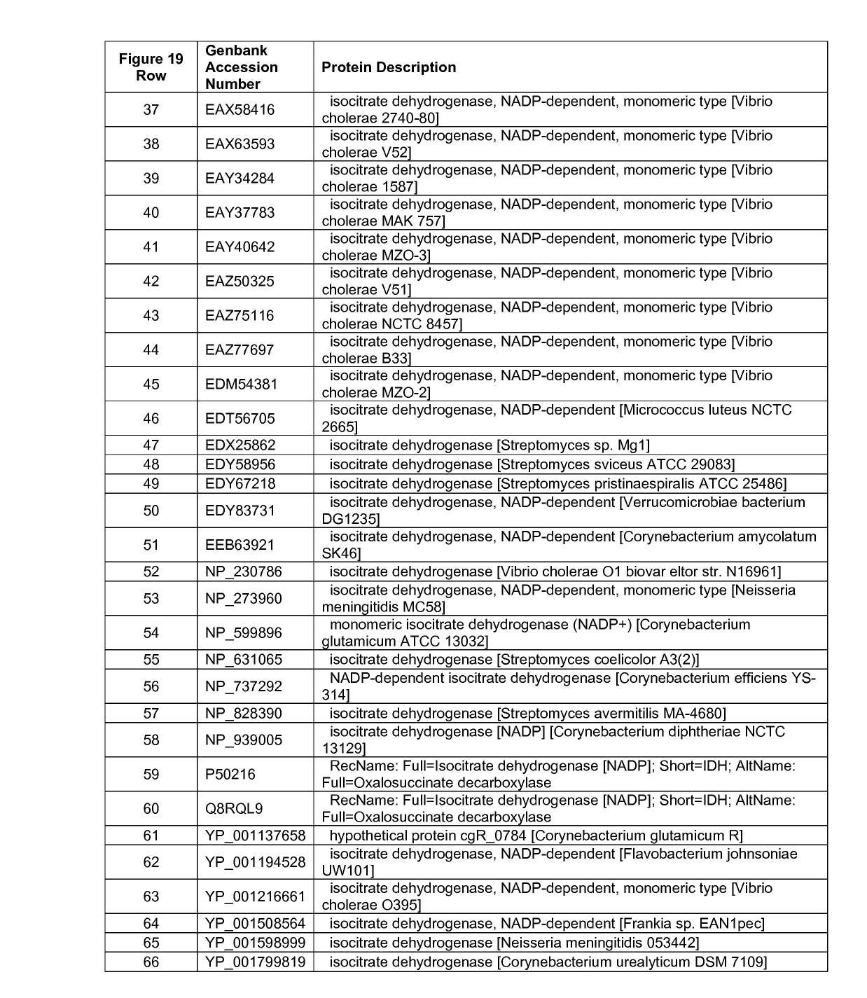

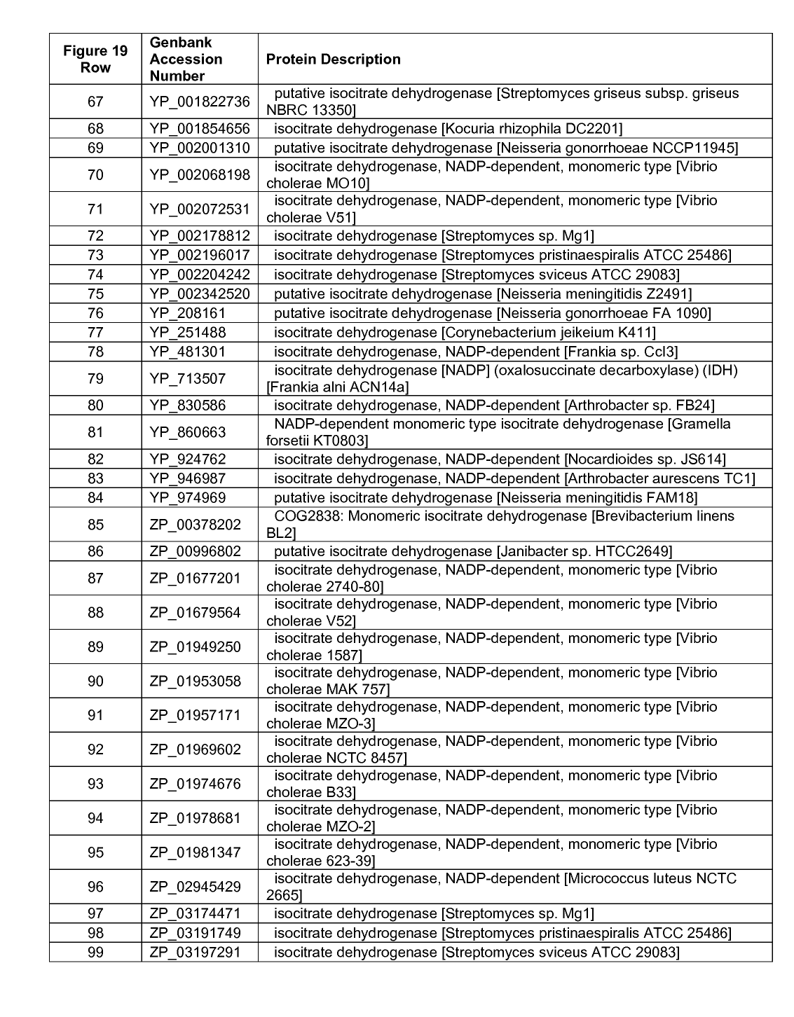

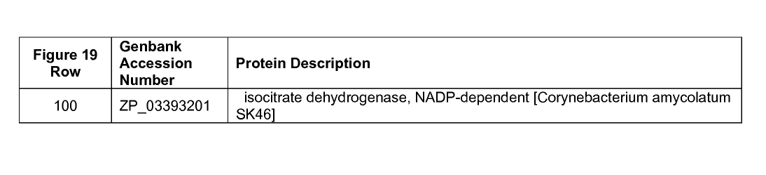

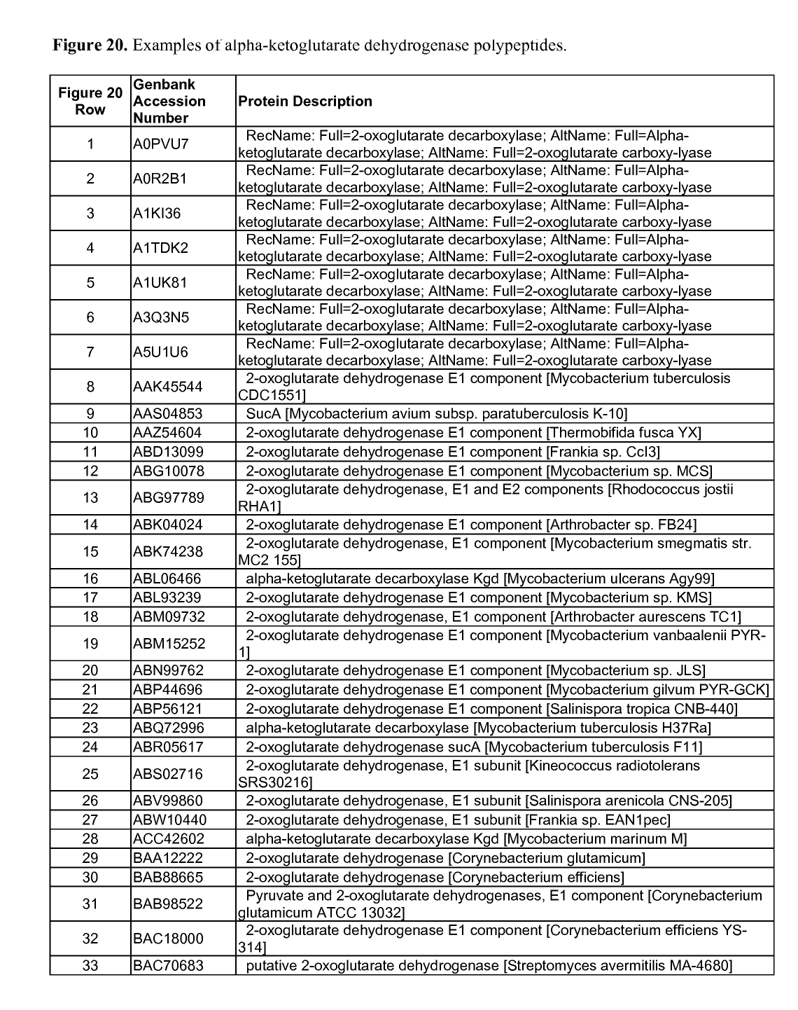

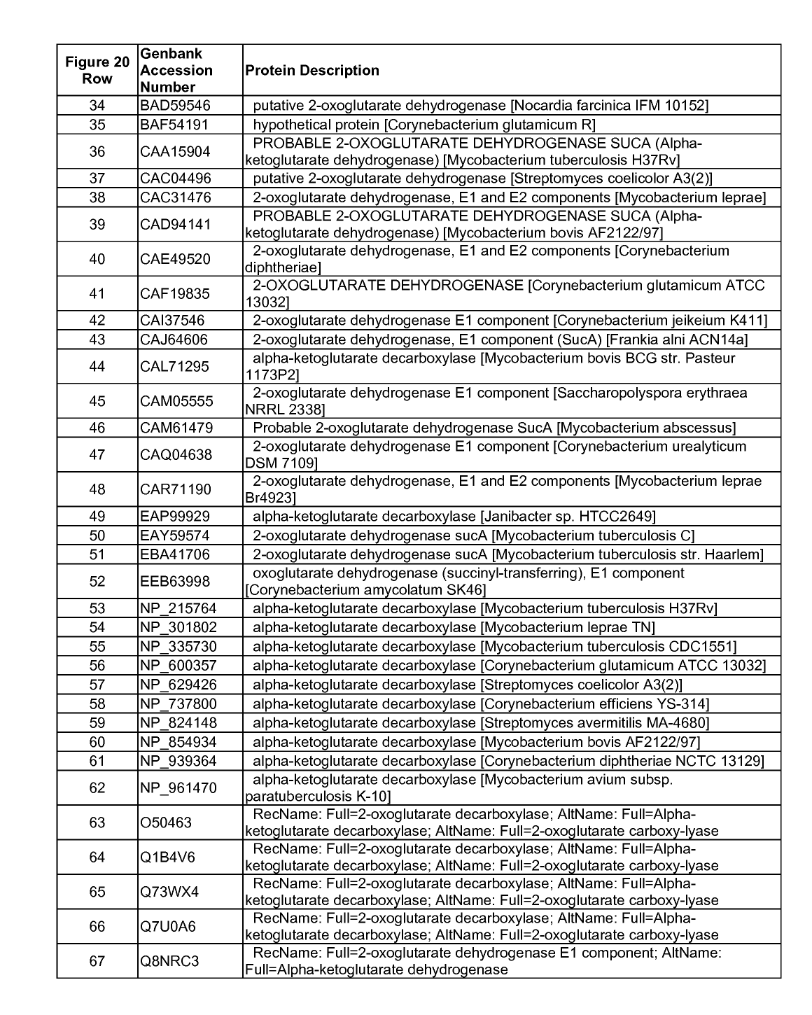

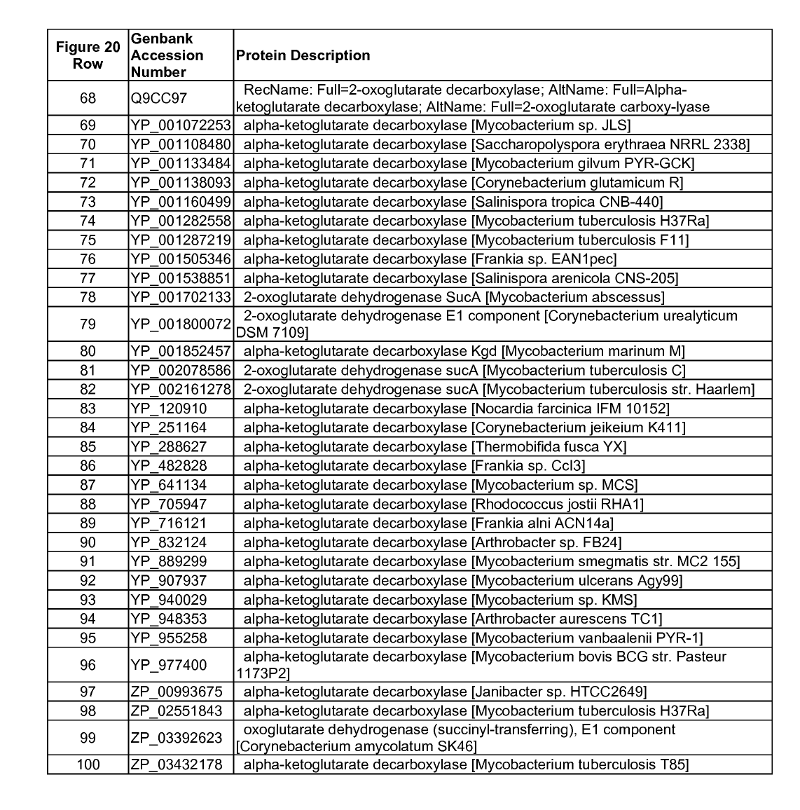

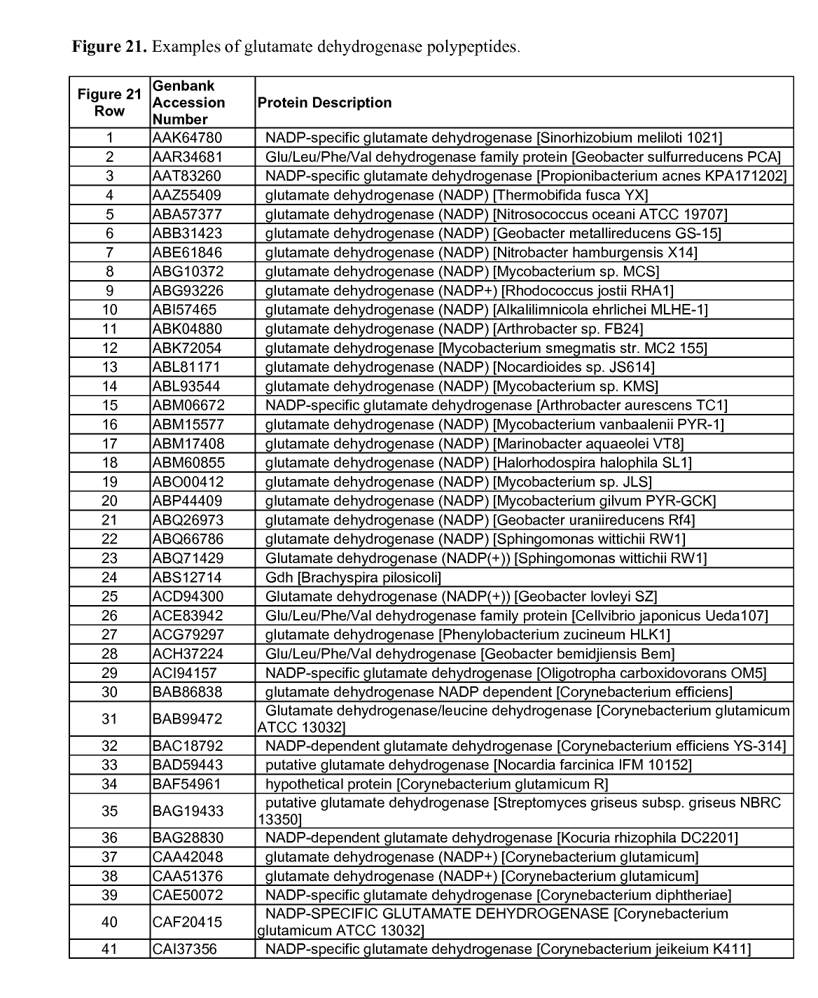

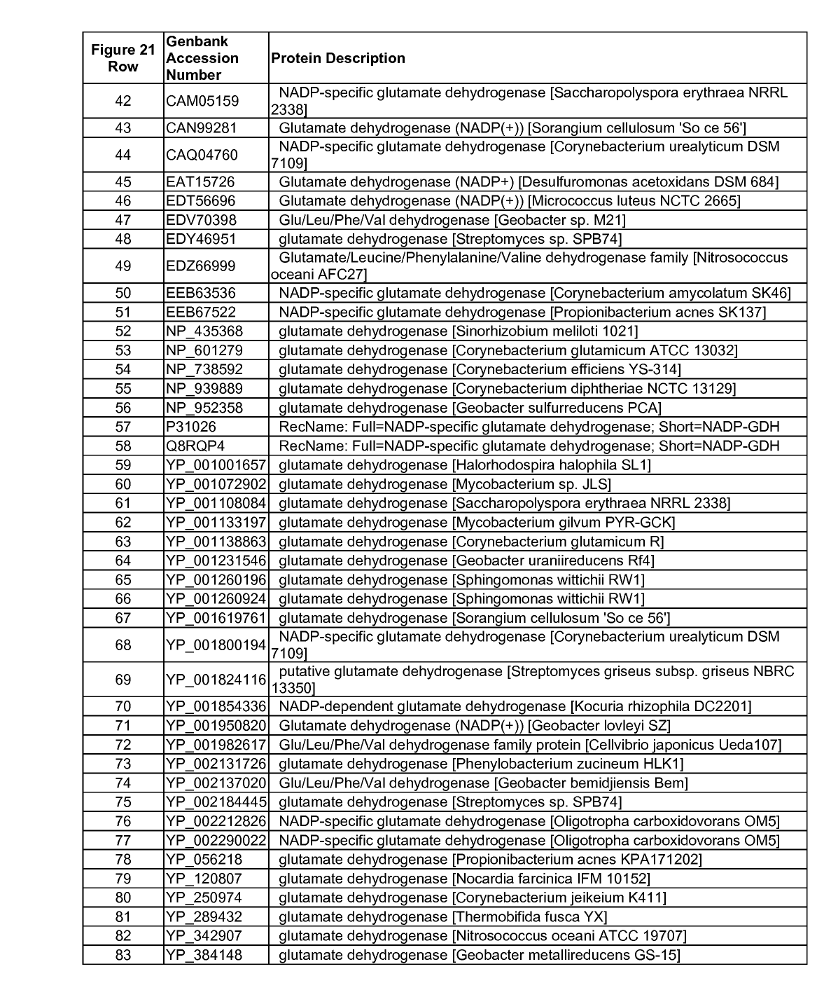

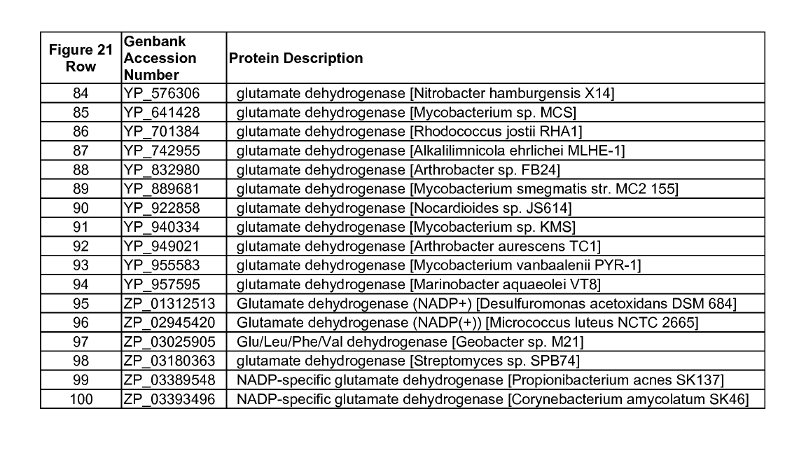

In some cases it is desirable to modify the recombinant cell to increase the production of one of the substrates used by reactions A1-A6 in addition to making one or more of the modifications that increase the activity or expression of polypeptides that catalyze reactions A1-A6. For example, a suitable modification can increase production of glutamate and/or proline. As another example, a modification can reduce the conversion of L-glutamate 5-semialdehyde to proline. Thus, the recombinant microbial cell can have a modification that decreases the activity or expression of one or more enzymes selected from: (a) an enzyme that catalyzes the conversion of alpha-ketoglutarate to isocitrate; (b) an enzyme that catalyzes the conversion of alpha-ketoglutarate to succinyl-CoA; (c) an enzyme that catalyzes the conversion of L-glutamate to alpha-ketoglutarate; (d) an enzyme that catalyzes the conversion of L-glutamate to L-glutamine; (e) an enzyme that catalyzes the conversion of L-glutamate to L-1-pyrroline 5-carboxylate; (f) an enzyme that catalyzes the conversion of L-proline to L-1-pyrroline 5-carboxylate (also called 1-pyrroline 5-carboxylate); and (g) an enzyme that catalyzes the conversion of L-1-pyrroline 5-carboxylate to L-proline. Another potentially useful modification is one that increases the activity or expression an enzyme that catalyzes the conversion of alpha-ketoglutarate to succinyl-CoA Other useful modifications include: a modification that increases the activity or expression of one or more enzymes selected from: (a) an enzyme that catalyzes the conversion of alpha-ketoglutarate to isocitrate; (b) an enzyme that catalyzes the conversion of alpha-ketoglutarate to succinyl CoA; and (c) an enzyme that catalyzes the conversion of L-glutamate to alpha-ketoglutarate. It can also be useful to modify the recombinant cell to increase expression or activity of one or more enzymes having at least about 75%, 85%, 90%, 95% or 100% identity to an enzyme represented by the Genbank Accession numbers in any of FIGS. 19-21.

### Engineering BDO Production

In most cases a host microorganism will need to be genetically modified to introduce one or more of: (a) a nucleic acid molecule encoding a polypeptide that catalyzes the conversion of L-glutamate to L-glutamate 5-phosphate; (b) a nucleic acid molecule encoding a polypeptide that catalyzes the conversion of L-glutamate 5-phosphate to L-glutamate 5-semialdehyde; (c) a nucleic acid molecule encoding a polypeptide that catalyzes the conversion of L-glutamate 5-semialdehyde to 5-hydroxy-L-norvaline; (d) a nucleic acid molecule encoding a polypeptide that catalyzes the conversion of 5-hydroxy-L-norvaline to 5-hydroxy-2-oxopentanoate; (e) a nucleic acid molecule encoding a polypeptide that catalyzes the conversion of 5-hydroxy-2-oxopentanoate to 4-hydroxybutanal; and (f) a nucleic acid molecule encoding a polypeptide that catalyzes the conversion of 4-hydroxybutanal to 1,4-butanediol. Thus, all of the nucleic acid molecules may be heterologous to the host. However, some host microorganisms may naturally harbor one or more such nucleic acid molecules, so that activity need not be introduced. In other such cases it may be desirable to modify the host nucleic acid sequence to increase expression or activity of the polypeptide. In other cases, the host organism may naturally harbor a nucleic acid molecule which, after nucleic acid sequence modification to alter the amino acid sequence, can catalyze the conversion of one or more of (a) L-glutamate to L-glutamate 5-phosphate; (b) L-glutamate 5-phosphate to L-glutamate 5-semialdehyde; (c) L-glutamate 5-semialdehyde to 5-hydroxy-L-norvaline; (d) 5-hydroxy-L-norvaline to 5-hydroxy-2-oxopentanoate; (e) 5-hydroxy-2-oxopentanoate to 4-hydroxybutanal; and (f) 4-hydroxybutanal to 1,4-butanediol. In yet other cases, a source organism may naturally harbor a nucleic acid molecule which, after nucleic acid sequence modification that alters the amino acid sequence, can catalyze the conversion of one or more of (a) L-glutamate to L-glutamate 5-phosphate; (b) L-glutamate 5-phosphate to L-glutamate 5-semialdehyde; (c) L-glutamate 5-semialdehyde to 5-hydroxy-L-norvaline; (d) 5-hydroxy-L-norvaline to 5-hydroxy-2-oxopentanoate; (e) 5-hydroxy-2-oxopentanoate to 4-hydroxybutanal; and (f) 4-hydroxybutanal to 1,4-butanediol. In these cases, the modified nucleic acid sequence can be introduced into the host organism to create an organism harboring a variant homologous nucleic acid molecule having the desired activity.

In general, any modification may be applied to a host cell to increase or impart production and/or accumulation of BDO or an intermediate in the production of BDO. In many cases, the modification comprises a genetic modification. In most cases, this will entail introducing into the host a nucleic acid molecule encoding a heterologous polypeptide that catalyzes one of conversions A1-A6 as shown in FIG. 1A. In general, genetic modifications may be introduced into cells by any available means of introducing nucleic acids (e.g., via transformation, transduction, mating, or conjugation). A nucleic acid to be introduced into a cell according to the present disclosure may be prepared by any available means. For example, it may be extracted from an organism's nucleic acids or synthesized by chemical means. Nucleic acids to be introduced into a cell may be, but need not be, in the context of a vector.

It is possible that a host cell will be modified to increase the activity or expression of a native polypeptide that catalyzes one or more of the conversions A1-A6 as shown in FIG. 1A.

Genetic modifications that increase activity of a polypeptide include, but are not limited to: introducing one or more copies of a gene encoding the polypeptide (which may differ from any gene already present in the host cell encoding a polypeptide having the same activity); altering a gene present in the cell to increase transcription or translation of the gene (e.g., altering, adding additional sequence to, deleting sequence from, replacement of one or more nucleotides, or swapping for example, a promoter, regulatory or other sequence); and altering the sequence (e.g. coding or non-coding) of a gene encoding the polypeptide to increase activity (e.g., by increasing catalytic activity, reducing inhibition, targeting a specific subcellular location, increasing mRNA stability, increasing protein stability, altering specificity, enhancing sensitivity to small molecule or other modulators of polypeptide activity).

Genetic modifications that decrease activity of a polypeptide include, but are not limited to: deleting all or a portion of a gene (e.g., all or a portion of the coding sequence or all or a portion of a regulatory sequence) encoding the polypeptide; inserting a nucleic acid sequence that disrupts a gene (e.g., disrupts the coding sequence of the gene or disrupts the regulatory sequence) encoding the polypeptide; altering a gene present in the cell to decrease transcription or translation of the gene or stability of the mRNA or polypeptide encoded by the gene (for example, by altering, adding additional sequence to, deleting sequence from, replacement of one or more nucleotides, or swapping for example, a promoter, regulatory or other sequence).

A vector for use in accordance with the present methods can be a plasmid, linear piece of DNA, a cosmid, or a yeast artificial chromosome, among others known in the art to be appropriate for use in microorganisms. A vector can comprise an origin of replication, which allows the vector to be passed on to progeny cells of a parent cell comprising the vector. As examples of episomal vectors, vectors can be constructed using derivatives of the cryptic Corynebacterium glutamicum low-copy pBL1 plasmid (see Santamaria et al. J. Gen. Microbiol. 130:2237-2246, 1984). These episomal plasmids contain sequences that encode a replicase and sequences corresponding to an origin of replication, which enable replication of the plasmid within C. glutamicum; therefore, these plasmids can be propagated without integration into the chromosome. Alternatively, if integration of the vector into the host cell genome is desired, the vector can comprise sequences that direct such integration (e.g., specific sequences or regions of homology, etc.). For example, vectors can be designed to inactivate native C. glutamicum genes by gene deletion. In some instances, these constructs both delete native genes and insert heterologous genes into the host chromosome at the locus of the deletion event. Deletion plasmids contain nucleotide sequences homologous to regions upstream and downstream of the gene that is the target for the deletion event; in some instances these sequences include small amounts of coding sequence of the gene that is to be inactivated. These flanking sequences are used to facilitate homologous recombination. Single cross-over events target the plasmid into the host chromosome at sites upstream or downstream of the gene to be deleted. Deletion plasmids also contain the sacB gene, encoding the levansucrase gene from Bacillus subtilis. During growth of transformants upon medium containing sucrose, sacB allows for positive selection for recombination events, resulting in either the restoration of the native locus or the desired deletion event which retains the cassette that directs the expression of a particular gene of interest (see Jager, W., et al. J. Bacteriol. 174:5462-5465, 1992).

Nucleic acids to be introduced into a cell may be so introduced together with at least one detectable marker (e.g., a screenable or selectable marker). In some embodiments, a single nucleic acid molecule to be introduced may include both a sequence of interest (e.g., a gene encoding a polypeptide of interest as described herein) and a detectable marker. In general, a detectable marker allows cells that have received an introduced nucleic acid to be distinguished from those that have not. For example, a selectable marker may allow transformed cells to survive on a medium comprising an antibiotic lethal to an untransformed microorganism, or may allow transformed cells to metabolize a component of the medium into a product not produced by untransformed cells, among other phenotypes.

As will be appreciated, nucleic acids can be introduced into cells by any available means including, for example, chemical-mediated transformation, particle bombardment, electroporation, etc.

Nucleic acids to be expressed in a cell are typically in operative association with one or more expression sequences such as, for example, promoters, terminators, and/or other regulatory sequences. Any such regulatory sequences that are active in the host cell (including, for example, homologous or heterologous host sequences, constitutive, inducible, or repressible host sequences, etc.) can be used.

A promoter, as is known, is a DNA sequence that can direct the transcription of a nearby coding region. A promoter can be constitutive, inducible or repressible. Constitutive promoters continually direct the transcription of a nearby coding region. Some inducible promoters can be induced by the addition to the medium of an appropriate inducer molecule, which will be determined by the identity of the promoter. Some repressible promoters can be repressed by the addition to the medium of an appropriate repressor molecule, which will be determined by the identity of the promoter. In some cases, a promoter can be induced or repressed by culturing the cells at a certain temperature (e.g., heat or cold regulated promoters) or by the exhaustion of a component of the medium. In some cases, a promoter can be induced or repressed by culturing when the cell culture reaches a certain growth phase (e.g., growth phase dependent promoters).

When the host cell is a fungus, representative useful promoters include, for example, the constitutive S. cerevisiae triosephosphate isomerase (TPI) promoter, the S. cerevisiae glyceraldehyde-3-phosphate dehydrogenase (isozyme 3) TDH3 promoter, the S. cerevisiae TEE1 promoter and the S. cerevisiae ADH1 promoter. Representative terminators for use in accordance with the present disclosure include, for example, S. cerevisiae CYC1.

When the host cell is a bacterium, representative useful promoters include, for example, the lac, trc, trcRBS, phoA, tac, or lambda P_{L}/lambda P_{R} promoter from E. coli (or derivatives thereof) or the phoA, gpd, rplM, or rpsJ promoter from a coryneform bacteria.

As noted above, a host cell can be engineered to harbor nucleic acid molecules encoding heterologous polypeptides. Any organism (“source organism”) that naturally contains a relevant polypeptide or genetic sequences may be used as source for the heterologous polypeptide. Representative source organisms include, for example, mammalian, insect, amphibian, plant, fungal, yeast, algal, bacterial, archaebacterial, cyanobacterial, and protozoan source organisms.

For example a source organism may be a fungus, including but not limited to of the genus Aspergillus, Aureobasidium, Brettanomyces, Candida, Cryptococcus, Debaromyces, Hansenula, Kloeckera, Kluyveromyces, Lipomyces, Nadsonia, Phaffia, Pichia, Rhodotorula, Saccharomyces, Schizosaccharomyces, Schwanniomyces, Torulopsis, Trichosporon, Trigonopsis, Yarrowia or Zygosaccharomyces. In certain embodiments, the source organism may be of the species Aspergillus niger, Aspergillus oryzae, Candida albicans, Candida albicans SC5314, Candida sphaerica, Hansenula anomala, Kluyveromyces lactis, Saccharomyces boulardii, Saccharomyces cerevisiae, Saccharomyces cerevisiae var bayanus (e.g. Lalvin DV10), Schizosaccharomyces malidevorans, Schizosaccharomyces pombe, Yarrowia lipolytica or Yarrowia lipolytica CLIB122.

For example a source organism may be a bacterium, including a gram positive, gram negative or archaebacterium, of the genus Achromobacter, Acinetobacter, Actinobacillus, Alcaligenes, Anabaena, Ancylobacter, Arthrobacter, Azotobacter, Bacillus, Brevibacterium, Cellulomonas, Clostridium, Corynebacterium, Deinococcus, Enterococcus, Erwinia, Escherichia, Klebsiella, Lactobacillus, Lactococcus, Methanomonas, Methanothermobacter, Methylobacterium, Microbacterium, Micrococcus, Nocardia, Nocardioides, Nostoc, Paenibacillus, Propionibacterium, Pseudomonas, Ralstonia, Renibacterium, Rhodococcus, Salmonella, Streptococcus, Streptomyces, Thermus, (Thermo)synechococcus or Zymomonas. In certain embodiments, the source organism may be of the species Achromobacter methanolophila, Achromobacter pestifer, Acinetobacter baumannii, Acinetobacter baumannii ACICU, Actinobacillus pleuropneumoniae, Actinobacillus succinogenes, Alcaligenes eutrophus, Anabaena variabilis, Anabaena variabilis (ATCC 29413), Arthrobacter aurescens, Arthrobacter aurescens TC1 (ATCC BAA-1386), Arthrobacter chlorophenolicus, Arthrobacter chlorophenolicus A6 (DSM 402-4171), Arthrobacter hydrocarboglutamicus, Arthrobacter paraffineus, Arthrobacter protophormiae, Arthrobacter roseoparaffinus, Azotobacter vinelandii, Azotobacter vinelandii AvOP, Azotobacter vinelandii AvOP (ATCC BAA-1303), Bacillus cereus, Bacillus cereus (ATCC 14579), Bacillus circulans, Bacillus licheniformis, Bacillus megaterium, Bacillus subtilis, Brevibacterium album, Brevibacterium cerinum, Brevibacterium immariophilium, Brevibacterium ketoglutamicum, Brevibacterium roseum, Brevibacterium saccharolyticum, Brevibacterium thiogenitalis, Citrobacter freundii, Citrobacter freundii (DSM 30040), Clostridium perfringens, Clostridium perfringens SM101, Corynebacterium acetoacidophilum, Corynebacterium acetoglutamicum, Corynebacterium alkanolyticum, Corynebacterium callunae, Corynebacterium glutamicum, Corynebacterium glutamicum (ATCC 13032), Corynebacterium herculis, Corynebacterium melassecola, Corynebacterium petrophilum, Corynebacterium urealyticum, Corynebacterium urealyticum (DSM 7109) (ATCC 43042), Deinococcus geothermalis, Deinococcus geothermalis DSM 11300, Enterococcus faecalis, Enterococcus faecalis V583, Enterococcus faecium, Enterococcus gallinarium, Erwinia carotovora, Erwinia chrysanthemi, Escherichia coli, Gluconacetobacter diazotrophicus, Gluconacetobacter diazotrophicus PAl 5, Lactobacillus casei, Lactobacillus casei (ATCC 334), Lactobacillus plantarum, Lactobacillus plantarum WCFS1 (ATCC BAA-793), Lactobacillus sakei, Lactobacillus sakei subsp. sakei 23K, Lactococcus lactis, Lactococcus lactis cremoris MG1363, Lactococcus lactis NIZO B 1157, Lactococcus lactis subsp. Lactis strain, Lactococcus lactis subsp. lactis strain IFPL730, Listeria monocytogenes EGD-e, Methanomonas methylovora, Methanothermobacter thermautotrophicus, Methanothermobacter thermautotrophicus str. Delta H, Microbacterium ammoniaphilum, Micrococcus luteus, Nocardia farcinica, Nocardia farcinica IFM 10152, Nocardia globerula, Nocardia sp. JS614, Nocardioides simplex, Nodularia spumigena, Nodularia spumigena CCY 9414, Nostoc punctiforme, Nostoc punctiforme PCC 73102 (ATCC 29133), Nostoc sp. (ATCC 53789), Nostoc sp. GSV224, Paenibacillus macerans, Propionibacterium acnes, Propionibacterium acnes KPA171202 (DSM 16379), Pseudomonas dacunhae, Pseudomonas fluorescens PfO-1, Pseudomonas insueta, Pseudomonas methanolica, Pseudomonas putida, Pseudomonas putida ATCC 12633, Pseudomonas sp., Ralstonia pickettii, Ralstonia pickettii 12J, Renibacterium salmoninarum, Renibacterium salmoninarum ATCC 33209, Rhodococcus equi, Rhodococcus erythropolis, Rhodococcus sp. RHA1, Staphylococcus epidermidis RP62A, Staphylococcus saprophyticus subsp. saprophyticus (ATCC 15305), Streptococcus bovis, Streptomyces coelicolor A3(2), Streptomyces tanashiensis, (Thermo)synechococcus vulcanus, Thermosinus carboxydivorans Nor1 (DSM 14886), Thermus thermophilus, Thermus thermophilus HB27, Zymomonas mobilis subsp. mobilis ZM4 (ATCC 31821) or Zymomonas mobilis.

For example a source organism may be a plant of the genus Arabidopsis, Brassica or Triticum. In certain embodiments, the source organism may be of the species Arabidopsis thaliana, Brassica napus or Triticum secale.

For example a source organism may be a mammal of the genus Rattus, Mus or Homo. In certain embodiments, the source organism may be of the species Rattus norvegicus, Mus musculus or Homo sapiens.

In some cases, where nucleic acid sequences originating from a source heterologous to the host cell are utilized, such sequences may be modified, for example, to adjust for codon preferences and/or other expression-related aspects (e.g., linkage to promoters and/or other regulatory sequences active in the host cell, removing or inserting specific restriction enzyme sites for more convenient cloning, etc.) of the host cell system.

Any of a variety of host cells that do not naturally produce BDO may be engineered to produce BDO. It will often be desirable to utilize cells that are amenable to manipulation, particularly genetic manipulation, as well as to growth on large scale and under a variety of conditions. In many cases, it will be desirable to utilize host cells that are amenable to growth under anaerobic conditions, microaerobic conditions, and/or under conditions of relatively low pH. In many cases, it will be desirable to utilize bacterial, yeast or fungal host cells. Bacterial cells that are glutamate producers, particularly those having high glutamate flux, are useful. It is also desirable for the host be tolerant to relatively high levels of BDO and/or one or more of: L-glutamate, L-glutamate 5-phosphate, 5-oxopyrrolidine-2-carboxylate, L-glutamate 5-semialdehyde, pyrroline-5-carboxylate, 5-hydroxy-L-norvaline, 5-hydroxy-2-oxopentanoate, and 4-hydroxybutanal. Particularly useful host organisms include C. glutamicum and E. coli.

Bacteria, for example, gram positive, gram negative or archaebacteria, can be used as a host organism, e.g., the genus can be Achromobacter, Acinetobacter, Actinobacillus, Alcaligenes, Anabaena, Ancylobacter, Arthrobacter, Azotobacter, Bacillus, Brevibacterium, Cellulomonas, Citrobacter, Clostridium, Corynebacterium, Deinococcus, Enterococcus, Erwinia, Escherichia, Gluconacetobacter, Klebsiella, Lactobacillus, Lactococcus, Listeria, Methanomonas, Methanothermobacter, Methylobacterium, Microbacterium, Micrococcus, Nocardia, Nocardioides, Nodularia, Nostoc, Paenibacillus, Propionibacterium, Pseudomonas, Ralstonia, Renibacterium, Rhodococcus, Salmonella, Staphylococcus, Streptococcus, Streptomyces, (Thermo)synechococcus, Thermosinus, Thermus, or Zymomonas. In certain embodiments, the host organism may be of the species Achromobacter methanolophila, Achromobacter pestifer, Acinetobacter baumannii, Acinetobacter baumannii ACICU, Actinobacillus pleuropneumoniae, Actinobacillus succinogenes, Alcaligenes eutrophus, Anabaena variabilis, Anabaena variabilis (ATCC 29413), Arthrobacter aurescens, Arthrobacter aurescens TC1 (ATCC BAA-1386), Arthrobacter chlorophenolicus, Arthrobacter chlorophenolicus A6 (DSM 402-4171), Arthrobacter hydrocarboglutamicus, Arthrobacter paraffineus, Arthrobacter protophormiae, Arthrobacter roseoparaffinus, Azotobacter vinelandii, Azotobacter vinelandii AvOP, Azotobacter vinelandii AvOP (ATCC BAA-1303), Bacillus cereus, Bacillus cereus (ATCC 14579), Bacillus circulans, Bacillus licheniformis, Bacillus megaterium, Bacillus subtilis, Brevibacterium album, Brevibacterium cerinum, Brevibacterium immariophilium, Brevibacterium ketoglutamicum, Brevibacterium roseum, Brevibacterium saccharolyticum, Brevibacterium thiogenitalis, Citrobacter freundii, Citrobacter freundii (DSM 30040), Clostridium perfringens, Clostridium perfringens SM101, Corynebacterium acetoacidophilum, Corynebacterium acetoglutamicum, Corynebacterium alkanolyticum, Corynebacterium callunae, Corynebacterium glutamicum, Corynebacterium glutamicum (ATCC 13032), Corynebacterium herculis, Corynebacterium melassecola, Corynebacterium petrophilum, Corynebacterium urealyticum, Corynebacterium urealyticum (DSM 7109) (ATCC 43042), Deinococcus geothermalis, Deinococcus geothermalis DSM 11300, Enterococcus faecalis, Enterococcus faecalis V583, Enterococcus faecium, Enterococcus gallinarium, Erwinia carotovora, Erwinia chrysanthemi, Escherichia coli (for example, including but not limited to E. coli K12 (ATCC 10798), E. coli 55-25 (ATCC 25208), E. coli W6 (ATCC25377) and E. coli W157 (ATCC 25378), Gluconacetobacter diazotrophicus, Gluconacetobacter diazotrophicus PAl 5, Lactobacillus casei, Lactobacillus casei (ATCC 334), Lactobacillus plantarum, Lactobacillus plantarum WCFS1 (ATCC BAA-793), Lactobacillus sakei, Lactobacillus sakei subsp. sakei 23K, Lactococcus lactis, Lactococcus lactis cremoris MG1363, Lactococcus lactis NIZO B1157, Lactococcus lactis subsp. Lactis strain, Lactococcus lactis subsp. lactis strain IFPL730, Listeria monocytogenes EGD-e, Methanomonas methylovora, Methanothermobacter thermautotrophicus, Methanothermobacter thermautotrophicus str. Delta H, Microbacterium ammoniaphilum, Micrococcus luteus, Nocardia farcinica, Nocardia farcinica IFM 10152, Nocardia globerula, Nocardia sp. JS614, Nocardioides simplex, Nodularia spumigena, Nodularia spumigena CCY 9414, Nostoc punctiforme, Nostoc punctiforme PCC 73102 (ATCC 29133), Nostoc sp. (ATCC 53789), Nostoc sp. GSV224, Paenibacillus macerans, Propionibacterium acnes, Propionibacterium acnes KPA171202 (DSM 16379), Pseudomonas dacunhae, Pseudomonas fluorescens PfO-1, Pseudomonas insueta, Pseudomonas methanolica, Pseudomonas putida, Pseudomonas putida ATCC 12633, Pseudomonas sp., Ralstonia pickettii, Ralstonia pickettii 12J, Renibacterium salmoninarum, Renibacterium salmoninarum ATCC 33209, Rhodococcus equi, Rhodococcus erythropolis, Rhodococcus sp. RHA1, Staphylococcus epidermidis RP62A, Staphylococcus saprophyticus subsp. saprophyticus (ATCC 15305), Streptococcus bovis, Streptomyces coelicolor A3(2), Streptomyces tanashiensis, (Thermo)synechococcus vulcanus, Thermosinus carboxydivorans Nor1 (DSM 14886), Thermus thermophilus, Thermus thermophilus HB27, Zymomonas mobilis subsp. mobilis ZM4 (ATCC 31821) or Zymomonas mobilis.

Fungi can be used as a host organism, e.g., the genus can be Aspergillus, Aureobasidium, Brettanomyces, Candida, Cryptococcus, Debaromyces, Hansenula, Kloeckera, Kluyveromyces, Lipomyces, Nadsonia, Phaffia, Pichia, Rhodotorula, Saccharomyces, Schizosaccharomyces, Schwanniomyces, Torulopsis, Trichosporon, Trigonopsis, Yarrowia or Zygosaccharomyces. In certain embodiments, the host organism may be of the species Aspergillus niger, Aspergillus oryzae, Candida albicans, Candida albicans SC5314, Candida sphaerica, Hansenula anomala, Kluyveromyces lactis, Saccharomyces boulardii, Saccharomyces cerevisiae, Saccharomyces cerevisiae var bayanus (e.g. Lalvin DV10), Schizosaccharomyces malidevorans, Schizosaccharomyces pombe, Yarrowia lipolytica or Yarrowia lipolytica CLIB122.

In certain cases, the host organism is Corynebacterium glutamicum, Corynebacterium glutamicum ATCC 13032, or is a glutamate or proline producing strain from TABLE 1. In other cases, the host organism is an E. coli strain, for example an E. coli strain from TABLE 1. In certain cases, the host organism can be further modified to have increased glutamate flux and/or increased tolerance to one or more of BDO, L-glutamate, L-glutamate 5-phosphate, 5-oxopyrrolidine-2-carboxylate, L-glutamate 5-semialdehyde, pyrroline-5-carboxylate, 5-hydroxy-L-norvaline, 5-hydroxy-2-oxopentanoate, and 4-hydroxybutanal.

TABLE 1
Potential Host Organisms available at the ATCC ® or NRRL

| Row | Strain | organism |
|---|---|---|
| 1 | ATCC 21275 | Achromobacter methanolophila |
| 2 | ATCC 15445 | Achromobacter pestifer |
| 3 | ATCC 15446 | Alcaligenes sp. |
| 4 | ATCC 21373 | Ancylobacter sp. |
| 5 | ATCC 15583 | Arthrobacter hydrocarboglutamicus |
| 6 | ATCC 15591 | Arthrobacter paraffineus |
| 7 | ATCC 19064 | Arthrobacter paraffineus |
| 8 | ATCC 19065 | Arthrobacter paraffineus |
| 9 | ATCC 21535 | Arthrobacter paraffineus |
| 10 | ATCC 17775 | Arthrobacter protophormiae |
| 11 | ATCC 15584 | Arthrobacter roseoparaffinus |
| 12 | ATCC 13403 | Bacillus circulans |
| 13 | ATCC 13402 | Bacillus megaterium |
| 14 | ATCC 15177 | Bacillus megaterium |
| 15 | ATCC 15781 | Bacillus megaterium |
| 16 | ATCC 13062 | Bacillus sp. |
| 17 | ATCC 15111 | Brevibacterium album |
| 18 | ATCC 15112 | Brevibacterium cerinum |
| 19 | ATCC 14068 | Brevibacterium immariophilium |
| 20 | ATCC 15587 | Brevibacterium ketoglutamicum |
| 21 | ATCC 15588 | Brevibacterium ketoglutamicum |
| 22 | ATCC 21533 | Brevibacterium ketoglutamicum |
| 23 | ATCC 13825 | Brevibacterium roseum |
| 24 | ATCC 14066 | Brevibacterium saccharolyticum |
| 25 | ATCC 19240 | Brevibacterium thiogenitalis |
| 26 | ATCC 31230 | Cellulomonas sp. |
| 27 | ATCC 31231 | Cellulomonas sp. |
| 28 | ATCC 31232 | Cellulomonas sp. |
| 29 | ATCC 13870 | Corynebacterium acetoacidophilum |
| 30 | ATCC 15806 | Corynebacterium acetoglutamicum |
| 31 | ATCC 21511 | Corynebacterium alkanolyticum |
| 32 | ATCC 15991 | Corynebacterium callunae |
| 33 | ATCC 13032 | Corynebacterium glutamicum |
| 34 | ATCC 13058 | Corynebacterium glutamicum |
| 35 | ATCC 13059 | Corynebacterium glutamicum |
| 36 | ATCC 13060 | Corynebacterium glutamicum |
| 37 | ATCC 13655 | Corynebacterium glutamicum |
| 38 | ATCC 13761 | Corynebacterium glutamicum |
| 39 | ATCC 13869 | Corynebacterium glutamicum |
| 40 | ATCC 14020 | Corynebacterium glutamicum |
| 41 | ATCC 15990 | Corynebacterium glutamicum |
| 42 | ATCC 21157 | Corynebacterium glutamicum |
| 43 | ATCC 21158 | Corynebacterium glutamicum |
| 44 | ATCC 21159 | Corynebacterium glutamicum |
| 45 | ATCC 21355 | Corynebacterium glutamicum |
| 46 | NRRL B-11294 | Corynebacterium glutamicum |
| 47 | NRRL B-11423 | Corynebacterium glutamicum |
| 48 | ATCC 13868 | Corynebacterium herculis |
| 49 | ATCC 17965 | Corynebacterium melassecola |
| 50 | ATCC 17966 | Corynebacterium melassecola |
| 51 | ATCC 19080 | Corynebacterium petrophilum |
| 52 | ATCC 12932 | Escherichia coli |
| 53 | ATCC 10798 | Escherichia coli K12 |
| 54 | ATCC 25208 | Escherichia coli 55-25 |
| 55 | ATCC 25377 | Escherichia coli W6 |
| 56 | ATCC 25378 | Escherichia coli W157 |
| 57 | ATCC 21369 | Methanomonas methylovora |
| 58 | ATCC 21370 | Methanomonas methylovora |
| 59 | ATCC 21371 | Methylobacterium |
| 60 | ATCC 21372 | Methylobacterium |
| 61 | ATCC 21969 | Methylobacterium |
| 62 | ATCC 15354 | Microbacterium ammoniaphilum |
| 63 | ATCC 15176 | Micrococcus luteus |
| 64 | ATCC 15076 | Nocardia globerula |
| 65 | ATCC 15799 | Nocardioides simplex |
| 66 | ATCC 21192 | Pseudomonas dacunhae |
| 67 | ATCC 21276 | Pseudomonas insueta |
| 68 | ATCC 21966 | Pseudomonas insueta |
| 69 | ATCC 21967 | Pseudomonas insueta |
| 70 | ATCC 21968 | Pseudomonas methanolica |
| 71 | ATCC 15175 | Pseudomonas putida |
| 72 | ATCC 15447 | Pseudomonas sp. |
| 73 | ATCC 15779 | Pseudomonas sp. |
| 74 | ATCC 15780 | Pseudomonas sp. |
| 75 | ATCC 15113 | Rhodococcus equi |
| 76 | ATCC 15592 | Rhodococcus sp. |
| 77 | ATCC 15968 | Rhodococcus sp. |
| 78 | ATCC 15108 | Rhodococcus sp. |
| 79 | ATCC 15109 | Rhodococcus sp. |
| 80 | ATCC 15110 | Rhodococcus sp. |
| 81 | ATCC 15582 | Rhodococcus sp. |
| 82 | ATCC 15589 | Rhodococcus sp. |
| 83 | ATCC 15960 | Rhodococcus sp. |
| 84 | ATCC 15961 | Rhodococcus sp. |
| 85 | ATCC 15962 | Rhodococcus sp. |
| 86 | ATCC 15963 | Rhodococcus sp. |
| 87 | ATCC 21402 | Rhodococcus sp. |
| 88 | ATCC 21403 | Rhodococcus sp. |
| 89 | ATCC 21512 | Rhodococcus sp. |
| 90 | ATCC 21534 | Rhodococcus sp. |
| 91 | ATCC 15238 | Streptomyces tanashiensis |

### Intermediates

The strains and methods described herein can be used to produce intermediates in the pathway to production of BDO as described in FIG. 1A. Thus, the strains can be used to produce: L-glutamate 5-phosphate; L-glutamate 5-semialdehyde; 5-hydroxy-L-norvaline; 5-hydroxy-2-oxopentanoate; and 4-hydroxybutanal, as well as cyclized forms of any of these intermediates. Strains producing an intermediate can contain nucleic acid molecules encoding the polypeptides capable of carrying out all or a subset of conversions A1-A6. In some cases, conversions beyond those required to make the selected intermediate are not carried out. For example, a strain producing 4-hydroxybutanal might carry out only conversions A1-A5. The intermediates can be isolated and partially or wholly purified.

### Additional Enzymes

There are other pathways for BDO production in microorganisms and enzymes carrying out conversions in these alternative pathways can also be expressed in recombinant microbes, including those described herein. One alternative pathway is described in WO 2008/115840. Useful additional enzymes can include: (1) succinyl-CoA synthetase; (2) CoA-independent succinic semialdehyde dehydrogenase; (3) [alpha]-ketoglutarate dehydrogenase; (4) glutamate: succinate semialdehyde transaminase; (5) glutamate decarboxylase; (6) CoA-dependent succinic semialdehyde dehydrogenase; (7) 4-hydroxybutanoate dehydrogenase; (8) [alpha]-ketoglutarate decarboxylase; (9) 4-hydroxybutyryl CoA: acetyl-CoA transferase; (10) butyrate kinase; (11) phosphotransbutyrylase; (12) aldehyde dehydrogenase; and (13) alcohol dehydrogenase. These enzymes and the nucleic acid molecules encoding them are known in the art.

### Production and Isolation of BDO

After a modified (e.g., recombinant) microorganism is obtained, it can be cultured in a medium. Culturing techniques and media are well known in the art. In one embodiment, culturing can be performed by aqueous fermentation in an appropriate vessel. The medium can comprise one or more carbon sources such as glucose, sucrose, fructose, high fructose syrup, invert sugar, lactose, galactose, starch, molasses, starch hydrolysate, or hydrolysates of vegetable matter, among others. In some cases, the medium can also comprise a nitrogen source as either an organic or an inorganic molecule. Alternatively or additionally, the medium can comprise components such as amino acids; purines; pyrimidines; corn steep liquor; yeast extract; protein hydrolysates; water-soluble vitamins, such as B complex vitamins; inorganic salts such as chlorides, hydrochlorides, phosphates, or sulfates of Ca, Mg, Na, K, Fe, Ni, Co, Cu, Mn, Mo, or Zn, among others. Further components known to one of ordinary skill in the art to be useful in microbe (e.g., fungus, yeast, bacteria) culturing or fermentation can also be included. The pH of the medium can be buffered but need not be. Considerations for selection of medium components include but are not limited to productivity, cost, and impact on the ability to recover desired products (e.g., BDO and/or BDO intermediates).

The modified cell can be cultured under conditions and for a time sufficient to convert all or a portion of a selected substrate to BDO. Thus, production of BDO can be assessed relative to a selected substrate, e.g., a carbon source. In certain cases, the carbon source used as a measure of BDO production is dextrose. For example, the BDO may accumulate to at least about 0.001 moles of BDO per mole of substrate, 0.01 moles of BDO per mole of substrate, 0.1 moles of BDO per mole of substrate, 0.5 moles of BDO per mole of substrate, at least about 0.6 moles of BDO per mole of substrate, at least about 0.7 moles of BDO per mole of substrate, at least about 0.8 moles of BDO per mole of substrate, at least about 0.9 moles of BDO per mole of substrate, or at least about 1.0 mole of BDO per mole of substrate.

The modified cell can be cultured under conditions and for a time sufficient to convert all or a portion of a selected substrate to a BDO intermediate. In certain cases the substrate is a carbon source. In certain cases, the carbon source is dextrose. For example, the BDO intermediate may accumulate to at least about 0.5 moles of BDO intermediate per mole of substrate, at least about 0.6 moles of BDO intermediate per mole of substrate, at least about 0.7 moles of BDO intermediate per mole of substrate, at least about 0.8 moles of BDO intermediate per mole of substrate, at least about 0.9 moles of BDO intermediate per mole of substrate, or at least about 1.0 mole of BDO intermediate per mole of substrate.

In certain cases, the BDO accumulates in the medium to at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, at least about 200 g/L.

In certain cases, the BDO intermediate accumulates in the medium to at least about 20 g/L, at least about 30 g/L, at least about 40 g/L, at least about 50 g/L, at least about 60 g/L, at least about 70 g/L, at least about 80 g/L, at least about 90 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, at least about 200 g/L.

After culturing has progressed for a sufficient length of time to produce a desired concentration of BDO, the BDO can be isolated. Specifically, the BDO can be brought to a state of greater purity by separation of the BDO from at least one other component of the cell or the medium. In some cases, the BDO is at least about 50%, 55%, 60%, 65%, 70%, 75%, 80%, 85%, 90%, 95%, 96%, 97%, 98%, 99%, 99.95% pure or more. In some cases, the isolated BDO is at least about 95% pure, such as at least about 99% pure.

Any available technique can be utilized to isolate the accumulated BDO. For example, the isolation can comprise purifying the BDO from the medium by micro- and/or nano-filtration or liquid-liquid extraction of the broth using a water immiscible organic solvent. The BDO (boiling point 228-229° C.) can be isolated from the organic solvent by subsequent distillation.

### EXEMPLIFICATION

All basic molecular biology and DNA manipulation procedures described herein are generally performed according to Sambrook et al., or Ausubel et al., (Sambrook J, Fritsch EF, Maniatis T (ed.) 1989, Molecular Cloning: A Laboratory Manual, Cold Spring Harbor Laboratory Press: New York; Ausubel F M, Brent R, Kingston R E, Moore D D, Seidman J G, Smith J A, Struhl K (ed.) 1998 Current Protocols in Molecular Biology, Wiley: New York).

A list of some of the candidate genes that may be applicable to the 1,4-butanediol (BDO) biosynthetic pathway described herein (depicted in FIG. 1A) is given in FIG. 1B and FIGS. 13 through 21. One or more genes encoding enzymes possessing the desired catalytic activity and substrate specificity (or characteristics similar to those required for a particular step in the pathway) were identified using literature resources as well as several public databases.

Each individual candidate gene is cloned and can be initially expressed in E. coli. The nucleotide sequence is subsequently confirmed, and enzyme activity and substrate specificity are analyzed. Each candidate enzyme is assessed by in vitro enzyme assay(s) as well as phenotypic screens or selections (when available). In cases where an enzyme lacks the desired activity, e.g. due to improper substrate specificity, the candidate gene is mutagenized and re-expressed in E. coli to identify variants that possess the desired BDO biosynthetic activity. Once confirmed in E. coli, candidate genes are subcloned into host strain expression vectors, if necessary. These constructs are then introduced into the desired host strain where their expression and activity is confirmed in the new strain environment. As an alternative approach, enzyme activities may be assessed after expression in the host strain. Thus, when C. glutamicum is used as the host strain, enzyme activities may be assessed after expression in C. glutamicum.

The final step in the strain construction process is to combine a complete set of BDO biosynthetic genes in the desired host strain to generate a production strain. For this process, individual genes may be combined into one or more operons which may be located chromosomally and/or on plasmids. Candidate production strains are compared to identify the gene combinations and genetic organization that result in the highest level of BDO or BDO intermediate production.

In this exemplification, C. glutamicum is used as an example host strain. However, one of skill in the art will recognize that a number of other microorganisms, including but not limited to those described herein, may also be used as host strains. One of skill in the art will also recognize that different host strains may require different molecular biology (for example transformation and expression vector construction protocols) and codon optimization techniques other than those that are described herein and which are known to those of skill in the art.

### Example 1A

### Isolation and Cloning of 1,4-Butanediol Pathway Gene Candidates in E. coli

Isolation of candidate genes and cloning in E. coli. DNA fragments containing individual BDO pathway candidate genes are obtained either by PCR cloning or in vitro DNA synthesis.

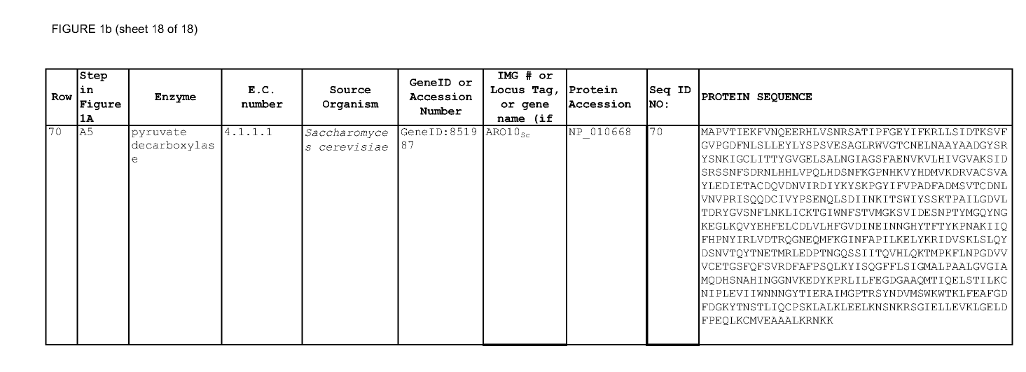

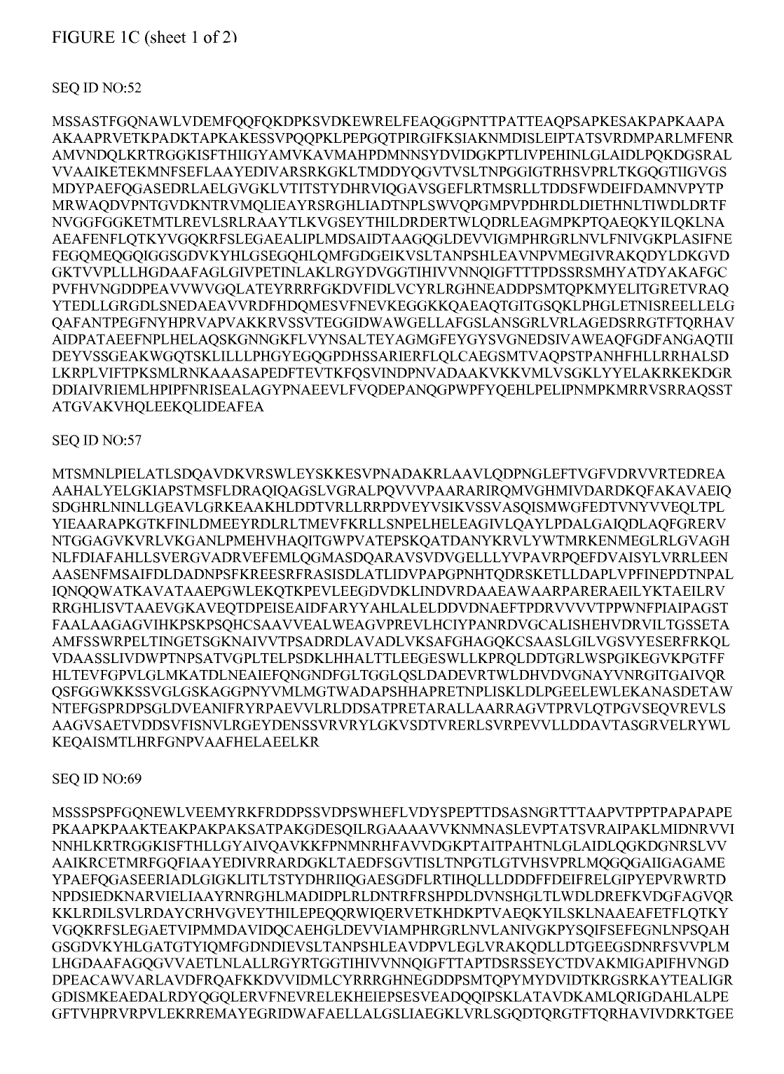

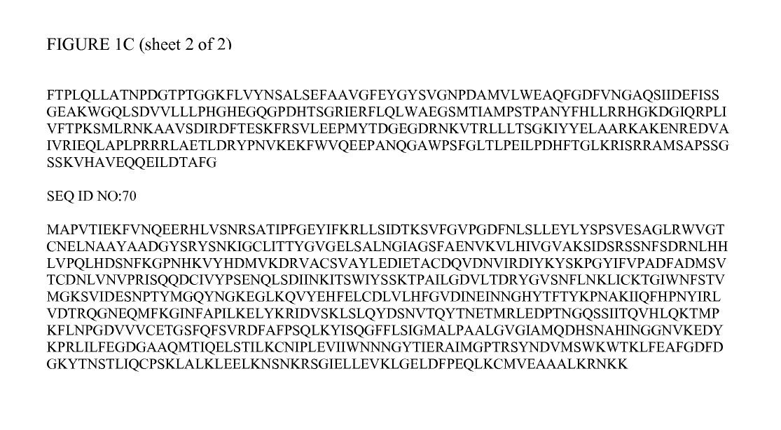

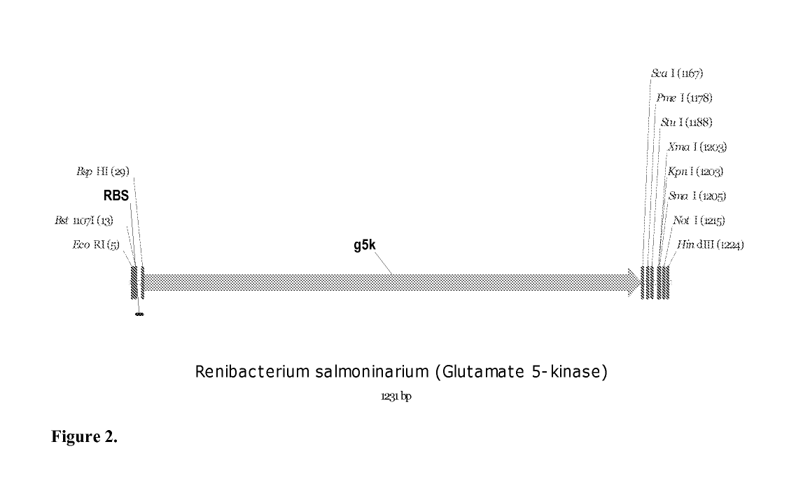

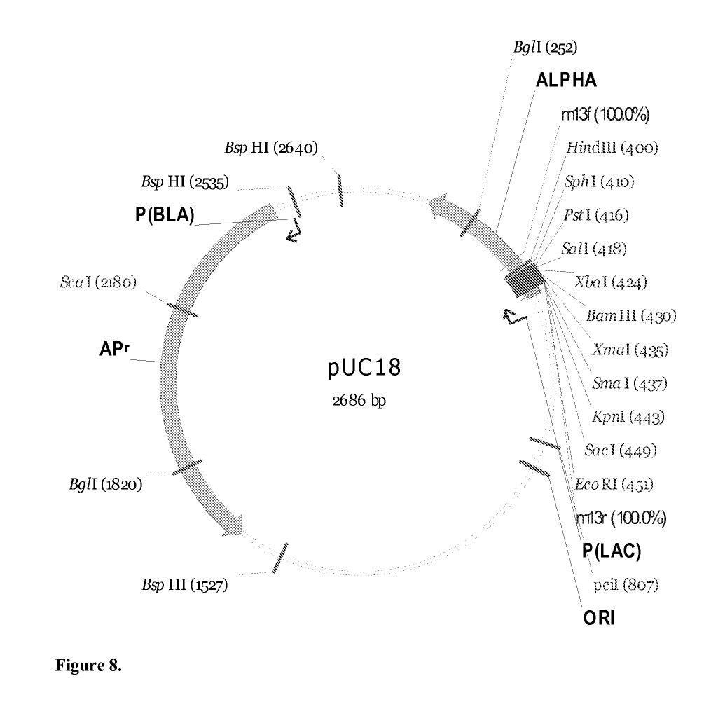

In some instances, candidate genes are isolated as PCR amplicons. The amplicons are generated using chromosomal DNA from a source organism (for example listed in FIG. 1B) as template and oligonucleotide PCR primers that hybridize to the 5′- and 3′-ends of the candidate gene. The primers have 5′-sequence tails containing a number of restriction sites that facilitate cloning of the amplicon into plasmid expression vectors. One example of the PCR cloning method is the cloning of the Renibacterium salmoninarum wild type gene for glutamate 5-kinase. Using PCR primers G5K-fw and G5K-rv (TABLE 2), the gene is PCR amplified using proofreading polymerase Pfu under reaction conditions specified by the manufacturer (Stratagene, La Jolla, Calif.). The resulting PCR fragment (FIG. 2) is gel purified and digested with EcoRI and HindIII. The digested fragment is gel purified again and ligated with pUC18 (FIG. 8) which has been digested with EcoRI and HindIII and gel purified. The Nostoc puntiforme nosE gene which encodes SEQ ID NO: 18 can be cloned using a similar strategy. Using PCR primers MO6578 and MO6579 (TABLE 2) the gene is PCR amplified with proofreading polymerase Pfu. The resulting PCR fragment is gel purified and digested with EcoRI and KpnI and ligated with a plasmid vector such as pUC18 (FIG. 8) or MB4124 which has also been digested with EcoRI and KpnI. One skilled in the art will appreciate that similar strategies and methods can be used to clone other genes as desired.

TABLE 2
PCR Primers

| Primer | Sequence * | Restriction Sites in Tails |
|---|---|---|
| G5K-fw | 5′-AAAGAATTCGTATACAAAGGAGGACAATCATGAGGTCTGA | EcoRI, Bst1107I, BspHI |
| | TAGCGTCG-3′ | |
| | (SEQ ID NO. [symbol] ) | |
| G5K-rv | 5′-TTTAAGCTTCCGCGGCCGCAGATCCCCGGGTACCATATAAT | ScaI, PmeI, StuI, KpnI, |
| | AGGCCTTTTGTTTAAACGTTTAGTACTTCAAATGAGAACTA | SmaI, NotI, HindIII |
| | AGTCATC-3′ | |
| | (SEQ ID NO. [symbol] ) | |
| MO6578 | 5′-AAAGAATTCGTATACAAAGGAGGACAATCATGCCCTTAGC | EcoRI, Bst1107I |
| | CGCTGTG-3′ | |
| | (SEQ ID NO. [symbol] ) | |
| MO6579 | 5′-TTTGGTACCAGGCCTGTTTAAACAGTACTTCAAGACAGATT | ScaI, KpnI, |
| | AGGTTG-3′ | |
| | (SEQ ID NO. [symbol] ) | |

* Nucleotides comprising 5′-primer tails are underlined.

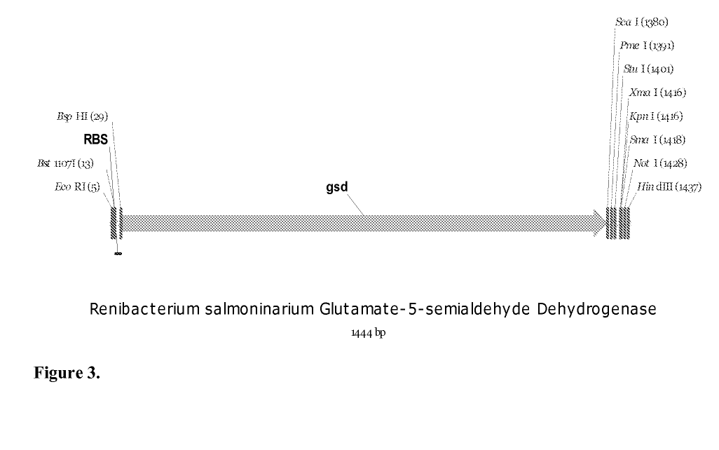

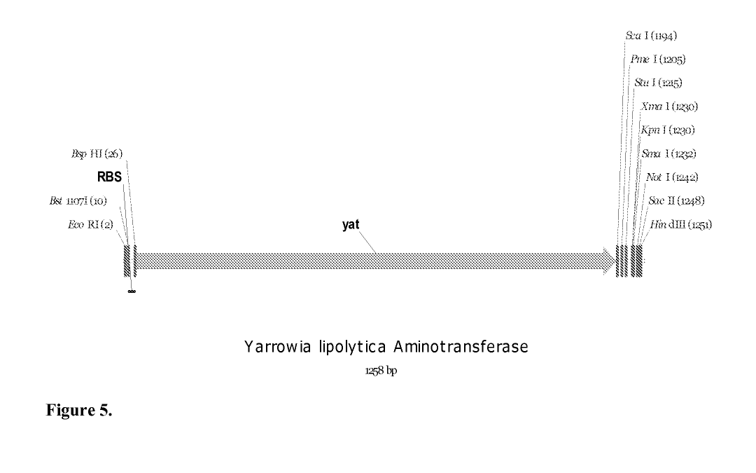

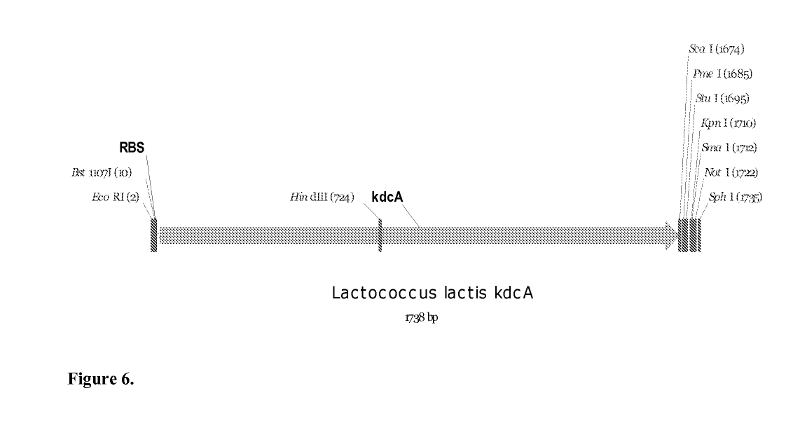

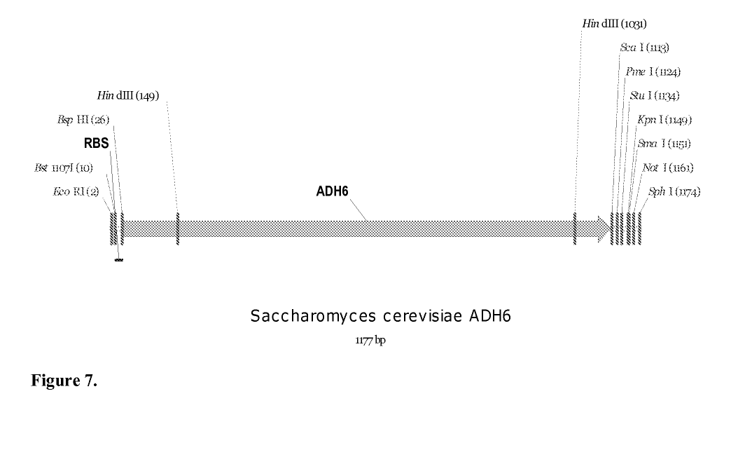

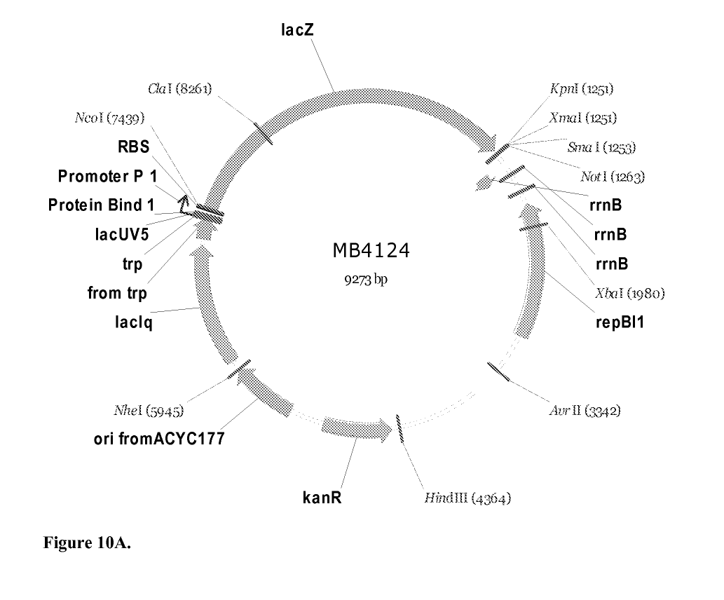

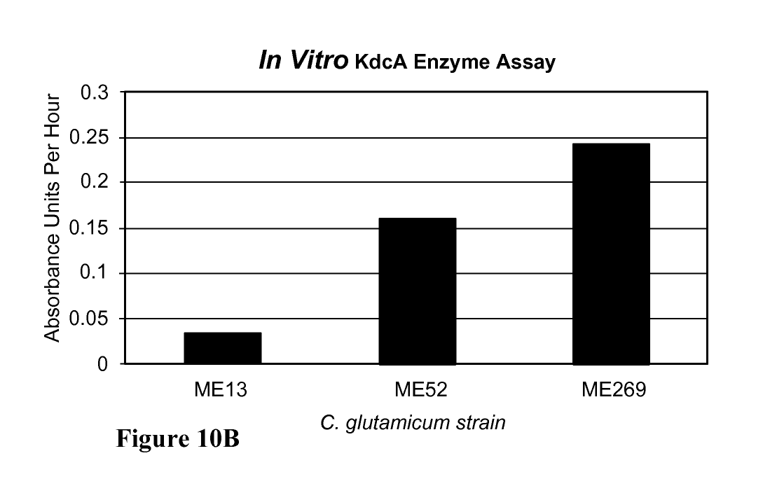

In other instances, candidate genes are generated by in vitro DNA synthesis (for example using the services of Blue Heron Biotechnology, Bothell, Wash.). This strategy is particularly useful for obtaining genes from sources in which the codon usage is vastly different relative to C. glutamicum because it allows for codon optimization (Malumbres et al., Gene 134:15-24. (1993)). DNA synthesis is also useful when a candidate gene contains restriction sites that are required for a given cloning strategy. In addition to containing a candidate gene, DNA fragments synthesized in vitro (FIGS. 3 through 7, TABLE 3A (steps A1-A6) and TABLE 3B (steps A5-A6) are designed to contain flanking restriction sites that facilitate their cloning into E. coli expression vectors (e.g., pUC18 (FIG. 8), MB4124 (FIG. 10)) and subsequently into C. glutamicum expression constructs as multigene operons (for example, as described in Example 5).

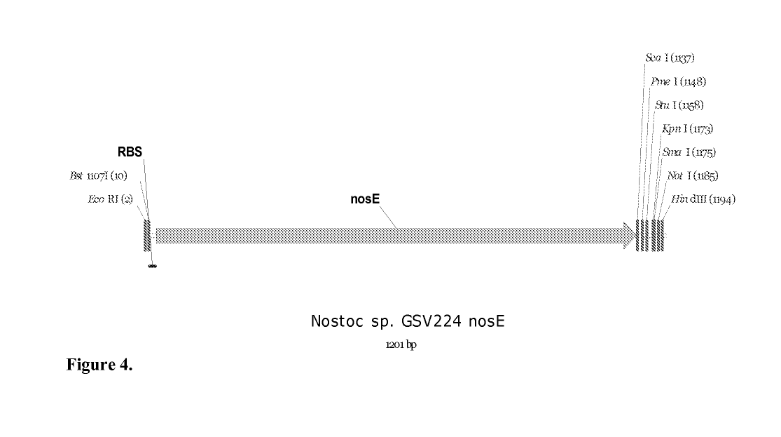

TABLE 3A lists six in vitro synthesized DNA fragments that constitute a complete complement of BDO biosynthetic genes, i.e., they encompass BDO pathway steps A1-A6. These genes are useful in one embodiment of the recombinant cells and methods described herein. TABLE 3B lists four DNA fragments that constitute BDO pathway steps A3-A6. These genes are useful one embodiment of the recombinant cells and methods described herein. This embodiment makes use of glutamate 5-kinase (step A1) and glutamate-5-semialdehyde dehydrogenase (step A2) activities that are native to certain host organisms. For example, when either C. glutamicum or E. coli is the production strain, their respective proB (step A1; see FIG. 1B, row 1) and proA (step A2, see FIG. 1B, row 10) genes can be employed. In some cases, these genes are genetically modified, for example by increasing or modifying expression for example, by altering the coding and/or regulatory sequences. In one case, the endogenous promoters of one or both of these genes can be replaced with an inducible promoter, for example, the trc promoter. In other cases, there is no modification of these endogenous genes or their regulatory sequences. TABLE 3A BDO pathway steps A3-A6 (FIGS. 4 through 7) and TABLE 3B BDO pathway steps A5-A6 are codon-optimized using the Optimizer application (Puigbó et al., Nuc. Acids Res.; 35(Web Server issue):W126-31. 2007) based on the codon usage frequencies calculated from the complete genome sequence of C. glutamicum ATCC 13032. In addition, the gene sequences for BDO pathway steps A1 and A2 in TABLE 3A are codon-altered to remove certain restriction enzyme digestion sites. For both TABLES 3A and 3B, the amino acid sequences encoded by the codon-altered and codon-optimized nucleotide sequences are identical to those encoded by the wild type genes.

Once a DNA fragment containing a candidate gene is generated, it is ligated into an E. coli expression vector such as pUC18 (FIG. 8) or a shuttle vector such as MB4124 (FIG. 10) and the resulting ligation is transformed into E. coli recipient cells (e.g., E. coli Turbo™ which possess lacl^{q}, the gene encoding the lac operon repressor [New England Biolabs, Beverly, Mass.] or E. coli ElectroMax™ DH5α[Invitrogen, Carlsbad, Calif.]). Plasmid-containing clones are selected on media containing ampicillin (Amp), IPTG and Xgal. Transformants harboring plasmids containing an insert can be identified on the basis of blue/white screening. Positive clones are purified and their plasmids are isolated using Qiagen plasmid isolation technology (Qiagen, Valencia, Calif.). In cases where white colonies do not arise or are present only in very low numbers, an aliquot of the transformation culture is re-plated on selective medium containing Amp only. Once colonies arise they are replica plated to medium containing Amp plus IPTG and Xgal to identify clones that contain a vector insert. In this way, constructs in which expression of the cloned gene is toxic to the host are allowed to arise in the absence of candidate gene expression before they are screened on the inducer/indicator medium. The relatively large inoculum that is transferred to the inducer/indicator plate via replica plating usually allows strains containing toxic products to grow enough to allow blue/white screening.

The nucleotide sequences of several isolates for each candidate construct are confirmed by DNA sequencing. One isolate for each candidate is then selected for further characterization.

Plasmid shuttle vectors for cloning candidate genes episomally in C. glutamicum. Plasmid vectors for expression of genes relevant to the production of 1,4-butanediol are described in US20070026505. These plasmids, which may either replicate autonomously or integrate (as described in Example 1B herein) into the host C. glutamicum chromosome, can be introduced into corynebacteria by electroporation as described (see Follettie, M. T., et al. J. Bacteriol. 167:695-702, 1993). All plasmids contain the kanR gene that confers resistance to the antibiotic kanamycin. Transformants are selected on media containing kanamycin (25 mg/L).

For expression from episomal plasmids, vectors are constructed using derivatives of the cryptic C. glutamicum low-copy pBL1 plasmid (see Santamaria et al. J. Gen. Microbiol. 130:2237-2246, 1984). Episomal plasmids contain sequences that encode a replicase and origin of replication, which enables replication of the plasmid within C. glutamicum. Therefore, these plasmids can be propagated without integration into the chromosome. Plasmid MB4124 (FIG. 10) is the vector backbone used to construct episomal expression plasmids described herein. Plasmid MB4124 is a derivative of vector MB4094 (described in US20070026505) that contains the trcRBS site (also described in US20070026505) as well as a lacZ gene downstream of the trc promoter. The presence of lacZ allows blue/white screening when candidate genes are used to replace lacZ. Genes of interest are inserted as fragments containing NcoI-NotI compatible overhangs, with the NcoI site adjacent to the start site of the gene of interest (additional polylinker sites such as KpnI can also be used instead of the NotI site). In addition, useful promoters such as the C. glutamicum gpd, rplM, and rpsJ promoters may be inserted into the MB4124 backbone on convenient restriction fragments, including NheI-NcoI fragments.

TABLE 3A
DNA sequences for in vitro synthesized genes encoding BDO biosynthetic
enzymes (A1-A6), including flanking sequences with restriction enzyme
sites. The coding sequence of each gene is underlined.

| | |
|---|---|
| Pathway | Source |

| | | | | |
|---|---|---|---|---|
| Step: | Enzyme | Organism | Nucleotide Sequence of Fragment | Method |

| | | | | |
|---|---|---|---|---|
| A1 | glutamate 5- | Renibacterium | AAAGAATTCGTATACAAAGGAGGACAATCATGAGAT | In vitro |
| | kinase | salmoninarum | CTGATAGCGTCGTCGTCGGCGGTCGCGAGCTCCTGA | synthesis |
| | [EC:2.7.2.11] | ATCC 33209 | AAACTGCCTCTCGCATCGTCGTTAAAGTCGGCTCTTC | (codon |
| | | | CTCACTCACCTCCGTATCCGGCGGCATCTCTGAGCAG | altered) |
| | | | GCGCTTTCCCAGCTTGTCGATGTATTGGCGGCTCGCA | |
| | | | AGAAGCAAGGCGCCGAGATTATTCTGGTCTCCTCAG | |
| | | | GTGCGATCGCGGCCGGCCTGGCCCCTTTGGGCTTGGC | |
| | | | CCGCCGTCCTAAGGATCTAGCGACCCAGCAGTCCGC | |
| | | | TGCGAGCGTGGGCCAGAGCCTGTTGATGGCCCGCTA | |
| | | | CGGTGCCGCTTTTGGTAGCCACAATGTCACCGTTTCC | |
| | | | CAGGTGCTGCTCACCGCAGAAGATCTCACCCGCCGC | |
| | | | ACCCAATATGCCAATGTGTACCGCGCATTGGAGCGC | |
| | | | CTACTTAATCTCGGTGTACTTCCCATCGTGAATGAAA | |
| | | | ATGACACCGTGGCAACCCACGAGATTCGCTTTGGTG | |
| | | | ACAACGATCGCCTGGCAGCTCTGGTGGCGCACCTCG | |
| | | | TCAAGGCCGACGCAATGGTGCTGCTTTCCGACGTCG | |
| | | | ATGCGGTCTACGACGGCCCGCCAAACACCGGAGCCC | |
| | | | GCCGCATTGAGCAGATCAGCAGCCCTGAGGATCTTG | |
| | | | CCGGAATCAAGATTGGCTCCGTCGGTTCCGCTGGAG | |
| | | | TGGGTACTGGCGGAATGGCGACCAAAGTCCAAGCTG | |
| | | | CGTCCATCGCGGCAGGCTTTGGCACCCCAGCTCTGGT | |
| | | | TACTTCAGCAGCGAATGCCGAGGCGGCTTTGGCCGG | |
| | | | TGCCGATGTGGGCACCTGGTTTTCCTTGCATGGCGAA | |
| | | | CGCCGTCCGATTCGCTTGCTATGGCTGGCACATCTGG | |
| | | | CTAGCACCCACGGAAAGCTGACTCTGGACGACGGCG | |
| | | | CCGTTGCTGCCATCCGCGAACGCCATAAATCCCTGTT | |
| | | | GCCAGCCGGAATCAAAGCAGTGTCCGGCGAGTTCGA | |
| | | | AGCGGGCGACGCCGTTGAACTGGTTGACCTGGCGGG | |
| | | | CCGCACCTTTGCGCACGGCTTGGTCAATTATGACGCC | |
| | | | GCGGAATTGCCAACCATGCTGGGCAAGAAAACTAAG | |
| | | | GATCTAGCCGGAGAATTGGGCCGCGACTACGTCCGC | |
| | | | GCCGTCGTCCATGTGGATGACCTGGTTCTCATTTGAA | |
| | | | GTACTAAACGTTTAAACAAAAGGCCTATTATATGGT | |
| | | | ACCCGGGGATCTGCGGCCGCGGAAGCTTAAA | |
| A2 | glutamate-5- | Renibacterium | AAAGAATTCGTATACAAAGGAGGACAATCATGACAG | In vitro |
| | semialdehyde | salmoninarum | AGCTCATCAGTTCAGCGACCGCCTCCACGGCAGATG | synthesis |
| | dehydrogenase | ATCC | ACGCCAAGCAGGACGGCGTGCAAAACTCTTCTCCGG | (codon |
| | [EC:1.2.1.41] | 33209 | CGGATAACGTGCCGGTCGAAGCTGCGGTCTTTGCGA | altered) |
| | | | TCGCGGACCGATCAAGGACTGCAGCCCGCAAACTTA | |
| | | | GTCGCGCTAATCGAGCCTGGAAAGACCGTGCCCTGT | |
| | | | TAGCTATTGCCCACGAGCTGTTGGCCCGCCAAGACA | |
| | | | ATGTGCTGTCAGCGAATGCGGCTGATATCGCGGCGG | |
| | | | CCAAGGAATCCGGCACTTCGGACGCATTGCTGGATC | |
| | | | GGATGTCGCTGAATCAGGAGCGGATAAAGAAACTTG | |
| | | | CGGTGGCATTGGAAAACCTTGCCGCACTGCCTGATC | |
| | | | CAGTGGGCACAGTTGTTCGCGGGCAGACCCTGCCAA | |
| | | | ACGGACTTCGGCTGCAACAGGTCAACGTGCCGATGG | |
| | | | GCGTAATAGCCGCGATTTACGAAGCGCGGCCCAACG | |
| | | | TCACGATAGATATTGCCGGTTTGGCGCTCAAGAGCG | |
| | | | GCAATGCCGTGATTTTGCGTGGCGGTAGCGCGGTGG | |
| | | | CGCAAACGAACACGGCATTACTGAATGTCATGCGGG | |
| | | | ATGCCTTGGAGAAGGTGGGGCTATCGGTTGACGCGG | |
| | | | TCCAAAGCGTCGATTCCTTTGGACGCGAGGGTGCTG | |
| | | | CTGCCTTGATGCGAGCTCGTGGACGAGTGGATGTGC | |
| | | | TGATTCCACGTGGTGGCCATGAGTTGATTCAGACGGT | |
| | | | CGTTGACAATTCGTCGGTGCCGGTGATCGAAACCGG | |
| | | | TGAGGGTAATGTGCACATTTTCATCGACGAATCTGCC | |
| | | | GCCGTCGAGTCTTCGGTCGAAATCTTATTAAACTCTA | |
| | | | AAACACAGCGGCCCAGTGTTTGCAACACGGTAGAGA | |
| | | | CACTTTTGGTGCACGAAGGCTCCAAGGCACTTGGGC | |
| | | | CAGTCCTGTCCGCTTTGGCCGCTGCTGGCGTGCGATT | |
| | | | GCACGTCGATGAGCGCATCATGGCAAAGCTCCCAGC | |
| | | | CGGGGTAGTGGCGGTGCTGGCTGACGACCACGACTG | |
| | | | GGCCACTGAATATATGGATCTTGATCTGGCCGTGGC | |
| | | | GATGGTCGATTCGCTTGATCAGGCCATCAAGCACATT | |
| | | | CGTCGCTGGACCACAGGTCATACCGAAGCAATCTTG | |
| | | | TCGAATAATCTGCTCAACACCGAGAAGTTCGTCGCT | |
| | | | GAGATTGATTCGGCGGCAGTAATCGTGAATGCGTCG | |
| | | | ACCAGATTTACCGACGGCGGTGAGTTTGGACTTGGC | |
| | | | GCCGAAGTGGGTATTTCTACCCAAAAGTTGCATGCC | |
| | | | CGCGGGCCTATGGGTTTGGCCGAATTGACCACAACC | |
| | | | AAGTGGATTGTGCATGGCGAGGGCCAAGTTCGCGGC | |
| | | | TAAAGTACTAAACGTTTAAACAAAAGGCCTATTATA | |
| | | | TGGTACCCGGGGATCTGCGGCCGCGGAAGCTTAAA | |
| A3 | Alcohol | Nostoc sp. | GAATTCGTATACAAAGGAGGACAATCATGCCTCTGG | In vitro |
| | Dehydrogenase | GSV224 | CTGCAGTGATGACCGCCCCTAACCAGCCTGTCGAAG | synthesis |
| | [EC 1.1.1.-] | | TTCAGCAACTCCCAGACCCAATCCTGGAGAAGGGCG | (codon |
| | | | GCATCATCCTGGAAACCCTGTACTCCGAGGTCTGTGG | optimized) |
| | | | CACTGACGTTCACCTTCTACACGGACGCCTCGAAGGT | |
| | | | GTTCCTTATCCAATCATCCCAGGTCACTTCTCCGTGG | |
| | | | GCCGCGTTGTTGAGACCGGTGGCGCTGTTTCCGATGT | |
| | | | AAACGGTAAGCTAATCCAGCCAGGCGCAATCGCGAC | |
| | | | TTTCCTTGATGTCCACGAAACTTGCTACAATTGCTGG | |
| | | | TACTGTCTTGTTGCCAAGGCTTCCACCCGCTGTCCTC | |
| | | | AGCGTAAGGTTTATGGCGTGACATACTCCGCAAAGG | |
| | | | ACGGCCTGTTGGGCGGCTGGTCCGAGCTTATTTACCT | |
| | | | GAAGCCGGGTGTCAAGGTTCTGACCCTGCCAGAAGA | |
| | | | AGTCTCTCCTAAGCAGTTCATCGCTGGTGGTTGCGCA | |
| | | | CTCCCAACCGCCCTGCACGCTATCGACCGTGCACAA | |
| | | | ATCCAGATCGGTGACGTCGTCGTCGTCCAGGGTTGTG | |
| | | | GACCAGTCGGTCTCTCCGCAGCAATTCTCGCTCTGCT | |
| | | | CTCCGGAGCTGGCAAGGTTATTGTGATCGATAAGTTC | |
| | | | GAGTCCCGCCTGGTCGTCGCAAAGAGCTTCGGCGTT | |
| | | | GACGAAACCCTTGCAATCAAGGCAGACGACCCACGT | |
| | | | CAGCACATCGAACGCGTGCTCGAGCTCACCAACGGA | |
| | | | CACGGTGCAGACGTTACCATCGAGGCCACCGGTATC | |
| | | | CCAATCGCAGTTAAGGAAGGTCTTAACATGACCCGC | |
| | | | AACGGTGGTCGCTACGTCATCGTTGGTCACTACACCA | |
| | | | ACACCGGTGAGATCCTTATCAACCCACACCTGGAAA | |
| | | | TCAACCTTAAGCACATCGACATCCGCGGCACCTGGG | |
| | | | GTATCGACTTCAGCCACTTCTACCGTATGATTGAGCT | |
| | | | ACTGAAGCGTCACTCCGACTCCTCCAAGAACATCGC | |
| | | | CTGGAGCTCCATTATCAGCCGTTCTTACACCCTGAAG | |
| | | | GAGATCAACCAGGCCCTCGCAGACGTTGAACAGGGC | |
| | | | TCCGTGCTTAAGGCTGTTATCCAGCCAAACCTGTCCT | |
| | | | GAAGTACTAAACGTTTAAACAAAAGGCCTATTATAT | |
| | | | GGTACCCGGGGATCTGCGGCCGCGGAAGCTTAAA | |
| A4 | Branched- | Yarrowia | GAATTCGTATACAAAGGAGGACAATCATGATTCGCA | In vitro |
| | chain-amino- | lipolytica | ACAACTTGCGCTCCCTTTCCCGCGCCTTCAGCACCTC | synthesis |
| | acid | CLIB122 | CTCCATGCGTCTGGGCGCCGGAATGGACGCCTCCAA | (codon |
| | transaminase | | GCTCCAGATCACCAAGACCAAGTCCCCAAAGGAAAA | optimized) |
| | [EC:2.6.1.42] | | GCAGGCCCCAAAGGATCTCATTTTCGGCCATACCTTC | |
| | | | ACCGACCACATGCTGACTGTCGAGTGGACTGCCAAG | |
| | | | GACGGCTGGGCTGCTCCACAGATCACCCCATACGGT | |
| | | | CCTCTTGAGTTAGACCCTTCCGCCGTCGTCCTGCACT | |
| | | | ACGCCTTTGAGTGTTTCGAGGGCCTCAAGGCTTACAA | |
| | | | GGACGAGTCTGGAAACGTGCGTCTGTTCCGCGTTGA | |
| | | | TAAGAACATGCACCGCATGAACACCTCCGCCGAGCG | |
| | | | CATCTGCCTGCCAGAGTTTGATGGCGCCGAGGCTGC | |
| | | | CAAGCTGATTGGCCAATTGGCCAAATTAGATTCCGCT | |
| | | | ATTCCTGAGGGACGCGGCTACTCCATGTACCTCCGCC | |
| | | | CTTCTCTGATTGGAACCACCGCCGCTCTCGGCGTCGG | |
| | | | AACCCCAGATAAGGCGCTCTTTTACGTCATTGCATCC | |
| | | | CCAGTCGGCCCATACTACCCTACCGGATTCAAGGCC | |
| | | | GTCAAGCTGGAGGCTACTGACTACGCTGTCCGCGCC | |
| | | | TGGCCTGGAGGAGTCGGAAACAAGAAGCTGGGAGC | |
| | | | CAACTACGCTCCATGTATCAAGCCTCAGCAGCAGGC | |
| | | | CGCTTCTCGCGGCTACCAGCAGAACCTGTGGCTGTTT | |
| | | | GGCGACGAGGGCAACATCACCGAGGTCGGGACCATG | |
| | | | AACGCCTTCTTTGTGTTTGAGCGCAACGGCAAGAAG | |
| | | | GAGCTTGTCACTGCTCCTTTGGACGGTACTATTTTAG | |
| | | | AAGGTGTCACTCGCGACTCCATTCTGGAGCTGGCTCG | |
| | | | CGAACGCTTGCCTTCTGCTGACTGGATCGTTTCCGAG | |
| | | | CGCTACTGCACTATTAAGGAGGTCGCGGAGGCTGCC | |
| | | | GAGAAGGGCGAGCTTGTTGAAGCCTTTGGAGCTGGT | |
| | | | ACTGCCGCTGTTGTCTCCCCTATCAAGGAGATTGGAT | |
| | | | GGGGAGAGAAGACTATTAACATTCCTCTCCAGCCTG | |
| | | | GCAAGGAGGCCGGTAAGCTGACTGAGACTGTTAATG | |
| | | | AGTGGATTGGAGATATCCAGTACGGTAAGGATGAAT | |
| | | | ACAAGGGATGGTCTAAGGTGGTCTAAAGTACTAAAC | |
| | | | GTTTAAACAAAAGGCCTATTATATGGTACCCGGGGA | |
| | | | TCTGCGGCCGCGGAAGCTTAAA | |
| A5 | branched-chain | Lactococcus | GAATTCGTATACAAAGGAGGACAATCATGTACACCG | In vitro |
| | alpha-keto acid | lactis | TTGGTGATTACCTCCTGGACCGCTTGCACGAACTAGG | synthesis |
| | decarboxylase | NIZO | CATCGAGGAGATCTTCGGCGTCCCAGGTGACTACAA | (codon |
| | EC 4.1.1.72 | B1157 | CCTGCAGTTCCTCGACCAAATCATCTCTCGCGAGGAC | optimized) |
| | | | ATGAAGTGGATCGGCAACGCAAACGAACTTAACGCA | |
| | | | AGCTACATGGCTGACGGCTACGCTCGCACCAAGAAG | |
| | | | GCCGCTGCATTCCTGACAACCTTCGGCGTCGGTGAGC | |
| | | | TTTCAGCTATCAATGGCCTTGCGGGATCCTACGCAGA | |
| | | | AAACCTGCCTGTCGTTGAGATTGTTGGTTCCCCTACT | |
| | | | TCCAAGGTTCAGAACGATGGTAAGTTCGTCCACCAC | |
| | | | ACCCTCGCTGACGGTGACTTCAAGCACTTTATGAAG | |
| | | | ATGCACGAACCAGTTACCGCGGCCCGCACCCTCTTG | |
| | | | ACCGCAGAAAACGCTACCTACGAAATCGACCGCGTT | |
| | | | CTTTCCCAGCTGCTGAAGGAACGCAAGCCAGTTTAC | |
| | | | ATCAACCTGCCCGTCGATGTTGCGGCAGCAAAGGCT | |
| | | | GAAAAGCCTGCTCTCTCACTCGAGAAGGAATCTTCT | |
| | | | ACCACCAACACCACCGAGCAAGTCATCCTGAGCAAG | |
| | | | ATTGAAGAATCTCTGAAGAACGCTCAGAAGCCAGTC | |
| | | | GTTATTGCAGGCCACGAGGTAATCTCCTTCGGTCTCG | |
| | | | AAAAGACCGTTACTCAGTTCGTTTCTGAGACCAAGCT | |
| | | | TCCAATCACCACACTGAACTTCGGTAAGAGCGCCGT | |
| | | | CGACGAAAGCCTTCCATCCTTCCTGGGTATCTACAAC | |
| | | | GGCAAATTGTCCGAAATTTCCCTGAAGAATTTCGTTG | |
| | | | AGTCTGCCGATTTTATCCTGATGCTGGGCGTGAAGCT | |
| | | | GACTGACTCTTCCACCGGTGCTTTCACTCACCACCTT | |
| | | | GACGAAAACAAGATGATTTCCCTTAACATCGATGAA | |
| | | | GGTATCATCTTCAACAAGGTGGTTGAGGACTTCGACT | |
| | | | TCCGCGCTGTTGTCTCCTCCCTTTCCGAGCTGAAGGG | |
| | | | AATTGAATACGAGGGCCAGTATATCGATAAGCAGTA | |
| | | | CGAGGAGTTCATCCCAAGCTCCGCCCCTCTGAGCCA | |
| | | | GGATCGTCTGTGGCAGGCGGTGGAATCCCTCACCCA | |
| | | | GTCCAACGAGACCATCGTGGCAGAGCAGGGAACCTC | |
| | | | CTTTTTCGGCGCTAGCACCATCTTTCTGAAGAGCAAC | |
| | | | TCTCGCTTCATCGGTCAGCCACTATGGGGATCTATCG | |
| | | | GATACACTTTCCCAGCAGCACTGGGCTCCCAGATCG | |
| | | | CTGATAAGGAATCCCGCCACCTCCTGTTCATCGGCGA | |
| | | | CGGTTCCCTGCAACTCACCGTGCAGGAGCTCGGCCTT | |
| | | | TCTATCCGCGAGAAGCTGAACCCAATCTGCTTCATCA | |
| | | | TCAACAACGACGGTTACACCGTTGAACGCGAAATCC | |
| | | | ACGGTCCAACCCAGTCCTACAACGATATCCCTATGTG | |
| | | | GAACTACTCTAAGCTCCCAGAAACCTTCGGCGCAAC | |
| | | | CGAGGATCGCGTTGTTTCCAAGATCGTTCGTACCGAG | |
| | | | AACGAGTTCGTTTCTGTGATGAAGGAAGCACAGGCA | |
| | | | GATGTGAACCGTATGTACTGGATCGAACTTGTCCTGG | |
| | | | AGAAGGAAGACGCTCCAAAGCTCCTCAAGAAGATGG | |
| | | | GTAAGCTCTTCGCAGAGCAGAATAAGTAGAGTACTA | |
| | | | AACGTTTAAACAAAAGGCCTATTATATGGTACCCGG | |
| | | | GGATCTGCGGCCGCGGGCATGCAAA | |
| A6 | Alcohol | Saccharomyces | GAATTCGTATACAAAGGAGGACAATCATGAGCTACC | In vitro |
| | Dehydrogenase | cerevisiae | CAGAGAAGTTCGAGGGTATTGCCATCCAGTCCCACG | synthesis |
| | [EC 1.1.1.-] | | AAGACTGGAAGAACCCAAAGAAGACCAAGTACGAC | (codon |
| | | | CCTAAGCCATTCTACGACCACGACATTGACATCAAG | optimized) |
| | | | ATCGAAGCTTGCGGAGTATGTGGTTCTGATATCCACT | |
| | | | GCGCTGCCGGCCACTGGGGCAATATGAAGATGCCTC | |
| | | | TCGTCGTCGGCCACGAGATTGTTGGTAAGGTTGTTAA | |
| | | | GCTCGGTCCTAAGTCTAACAGCGGTCTGAAGGTTGG | |
| | | | CCAGCGTGTGGGTGTTGGTGCACAGGTGTTTTCCTGC | |
| | | | CTTGAGTGTGACCGTTGTAAGAACGACAACGAGCCA | |
| | | | TACTGCACCAAGTTCGTGACCACCTACTCCCAGCCTT | |
| | | | ACGAGGATGGCTACGTCTCCCAGGGCGGCTACGCCA | |
| | | | ACTACGTACGCGTTCACGAGCACTTCGTCGTACCAAT | |
| | | | CCCTGAGAACATCCCAAGTCACCTCGCTGCTCCTCTG | |
| | | | CTCTGCGGAGGTTTGACCGTGTACTCTCCTCTGGTCC | |
| | | | GTAACGGATGTGGCCCAGGCAAGAAGGTCGGCATTG | |
| | | | TCGGTCTAGGCGGCATCGGTTCAATGGGAACCCTCA | |
| | | | TCTCCAAGGCAATGGGCGCTGAAACCTACGTTATCA | |
| | | | GCCGTTCTTCCCGCAAGCGCGAAGACGCTATGAAGA | |
| | | | TGGGCGCTGATCACTACATCGCCACCCTGGAGGAGG | |
| | | | GCGATTGGGGAGAGAAATACTTCGACACCTTCGATC | |
| | | | TTATCGTTGTTTGCGCTTCCTCTCTGACCGATATCGAT | |
| | | | TTCAACATCATGCCTAAGGCCATGAAGGTTGGAGGC | |
| | | | CGCATCGTTTCCATCTCCATCCCAGAGCAGCACGAG | |
| | | | ATGCTGTCCCTGAAGCCATACGGTCTGAAGGCTGTTT | |
| | | | CCATCTCCTACTCAGCACTGGGCAGCATCAAGGAAC | |
| | | | TTAACCAGCTGCTGAAGCTGGTGTCCGAAAAGGACA | |
| | | | TCAAGATCTGGGTTGAGACCCTGCCAGTCGGCGAAG | |
| | | | CGGGCGTGCACGAAGCTTTTGAACGTATGGAAAAGG | |
| | | | GTGACGTGCGCTACCGCTTCACCCTGGTCGGATACG | |
| | | | ACAAGGAGTTCTCCGACTAGAGTACTAAACGTTTAA | |
| | | | ACAAAAGGCCTATTATATGGTACCCGGGGATCTGCG | |
| | | | GCCGCGGGCATGCAAA | |

TABLE 3B
DNA sequences for BDO biosynthetic enzymes (A3-A6), including
flanking sequences with restriction enzyme sites. The coding
sequence of each gene is underlined. This strategy relies on
enzymes native to the host production strain for the glutamate
5-kinase (step A1) and glutamate-5-semialdehyde dehydrogenase
(step A2) activities.

| Pathway | | Source | | |
|---|---|---|---|---|
| Step: | Enzyme | Organism | Nucleotide Sequence of Fragment | Method |
| A3 | Alcohol | Nostoc | ATGCCCTTAGCCGCTGTGATGACTGCACCTAAT | PCR cloning |
| | Dehydrogenase | punctiforme | CAACCAGTTGAAGTGCAACAATTACCAGAGCC | of nosE |
| | [EC 1.1.1.-] | PCC 73102 | GATTCTGGAAAAGGGTGGAATCATTATTGAAAC | gene from |
| | | (ATCC | CTTATATTCTGAGGTTTGTGGAACTGATGTACA | Nostoc |
| | | 29133) | TTTATTACATGGGCGTTTAGAGGGAGTACCATA | punctiforme |
| | | | TCCTATCATCCCTGGACACTTCTCAGTTGGTCG | genomic DNA |
| | | | TGTGGTGGAAACAGGTGGAGCAGTTAGTGATGT | |
| | | | CAATGGTAAATTAATTCAGCCTGGAGCGATCGC | |
| | | | TACTTTTTTAGATGTCCACGAAACCTGTTACAA | |
| | | | CTGCTGGTATTGTCTAGTAGCCAAAGCATCCAC | |
| | | | TCGCTGTCCAAAACGTAAAGTTTATGGTGTCAC | |
| | | | CTACTCAGCCAAAGATGGGTTGCTGGGTGGGTG | |
| | | | GTCTGAATTAATTTACCTCAAACCAGGGGTTAA | |
| | | | AGTTCTCACTTTACCCGAAGAAGTCTCACCAAA | |
| | | | GCAATTCATTGCTGGAGGATGTGCTTTACCAAC | |
| | | | TGCACTACACGCTATTGATCGAGCGCAGATTCA | |
| | | | AATTGGTGATGTCGTCGTGGTGCAGGGTTGCGG | |
| | | | ACCTGTGGGTTTGAGTGCGGCAATTCTAGCTTT | |
| | | | GCTCTCAGGTGCGGGCAAAGTAATTGTAATTGA | |
| | | | TAAATTTGAGAGCCGATTAGTAGTTGCAAAATC | |
| | | | TTTCGGTGTAGATGAAACCCTTGCAATTAGGGC | |
| | | | TGATGATCCACGACAACATATTGAGCGAGTTTT | |
| | | | GGAATTAACCAACGGACACGGCGCTGATGTCA | |
| | | | CTATTGAAGCTACAGGCATTCCTATTGCTGTCA | |
| | | | AAGAGGGCTTAAATATGACCAGAAATGGAGGT | |
| | | | CGCTATGTTATTGTCGGGCATTACACAAACACG | |
| | | | GGTGAGATTCTCATCAATCCACACTTAGAGATT | |
| | | | AATCTAAAGCATATTGATATTCGCGGAACTTGG | |
| | | | GGAATTGATTTCAGCCATTTTTATAGAATGATT | |
| | | | GAATTACTAAAACGTCATAGCGATTCCAGTAAA | |
| | | | AATATTGCTTGGTCAAGCATAATTAGTCGTTCA | |
| | | | TATACACTAAAAGAAATTAATCAAGCACTCGTA | |
| | | | GATGTAGAGCAAGGCTCTGTATTAAAAGCTGTG | |
| | | | ATTCAACCTAATCTGTCTTGA | |
| A4 | Branched-chain- | P. | ATGGGTAACGAAAGCATCAACTGGGACAAGCT | PCR cloning |
| | amino-acid | fluorescens | GGGTTTTGACTACATCAAGACCGACAAGCGGTT | of bcaT |
| | transaminase | (ATCC | TCTCCAGGTCTGGAAAAACGGCGAATGGCAAG | gene from |
| | [EC:2.6.1.42] | 17634) | AAGGCACCCTGACCGACGACAACGTGCTGCAC | Pseudomonas |
| | | | ATCAGCGAGGGCTCCACCGCCCTGCACTACGGC | fluorescens |
| | | | CAGCAATGCTTTGAAGGCCTCAAGGCTTACCGC | genomic DNA |
| | | | TGCAAGGACGGTTCGATCAACCTGTTCCGCCCG | |
| | | | GACCAGAACGCCGCCCGCATGCAACGCAGCTG | |
| | | | CGCGCGCCTGCTGATGCCGCATGTGCCGACCGA | |
| | | | CGTGTTCATCGACGCCTGCAAACAAGTGGTCAA | |
| | | | GGCCAACGAACATTTCATCCCGCCGTACGGCAG | |
| | | | CGGCGGCGCGCTGTACCTGCGCCCGTTCGTGAT | |
| | | | CGGCACCGGTGACAACATCGGCGTGCGCACCG | |
| | | | CGCCGGAGTTCATCTTCTCCGTGTTCTGCATCCC | |
| | | | GGTCGGCGCCTACTTCAAAGGCGGCCTGGTGCC | |
| | | | ACACAACTTCCAGATCTCCACCTTCGACCGCGC | |
| | | | CGCGCCACAAGGCACCGGTGCCGCCAAGGTCG | |
| | | | GTGGCAACTACGCCGCCAGCCTGATGCCAGGTT | |
| | | | CCGAAGCGAAGAAAGCCGGTTTTGCCGACGCG | |
| | | | ATCTACCTGGACCCGATGACCCACTCGAAAATC | |
| | | | GAAGAAGTCGGCTCGGCCAACTTCTTCGGGATC | |
| | | | ACCCACGACAACCAGTTCATCACGCCGAAGTCG | |
| | | | CCTTCGGTGCTGCCAGGCATCACCCGCCTGTCG | |
| | | | CTGATCGAACTGGCCAAGACCCGCCTGGGTCTG | |
| | | | GACGTGGTCGAGGGCGAAGTGTTCATCGACAA | |
| | | | ACTGGACCAGTTCAAGGAAGCCGGCGCCTGCG | |
| | | | GTACGGCTGCGGTGATCTCGCCGATCGGCGGCA | |
| | | | TCCAGTACAACGGCAAGCTGCACGTGTTCCACA | |
| | | | GCGAGACCGAAGTCGGCCCGATCACCCAGAAG | |
| | | | CTCTACAAAGAGCTGACCGGTGTGCAGACTGGT | |
| | | | GACGTTGAAGCGCCGCAAGGCTGGATCGTCAA | |
| | | | GGTTTGA | |
| A5 | branched-chain | Lactococcus | GAATTCGTATACAAAGGAGGACAATCATGTAC | In vitro |
| | alpha-keto acid | lactis NIZO | ACCGTTGGTGATTACCTCCTGGACCGCTTGCAC | synthesis |
| | decarboxylase EC | B1157 | GAACTAGGCATCGAGGAGATCTTCGGCGTCCCA | (codon |
| | 4.1.1.72 | | GGTGACTACAACCTGCAGTTCCTCGACCAAATC | optimized) |
| | | | ATCTCTCGCGAGGACATGAAGTGGATCGGCAAC | |
| | | | GCAAACGAACTTAACGCAAGCTACATGGCTGA | |
| | | | CGGCTACGCTCGCACCAAGAAGGCCGCTGCATT | |
| | | | CCTGACAACCTTCGGCGTCGGTGAGCTTTCAGC | |
| | | | TATCAATGGCCTTGCGGGATCCTACGCAGAAAA | |
| | | | CCTGCCTGTCGTTGAGATTGTTGGTTCCCCTACT | |
| | | | TCCAAGGTTCAGAACGATGGTAAGTTCGTCCAC | |
| | | | CACACCCTCGCTGACGGTGACTTCAAGCACTTT | |
| | | | ATGAAGATGCACGAACCAGTTACCGCGGCCCG | |
| | | | CACCCTCTTGACCGCAGAAAACGCTACCTACGA | |
| | | | AATCGACCGCGTTCTTTCCCAGCTGCTGAAGGA | |
| | | | ACGCAAGCCAGTTTACATCAACCTGCCCGTCGA | |
| | | | TGTTGCGGCAGCAAAGGCTGAAAAGCCTGCTCT | |
| | | | CTCACTCGAGAAGGAATCTTCTACCACCAACAC | |
| | | | CACCGAGCAAGTCATCCTGAGCAAGATTGAAG | |
| | | | AATCTCTGAAGAACGCTCAGAAGCCAGTCGTTA | |
| | | | TTGCAGGCCACGAGGTAATCTCCTTCGGTCTCG | |
| | | | AAAAGACCGTTACTCAGTTCGTTTCTGAGACCA | |
| | | | AGCTTCCAATCACCACACTGAACTTCGGTAAGA | |
| | | | GCGCCGTCGACGAAAGCCTTCCATCCTTCCTGG | |
| | | | GTATCTACAACGGCAAATTGTCCGAAATTTCCC | |
| | | | TGAAGAATTTCGTTGAGTCTGCCGATTTTATCCT | |
| | | | GATGCTGGGCGTGAAGCTGACTGACTCTTCCAC | |
| | | | CGGTGCTTTCACTCACCACCTTGACGAAAACAA | |
| | | | GATGATTTCCCTTAACATCGATGAAGGTATCAT | |
| | | | CTTCAACAAGGTGGTTGAGGACTTCGACTTCCG | |
| | | | CGCTGTTGTCTCCTCCCTTTCCGAGCTGAAGGG | |
| | | | AATTGAATACGAGGGCCAGTATATCGATAAGC | |
| | | | AGTACGAGGAGTTCATCCCAAGCTCCGCCCCTC | |
| | | | TGAGCCAGGATCGTCTGTGGCAGGCGGTGGAAT | |
| | | | CCCTCACCCAGTCCAACGAGACCATCGTGGCAG | |
| | | | AGCAGGGAACCTCCTTTTTCGGCGCTAGCACCA | |
| | | | TCTTTCTGAAGAGCAACTCTCGCTTCATCGGTC | |
| | | | AGCCACTATGGGGATCTATCGGATACACTTTCC | |
| | | | CAGCAGCACTGGGCTCCCAGATCGCTGATAAGG | |
| | | | AATCCCGCCACCTCCTGTTCATCGGCGACGGTT | |
| | | | CCCTGCAACTCACCGTGCAGGAGCTCGGCCTTT | |
| | | | CTATCCGCGAGAAGCTGAACCCAATCTGCTTCA | |
| | | | TCATCAACAACGACGGTTACACCGTTGAACGCG | |
| | | | AAATCCACGGTCCAACCCAGTCCTACAACGATA | |
| | | | TCCCTATGTGGAACTACTCTAAGCTCCCAGAAA | |
| | | | CCTTCGGCGCAACCGAGGATCGCGTTGTTTCCA | |
| | | | AGATCGTTCGTACCGAGAACGAGTTCGTTTCTG | |
| | | | TGATGAAGGAAGCACAGGCAGATGTGAACCGT | |
| | | | ATGTACTGGATCGAACTTGTCCTGGAGAAGGAA | |
| | | | GACGCTCCAAAGCTCCTCAAGAAGATGGGTAA | |
| | | | GCTCTTCGCAGAGCAGAATAAGTAGAGTACTAA | |
| | | | ACGTTTAAACAAAAGGCCTATTATATGGTACCC | |
| | | | GGGGATCTGCGGCCGCGGGCATGCAAA | |
| A6 | Alcohol | Saccharomyces | GAATTCGTATACAAAGGAGGACAATCATGAGC | In vitro |
| | Dehydrogenase | cerevisiae | TACCCAGAGAAGTTCGAGGGTATTGCCATCCAG | synthesis |
| | [EC 1.1.1.-] | | TCCCACGAAGACTGGAAGAACCCAAAGAAGAC | (codon |
| | | | CAAGTACGACCCTAAGCCATTCTACGACCACGA | optimized) |
| | | | CATTGACATCAAGATCGAAGCTTGCGGAGTATG | |
| | | | TGGTTCTGATATCCACTGCGCTGCCGGCCACTG | |
| | | | GGGCAATATGAAGATGCCTCTCGTCGTCGGCCA | |
| | | | CGAGATTGTTGGTAAGGTTGTTAAGCTCGGTCC | |
| | | | TAAGTCTAACAGCGGTCTGAAGGTTGGCCAGCG | |
| | | | TGTGGGTGTTGGTGCACAGGTGTTTTCCTGCCTT | |
| | | | GAGTGTGACCGTTGTAAGAACGACAACGAGCC | |
| | | | ATACTGCACCAAGTTCGTGACCACCTACTCCCA | |
| | | | GCCTTACGAGGATGGCTACGTCTCCCAGGGCGG | |
| | | | CTACGCCAACTACGTACGCGTTCACGAGCACTT | |
| | | | CGTCGTACCAATCCCTGAGAACATCCCAAGTCA | |
| | | | CCTCGCTGCTCCTCTGCTCTGCGGAGGTTTGACC | |
| | | | GTGTACTCTCCTCTGGTCCGTAACGGATGTGGC | |
| | | | CCAGGCAAGAAGGTCGGCATTGTCGGTCTAGGC | |
| | | | GGCATCGGTTCAATGGGAACCCTCATCTCCAAG | |
| | | | GCAATGGGCGCTGAAACCTACGTTATCAGCCGT | |
| | | | TCTTCCCGCAAGCGCGAAGACGCTATGAAGATG | |
| | | | GGCGCTGATCACTACATCGCCACCCTGGAGGAG | |
| | | | GGCGATTGGGGAGAGAAATACTTCGACACCTTC | |
| | | | GATCTTATCGTTGTTTGCGCTTCCTCTCTGACCG | |
| | | | ATATCGATTTCAACATCATGCCTAAGGCCATGA | |
| | | | AGGTTGGAGGCCGCATCGTTTCCATCTCCATCC | |
| | | | CAGAGCAGCACGAGATGCTGTCCCTGAAGCCAT | |
| | | | ACGGTCTGAAGGCTGTTTCCATCTCCTACTCAG | |
| | | | CACTGGGCAGCATCAAGGAACTTAACCAGCTGC | |
| | | | TGAAGCTGGTGTCCGAAAAGGACATCAAGATCT | |
| | | | GGGTTGAGACCCTGCCAGTCGGCGAAGCGGGC | |
| | | | GTGCACGAAGCTTTTGAACGTATGGAAAAGGGT | |
| | | | GACGTGCGCTACCGCTTCACCCTGGTCGGATAC | |
| | | | GACAAGGAGTTCTCCGACTAGAGTACTAAACGT | |
| | | | TTAAACAAAAGGCCTATTATATGGTACCCGGGG | |
| | | | ATCTGCGGCCGCGGGCATGCAAA | |

### Example 1B

### Generation of Chromosomal Deletions and Insertions in the C. Glutamicum Chromosome

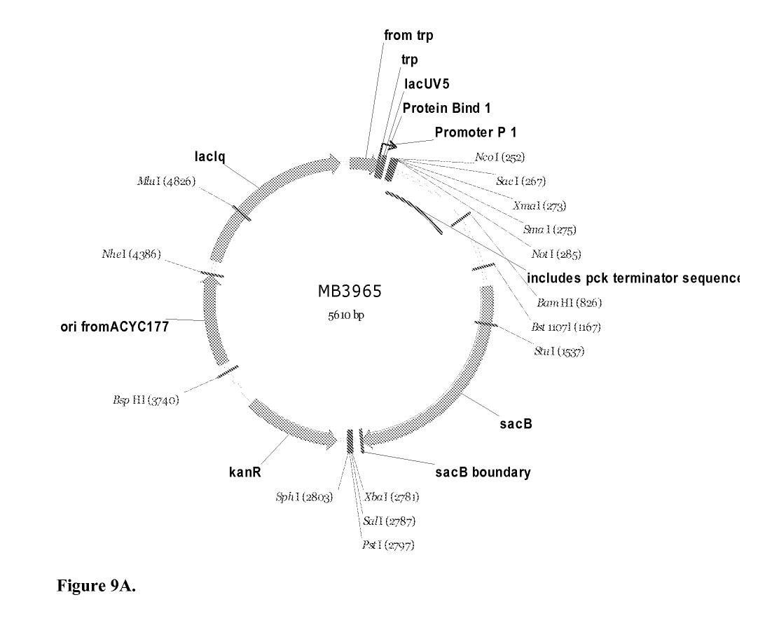

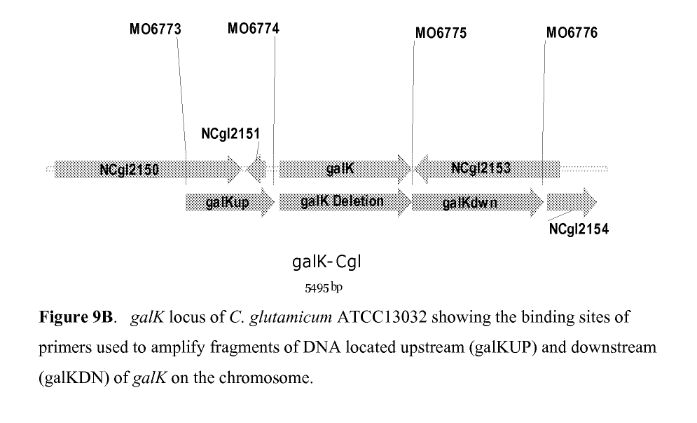

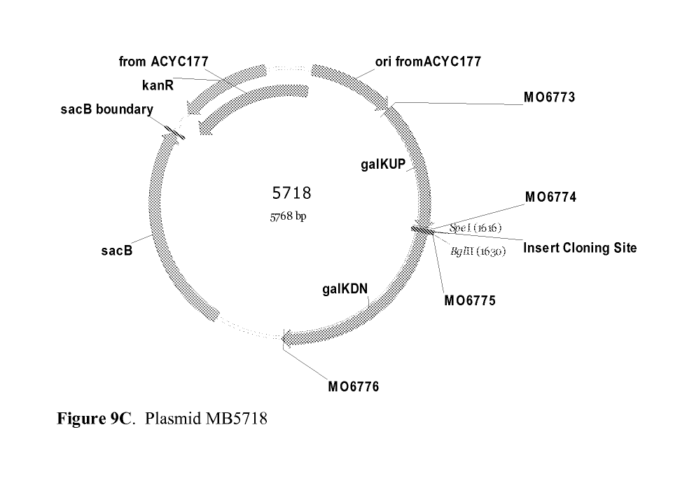

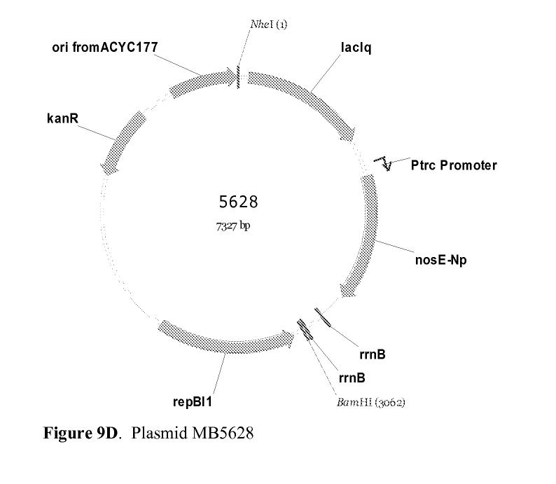

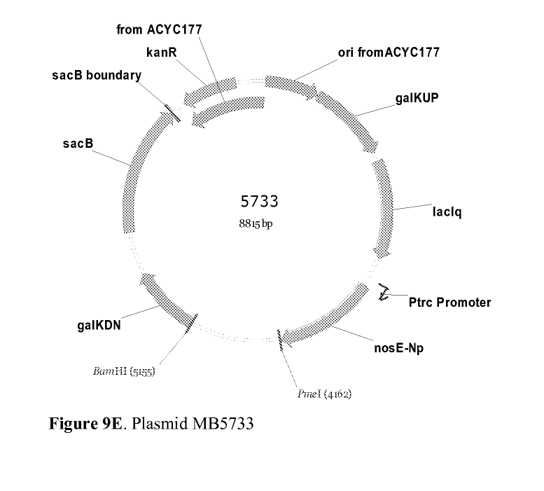

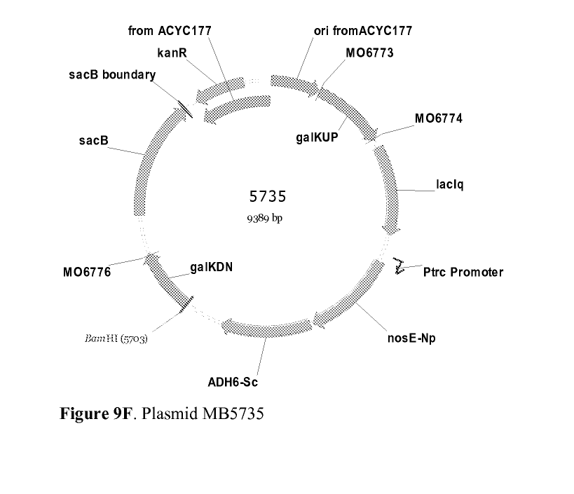

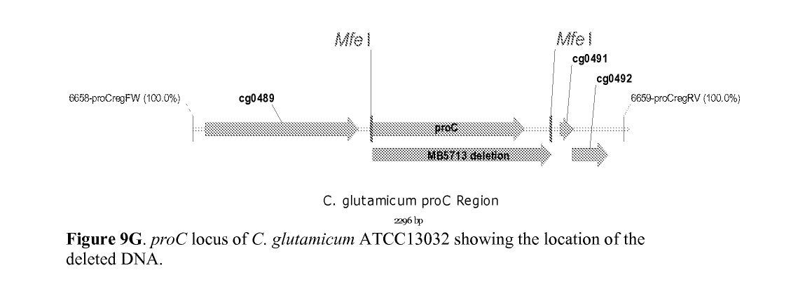

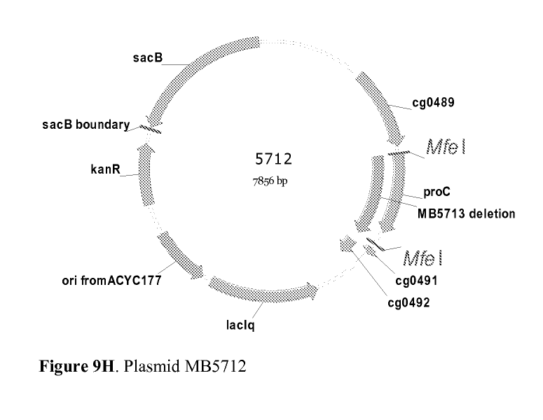

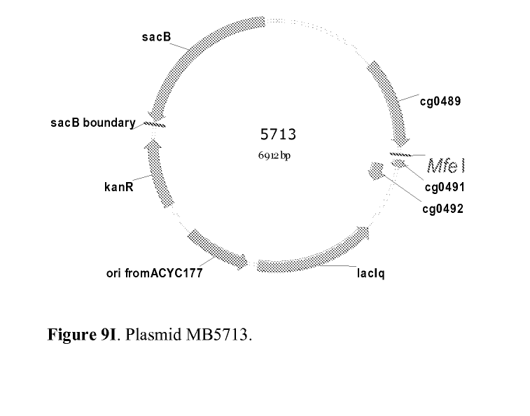

Vector MB3965 (FIG. 9A) is an example of a plasmid backbone useful for generating deletion/integration constructs. MB3965 contains an antibiotic resistance gene (KanR) and an origin of replication that allows it to replicate in E. coli but not in C. glutamicum. Fragments of C. glutamicum chromosomal DNA are cloned into MB3965 and the resulting plasmid is transformed into a C. glutamicum recipient. Since the plasmid is unable to replicate episomally in C. glutamicum, selecting for kanamycin resistant colonies allows the isolation of recombinant strains in which the circular plasmid has integrated into the recipient's chromosome via recombination between the chromosome and the homologous DNA insert on the plasmid. MB3965 also contains the sacB gene, encoding the levansucrase gene from Bacillus subtilis. The sacB gene allows for positive selection of recombinants in which the vector sequence has been lost. Transformants containing integrated plasmids are streaked to Brain Heart Infusion (BHI) (BD-Diagnostics, Franklin Lakes, N.J. USA 07417) medium lacking kanamycin. After 1 day, colonies are streaked onto BHI medium containing 10% sucrose. This protocol selects for strains in which the sacB gene has been excised, since it polymerizes sucrose to form levan, which is toxic to C. glutamicum (see Jager, W., et al. J. Bacteriol. 174:5462-5465, 1992). During growth of transformants upon medium containing sucrose, sacB allows for positive selection for recombination events, resulting in either the restoration of the native locus or the desired deletion event which retains the cassette that regulates the inducible expression of a particular gene of interest (see Jager, W., et al. J. Bacteriol. 174:5462-5465, 1992). PCR, together with growth on diagnostic media (if available), is used to verify that expected recombination events have occurred in sucrose-resistant colonies.

In some instances, constructs can be generated which will both delete native genes and insert heterologous genes into the host chromosome at the deletion event locus. Such plasmids contain nucleotide sequences homologous to regions upstream and downstream of the target deletion sequence and have convenient restriction sites for cloning heterologous genes between the homologous upstream and downstream regions. An example of such an integration vector is based on the galK gene of C. glutamicum (FIG. 9B). The vector was constructed by amplifying DNA sequences located upstream and downstream of galK using PCR primer pairs MO6773 (5′-cacacggtctccctaggacgctctcgatgaggag-3′)/MO6774 (5′-cacacggtctcaagagacactagtcagacccactctagccgttg-3′) and MO6775 (5′-cacaccgtctcactctacagatctgcacgcctacttaaccagcct-3′)/MO6776 (5′-cacaccgtctcagatctcctgcacatgcccttt-3′) respectively. The two DNA fragments generated using these PCR primer pairs were ligated together into MB3965 using engineered tails that resulted in a vector, MB5718 (FIG. 9C), in which heterologous genes could be cloned in between the two fragments using SpeI (compatible with NheI overhangs) and BglII (compatible with BamHI overhangs) restriction sites. Genes cloned into MB5718 can be inserted into the chromosome at the galK locus allowing them to be stably expressed without the requirement for antibiotic selection. Those skilled in the art will recognize that the same cloning strategy can be used to generate similar integration/deletion vectors at other sites in the C. glutamicum chromosome.

Construction of ΔgalK integration plasmids. The galK integration vector (MB5718) was used to construct a galK:: nosE_{Np} integration plasmid. First, MB5718 was digested with SpeI and BglII which cut in between the galKUP (upstream) and galkDN (downstream) portions of the vector. Next, plasmid MB5628 (FIG. 9D) was digested with NheI and BamHI to release a 3061-bp DNA fragment containing nosE_{Np} (see SEQ ID NO:18, the amino acid sequence for nosE from Nostoc punctiforme), expressed from the trc promoter. This fragment, which also contained trc repressor gene, lacl^{q}, was gel purified and ligated with the SpeI and BglII-digested MB5718 fragment to create galK::nosE_{Np} plasmid MB5733 (FIG. 9E).

A second galK integration plasmid was constructed by digesting MB5733 with PmeI and BamHI and inserting a 1567-bp fragment containing ADH6_{sc}, the gene for step A6 of the pathway (FIG. 1A; TABLE 3A, step A6). The resulting plasmid was designated MB5735 (FIG. 9F). MB5735 allows one to generate C. glutamicum strains in which the genes for steps A3 and A6 of the BDO pathway (FIG. 1A) are inducibly expressed from a chromosomal locus.

Construction of a C. glutamicum proC deletion strain. C. glutamicum BDO production strains will likely require genetic alterations that prevent or reduce the flux of GSA (L-glutamate 5-semialdehyde)/P5C (L-1-pyrroline 5-carboxylate) into the final step of the proline biosynthetic pathway (see FIG. 1A). One such alteration would be the deletion of proC, the gene encoding pyrroline 5-corboxylate reductase (FIG. 1A, step B5).

A 2296 bp fragment of the C. glutamicum proC locus (FIG. 9G) is PCR amplified using primers MO6658 (5′AAAACTTAAGCCAGGATCGACAAAGGACTCGAG-3′) and MO6659 (5′AAAAGAGCTCCAAAATCATCATGCCGGGCG-3′). MO6658 and MO6659 have tails which include the restriction sites AflI and SacI respectively (restriction sites are underlined). The PCR fragment is digested with those enzymes and ligated into plasmid MB3965 (FIG. 9A), cut with the same enzymes. The resulting plasmid, MB5712 (FIG. 9H), is digested with MfeI and a 944 bp fragment of DNA containing all but the first 7 nucleotides of the proC gene and 144 bp of the downstream intergenic region is separated from the rest of the plasmid by gel electrophoresis. The remaining 5576 bp fragment, which includes the vector and the DNA sequences that flank proC on the C. glutamicum chromosome is recircularized by ligation resulting in plasmid MB5713 (FIG. 9I). MB5713 is made up of the integration vector (MB3965) containing an insert consisting of 937 bp of chromosomal DNA that flanks proC upstream on the C. glutamicum chromosome and 409 bp of the downstream flanking DNA.

Plasmid MB5713 cannot replicate in C. glutamicum. Therefore, by transforming the plasmid into the bacterium and selecting for kanamycin resistant colonies, one can isolate integrant strains in which the plasmid has undergone recombination between the plasmid insert DNA and its identical chromosomal counterpart located upstream or downstream of the recipient cells' proC allele. Integrant strains are then subjected to the sacB counter-selection as described above to generate strains in which the integrated sequence containing the sacB gene is excised from the chromosome via homologous recombination between the plasmid insert sequence and the chromosomal DNA flanking proC. The resulting strains are then screened to identify proline auxotrophs in which the excision event removed the chromosomal proC allele.

Pyrroline-5-carboxylate production was observed to be an unstable phenotype in some ΔproC strains generated as described above, so an alternative method for generating ΔproC C. glutamicum strains was carried out. Strain ATCC 13032 was first rendered pro⁻ by introduction of a point mutation (K220S) in the wildtype sequence of proB which rendered the protein inactive. The resulting strain, ME222 was subsequently transformed with MB5713 and treated as described above to remove the chromosomal proC allele. A successful deletion was identified by failure to grow on minimal medium supplemented with pyrroline-5-carboxylate and denoted ME241. ProB activity was reintroduced to this strain by transformation with the integrating plasmid MB5858 which harbors the proB gene under control of the trc promoter, KanR, sacB, and lacl^{q}.

### Example 2A

### Bioanalytical Methods for Measuring 1,4-Butanediol and Metabolic Precursors

This Example describes methods and protocols used to measure BDO pathway metabolites intracellularly and in culture supernatants.

Samples are prepared for HPLC analysis by centrifuging (30,000×g) harvested shake flask cultures and then transferring supernatant to a fresh Eppendorf tube. Samples are diluted 50-fold into mobile phase in a 1 mL 96 well plate and the resulting preparations are loaded onto a Gilson auto sampler, which is maintained at ambient temperature. 10 μL of diluted sample is used for instrument injection.

1,4-butanediol, 1,4-butanediol intermediates (including cyclized intermediates) and glucose levels are quantified from broth samples using HPLC analysis. A Gilson 215 auto sampler fronting either a Waters 515 pump or a Waters 1525 pump is used for detection. Both the pump and auto sampler are coupled to a Waters 2487 UV detector and a Waters 2414 refractive index (RI) detector (in series). An Aminex HPX-87H Ion Exclusion Column (300 mm×7.8 mm, Bio-Rad) with an Aminex Cation H Cartridge guard column, (30×4.6 mm, Bio-Rad), is used for separation.

An isocratic separation is performed at 30° C. using 0.05% trifluoracetic acid in deionized water as the mobile phase at a flow rate of 0.4 mL/min (1400 PSI as high pressure limit).

### Example 2B

Fermentation broth, containing 1,4-butanediol, other diols, residual sugars and organic acids is separated by liquid chromatography and the compounds are quantified. The components are separated on a resin based column in the hydrogen form using dilute sulfuric acid as the eluant. The separation is based partly on size exclusion (larger sugars elute first) and also on the ligand-ligand interaction between the hydroxyl groups on the compounds and the counter ion on the column packing.

Dual detectors are used with this system to quantify compounds that can not be seen with only one detector. The carbohydrates and diols are quantified by refractive index detection; the organic acids and aromatic compounds are quantified by UV at 210 nm. Standards of the components of interest are prepared and injected. The area under the peak for each pure compound is integrated and an area unit per gram per liter Response Factor is calculated for each component.

Response Factor (RF)=(area of component/amount of component in calibration standard)

$$\underline{Gram \text{/} Liter Component} = \frac{area of component}{Response Factor} \times \frac{dilution}{sample amount}$$

### Apparatus

Waters 515 HPLC Pump—Waters Chromatography Division, Catalog No. WAT020700 Waters—WISP 717 Autosampler or equivalent. Catalog No. WAT078500 Model 410 Differential Refractometer with IEEE Capability and External Temperature Control—Waters Chromatography Division, Catalog No. WAT070390

Waters—2487 Dual Wavelength UV/Vis Detector Catalog No. WAT081110 Heating Chamber—Waters Chromatography Division, Catalog No. WAT38040 2 Meta Carb 87H organic analysis columns, MetaChem Catalog No. 5210 or equivalent MetaChem 87H Guard Column, MetaChem Catalog No. 5212 or equivalent

### Instrument Parameters:

### Waters System

Column: 2-30 cm L×7.8 mm I.D Meta Carb 87H in series

### Column Temp: 47° C.

### Solvent: 0.0098 N H2SO4

Flow Rate: 0.6 ml/minute

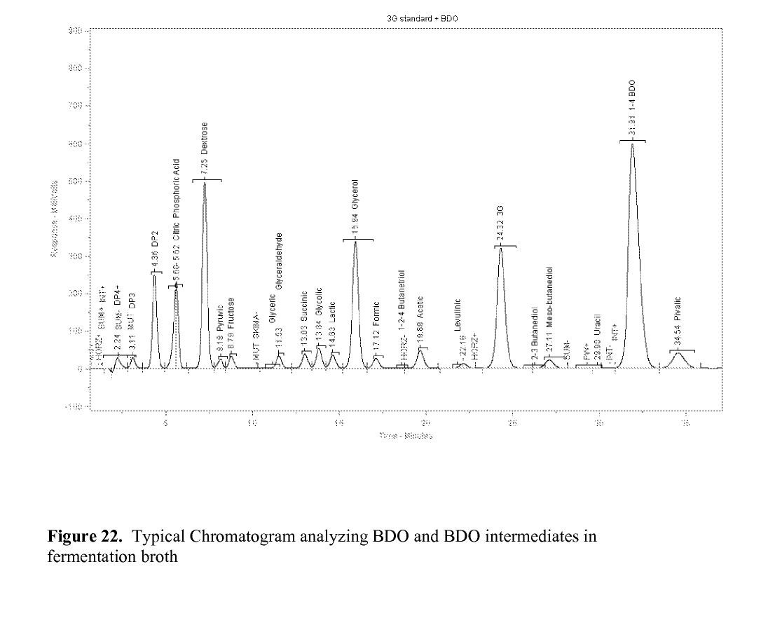

An expected typical chromatogram is shown in FIG. 22.

### Example 2C

BDO, intermediates and amino acids can also be analyzed by liquid chromatography/mass spectrometry (LCMS). 20 μA of broth is diluted 1:50 in aqueous 1% formic acid with 5% acetonitrile prior to centrifugation (5000×g, 10 m). The supernatant is removed and injected in 35 μl portions onto a reverse phase HPLC column (Waters Atlantis C18, 2.1×150 mm). Compounds are eluted isocratically at a flow rate of 0.35 ml min⁻¹, using 0.1% formic acid with 5% acetonitrile. Eluting compounds are detected with a triple quadropole mass spectrometer using positive or negative electrospray ionization. The instrument is operated in MRM mode to detect all compounds (1,4-butanediol (90.8>72.8), 4-hydroxybutanal (88.9>70.9), 5-hydroxy-2-oxopentanoate (131>87), 5-hydroxynorvaline (133.9>70.9), 2-pyrroline-5-carboxylate (113.9>67.9), glutamate (148>102), alpha-ketoglutarate (144.8>56.8), proline (116>70)). Individual compounds are quantified by comparison with standards injected under identical conditions.

### Example 3

### Evaluation of Enzyme Expression, Substrate Specificity and Activity

This Example describes the assays and methods used to evaluate the activity of enzymes encoded by individual candidate genes.

Plate based screening for enzyme activity leading to the production of an aldehyde. In certain cases, candidate enzyme activity can be qualitatively assessed by analyzing strains which overexpress the candidate enzyme using phenotypic screening analysis. For example, Schiff Aldehyde Indicator (SAI) plates which contain pararosaniline and bisulfite (see Conway et al. J. Bacteriol. 169(6):2591-2597, 1987) are useful for identifying strains which secrete aldehydes. When grown on SAI plates, strains which secrete carbonyl compounds, such as aldehydes, appear as red colonies and/or have red halos surrounding them due to the formation of the bright red Schiff base. SAI plates can be used to assess E. coli clones expressing the enzymes for step A2 (plates are supplemented with L-glutamate) and step A5 (plates are supplemented with 5-hydroxy-2-oxopentanoate) in the pathway depicted in FIG. 1A.

Use of 5-hydroxynorvaline as a nitrogen source to select aminotransferase clones. Selection of an aminotransferase that catalyzes a transamination using 5-hydroxynorvaline (5-HNV) as an amino donor is accomplished by requiring cells to grow using 5-HNV as the sole source of nitrogen. Under these conditions, only cells that can transfer the amino group from 5HNV to a central metabolite will grow. The enzymatic function most likely to be selected is one that can convert 5HNV and α-ketoglutarate to 5-hydroxy-2-oxopentanoate and glutamate.

The enzyme selection method is derived from one described in US2007076252. Plasmids containing cloned aminotransferase genes are transformed into E. coli strain BW25113, which has a deletion of the gabT gene encoding 4-aminobutyrate aminotransferase (Datsenko and Wanner, Proc. Natl. Acad. Sci. (USA) 97:6640-5, 2000). The gabT deletion prevents any utilization of 5-HNV by the host strain 4-aminobutyrate aminotransferase. Transformants are grown for 1.5 hours in SOC media (2% w/v bacto-tryptone, 0.5% w/v bacto-yeast extract, 8.56 mM NaCl, 2.5 mM KCl, 10 mM MgCl₂ and 20 mM glucose), centrifuged, washed with 0.85% NaCl, and resuspended in 0.75 ml of 0.85% NaCl to remove traces of nitrogen sources. Ten to 200 μl aliquots are spread onto selection plates (M9 minimal medium plates (see Example 7a below) without NH₄Cl, supplemented with 0.5% glycerol, 50 μM pyridoxine-HCl, 100 μM IPTG, 100 mM MOPS pH 7.0, 40 μg/mL kanamycin, and 5 mM 5-HNV). Plates are incubated at 37° C. Colonies which grow on the selection plates are restreaked onto a second selection plate to confirm the phenotype. Colonies from the second selection plate are used to inoculate individual 5-ml LB culture containing 40 μg kanamycin. The cultures are grown overnight at 37° C. and then subjected to plasmid isolation (Qiagen).

To confirm that 5-HNV utilization is conferred by the cloned aminotransferase gene and not a secondary mutation in the E. coli recipient, the plasmid with the cloned aminotransferase gene is isolated and transformed into E. coli strain BW25113 which has a deletion of the gabT gene. Transformants are plated on the selection plates described above, and the efficiency of colony formation is compared to control transformations with empty vectors.

Alternatively or additionally, PCR mutagenesis (see Example 4 below) followed by iterative enrichment can be performed as described in US2007076252 to either identify aminotransferases that catalyze the reaction shown in FIG. 1A, step A4, or to identify variants where this activity is increased and/or optimized. The method used is essentially that described in US2007076252 except 5-HNV is used as the sole source of nitrogen instead of beta-alanine

Enzyme expression and crude lysate preparation. The enzymatic activity of each candidate enzyme is evaluated, first in E. coli and subsequently in C. glutamicum. It will be apparent to one of skill in the art that the expression and lysate preparations may need to be modified depending on the host cell and recombinant gene of interest.

E. coli: E. coli strains transformed with expression clones, for example, pUC18 or MB4124 based clones having a medium strong constitutive promoter regulating the recombinant gene of interest, are grown aerobically in LB medium (Difco) containing the appropriate antibiotic(s). At an OD₆₀₀ of 0.5, IPTG is added to a final concentration of 0.25 mM. The cultures are grown to a final OD₆₀₀ of 1.5 at which time the cells are collected and harvested by centrifugation. Cell pellets are resuspended in BugBuster (Novagen) reagent containing benzonase and lysozyme. Alternatively, E. coli strains transformed with expression clones, are grown overnight aerobically at 37° C. in BHI containing the appropriate antibiotic(s). 300 μL of fresh overnight culture is used to inoculate 2.7 mL BHI containing 0.25 mM IPTG and the appropriate antibiotic. The cultures are grown aerobically for 4-5 hours at 37° C. at which time the cells are collected and harvested by centrifugation. Cell pellets are frozen at −80° C. for at least 15 minutes, then thawed and resuspended in BugBuster (Novagen) reagent. Lysis and lysate collection are performed according to the manufacturer's instructions and, for example, may involve use of a sonicator and/or French press. Cleared lysates are stored on ice until enzyme assays are performed.

C. glutamicum: C. glutamicum strains transformed with MB4124 expression clones containing the recombinant gene of interest are grown aerobically in BHI broth supplemented with the proper antibiotic(s). At an OD₆₀₀ of 0.5, IPTG is added to a final concentration of 0.25 mM. The cultures are grown to a final OD₆₀₀ of 1.5 at which time the cells are collected and harvested by centrifugation. The pellet is washed once in one volume of water and resuspended in lysis buffer (1.0 ml 1.0 M HEPES buffer, pH 7.5, 0.5 ml 1M KOH, 10 μl 0.5M EDTA, protease inhibitor cocktail, water to 5 ml; the final pH will vary depending on the individual enzyme being tested) and one volume of 0.1 mm acid washed glass beads. The mixture is repeatedly alternately vortexed and held on ice for 15 seconds, eight times. After centrifugation for 5 min at 4,000 rpm, the supernatant lysate is removed and respun for 20 min at 10,000 rpm. Alternatively, C. glutamicum strains transformed with MB4124 expression clones containing the recombinant gene of interest are grown aerobically overnight in BHI broth supplemented with the proper antibiotic(s). Five mL of fresh overnight culture is used to inoculate 200 mL BHI containing 0.25 mM IPTG and the appropriate antibiotic. The cultures are grown aerobically at 30° C. for 5-7 hours at which time the cells are collected and harvested by centrifugation at 4° C. The pellet is resuspended in 15 mL ice cold 39 mM KH₂PO₄, 61 mM K₂HPO₄, 0.5 M KCl and lysed using a BioSpec BeadBeater and following the manufacturer's instructions. The lysate is centrifuged at 16,000 RPM at 4° C. for 30 minutes and the supernatant retained on ice as the extract.

Protein Assays. Protein concentrations in lysates are determined according to the methods of Bradford et al. (Anal. Biochem. 72:248-254 [1976]) or by the method of Smith et al. (Anal. Biochem. 150(1):76-85 [1985]) and lysates are assayed according to the methods below.

Enzyme Assays. Enzyme assays are performed on the cell lysates prepared above. The activity for each recombinant enzyme is compared to a lysate from a vector only control culture which is generated in a fashion identical to the recombinant enzyme lysate. Enzymes for which activity towards a non-natural substrate is desired are assayed using both the natural substrate and the BDO pathway metabolite for which activity is sought.

Glutamate 5-kinase (Step A1): ATP-dependent activation of glutamate is estimated in whole cell extracts by the hydroxymate method as described by Hayzer and Leisinger (J. Gen. Microbiol. 118:287-293 [1980]). This method is based on the fact that in the presence of excess hydroxylamine, the unstable enzymatic product of the glutamate 5-kinase reaction, glutamate 5-phosphate, is rapidly converted to a stable end product, glutamate 5-hydroxamate. Hydroxamic acids such as glutamate hydroxymate turn purple in the presence of ferric chloride. Therefore the amount of glutamate 5-hydroxymate produced can be estimated by measuring the absorbance of the solution in a spectrophotometer at a wavelength of 540 nm and using the extinction coefficient of the Fe³⁺-hydroxymate product (250 mol^{-l} cm⁻¹) (from Kawahara et al. Agric. Biol. Chem. 53(9), 2475, 1989) or by generating a standard curve using purified glutamate 5-hydroxymate. One unit of activity is defined as the amount of enzyme required to produce 1 μmol of γ-glutamyl hydroxamate per min.

Glutamate 5-semialdehyde dehydrogenase (Step A2): Glutamate 5-semialdehyde dehydrogenase activity is measured using the assay of Hayzer and Leisinger (J. Gen. Microbiol. 118:287-293 [1980]). This method measures the phosphate dependent reduction of NADP+ to NADPH with glutamate 5-semialdehyde (derived from equilibrium with 1-pyrroline-5-carboxylate) as the substrate. The assay measures the reverse (i.e. non-biosynthetic) reaction for the enzyme because the labile nature of glutamate 5-phosphate precludes its use. The assay is spectrophotometric, measuring the increase in absorbance at a wavelength of 340 nm. One unit of glutamate 5-semialdehyde dehydrogenase is defined as the amount of enzyme necessary to produce 1 μmol of NADPH per min. The mM extinction coefficient of NADPH at 340 nm with a 1 cm light path is 6.27.

Oxidoreductase (Step A3): The activity is determined spectrophotometrically at a wavelength of 340 nm. At this wavelength, the decrease or increase in absorbance reflects the oxidation and reduction of NAD(P)H and NAD(P)⁺, respectively. One unit of enzyme activity is defined as the amount of enzyme that catalyzes the reduction or oxidation of 1.0 μmol of NAD⁺ and 1.0 μmol of NADH, respectively, per min at 30° C. (Kotani et al., J. Bacteriol. 185(24): 7120-7128. (2003)). The specific reaction mixture used to identify an oxidoreductase capable of reducing L-glutamate 5-semialdehyde (GSA) includes 1.1 mM L-1-pyrroline-5-carboxylate (used as a source of GSA), 400 mM NAD(P)H, 500 mM KCl, 100 mM KPO₄ buffer (pH 7.0) and cell extract at a concentration of 0.12 mg/ml. The reaction is carried out at 25° C.

Aminotransferase (Step A4): For routine uses such as screening mutant libraries, expression clones, or any experiment in which relatively large numbers of aminotransferase (AT) assays are performed, the protocol is based on that of Der Garabedian and Vermeersch (Eur. J. Biochem. 167:114-147. [1987]). This protocol includes a coupled assay in which glutamate formed in the transamination of aketoglutarate by an L-amino acid is measured by a secondary assay. In a second assay, the glutamate formed during the AT reaction is measured using a colorimetric assay kit from BioVision Research Products, Moutain View, Calif. (Catalog No. K629-100). When more precise measurements of activity are required, an HPLC method such as the one described by Marienhagen et al. (J. Bacteriol. 187(22):7639-7646 (2005)) can be used. Alternatively, the in vitro assays are analyzed by LC-MS for the desired product, 5-hydroxy 2-oxopentanoate.

Decarboxylase (Step A5): Alcohol Dehydrogenase linked reaction: In one example of a coupled assay, the decarboxylation of a substrate is linked to the activity of an alcohol dehydrogenase reaction that converts the aldehyde product to an alcohol with the concomitant oxidation of NAD(P)H (see Pohl et al., Eur. J. Biochem. 224:651-661 [1994]). The overall activity of the coupled reactions is assessed spectrophotometrically by measuring the reduction in absorbance at 340 nm which corresponds to the NAD(P)H oxidation. One enzyme unit is defined as the amount of enzyme that catalyzes the decarboxylation of 1 μmol of substrate per minute. Note: the coupled nature of the assay means that a functional enzyme catalyzing Step A6, the final step in the pathway, is required.

Aldehyde Dehydrogenase linked reaction: In another example of a coupled assay, the decarboxylation of a substrate is linked to the activity of an aldehyde dehydrogenase (Sigma catalog number A6338) reaction that converts the aldehyde product to a carboxylic acid with the concomitant reduction of NAD+. The overall activity of the coupled reactions is assessed spectrophotometrically by measuring the increase in absorbance at 340 nm which corresponds to the NAD+ reduction. One enzyme unit is defined as the amount of enzyme that catalyzes the decarboxylation of 1 μmol of substrate per minute.

Oxidoreductase (Step A6): Assays to determine the activity of candidates for the A6 step of the pathway depicted in FIG. 1A are assayed using the following assay mixture: 33 mM potassium phosphate buffer (pH 7.0), 1.0 mM substrate (e.g. 4-hydroxybutanal), 0.5 mM NADPH and 0.06-0.12 mg/ml cell extract. Activity is measured spectrophotometrically as the decrease in absorbance at 340 nm that occurs as NADPH is oxidized. One unit of enzyme activity is defined as the amount of enzyme that catalyzes the oxidation of 1.0 μmol of NADPH per min at 30° C. (Kotani et al., J. Bact. 185(24): 7120-7128. (2003)). When more precise measurements of activity are required, the assay reaction can be terminated by diluting the assay mixture 1:50 in HPLC diluent (5% acetonitrile, 0.1% formic acid) and the sample can be analyzed by LC-MS to determine the amount of BDO that has been formed from 4-hydroxybutanal.

In Vitro Substrate Conversion. Most of the enzyme assays described above are based on recording changes in absorbance at a given wavelength. In addition to such spectrophotometric readouts, these assays can also be analyzed by more direct methods using an endpoint assay where the reaction is terminated after a given period of time and the mixture is analyzed via HPLC or LC-MS methods.

Bioconversion assays. The activity of a candidate enzyme towards a given substrate can also be evaluated in vivo using a simple feeding assay. A strain expressing the candidate enzyme is grown in the presence of the substrate for which activity is desired. Samples of the culture are removed periodically and the cells are removed via centrifugation. The culture supernatant is then analyzed (e.g., by LC-MS) to determine how much of the substrate has been removed and how much of a given product is present. Because this type of assay relies on both transport of the substrate into the host cell and export of the enzymatic product out of the cell, it is not a viable option for all compounds, particularly those that do not readily diffuse across the membrane.

TABLE 3C shows the results of testing multiple candidate enzymes for BDO biosynthetic activity (steps A3-A6 of the pathway depicted in FIG. 1A) in the enzyme, in vitro substrate conversion and bioconversion assays described in this example. The aldehyde dehydrogenase linked reaction was used for step A5.

TABLE 3C
Activity Assays

| | BDO | | | | In Vitro | |
|---|---|---|---|---|---|---|
| | Biosynthetic | | SEQ ID NO. | | Substrate | |
| | Pathway Step | Enzyme | (also see | | Conversion | Bioconversion |
| Row | (see FIG. 1A) | Candidate | FIG. 1B) | Enzyme Assay | Assay | Assay |
| 1 | A3 | NosE_{N} | 16 | − | + | + |
| 2 | A3 | NosE_{Np} | 18 | + | + | + |
| 3 | A3 | NosE_{Av} | 20 | − | + | + |
| 4 | A3 | Hom_{Cg} | 22 | − | ND | ND |
| 5 | A3 | Hom_{Au} | 24 | − | ND | ND |
| 6 | A4 | IlvE_{Cg} | 28 | − | ND | ND |
| 7 | A4 | BioA_{Cg} | 29 | − | ND | ND |
| 8 | A4 | BATl_{Y1} | 31 | − | ND | ND |
| 9 | A4 | BcaT_{Lp} | 34 | − | ND | ND |
| 10 | A4 | BcaT_{Pf} | 58 | + | + | + |
| 11 | A5 | KdcA_{Ll} | 36 | + | + | + |
| 12 | A5 | Pdc_{Zm} | 38 | − | − | − |
| 13 | A5 | Pdc_{Ss} | 41 | − | − | − |
| 14 | A5 | Pdc_{Bc} | 42 | − | − | − |
| 15 | A5 | YeaU_{Ec} | 59 | − | ND | ND |
| 16 | A5 | PoxB_{Ec} | 60 | − | ND | ND |
| 17 | A5 | PanD_{Ec} | 61 | − | ND | ND |
| 18 | A5 | LdcC_{Ec} | 62 | − | ND | ND |
| 19 | A5 | SpeF_{Ec} | 63 | − | ND | ND |
| 20 | A5 | GadB_{Ec} | 64 | − | ND | ND |
| 21 | A5 | MenD_{Ec} | 65 | − | ND | ND |
| 22 | A5 | LysA_{Ec} | 66 | − | ND | ND |
| 23 | A5 | YiaQ_{Ec} | 67 | − | ND | ND |
| 24 | A5 | UlaD_{Ec} | 68 | − | ND | ND |
| 25 | A5 | Kgd_{Ms} | 69 | − | ND | ND |
| 26 | A5 | ARO10_{Sc} | 70 | − | ND | ND |
| 27 | A6 | ADH6_{Sc} | 47 | + | + | + |
| 28 | A6 | DhaT_{Cf} | 48 | − | ND | ND |
| 29 | A6 | AdhB_{Zm} | 50 | + | ND | ND |

“+” - measureable activity detected in the assay
“−” - no measurable activity detected in the assay
“ND” - not determined

### Example 4A

### Optimization of Recombinant 1,4-Butanediol Biosynthetic Enzyme Activities

As described above, candidate enzymes are assayed using both their native substrates and, when applicable, their non-native BDO pathway substrate. In situations where the enzyme lacks activity towards both substrates, the candidate strain is analyzed (as described below) using real time RT-PCR and Western blot analysis to ensure that the absence of activity is not due to a lack of mRNA or protein expression.

Confirmation of gene transcription and protein expression. Candidate constructs for which enzyme activity is not observed are examined by real time RT-PCR and Western blot analysis (using standard methods) to confirm that the gene is being transcribed and its expected product is being produced.

If the results of these analyses indicate that both transcription and translation are occurring as expected, the candidate gene can be cloned under a different promoter, the promoter can be modified, (in an attempt to alter the dynamic flux between translation and protein folding) or the gene can be randomly mutagenized. Mutagenesis will often result in nucleotide substitutions that either cause silent codon alterations or which alter the amino acid sequence slightly without affecting enzyme activity. Such changes can influence the overall process of transcription, translation and folding in such a way that a functional enzyme results.

PCR mutagenesis. Enzymes which are engineered to catalyze the conversion of non-native substrates may do so very inefficiently, if at all. Candidate genes encoding enzymes with suboptimal substrate specificity or catalytic activity may be modified by mutagenesis. In random PCR mutagenesis, the gene is subjected to error-prone PCR using the GeneMorph® Random Mutagenesis kit (Stratagene, La Jolla, Calif.). According to the manufacturer's instructions, oligonucleotide primers pairs with restriction sites that allow DNA fragments to be cloned directionally into pUC18 are used to amplify the candidate gene from template DNA. PCR mutagenized clones are transformed into the appropriate host and either selection or screening methods, for example, as described herein, are used to determine if the enzymatic activity of interest is present.

Site Directed Saturation Mutagenesis. Saturation mutagenesis can be performed to generate mutant genes having codons at one or more positions randomized. This technique is particularly useful when information about the three-dimensional structure of the encoded enzyme is available so that codons for amino acids that are known to be important for substrate binding can be targeted. Saturation mutagenesis can be carried out, for example, using the QuikChange Lightning Multi Site-Directed Mutagenesis Kit™ (Stratagene, La Jolla, Calif.). Using such a system, mutant alleles of the gene of interest can be generated in three steps using a single oligonucleotide in which the sequence corresponding to the targeted codon is randomized.

Using the primers in TABLE 3D, the QuikChange Lightning Multi Site-Directed Mutagenesis Kit was used to perform saturation mutagenesis on several codons in the synthetically synthesized kdcA (TABLES 3A and 3B, step A5) in an effort to increase its activity toward the substrate 5-hydroxy-2-oxopentanoic acid (5-HOP). The amino acids targeted by the TABLE 3D primers were chosen because they appeared to be part of the active site of KdcA based on analysis of the KdcA crystal structure (see e.g., Berthold et al. Acta Crystallogr D Biol Crystallogr 63:1217-24 [2007] and Yep et al. Bioorg Chem 34:325-36 [2006]). Mutagenized clones are transformed into the appropriate host and either selection or screening methods, for example, as described herein, are used to determine if the enzymatic activity of interest is present. Those with knowledge of the art will recognize that a similar strategy can be used to mutagenize other enzyme candidates for which structure-function information is available.

TABLE 3D
kdcA Saturation Mutagenesis Primers

| Primer | |
|---|---|
| Name* | Oligonucleotide Sequence** |
| E376 | 5′-caacgagaccatcgtggcannncagggaacctccttttt |
| | cg-3′ |
| Q377 | 5′-gagaccatcgtggcagagnnnggaacctcctttttcggc-3′ |
| G378 | 5′-ccatcgtggcagagcagnnnacctcctttttcggcgc-3′ |
| T379 | 5′-accatcgtggcagagcagggannntcctttttcggcgc-3′ |
| F381 | 5′-cagagcagggaacctccnnnttcggcgctagcaccat-3′ |
| F382 | 5′-tggcagagcagggaacctcctttnnnggcgctagcacc-3′ |
| P399 | 5′-aactctcgcttcatcggtcagnnnctatggggatctatc |
| | ggatac-3′ |
| G402 | 5′-ttcatcggtcagccactatggnnntctatcggatacact |
| | ttccca-3′ |
| S403 | 5′-cggtcagccactatggggannnatcggatacactttccc |
| | ag-3′ |
| 1404 | 5′-ggtcagccactatggggatctnnnggatacactttccca |
| | gc-3′ |
| V461 | 5′-caacaacgacggttacaccnnngaacgcgaaatccacgg |
| | tc-3′ |
| E462 | 5′-tcaacaacgacggttacaccgttnnncgcgaaatccacg |
| | gtc-3′ |
| 1465 | 5′-acggttacaccgttgaacgcgaannncacggtccaacc |
| | c-3′ |
| L535 | 5′-aaggaagacgctccaaagctcnnnaagaagatgggtaag |
| | ctcttc-3′ |

*Primer names indicate the number of the amino acid targeted for mutagenesis and the wild type residue located at that position. Amino acid numbering corresponds to that of GenBank Accession number AAS49166.
**Oligonucleotide sequences targeted for codon optimized kdcA gene (Step A5 in TABLES 3A and 3B)

### Example 4B

### Analysis of Step KdcA Mutant Activity

The KdcA site directed mutagenesis as described above yielded a mutant comprising the phenyalanine at position 382 mutated to a tryptophan (F382W). In addition, this mutant contained a second mutation that changed the glutamine at position 362 to a lysine (Q362K). To analyze the activity of this Q362K F382W double mutant, C. glutamicum strain ATCC 13032 was transformed with three different plasmids. Strain ME52 contained plasmid MB5640 which expresses the wild type kdcA allele, strain ME269 expresses the kdcA double mutant (Q362K, F382W) and strain ME13 contains only the plasmid vector, MB4124. The three strains were cultured and cell extracts and decarboxylase assays were performed as described in EXAMPLE 3 (step A5 activity) above. FIG. 10B shows that the strain expressing the Q362K F382W double mutant (ME269) exhibited increased in vitro KdcA enzyme activity as compared to strains expressing wild-type KdcA (ME52) or vector only (ME13).

### Example 5

### Cloning of Multigene Operon Constructs

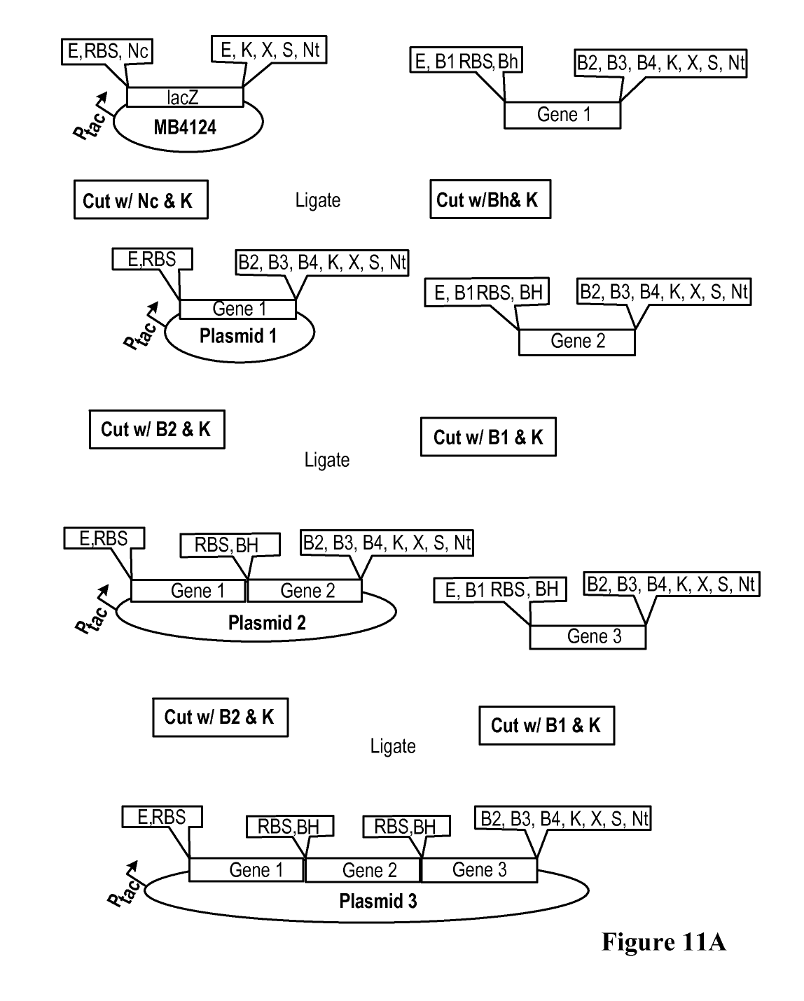

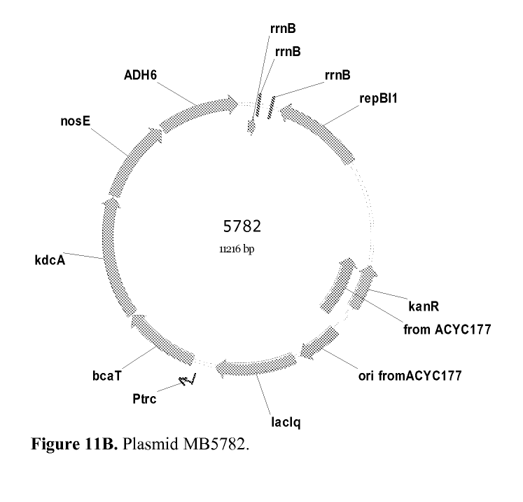

When engineering a biosynthetic pathway that contains multiple steps, it is often beneficial to express the genes in a single operon. FIG. 11A depicts a cloning strategy that allows multiple genes to be cloned together under a single promoter. In the depicted example strategy, each of the six BDO pathway genes in TABLE 3A are located on a DNA cassette or fragment (FIGS. 2 through 7) that contains a C. glutamicum ribosome binding site (RBS) upstream of the ORF with one or more blunt cutter restriction sites located both upstream and downstream of the gene. The strategy utilizes the blunt sites in a way that allows genes to be added to a construct, one at a time, each with its own C. glutamicum ribosomal binding site upstream. Using two different blunt cutter enzyme sites to form the junction between each successive gene insert allows use of the same enzymes for each step since both sites are lost during the previous ligation. Most of the gene cassettes used in this study have 3 different blunt cutter sites downstream of the ORFs so that the intergenic spacing can be varied if need be. Using the blunt cutters in combination with a “sticky-end” restriction site at the other end of the cassette genes (the compatible overhangs of NcoI on the vector and BspHI on the gene cassette) ensures that each gene will be added to the operon in a directed fashion. Persons having ordinary skill in the art will recognize that a similar strategy can be employed with other BDO pathway genes and that not all genes need to be part of the same cistron.

Plasmid containing 4-gene operon encoding BDO pathway steps A3 to A6. Plasmid MB5782 (FIG. 11B) contains a 4-gene operon comprised of (in order starting with gene closest to promoter) genes encoding: bcaT_{pf} (SEQ ID NO: 58), kdcA_{L1}(SEQ ID NO: 36), nosE_{Np} (SEQ ID NO:18) and ADH6_{sc} (SEQ ID NO: 47) carried on the vector MB4124 (FIG. 10). All 4 genes are transcribed from a single trc promoter and each gene is preceded by a C. glutamicum RBS. As described in Example 1A, MB4124 is a shuttle vector capable of replicating in both C. glutamicum and E. coli. C. glutamicum and E. coli host strains inactivated for the proC gene, encoding pyrroline-5-carboxylate reductase (EC 1.5.1.2) (step B5 in FIG. 1A; see Example 1B), can be made to produce BDO from glucose by transforming them with a plasmid that contains the genes required to catalyze steps A3-A6 of the pathway shown in FIG. 1A. Those skilled in the art will recognize that improved BDO producing strains can likely be generated by replacing one or more of the genes located on MB5782 with genes (e.g. mutant variants or other homologs) which have improved activity towards the desired pathway intermediate.

### Example 6

### Analysis of the Ability of Various Microbes to Grow in the Presence of BDO

Strains were obtained from ATCC except the two S. cerevisiae strains (CEN.PK: described in U.S. Pat. No. 7,049,108; Fermentis: a commercially available ethanol producer). TABLE 4 summarizes the media and growth conditions used in these studies. For each experiment, 50 mL of media was filter-sterilized before adding to pre-sterilized 250 mL triple baffled shake flasks. BDO, obtained from SABIC Innovative Plastics, had a density of 1.021 g/mL and was used at 99.5% minimum (wt. %) purity. Each strain was grown in four different shake flasks. The control had no BDO and the other three flasks contained 20 g/L (0.22 M), 90 g/L (1 M), and 135 g/L (1.5 M) of BDO, respectively.

Inocula were freshly grown on agar plates, cotton swabbed into a sterile Falcon® tube containing the same medium used in the shake flask and mixed well. The cell suspension was then inoculated into the prepared shake flasks to initiate the growth evaluation. The growth for each strain was monitored by measuring the OD (at 660 nm) and pH over the time course. If the medium contained glucose, such as Difco® Yeast Malt medium (YM), glucose concentration was monitored by using a YSI 2700 Select Biochemistry Analyzer.

TABLE 4
Strains and growth conditions used in the tolerance test.

| | | growth | |
|---|---|---|---|
| | | temperature | aeration |
| Strain | Media* | (° C.) | (rpm) |
| E. coli K12 (ATCC 25404) | NB | 30 | 175 |
| Corynebacterium glutamicum | NB | 37 | 175 |
| (ATCC 13032) | | | |
| Pseudomonas putida (ATCC 700801) | TSB | 30 | 175 |
| Zymomonas mobilis (ATCC 31821) | RM | 30 | 175 |
| S. cerevisiae (CEN.PK strain) | YM | 30 | 150 |
| S. cerevisiae (Fermentis strain) | YM | 30 | 150 |
| Yarrowia lipolytica (ATCC 20228) | YM | 30 | 175 |
| Zygosaccharomyces bailii | YM | 30 | 175 |
| (ATCC 60594) | | | |

*NB = Nutrient broth; TSB = Trypticase soy broth; RM = ATCC #1341 medium;
YM = Difco ®
Yeast Malt medium.

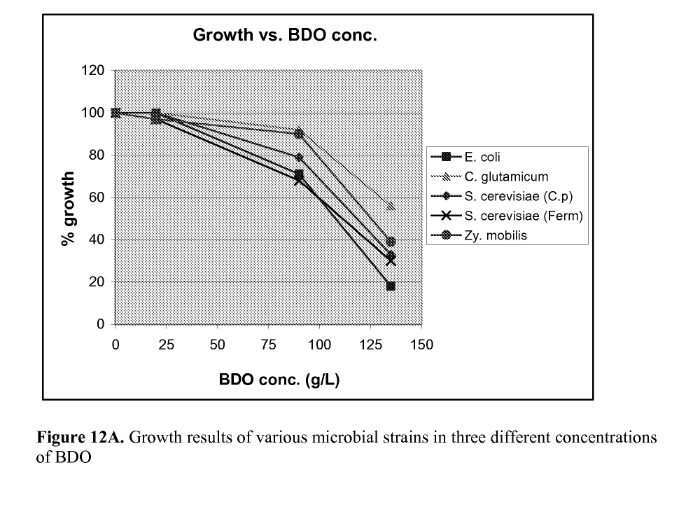

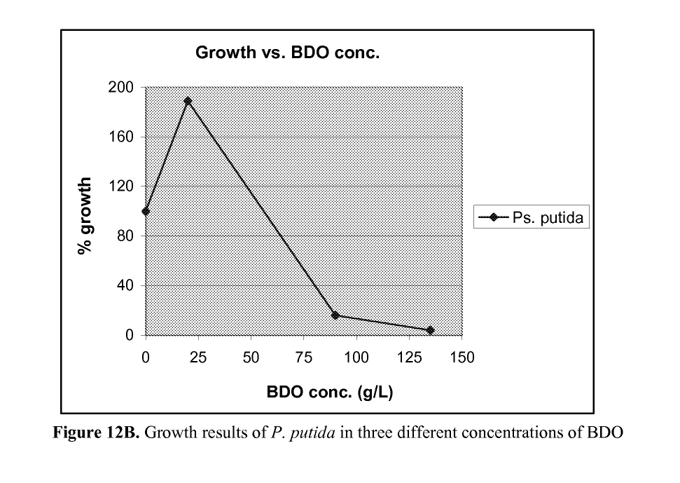

Since different media and growth conditions were used, results were expressed as % of growth where the control, no BDO flask, represented 100% growth. FIGS. 12A and 12B show the fully compiled results for all strains except Y. lipolytica (ATCC 20228) and Z. bailii (ATCC 60594).

Overall, two bacteria, Corynebacterium glutamicum (ATCC 13032) and Zymomonas mobilis (ATCC 31821) grew better than the rest of strains in the presence of 90 g/L and 135 g/L of BDO. C. glutamicum grew better than Z. mobilis when the BDO concentration was increased to 135 g/L (FIG. 12A).

Pseudomonas putida (ATCC 700801) grew poorly in higher concentrations of BDO (90 g/L and 135 g/L) (FIG. 12B). However, at 20 g/L of BDO, this strain grew to a higher cell density (approximately twice of the control). One explanation for this observation is that at 20 g/L BDO, Pseudomonas putida degrades BDO and uses it as an extra carbon source for growth. Thus, before using P. putida as a host strain, appropriate modifications may be required in order to decrease P. putida utilization of BDO as a carbon source.

The two S. cerevisiae strains (CEN.PK and Fermentis) and the E. coli K-12 strain demonstrated similar ability to tolerate 90 g/L BDO; however, S. cerevisiae showed greater tolerance than E. coli in the presence of 135 g/L of BDO.

Both Yarrowia lipolytica (ATCC 20228) and Zygosaccharomyces bailii (ATCC 60594) were only able to grow in the presence of 20 g/L of BDO. The presence of 90 g/L and 135 g/L of BDO in the medium completely inhibited growth and resulted in the loss of cell viability.

### Example 7A

### M9 Medium (for 1 Liter)

| | |
|---|---|
| Na₂HPO₄ | 6 grams |
| KH₂PO₄ | 3 grams |
| NaCl | 0.5 gram |
| NH₄Cl | 1 gram |
| ddH₂O to 940 ml | |

Autoclave and then add the following filter sterilized solutions:

| | |
|---|---|
| MgSO₄•7H₂O (16 mg/ml) | 15.4 ml |
| Thiamine-HCl (1 mg/ml) | 0.45 ml |
| Biotin (50 mg/L) | 9.0 ml |
| Pantothenic Acid (50 mg/L) | 10.0 ml |
| Glucose (40%) | 25.0 ml |

### Example 7B

### Growth Media for BDO Production

AZ defined medium
35 g glucose

### 2 g NaCl

3 g sodium citrate, dihydrate
0.1 g calcium chloride dihydrate
0.45 g magnesium sulfate heptahydrate
10 ml of 75 mg/L Na₂EDTA dihydrate
20 ml of 2.5 g/L FeSO₄ heptahydrate
20 ml 100× Salts (see below)
4 g potassium phosphate dibasic
2 g potassium phosphate monobasic
7.5 g ammonium sulfate
3.75 g urea
0.045 ml of 10 mg/ml thiamine
9 ml of 50 mg/L biotin
10 ml of 450 mg/L pantothenic acid

### For 100× Salts:

200 mg manganese sulfate
20 mg sodium tetraborate decahydrate
10 mg ammonium molybdate tetrahydrate
200 mg ferric chloride hexahydrate
50 mg zinc sulfate heptahydrate
20 mg cupric chloride dihydrate
2.5 g magnesium sulfate heptahydrate
Up to 1 L with deionized water

### T&L Medium (Per Liter)

25 g glucose
5 g ammonium chloride
100 mg potassium phosphate monobasic
150 mg potassium phosphate dibasic
50 mg magnesium sulfate heptahydrate
2 mg FeSO₄ heptahydrate
3 mg manganese sulfate heptahydrate
2 mg zinc sulfate heptahydrate
100 μg biotin
2 mg thiamine-HCl
1 g yeast extract

### 500 mg Urea

1 g calcium carbonate
Up to 1 L with deionized water

### Example 7C

### BDO Production in Corynebacterium glutamicum

In order to produce BDO via the pathway shown in FIG. 1A, a strain must produce glutamate 5-semialdehyde (GSA) at levels high enough to allow flux through steps A3-A6. In many cells, GSA is formed from L-glutamate as a metabolite during proline biosynthesis via a two step process catalyzed by ProB and ProA. Under cellular conditions GSA rapidly cyclizes to form pyrroline 5-carboxylate (P5C) which is then converted to proline by ProC. GSA and P5C usually exist in a state of tautomeric equilibrium. Therefore, cells in which the proC is inactivated will accumulate GSA and P5C in their cytoplasm making GSA available for the BDO pathway.

The 4 DNA sequences (encoding enzymes for BDO pathway steps A3-A6; nosE_{Np} (SEQ ID NO:18), bcaT_{pf} (SEQ ID NO:58), kdcA_{L1} (SEQ ID NO:36), and ADH6_{Sc} (SEQ ID NO:47)) in TABLE 3B were introduced into C. glutamicum. C. glutamicum strain ME114 is a proC deletion (ΔproC) strain with a 2-gene operon comprising nosE_{Np} and ADH6_{Sc} integrated into a galK chromosomal deletion. ME114 was used to generate three BDO production strains, ME120, ME124 and ME220. Each production strain contains a different episomal plasmid. ME120 has a plasmid (MB5721) with a 2 gene operon comprising bcaT_{Pf} and kdcA_{L1}. ME124 contains a plasmid (MB5748) with a 3 gene operon comprising bcaTp_{Pf}, kdcA_{L1} and nosE_{Np}. Lastly, strain ME220 has a plasmid (MB5782; see example 5 herein) with a 4 gene operon comprising bcaT_{Pf}, kdcA_{L1}, nosE_{Np} and ADH6_{Sc} Thus, ME124 and ME220 have both episomal and chromosomal copies of nosE_{Np} and, in addition, ME220 has both episomal and chromosomal copies of ADH6_{Sc}.

All of the BDO biosynthetic genes in plasmids MB5721, MB5748 and MB5782 are regulated by the IPTG inducible E. coli trc promoter and contain the kanR selectable marker gene. The genotypes of C. glutamicum strains ME114, ME120, ME124 and ME220 are summarized in TABLE 5. All strains are proline auxotrophs and therefore require proline supplemented media for growth.

TABLE 5
Corynebacterium control and BDO Production Strains

| Strain | Relevant Genotype |
|---|---|
| ME114 (control) | ΔproC, ΔgalK::nosE_{Np} + ΔDH6_{Sc} |
| ME120 | ΔproC, ΔgalK::nosE_{Np} + ΔDH6_{Sc}, plasmid MB5721[bcaT_{pf} + kdcA_{Ll}] |
| ME124 | ΔproC, ΔgalK::nosE_{Np} + ΔDH6_{Sc}, plasmid MB5748[bcaT_{pf} + kdcA_{Ll} + |
| | nosE_{Np}] |
| ME220 | ΔproC, ΔgalK::nosE_{Np} + ΔDH6_{Sc}, plasmid MB5782[bcaT_{pf} + kdcA_{Ll} + |
| | nosE_{Np} + ADH6_{Sc}] |

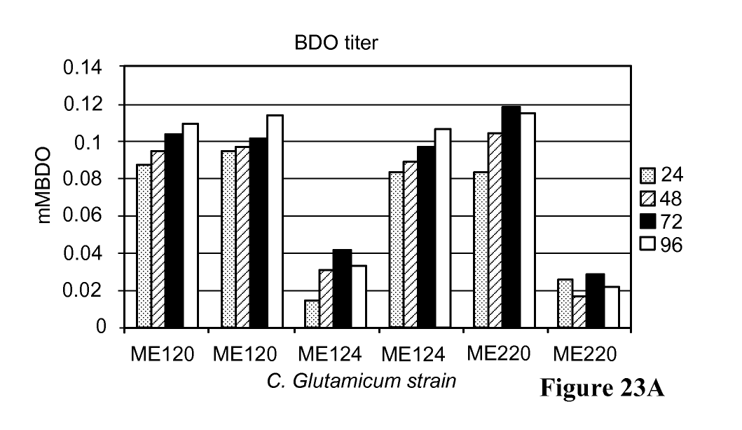

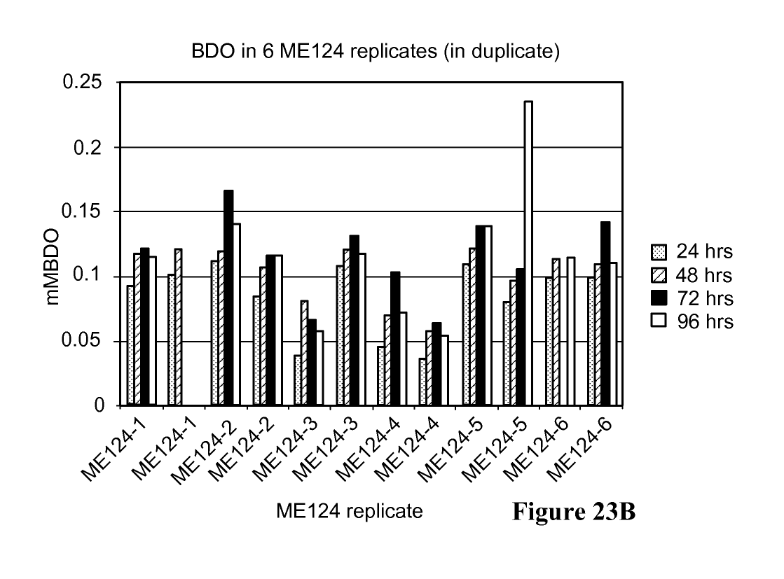

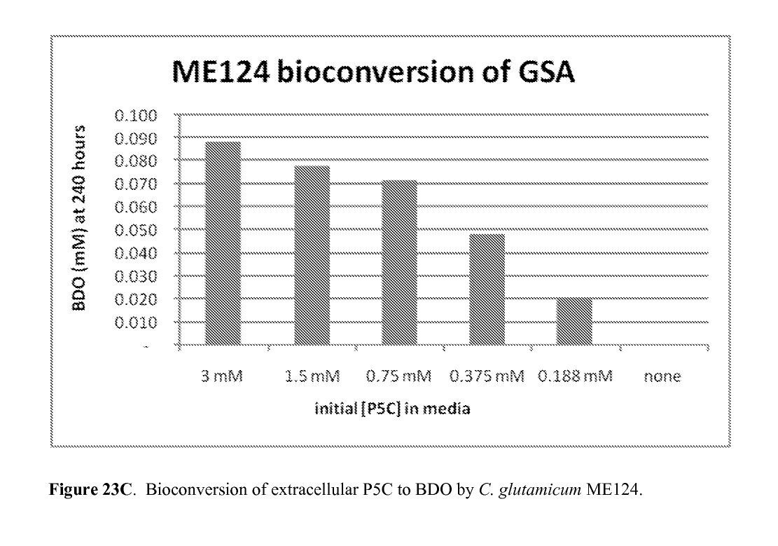

Two independent cultures of control parental strain ME114 and each of the three BDO production strains were grown in AZ defined medium (Example 7A herein) and analyzed for BDO production as described in Example 2c herein after 24, 48, 72 and 96 hours of growth. Strain ME114 exhibited no BDO production at any time point (data not shown). ME120, ME124 and ME220 all produced some BDO at each time point (FIG. 23A). Even though the strains had different genotypes, there was no obvious difference in BDO production among the three strains. There was some variability in BDO production for the independent cultures of strains ME124 and ME220.

To examine the variability in independent cultures of an individual strain, ME124 was grown overnight at 30° C. on a BHI plate containing kanamycin. The next day, six single ME124 colonies were each inoculated into separate 3-ml BHI cultures and grown overnight at 30° C. in a rolling incubator. 300 [symbol] 1 of each of the six overnight cultures was then inoculated into two replicate cultures of 3-ml AZ defined medium supplemented with 1.0 mM proline, 0.25 mM IPTG and 10 mg/ml kanamycin (150 μl overnight culture per 3-ml AZ culture; see Example 7B herein for AZ recipe). These twelve cultures were grown at 30° C. in rolling incubator. 100 μl aliquots were removed and analyzed by LC-MS (see Example 2 herein) for the presence of BDO at 24, 48, 72 and 96 hours. On average, ME124 produced between 0.05-0.15 mM BDO, most of which appeared during the first 24 hours of culturing (FIG. 23B). The negative control, ME33, a wild type C. glutamicum parent strain having only episomal vector (MB4124) was grown under identical conditions and did not produce BDO at any time point (data not shown).

The variability in BDO production observed in FIGS. 23A and 23B for individual strains may be due, in part, to insufficient levels of GSA flux into the last four steps of the pathway. Proline biosynthesis in many bacteria is subject to feedback inhibition of ProB, the gene encoding glutamate 5-kinase. Therefore, in ΔproC BDO production strains that do not have feedback resistant ProB alleles, the level of proline in the medium used to culture the strains needs to be high enough to allow growth but low enough so as not to inhibit the first step in the pathway catalyzed by ProB. Therefore, in order to assess the effect of GSA levels on BDO production in strains with a wild type ProB, a feeding experiment was conducted by growing ME124 in varying levels of GSA/P5C.

ME124 was inoculated into 5 ml cultures of T&L medium (see Example 7B herein) containing 1.0 mM proline, 0.3 mM IPTG, 10 [symbol] g/ml kanamycin and varying concentrations of P5C (used as a source of GSA) and incubated at 30° C. on a roller. The cultures were analyzed by LC-MS for BDO production. As shown in FIG. 23C, at 240 hours, BDO production levels directly correlated to the GSA/P5C starting concentration.

### Example 8A

### E. coli as a Production Organism

Those skilled in the art will appreciate that the basic strategy employed to generate a C. glutamicum BDO producing strain can also be used to engineer an E. coli strain which produces BDO and/or BDO intermediates. It is expected that the complement of polypeptides (for example polypeptides disclosed in FIGS. 1B and 13-18, including polypeptides that share at least 80% identity to these polypeptides) required to produce BDO from glutamate (steps A1-A6; FIG. 1A) in E. coli will be the same or similar to those required in C. glutamicum.

Unlike C. glutamicum, E. coli does not produce large amounts of L-glutamate naturally. Thus, for optimal production of BDO and or intermediates, E. coli production strains may optionally be engineered to contain mutations in one or more endogenous genes that result in increased the glutamate production. Examples of such mutations include those in sucA which decrease or abolish α-ketoglutarate dehydrogenase activity thereby increasing the amount of α-ketoglutarate that is available for conversion to glutamate. Strains may also be modified to increase phosphoenolpyruvate carboxylase and/or glutamate dehydrogenase activities, both of which have been shown to increase glutamate production.

E. coli production strains may also be optionally modified to alleviate unfavorable regulatory mechanisms or to reduce the draining of metabolites by competing pathways. Examples of such mutations include mutations in proB that result in the expression of a glutamate 5-kinase which is not sensitive to inhibition by L-proline, and mutations in proC which reduce or abolish the activity of pyrroline 5-carboxylate reductase and result in higher levels of glutamate 5-semialdehyde (which is at equilibrium with 1-pyrroline-5-carboxylate) in the cell.

Cloning vectors, both episomal and integrative, for expressing heterologous genes in E. coli are abundant and well know within the art. Therefore, a complete set of BDO pathway genes can be cloned and expressed in E. coli in the same manner as described for C. glutamicum.

### Example 8B

### Production of BDO by E. Coli

An E. coli strain (CGSC# 4515) in which proC has been inactivated was acquired from the “Coli Genetic Stock Center” at Yale University. The strain was transformed with plasmid MB5782 (see Example 5 herein) which contains the four genes required for steps A3 through A6 of the BDO pathway (FIG. 1A) and the resulting transformant was designated ME140. To assess the ability of E. coli to produce BDO, ME140 was compared to that of a control strain (ME139) which is CGSC# 4515 transformed with the vector MB4124 (FIG. 10). The strains were grown in M9 medium (see Example 7A herein) containing, 1% glucose, 1.0 mM proline, 5 r g/ml kanamycin and 0.2 mM IPTG at 37° C. At 24 hr intervals, aliquots of the cultures were removed and the BDO levels were quantified by LC-MS (as in EXAMPLE 2 herein). BDO production by strain ME140 was observed between 48 and 72 hours at concentrations between 0.02 mM to 0.07 mM. BDO was never observed in the medium of the control strain, ME139, grown under the same conditions.

### Example 9

### Analysis of Step as Decarboxylase Activity of Lactococcus Lactis KdcA

KdcA_{L1} from Lactococcus lactis was able to decarboxylate 5-hydroxy-2-oxopentanoate (5-HOP) in the assays described in example 3 herein (see TABLE 3C; step AS Aldehyde dehydrogenase linked reaction). To study this activity further, the KdcA_{L1} protein was expressed in E. coli, purified and analyzed.

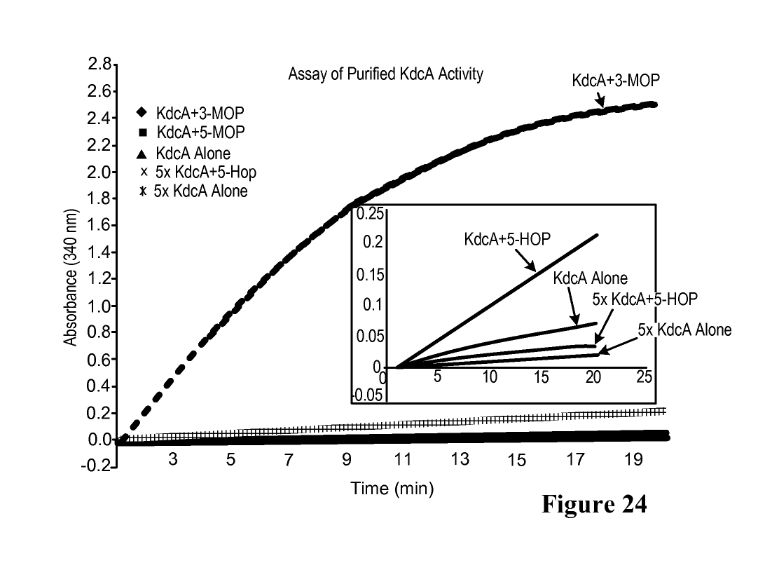

Standard molecular biology techniques were used to create an E. coli expression construct encoding KdcA_{L1} having three histidine (His) residues attached to its C-terminus. This His-tagged enzyme was over-expressed in E. coli, purified and assayed to assess its activity towards a natural substrate, 3-methyl-2-oxopentanoate (3-MOP) and its BDO pathway substrate, 5-hydroxy-2-oxopentanoate (5-HOP). The purified enzyme was assayed as described in example 3 above (step A5, Aldehyde dehydrogenase linked reaction). FIG. 24 shows that KdcA_{L1} has activity towards 5-HOP as a substrate with a Vmax of approximately 0.15 U/mg. This Vmax is about 10 times lower than the rate of 3-MOP activity. The affinity of KdcA_{L1} for 5-HOP (Km=49 mM) is less than its affinity for 3-MOP (Km=1.3 mM).

### Example 10

### Analysis of Step A3 Oxidoreductase Activity of Nostoc punctiforme NosE

NosE_{Np} from Nostoc Punctiforme was able to reduce L-1-pyrroline-5-carboxylate (P5C; a source of L-glutamate 5-semialdehyde) to 5-hydroxynorvaline in the assays described in example 3 herein (see TABLE 3C; step A3 Oxidoreductase activity). To study this activity further, the NosE_{Np} protein was expressed in E. coli, purified and analyzed.

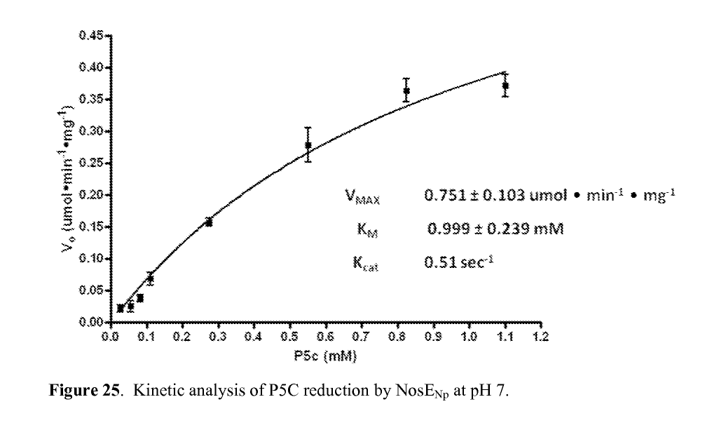

Standard molecular biology techniques were used to create an E. coli expression construct encoding NosE_{Np} having three histidine (His) residues attached to its C-terminus. This His-tagged enzyme was over-expressed in E. coli, purified and assayed to assess its oxidoreductase activity as described in example 3 above (step A3 Oxidoreductase activity). FIG. 25 shows the results of a kinetic analysis that was performed for the activity of purified NosE_{Np} on increasing amounts of the substrate, P5C.

What is claimed is:

1. A recombinant microbial cell comprising at least two nucleic acid molecules selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol,
wherein either: (i) the recombinant cell has been modified to reduce the expression or activity of a polypeptide that catalyzes the conversion of pyrroline 5-carboxylate to proline or (ii) the recombinant cell does not comprise a nucleic acid molecule encoding active pyrroline 5-carboxylate reductase; and
wherein the cell produces at least one of 5-hydroxy-L-norvaline, 5-hydroxy-2-oxopentanoate, 4-hydroxybutanal and 1,4-butanediol.

2. The recombinant microbial cell of claim 1 comprising at least three nucleic acid molecules selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol.

3. The recombinant microbial cell of claim 1 comprising at least four nucleic acid molecules selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol.

4. The recombinant microbial cell of claim 1 comprising at least five nucleic acid molecules selected from:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol.

5. The recombinant microbial cell of claim 1 comprising:
(a) a nucleic acid molecule encoding a polypeptide that catalyzes the conversion of L-glutamate to L-glutamate 5-phosphate;
(b) a nucleic acid molecule encoding a polypeptide that catalyzes the conversion of L-glutamate 5-phosphate to L-glutamate 5-semialdehyde;
(c) a nucleic acid molecule encoding a polypeptide that catalyzes the conversion of L-glutamate 5-semialdehyde to 5-hydroxy-L-norvaline;
(d) a nucleic acid molecule encoding a polypeptide that catalyzes the conversion of 5-hydroxy-L-norvaline to 5-hydroxy-2-oxopentanoate;
(e) a nucleic acid molecule encoding a polypeptide that catalyzes the conversion of 5-hydroxy-2-oxopentanoate to 4-hydroxybutanal;
and
(f) a nucleic acid molecule encoding a polypeptide that catalyzes the conversion of 4-hydroxybutanal to 1,4-butanediol.

6. A recombinant microbial cell comprising at least two nucleic acid molecules selected from:
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
(c) a nucleic acid molecule encoding a polypeptide that catalyzes the conversion of 5-hydroxy-2-oxopentanoate to 4-hydroxybutanal;
and
(d) a nucleic acid molecule encoding a polypeptide that catalyzes the conversion of 4-hydroxybutanal to 1,4-butanediol
wherein either: (i) the recombinant cell has been modified to reduce the expression or activity of a polypeptide that catalyzes the conversion of pyrroline 5-carboxylate to proline or (ii) the recombinant cell does not comprise a nucleic acid molecule encoding active pyrroline 5-carboxylate reductase; and
wherein the cell produces at least one of 5-hydroxy-L-norvaline, 5-hydroxy-2-oxopentanoate, 4-hydroxybutanal and 1,4-butanediol.

7. The recombinant microbial cell of claim 6 comprising at least three nucleic acid molecules selected from:
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
(c) a nucleic acid molecule encoding a polypeptide that catalyzes the conversion of 5-hydroxy-2-oxopentanoate to 4-hydroxybutanal;
and
(d) a nucleic acid molecule encoding a polypeptide that catalyzes the conversion of 4-hydroxybutanal to 1,4-butanediol.

8. The recombinant microbial cell of claim 6 comprising
(a) a nucleic acid molecule encoding a polypeptide that catalyzes the conversion of L-glutamate 5-semialdehyde to 5-hydroxy-L-norvaline;
(b) a nucleic acid molecule encoding a polypeptide that catalyzes the conversion of 5-hydroxy-L-norvaline to 5-hydroxy-2-oxopentanoate;
(c) a nucleic acid molecule encoding a polypeptide that catalyzes the conversion of 5-hydroxy-2-oxopentanoate to 4-hydroxybutanal;
and
(d) a nucleic acid molecule encoding a polypeptide that catalyzes the conversion of 4-hydroxybutanal to 1,4-butanediol.

9.-42. (canceled)

43. The recombinant microbial cell of claim 1 wherein:
(a) the polypeptide that catalyzes the conversion of L-glutamate to L-glutamate 5-phosphate is at least 80% identical to any of SEQ ID NOs:1-9 or any of the sequences represented by the Genbank Accession numbers in FIG. 13;
(b) the polypeptide that catalyzes the conversion of L-glutamate 5-phosphate to L-glutamate 5-semialdehyde is at least 80% identical to any of SEQ ID NOs:10-15 or any of the sequences represented by the Genbank Accession numbers in FIG. 14;
(c) the polypeptide that catalyzes the conversion of L-glutamate 5-semialdehyde to 5-hydroxy-L-norvaline is at least 80% identical to any of SEQ ID NOs:16-27 or any of the sequences represented by the Genbank Accession numbers in FIG. 15;
(d) the polypeptide that catalyzes the conversion of 5-hydroxy-L-norvaline to 5-hydroxy-2-oxopentanoate is at least 80% identical to any of SEQ ID NOs:28-35, 58 or any of the sequences represented by the Genbank Accession numbers in FIG. 16;
(e) the polypeptide catalyzes the conversion of 5-hydroxy-2-oxopentanoate to 4-hydroxybutanal is at least 80% identical to any of SEQ ID NOs:36-43, 59-70 or any of the sequences represented by the Genbank Accession numbers in FIG. 17;
and
(f) the polypeptide that catalyzes the conversion of 4-hydroxybutanal to 1,4-butanediol is at least 80% identical to any of SEQ ID NOs:44-50 or any of the sequences represented by the Genbank Accession numbers in FIG. 18.

44. The recombinant microbial cell of claim 6 wherein:
(a) the polypeptide that catalyzes the conversion of L-glutamate 5-semialdehyde to 5-hydroxy-L-norvaline is at least 80% identical to any of SEQ ID NOs:16-27 or any of the sequences represented by the Genbank Accession numbers in FIG. 15;
(b) the polypeptide that catalyzes the conversion of 5-hydroxy-L-norvaline to 5-hydroxy-2-oxopentanoate is at least 80% identical to any of SEQ ID NOs:28-35, 58 or any of the sequences represented by the Genbank Accession numbers in FIG. 16;
(c) the polypeptide catalyzes the conversion of 5-hydroxy-2-oxopentanoate to 4-hydroxybutanal is at least 80% identical to any of SEQ ID NOs:36-43, 59-70 or any of the sequences represented by the Genbank Accession numbers in FIG. 17;
and
(d) the polypeptide that catalyzes the conversion of 4-hydroxybutanal to 1,4-butanediol is at least 80% identical to any of SEQ ID NOs:44-50 or any of the sequences represented by the Genbank Accession numbers in FIG. 18.

45.-68. (canceled)

69. The recombinant cell of claim 1 wherein the polypeptide that catalyzes the conversion of L-glutamate to L-glutamate 5-phosphate is a variant having one or more amino acid changes that confer reduced inhibition compared to an otherwise identical polypeptide lacking the one or more amino acid changes.

70.-72. (canceled)

73. The recombinant microbial cell of claim 1 wherein the microbial cell is a bacterium of species Corynebacterium glutamicum, Corynebacterium acetoglutamicum, Corynebacterium melassecola, Corynebacterium thermoaminogenes, Brevibacterium lactofermentum, Brevibacterium lactis, Brevibacterium ketoglutamicum, Brevibacterium saccharolyticum or Brevibacterium flavum bacterium.

74.-76. (canceled)

77. The recombinant microbial cell of claim 1 wherein at least two, three, four or five of the polypeptides are heterologous to the cell.

78. The recombinant microbial cell of claim 6 wherein at least two, three or four of the polypeptides are heterologous to the cell.

79. The recombinant microbial cell of claim 1 or claim 6 wherein at least four of the polypeptides are heterologous to the cell.

80. The recombinant microbial cell of claim 1 or claim 6 wherein at least five of the polypeptides are heterologous to the cell.

81.-99. (canceled)

100. A method for the production of 1,4-butanediol comprising:
providing a host cell according to claim 1; and
culturing the host cell under conditions whereby 1,4-butanediol is produced.

101. The method of claim 100 further comprising isolating the produced 1,4-butanediol.

102. The method of claim 100 wherein the cell is cultured in glucose.

103. The method of claim 100 wherein the cell produces 1 mM of 1,4-butanediol per mole of glucose.

104.-106. (canceled)

107. The recombinant microbial cell of claim 1 wherein the expression of at least one of the nucleic acids of (a) through (f) is under the control of an inducible promoter.

108. The recombinant microbial cell of claim 6 wherein the expression of at least one of the nucleic acids of (a) through (f) is under the control of an inducible promoter.

109. The recombinant cell of claim 6 wherein the polypeptide that catalyzes the conversion of L-glutamate to L-glutamate 5-phosphate is a variant having one or more amino acid changes that confer reduced inhibition compared to an otherwise identical polypeptide lacking the one or more amino acid changes.

110. The recombinant microbial cell of claim 6 wherein the microbial cell is a bacterium of species Corynebacterium glutamicum, Corynebacterium acetoglutamicum, Corynebacterium melassecola, Corynebacterium thermoaminogenes, Brevibacterium lactofermentum, Brevibacterium lactis, Brevibacterium ketoglutamicum, Brevibacterium saccharolyticum or Brevibacterium flavum bacterium.


SEQUENCE LISTING (104 sequences)

SEQ ID NO: 1 - protein, 409 aa, Corynebacterium glutamicum
MAPVTGLPVTPYSQEASIGASFPAVDPDTKDSAAYGHESGMRERISNAKRVVVKIGSSSLTNDEDGHTVDPNRINTIVNALQARMEAGSDLIVVSSGAVAAGMAPLGLSTRPTELAVKQAAAAVGQVHLMHQWGRSFARYGRPIGQVLLTAADAGKRDRARNAQRTIDKLRILGAVPIVNENDTVATTGVNFGDNDRLAAIVAHLVSADALVLLSDVDGLFDKNPTDPTAKFISEVRDGNDLKGVIAGDGGKVGTGGMASKVSAARLASRSGVPVLLTSAANIGPALEDAQVGTVFHPKDNRLSAWKFWALYAADTAGKIRLDDGAVEAVTSGGKSLLAVGITEIIGDFQQGEIVEILGPAGQIIGRGEVSYDSDTLQSMVGMQTQDLPDGMQRPVVHADYLSNYASRA

SEQ ID NO: 2 - protein, 366 aa, Nocardia farcinica
MSAARQAIASARSVVVKIGSSALTSLEGGLDTTRLDRLADAVEARMRAGSDVVVVSSGAIGAGLAPLGLSRRPRDLATKQAAASVGQLALAHAWGTSFARYGRTVGQVLLSADDFSRREHHRNAQRTLDRLRSLGAVAVVNENDTVATEEIRFGDNDRLAALVAHLVGADALILLSDVEGLYDGDPRKGAATFIPEVRSSADLDGVIAGSGGVLGTGGMASKLSAARLAADAGVPVLLAAAEQAATALGSGTVGTAFAARPVRLSARKFWVRHAADSRGALVLDDGAVQAVAQRRRSLLAAGITAVRGRFHGGDVVDLLAADQRLVARGVVEYDSTELSTMLGRSTTELPDTMQRPVIHADDLVKV

SEQ ID NO: 3 - protein, 366 aa, Rhodococcus sp.
MSATRRTIASAGSIVVKIGSSALTSLVGGLDVGRLDALADAIEDRMRAGSDVVVVSSGAVGAGLAPLGLTKRPRDLATKQAAASVGQLALAHAWGTSFARYGRTVGQVLLTADDIARRAQHRNAQRTLDRLRALHAVAIVNENDTVATAELRFGDNDRLAALVAHLVGADALVLLSDVDGLYDGDPRKGNATLIPEVNSPEDLDGVVAGSGGALGTGGMASKLSAARLAADAGVPVLLAAASDAGAALRDAGVGTAFVARPSRLSARKFWVRHAADEQGILHIDEGAVRAVVTKRRSLLPAGISAVSGRFYGGDVVSLLGPDERPVARGVVAYDSAEISDILGKSTQELPAEMQRPVVHADDLVPL

SEQ ID NO: 4 - protein, 377 aa, Renibacterium salmoninarum
MRSDSVVVGGRELLKTASRIVVKVGSSSLTSVSGGISEQALSQLVDVLAARKKQGAEIILVSSGAIAAGLAPLGLARRPKDLATQQSAASVGQSLLMARYGAAFGSHNVTVSQVLLTAEDLTRRTQYANVYRALERLLNLGVLPIVNENDTVATHEIRFGDNDRLAALVAHLVKADAMVLLSDVDAVYDGPPNTGARRIEQISSPEDLAGIKIGSVGSAGVGTGGMATKVQAASIAAGFGTPALVTSAANAEAALAGADVGTWFSLHGERRPIRLLWLAHLASTHGKLTLDDGAVAAIRERHKSLLPAGIKAVSGEFEAGDAVELVDLAGRTFAHGLVNYDAAELPTMLGKKTKDLAGELGRDYVRAVVHVDDLVLI

SEQ ID NO: 5 - protein, 411 aa, Propionibacterium acnes
MTCAESRGAPVLPKRSGSRAVTDQRGAICGAATLVVKVGSSSLTLPGGGIDVRRVDDLVDALSEVIAVGRRVVLVSSGAIATGFPAMGITHRPRTLAGKQAAASVGQGILLAHYASRFASHGLRVGQVLLTVNDLVRPTSYRNAWSTLDTLLGLGVVPIVNENDTVATGEIRFGDNDRLAALVAELVRAQALILLSDVDALYTAHPDSPDARRVEVVEDIDTLDVDTHKAGSGVGTGGMTTKLEAARMATCAGVPVVLAAAVDAPDVLAGVPVGTYFRPLATRRPRRLLWLADAATPQGQIVIDDGAVEALTQRHSSLLAVGVTRVHGDFQAGDPVTILASDGRVVGRGIAQFSHDEVRVMRGRSSAWLAAEMGPAASREIIHRDAMVLSRRRKAEPSSRNQKSSGSRVTS

SEQ ID NO: 6 - protein, 276 aa, Listeria monocytogenes
MRESLKNSKRLVIKVGTSTLMYGNGHINLRTIEKLAMVLSDLRNEGKEVILVSSGAIGVGCHKLQLPVRPTSIPEQQAVASVGQSELMHIYSKFFGEYGQVVGQVLLTRDVTDFPISRENVMNTLESLLERGIIPIVNENDTVAVEELEHVTKYGDNDLLSAIVAKLVQADLLIMLSDIDGFYGSNPSTDPDAVMFSEINQITPEIEALAGGKGSKFGTGGMLTKLSAASYCMNANQKMILTNGKNPTIIFDIMQGEQIGTLFASKKEEFSHDGTH

SEQ ID NO: 7 - protein, 271 aa, Enterococcus faecalis
MRNKLQQAKRIVIKVGTSSLIYPNGNINLKAIDQLAFTLSDLSNQGKEIILVSSGAIGVGLNKLNLSVRPTTIPEQQAVAAVGQAELMNIYNQRFSTYSQQMAQVLLTRDVIEYPESRNNVTNTFEQLLKMNIIPIVNENDTVAIEELDHLTKFGDNDQLSAIVCQIVQADLLVMLSDIDGFFSDNPTVNKEATLFSEINEINEDLFQLAGGKGSRFGTGGMSSKLKAAERVLANQQAMILANGKQPKIIFEILEGKDIGTLFIKGGHESD

SEQ ID NO: 8 - protein, 268 aa, Lactobacillus plantarum
MAVVKVIRKLKAKRVVIKVGTSTLVYPDGSINLRAIDQLAFVISAMRHRGREVVLVSSGAIGVGLNYMGLKQRPKAIPEQQAIAAIGQTELISIYKERFAIYEQKIGQLLLTRDIFVYPKSHHNVLNTFEALLKQNVVPIVNENDTVAVDELDHETKFGDNDQLSAIVATNIGADLLIMLSDIDGFYDGNPNADARAQLLGHIEKVDQHTYKLAGGSGSRFGTGGMVTKLKAAERMIDANGQMILANGADPTIIFQILAGEPVGTLFG

SEQ ID NO: 9 - protein, 376 aa, Corynebacterium urealyticum
MRHRIANAKRLVVKIGSSSLTDESGRVDPARIDTIADALEARMSDDTDLIVVSSGAVASGMDPLGLRQRPSDLATKQAAASVGQVLLAQEWGRSFARYGRTIGQVLLTAADAGRRDRARNAQRTIDRLRQLSAVPIVNENDTVATSEMRFGDNDRLAAIVSHLVFADALVLLSDVDGLYDRNPNEPDAQFVSEVRSSNDLSGVAAGDGGRLGTGGMAAKVTAARLASRAGVPVLLTSTENIAPALESASVGTCFWPEEDRLSAWKFWVLYAAESRGALQLDDGAKKAVVDGNKSLLAVGVTQVMGDFNRGDIVDLVDSEGKIFGRGEVNYDATDLAPMLGRHTSELPGDAQRPVIHTDYLSYYANRQPPRRQQLPI

SEQ ID NO: 10 - protein, 432 aa, Corynebacterium glutamicum
MSSTTLTDDQIRDNERTEVLAKATAAKNIVPDIAVLGTGPKNAILRAAADELVARSAEIIEANASDIEAGRANGMEESMIDRLALDESRIEGIAGGLRQVAGLTDPVGEVLRGHVMENGIQMKQVRVPLGVMGMVYEARPNVTVDAFGLALKSGNVALLRGSSTAVHSNTKLVEILQDVLERFELPRETVQLLPCQTRGSVQDLITARGLVDVVIPRGGAGLINAVVTGATVPTIETGTGNCHFYIDAEAKLDQAIAMVINGKTRRCSVCNATETALLDAALSDSDKLAVVQALQEAGVTIHGRVAELEAFGATDVVEATETDWDSEYLSFDIAVAVVDGVDGALAHIAKYSTKHTEAIATQNIETAQRFADRVDAAAVMINASTAYTDGEQYGMGAEIGISTQKLHARGPMALPELTSTKWILQGTGQIRP

SEQ ID NO: 11 - protein, 421 aa, Nocardia farcinica
MTVETTAEFDVREAVHEAARRARVASRALAQLTTAQKNDALHAAADTLLAAADTVLAANAEDIAAAEAAGTEASLLDRLRLTKARIDGIASGLRQVAGLPDPVGGVVRGSTLPNGLEIRQVRVPLGVVGMVYEARPNVTVDAFGLALKSGNAALLRGSSSAAKSNAALVEILREALRAQQIPADAVQLLPSHDRSSVTHLIQARGLVDVVIPRGGAGLINAVVRDAIVPTIETGTGNCHVYVHAAADLDMAEQILINAKTRRPSVCNAAETVLIDRAVADTAVPRLTAALREHGVTIHGDLPGLVPATDTDWGEEYLTLDIALKVVDNLDAAVEHINTWGTGHTEAIVTGDLAAAREFTSRVDAAAVMVNASTAFTDGEQFGFGAEIGISTQKLHARGPMALPELTSTKWIVWGEGQIRPV

SEQ ID NO: 12 - protein, 413 aa, Rhodococcus sp.
MDTREAVHAAARRARTASRVLALLTTAQKDAALHAAADAVLAASADILAANAADIETARASGTEDSLLDRLRLTPERIEGIASGLRQVAGLPDPVGEVVRGSTLPNGLELRQLRVPLGVVGMVYEARPNVTVDAFGLALKSGNAALLRGSSSAAKSNAALVVALRTSLAAQQLPEDAVQLLPSDDRSSVTHLIQARGLVDVVIPRGGAGLISAVVRDATVPTIETGVGNCHVYVHSAADLEMAERILLNSKTRRPSVCNTAETVLIDAAIAETAVPKLLQALQTHSVTVHGDLPGLVPATESDWSEEYLTLDVALKVVDDLNAAVEHIDRYGTGHTEAIVTSDLAAAREFTARVDAAAVMVNASTAFTDGEQFGFGAEIGISTQKLHARGPMGLPELTSTKWIVWGDGHTRPV

SEQ ID NO: 13 - protein, 448 aa, Renibacterium salmoninarum
MTELISSATASTADDAKQDGVQNSSPADNVPVEAAVFAIADRSRTAARKLSRANRAWKDRALLAIAHELLARQDNVLSANAADIAAAKESGTSDALLDRMSLNQERIKKLAVALENLAALPDPVGTVVRGQTLPNGLRLQQVNVPMGVIAAIYEARPNVTIDIAGLALKSGNAVILRGGSAVAQTNTALLNVMRDALEKVGLSVDAVQSVDSFGREGAAALMRARGRVDVLIPRGGHELIQTVVDNSSVPVIETGEGNVHIFIDESAAVESSVEILLNSKTQRPSVCNTVETLLVHEGSKALGPVLSALAAAGVRLHVDERIMAKLPAGVVAVLADDHDWATEYMDLDLAVAMVDSLDQAIKHIRRWTTGHTEAILSNNLLNTEKFVAEIDSAAVIVNASTRFTDGGEFGLGAEVGISTQKLHARGPMGLAELTTTKWIVHGEGQVRG

SEQ ID NO: 14 - protein, 415 aa, Propionibacterium acnes
MAMSSRVIEQAIAARSAATVLRRLRRIDKDDLLAAMGIGLRKGVDEILAANGADVDRGRDAGMDEALLDRLTLTPERIEGMAVGLEGVAHLDDPVGEVVRGWHTPLGVKIEQVRVPIGVIGIIYEARPNVTSDAAGICLKSGNACLLRGSSSALESNRAVIAAMRSRLPEEIREAVQLVEGGHEVTDELMTARGYVDLLIPRGGAGLINAVVTGAKVPVIQTGTGNCHLVIDVSADVEMAVNIAVNAKTQRPSVCNAIETVLVHRGIADSAVRPLVDAMSERGVTVHGDEESIRLDPRIVPAAPDEYDNEYLSLDLALRIVDDLEEAVDHIAKHGSGHSETIVTKSMSSQEFFASSVDAAAVLVNCSSRFVDGGEFGFGAEIGISTQKLHARGPMGLREMTTTTYVLRGDGQTRP

SEQ ID NO: 15 - protein, 429 aa, Corynebacterium urealyticum
MSEKIDNLSPQRQAEREEVLEKAAKAKQASRVVLTTQQKNDLLLAAADALIANTDAIIEANAKDIAAGRGAGFDDALIDRLALDAGRIEGIAGGLRQIVGLPDPVGEVVRGHTRPNGIDLKQVRTPIGVMGMVYEARPNVTVDAFGLAIKSGNVPLLRGSRSARQTNEKLVELLQDVAAEQGLSRDIVQLLPCDSHDSVQDLVTARGLVDVVIPRGGARLIEAVVLNATVPAIETGTGKCHFYIDASADLDEAIQLLLNGKTRRCSVCNATETVLLDAALSEGAKKRVFAELSAAGVRIHGVKAELDSLAEDVVEATEEDWTDEYLSFDIAAAIVDGVHGAVEHITTYSSGHTEGISARDYEVVRYFEKHVDAAALSLNTSTAWTDGEEFGFGAEIGISTQKLHARGPMGLPELTSTKWVLTGEGQTRP

SEQ ID NO: 16 - protein, 368 aa, Nostoc sp.
MPLAAVMTAPNQPVEVQQLPDPILEKGGIILETLYSEVCGTDVHLLHGRLEGVPYPIIPGHFSVGRVVETGGAVSDVNGKLIQPGAIATFLDVHETCYNCWYCLVAKASTRCPQRKVYGVTYSAKDGLLGGWSELIYLKPGVKVLTLPEEVSPKQFIAGGCALPTALHAIDRAQIQIGDVVVVQGCGPVGLSAAILALLSGAGKVIVIDKFESRLVVAKSFGVDETLAIKADDPRQHIERVLELTNGHGADVTIEATGIPIAVKEGLNMTRNGGRYVIVGHYTNTGEILINPHLEINLKHIDIRGTWGIDFSHFYRMIELLKRHSDSSKNIAWSSIISRSYTLKEINQALADVEQGSVLKAVIQPNLS

SEQ ID NO: 17 - protein, 368 aa, Nostoc sp.
MPLAAVMTAPNQPVEVQQLPDPVLEKGGIILETLYSEVCGTDVHLLHGRLEGVPYPIIPGHFSVGRVVETGGAVSDVNGKLIQPGAIATFLDVHETCYNCWYCLVAKASTRCPKRKVYGVTYSAKDGLLGGWSELIYLKPGVKVLTLPEEVSPKQFIAGGCALPTALHAIDRAQIQIGDVVVVQGCGPVGLSVAILALLSGAGKVIVIDKFESRLVVAKSFGVDETLAIKADDPRQHIERVLELTNGHGADVTIEATGIPIAVKEGLNMTRNGGRYVIVGHYTNTGEILINPHLEINLKHIDIRGTWGIDFSHFYRMIELLKRHSDSSKNIAWSSIISRSYTLQQINQALADVEQGSVLKAVIQPNLS

SEQ ID NO: 18 - protein, 368 aa, Nostoc punctiforme
MPLAAVMTAPNQPVEVQQLPEPILEKGGIIIETLYSEVCGTDVHLLHGRLEGVPYPIIPGHFSVGRVVETGGAVSDVNGKLIQPGAIATFLDVHETCYNCWYCLVAKASTRCPKRKVYGVTYSAKDGLLGGWSELIYLKPGVKVLTLPEEVSPKQFIAGGCALPTALHAIDRAQIQIGDVVVVQGCGPVGLSAAILALLSGAGKVIVIDKFESRLVVAKSFGVDETLAIRADDPRQHIERVLELTNGHGADVTIEATGIPIAVKEGLNMTRNGGRYVIVGHYTNTGEILINPHLEINLKHIDIRGTWGIDFSHFYRMIELLKRHSDSSKNIAWSSIISRSYTLKEINQALVDVEQGSVLKAVIQPNLS

SEQ ID NO: 19 - protein, 367 aa, Nodularia spumigena
MPLAAVMSAPNQPVEVQQLPEPILETGGIVIETLYSEVCGTDVHLLHGRLEGVPYPIIPGHFSVGRVVETGGQVRDVNGKLIRPGAIATFLDVHETCYNCWYCLVAKSSTRCPQRKVYGVTYSAKDGLLGGWSQLIYLKPGVKVITLPAEVSPKQFIAGGCALPTAIHAIDRAQIQIGDNVVVQGSGPVGLSAAILALLSGAGKVIVIDKFESRLKVAQSFGVDETLIIETDNPRQHIERVLELTNGHGADVTIEATGVPIAVKEGLAMTRNGGRYVIVGHYTNTGEILINPHLEINLKHIDIRGTWGIDFSHFYRMIELLKRHSDSRKNIAWENLISRAYTLKEINQALADVEHGYVLKAVIQPNS

SEQ ID NO: 20 - protein, 368 aa, Anabaena variabilis
MPKAAVMSAPNQPVVVQQLPDPILEKGGIIVETLYSEVCGTDVHLLHGRLEGVPYPIIPGHFSVGRVVETGGAVSDVNSNLIQPGAIATFLDVHETCYNCWYCLVAKASTRCPQRKVYGVTYSAKEGLLGGWSELIYLKPGVKVLTLPAEVSPKQFIAGGCALPTALHAIDRAQIQIGDVVVVQGCGPVGLSVAILALLSGAGKVIVIDKFESRLAVAKSFGVDETLAIKADDPRQHIERVLELTNGHGADVIIEATGIPIAVKEGLNMTRNGGRYVIVGHYTNTGEILINPHLEINLKHIDIRGTWGIDFSHFYRMIELLKRHSDPKKNIAWENLISRSYTLNEINQALTDVERGSVLKAVIQPNLS

SEQ ID NO: 21 - protein, 365 aa, Thermosinus carboxydivorans
MVRAALMEKPLAKVRIVDLPEPKLETGAALLETIYSEVCGTDVHLFHGRLAGVPYPIIPGHINVGRIMQTNGPVYDVDGNLLHSGDVVTFLDVHETCHNCWYCLVAKASTRCPHRKVYGITYGVQDGILGGWSEKIYLKPGVKIVKLPEQVKPEAFIGGGCGLPTAFHAVEQAGIKLGDNVVIQGCGPVGLNAAILAQLAGALQVIVVGGPALRLNLAKDFGVDAVINIENMDAADRISAVKELTGGRGADVVIEATGVPAAIKEGMAMVRDAGTYVIVGQYTDNGEVNFNPHLDLNKKHMTLKGVWGIDLSHFYRSIQIMAKYHQRFAWERLISRHYSLADINQALDDVEYCRVIKAVIAPNGN

SEQ ID NO: 22 - protein, 445 aa, Corynebacterium glutamicum
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDARLIVVTHSALESDLSRTVELLKAKPVVKAINSVIRLERD

SEQ ID NO: 23 - protein, 446 aa, Corynebacterium urealyticum
MTDASAPTIRPGKGLDATIGVALLGMGTVGAEVYRLLNERADEFAARIGGPLEVRGVAVSDLSRPREGVDPELLTDDALSLVQREDIDLVIEVIGGIDYPRQVVNAALKAGKSVVTANKALIAAHSGELSEAAESTGADLFFEAAVAAAIPVVGPLLRSLAGDKIESVMGIVNGTTNYILDAMYERGASYDEMLAEATELGYAEADPSADVDGHDAASKAAILASLAFHSRVSSEDVHTEGIRNITTQDIEAARAAGCTIKLLALCDRVTQADGTPGVSARVYPALVPLSHPLASVSESYNAIFVEAESAGRLMFYGNGAGGGPTASAVLGDVVAVARNIIHGGRGPRESTYAALPIADFGDVVTRYHVDMLVEDRVGVLAEISNAFSKHNVSLKTVRQENKDDLGARLVVVTHSATEKDLEETVATLNELDAVREVHSVIRMEGK

SEQ ID NO: 24 - protein, 438 aa, Arthrobacter aurescens
MSEMRTLKVALLGCGNVGAQVARILIDDAEALAARSGARLELSGIAVRNVDSPRDVELPRELFTTDADTLVKDADLVIELMGGIEPARSLILTAIQNGACVVTGNKALLAQDGPTLYEEADKAGVQLSYEAAVAGAIPILRPIRDSLSGDRITRVLGIVNGTTNFILDQMDSTGAQFADALAEAQRLGYAEADPTADVEGHDAAAKAAILASLSFHTRFSLDDVYCEGITKVSAADIASAKEAGFVIKLLAIAEKIDSSDNGSGISVRVHPTLLPREHPLAAVRGAFNAVFIEAENAGELMFYGQGAGGTPTASAVLGDLVSAARRLVLGGPGRTETTTGHVPALAIDASNTSYYIGLDVADQAGVLARIAHIFAENGVSIEIMRQTIHRDSASNVESAELKIVTHRASEAALAATVEAVKGLDVINSVTSVLRVEGV

SEQ ID NO: 25 - protein, 429 aa, Streptomyces coelicolor
MRTRPLKVALLGCGVVGSKVARIMTTHAADLAARIGAPVELAGVAVRRPDKVREGIDPALVTTDATALVKRGDIDVVVEVIGGIEPARTLITTAFAHGASVVSANKALIAQDGAALHAAADEHGKDLYYEAAVAGAIPLIRPLRESLAGDKVNRVLGIVNGTTNFILDAMDSTGAGYQEALDEATALGYAEADPTADVEGFDAAAKAAILAGIAFHTRVRLDDVYREGMTEVTAADFASAKEMGCTIKLLAICERAADGGSVTARVHPAMIPLSHPLANVREAYNAVFVESDAAGQLMFYGPGAGGSPTASAVLGDLVAVCRNRLGGATGPGESAYAALPVSPMGDVVTRYHISLDVADKPGVLAQVATVFAEHGVSIDTVRQSGKDGEASLVVVTHRASDAALGGTVEALRKLDTVRGVASIMRVEGE

SEQ ID NO: 26 - protein, 431 aa, Renibacterium salmoninarum
MLGCGNVGTEVARILLKDADSLAARTGARLDLVGIAVRNLDAPRSADIPRELLTTDAESLVKNADLVIELLGGIEPARSLIMLAIEHNACVVSGNKALLASDGPRLYELADAKGVQLSYEAAVAGAIPILRPIRDSLAGDKITRVLGIVNGTTNYILDQMDSTGADFNSALAEAQRLGYAEADPTADVEGRDAAAKAAILASLSFHSSFSLDDAYCEGISSITASDIEAAREAGLVIKLLAIAEKVTATDGAQDGVSVRVHPTLLPREHPLAAVRGAFNAVFVESEYAGELMFYGRGAGGTPTAAAVLGDLVSAARRIVLGGPGRTETTAGRVPVLSIDAVQTSYCIGLEVADESGVLARIAQIFATQGVSIETMRQTIHPADDQDQQQQYAELRIVTHRAAESALAATVAQIRDLDVTTAVTSVLRVEGV

SEQ ID NO: 27 - protein, 437 aa, Rhodococcus sp.
MTSESAHRAIGVAVLGLGNVGSEVVRILREHSEDLEARVGAPLELRGVAVRRPGKDRGIPEELQTTDAESLVARDDVDIVVEVIGGMDLPRKLILSALNSGKSVVTANKALLADHTGELAEAAELHNVDLYFEAAVAGAIPVIRPLIQSLAGDRVNKVAGIVNGTTNFILSAMDETGADYAATLAEAGRLGYAEADPTADVEGYDAAAKAAILASIAFHTRVTAADVYREGISKITAADLTSARALDCTIKLLSICERIATPEGKERISARVYPALVPLEHPLASVNGAFNAVVVEAENAGRLMFYGQGAGGAPTASAVMGDLVMAARNKVNGGRGPRESKYAKLKIAPIGDIPTRYYVNMQVTDKAGVLSAVAAEFSQHGVSISTVRQEGAGDGARLVVVTHVATDSALSETVEALSKLEFVTAVTSVLRLEGTEQ

SEQ ID NO: 28 - protein, 379 aa, Corynebacterium glutamicum
MYLSGSRCTLKSMTSLEFTVTRTENPTSPDRLKEILAAPKFGKFFTDHMVTIDWNESEGWHNAQLVPYAPIPMDPATTVFHYGQAIFEGIKAYRHSDETIKTFRPDENAERMQRSAARMAMPQLPTEDFIKALELLVDADQDWVPEYGGEASLYLRPFMISTEIGLGVSPADAYKFLVIASPVGAYFTGGIKPVSVWLSEDYVRAAPGGTGDAKFAGNYAASLLAQSQAAEKGCDQVVWLDAIEHKYIEEMGGMNLGFIYRNGDQVKLVTPELSGSLLPGITRKSLLQVARDLGYEVEERKITTTEWEEDAKSGAMTEAFACGTAAVITPVGTVKSAHGTFEVNNNEVGEITMKLRETLTGIQQGNVEDQNGWLYPLVG

SEQ ID NO: 29 - protein, 423 aa, Corynebacterium glutamicum
MENPSLRELDHRNIWHPYAAPGVRNRLVTKTDGVFLTLEDGSTVIDAMSSWWSAIHGHGHPRLKAAAQKQIDTMSHVMFGGLTHEPAIKLTHKLLNLTGNSFDHVFYSDSGSVSVEVAIKMALQASKGQGHPERTKLLTWRSGYHGDTFTAMSVCDPENGMHSLWKGTLPEQIFAPAPPVRGSSPQAISEYLRSMELLIDETVSAIIIEPIVQGAGGMRFHDVALIEGVATLCKKHDRFLIVDEIATGFGRTGELFATLSNGLQPDIMCVGKALTGGFMSFAATLCTDKVAQLISTPNGGGALMHGPTFMANPLACAVSHASLEIIETGMWQKQVKRIEAELIAGLSPLQHLPGVADVRVLGAIGVIEMEQNVNVEEATQAALDHGVWIRPFGRLLYVMPPYITTSEQCAQICTALHAAVKGK

SEQ ID NO: 30 - protein, 369 aa, Candida albicans
MSAPLDASKLEITKTTKPSEPLPKEELVFGKSFTDHILEVEWTAEKGWGVPTIKPYHNFSLDPATCVLHYSFELFEGLKAYRDSNGKIRTFRPDKNMERMNRSAKRAALPTFDGEEFIKLVDQFLLIEERFVPTGYGYSLYLRPTLIGTSIGLGVSAPTKALLYLIASPVGPYFSGGFKPVSLEATDYAVRAWPKGVGSYKLGANYVSCIEPQMEAAKRGHSQNLWLFGEEGYITEVGAMNVFFAFKNADGTKELVTPPLDGMILPGVTRDSTLELAKSKLPSDWTVNERKLTIHEVKERAAKGELVEAFGTGTAAIVSPIDNIEFQGEQIKVPVSAGSSGEIALKINDWIKAIQYGDESFKNWSRVAQ

SEQ ID NO: 31 - protein, 388 aa, Yarrowia lipolytica
MLRNNLRSLSRAFSTSSMRLGAGMDASKLQITKTKSPKEKQAPKDLIFGHTFTDHMLTVEWTAKDGWAAPQITPYGPLELDPSAVVLHYAFECFEGLKAYKDESGNVRLFRVDKNMHRMNTSAERICLPEFDGAEAAKLIGQLAKLDSAWIPEGRGYSMYLRPSLIGTTAALGVGTPDKALFYVIASPVGPYYPTGFKAVKLEATDYAVRAWPGGVGNKKLGANYAPCIKPQQQAASRGYQQNLWLFGDEGNITEVGTMNAFFVFERNGKKELVTAPLDGTILEGVTRDSILELARERLPSADWIVSERYCTIKEVAEAAEKGELVEAFGAGTAAVVSPIKEIGWGEKTINIPLQPGKEAGKLTETVNEWIGDIQYGKDEYKGWSKVV

SEQ ID NO: 32 - protein, 339 aa, Pseudomonas fluorescens
MGNESINWDKLGFDYIKTDKRYLSYFRNGEWDKGTLTEDNVLHISEGSTALHYGQQCFEGLKAYRCKDGSINLFRPDQNAARMQRSCARLLMPHVSTEQFIEACKEVVRANERFIPPYGTGGALYLRPFVIGVGDNIGVRTAPEFIFSVFAIPVGAYFKGGLTPHNFQISTFDRAAPQGTGAAKVGGNYAASLMPGSQAKKAHFADAIYLDPLTHKKIEEVGSANFFGITHDNKFVTPNSPSVLPGITRLSLIELAKSRLGLEVVEGDVFIDKLSDFKEAGACGTAAVITPIGGISYNDHLHVFHSETEVGPVTQKLYKELTGVQTGDVEAPAGWIVKV

SEQ ID NO: 33 - protein, 343 aa, Lactobacillus casei
MSVNIDWNNLGFDYMQLPYRYVAHWKDGAWDEGKLSTDPNLTMNEGSPILHYGQGAFEGMKAYRTKSGKIQLFRPDQNAHRLHNSADKLLMPPFPEDRFIDAVKQVVAANHEYVPPYGTGATLYLRPILIGVGPNIGVAPAKEYIFDVFAMPVGPYFKGGMVPTKFIVADQFDRAAHYGTGQSKVGGNYAASLQAGKFAHEHGYGDAIYLDPIEHKYIEEVGSANFFGISKDGKTLKTPKSPSILPSITKYSILALAHDRFGMTTEETKIAITDLDQFGEAGACGTAAVITPIASITYEDHEHVFYSETKVGPYTQKLYDELTGIQFGDVPAPEGWVVDVPFN

SEQ ID NO: 34 - protein, 342 aa, Lactobacillus plantarum
MADVQLDWNNLGFEYRNLPYRYRAYWKDGAWYKKELTGDATLHISEGSTALHYGQQDFEGLKAYRTKDGSVQLFRPDRNAARMQTSCERLLMPQVPTDMFVDAVKQVVKANQDYVPPYGTGATLYLRPLMIGVGGNIGVHPAQEYIFTVFAMPVGSYFKGGMTPTNFTTSEYDRAAHKGTGAYKVGGNYAASLFPGQEAHANGFSDCVYLDPVEHRKIEEVGSANFFGITKDGTFVTPKSPSILPAVTKYSLLYLAEHKFGMKTEQGDVYIDDLDRFAEAGACGTAAVISPIGGLEHQGKLHVFYSETEVGPVTKKLYDELTGIQFGDREAPEGWVQKVELD

SEQ ID NO: 35 - protein, 340 aa, Lactococcus lactis
MAINLDWENLGFSYRNLPFRYIARFKDGKWSAGELTGDNQLHISESSPALHYGQQGFEGLKAYRTKDGSIQLFRPDQNAARLQKTARRLCMAEVSTEMFIDAVKQVVKANKDFVPPYGTGATLYLRPLLIGVGDVIGVKPADEYIFKVFAMPVGSYFKGGLAPSKFVISREYDRAAPLGTGGAKVGGNYAASLQAEVGAKASGYADAIYLDPSTHTKIEEVGAANFFGITADNEFITPLSPSILPSITKYSLLYLAEHRLGLKAIEGEVYAKDLGKFVEAGACGTAAIISPIGRIDDGEDSYIFHSETEVGPTVKRLYDELVGIQFGDVEAPEGWIVKVD

SEQ ID NO: 36 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 37 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 38 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 39 - protein, 528 aa, Pseudomonas putida [sequence omitted]

SEQ ID NO: 40 - protein, 549 aa, Staphylococcus epidermidis
MKQRVGQYLMDAVYSAGADKVFGVPGDFNLAFLDDIIAHNDIKWIGNTNELNASYAADGYARINGIGAMVTTFGVGELSAVNGIAGSYAERVPVIAITGAPTRAVEQEGKYVHHSLGEGTFDDYRKMFEPITTAQAYITPDNATTEIPRVINTALQQRRPVHIHLPIDVALTEIEISNPFKPEVEPQKNVQSYINMVQDKLESASQPVIITGHEINSFHLHKELEQFVNQTQIPVVQLSLGKGAFNEENPYYMGIFDGSIAEQDIQDYVNQSDAILNIGAKLTDSATAGFSYQFDINEVIMLNHNEFKINDTCIEAFSLPNILNGLNKYIHYKNTNDFPQYERPQSHNYELSDQPLTQETYFNMMQDFLQQDDILIAEQGTSFFGAYDLALYKDNTFIGQPLWGSIGYTLPATLGTQIANPYRRNILLIGDGSLQLTVQSLSTMIRQNLNPVIFVINNDGYTVERMIHGMKEPYNDIRMWDYKSLPSVFGGDNVLVHDVNTSEELMLTFENIKSNSDRMHFVEVKMAVEDAPVKLSNIAKAFASQNKSS

SEQ ID NO: 41 - protein, 547 aa, Staphylococcus saprophyticus
MKKRVGQYLMDCISDVGVDKVFGVPGDFNLTFLDDIISRDDMEWIGNTNELNASYAADGYARMKGIAAMVTTFGVGELSAVNGIAGSYAERVPVIQITGAPTRAVESAGKYVHHSLGEGNFDDYRNMYASITTAQAYITPENAQSEIPRVINAALYEKRPVHIHLPIDVANSEIDVATPFEIEQRPQTDVTKYMTMVKDKLQSADKPVIITGHEINSFKLHEKLEQFVKQSQIPVVQLSLGKGAFNETSPYYMGIYDASLAEEDIRNYVDQSDAILNIGAKLTDSATAGYSYQFDIDDVVMINHHHFKMNETKDTEVSLVDLLDGLNAINYVNNAEFPKFKQPKAHDYDLTDEPLTQETYFKMMQDFIREEDVILAEQGTSFFGAYDLALKHNNKFIGQPLWGSIGYTLPATLGTQMADTSRRNLLLIGDGSLQLTVQEISTMIREKIKPIIFVINNDGYTVERKIHGENAMYNDIKMWDYKLLPTVFGGKDEVQVHDVNTSEAFQKVLLQIEAQPDTMHFIEVKMGVHDAPHKLNAIGQAFAKQNG

SEQ ID NO: 42 - protein, 558 aa, Bacillus cereus
MKKQYTVSTYLLDRLHELGIEHIFGVPGDYNLAFLDDVVAHENLKWIGNCNELNAAYAADGYARIKGIAALITTFGVGELSAINGIAGSYAENVPVIKITGTPPTKVMENGAIVHHTLGDGKFDHFSNMYREITVAQTNVTPEHAAEEIDRVLRACWNEKRPVHINLPIDVYNKPINKPTEPIINKPILSNKEALNKMLLHAISKINSAKKPVILADFEVDRFHAKENLHQFVEKTGFPIATLSMGKGIFPEKHPQFIGIYTGDVSSPYLRKRIDESDCIISIGVKLTDTITGGFTQGFTKEQVIEIHPYTVKIIDKKYGPVVMQDVLQHLSDSIEHRNKDTLDVKPFILESSPFTEEFIPKAQMVTQKRFWQQMYHFLQENDVLIVEQGTPFFGSSAIPLPNNTAYVGQPLWGSIGYTLPALLGTQLANLSRRNILIIGDGSFQVTAQELSTILRQNLKPIIFLINNNGYTVERAIHGQNQLYNDIQMWDYNKLSMVFGAEEKSLTFKVENEAELAAVLTNITFNKNQLIFIEVIMSQSDQPELLAKLGKRSGQQNS

SEQ ID NO: 43 - protein, 558 aa, Gluconacetobacter diazotrophicus
MTYTVGRYLADRLAQIGLKHHFAVAGDYNLVLLDQLLLNTDMQQIYCSNELNCGFSAEGYARANGAAAAIVTFSVGALSAFNALGGAYAENLPVILISGAPNANDHGTGHILHHTLGTTDYGYQLEMARHITCAAESIVAAEDAPAKIDHVIRTALREKKPAYLEIACNVAGAPCVRPGGIDALLSPPAPDEASLKAAVDAALAFIEQRGSVTMLVGSRIRAAGAQAQAVALADALGCAVTTMAAAKSFFPEDHPGYRGHYWGEVSSPGAQQAVEGADGVICLAPVFNDYATVGWSAWPKGDNVMLVERHAVTVGGVAYAGIDMRDFLTRLAAHTVRRDATARGGAYVTPQTPAAAPTAPLNNAEMARQIGALLTPRTTLTAETGDSWFNAVRMKLPHGARVELEMQWGHIGWSVPAAFGNALAAPERQHVLMVGDGSFQLTAQEVAQMIRHDLPVIIFLINNHGYTIEVMIHDGPYNNVKNWDYAGLMEVFNAGEGNGLGLRARTGGELAAAIEQARANRNGPTLIECTLDRDDCTQELVTWGKRVAAANARPPRAG

SEQ ID NO: 44 - protein, 371 aa, Clostridium perfringens
MKLLNLNTEILGFDTSSEFAKELNINGKDLVFTNRRIYEGSFKSLDLKCNYVLKDKYNFNEPNDEIIDEIFKDIKNIDFNRVIAIGGGSILDVGKLLALKDVSSTKKLFKRELPIIRDKELIIVPTTCGTGSEVTNISIVEIKSENTKLGLAVDELYADKGVLIPELCKTMPYKAFVYSSIDALIHSIEGFLSPNSTPYTDLFAFKAIEMILSGYKEILEKGEEYRNTIMDKFLIASNYAGIAFGNAGVGAVHALSYPLGGKYHVPHGEANYQFLMAVLNFYKKNNGDGKIIELTRLISSIINAKEEDCYLELENLLNNLIRRKNLKEYGMTIDEIEKFSHSVMKNQQRLLKNNYVSMEIKDMEEIYKYLY

SEQ ID NO: 45 - protein, 389 aa, Lactobacillus sakei
MKTNFDFLMPSVNFFGPGVIEKIGDRTKMLNITKPLIVTDTFLEGVPDGPVAQTLASLDQAGVQYSIFNGVEPNPKVRNIQAGKAQYLADGCDGLITVGGGSAHDCGKGIGILLTNGDDITKLAGIETLENALPPLLAVNTTAGTGSELTRHCVITNEETHLKFVVVSWRNIPLVSFNDPMLMLDVPAKLTAATGMDAFVQAIEPYVSTNRNAITDGQCLQAIKLIEESLREAVANGHNIEARTKMCEAEMLAGMAFNNADLGYVHAMAHQLGGQYDAPHGVCCALLLPIVEEFNIVACPDRFAELARAMGENTDALSTRDAAELAIKGMRQMAADVGIPTSIKAIGAKPADFEMMAENALKDGNAFSNPRKATKAEIVALFQKAYDAE

SEQ ID NO: 46 - protein, 400 aa, Rhodococcus sp.
MGVGAHDIIGVEAKNLGFKRTLLMTTGLRGSGIIEELVGKIEYQGVEVVLYDKVESNPKDYNVMEAAALYQKEKCDSIISVGGGSSHDAAKGARVVVAHDGRNINEFEGFAKSTNKENPPHIAVSTTAGTGSETSWAYVITDTSDMNNPHKWVGFDEATIVTLAIDDPLLYYTCPQHFTAYCGFDVLAHGSEPYVSRLDFAPSLGNALYSVELVAKNLREAVFEPRNLKAREGMMNAQYIAGQAFNSGGLGIVHSISHAVSAFFDSHHGLNNAIALPRVWEYNLPSRYERYAQLAGALGVDTRNLTTVQAADAAVEAAIRLAKDVGIPDNFGQVRTDSYDKNQMNTKKYEGRGDTIRGDEKTVRAISEHIQGDWCTPGNPREVTVESMLPVVDHAINSAY

SEQ ID NO: 47 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 48 - protein, 387 aa, Citrobacter freundii
MSYRMFDYLVPNVNFFGPNAISVVGERCKLLGGKKALLVTDKGLRAIKDGAVDKTLTHLREAGIDVVVFDGVEPNPKDTNVRDGLEVFRKEHCDIIVTVGGGSPHDCGKGIGIAATHEGDLYSYAGIETLTNPLPPIVAVNTTAGTASEVTRHCVLTNTKTKVKFVIVSWRNLPSVSINDPLLMLGKPAPLTAATGMDALTHAVEAYISKDANPVTDAAAIQAIRLIARNLRQAVALGSNLKARENMAYASLLAGMAFNNANLGYVHAMAHQLGGLYDMPHGVANAVLLPHVARYNLIANPEKFADIAEFMGENTDGLSTMDAAELAIHAIARLSADIGIPQHLRDLGVKEADFPYMAEMALKDGNAFSNPRKGNEKEIAEIFRQAF

SEQ ID NO: 49 - protein, 382 aa, Azotobacter vinelandii
MSSTFFIPAVNMMGIGSLDEAMGAIKNHGFRKALIVTDAGLAKAGVADRVSQLLAQQDIASVLYDGAKPNPTVSNVENGLALLKSSGADFVISLGGGSPHDCAKGIALVAANGGHISDYEGVDRSARPQLPLVSINTTAGTASEMTRFCIITDEARHVKMAIVDRNVTPLLSVNDPSLMAAMPKGLTAATGMDALTHAIEAYVSTAATPITDACALKAVELISANLRNAVANGGDMTARENMAYAQFLAGMAFNNASLGYVHAMAHQLGGFYDLPHGVCNAVLLPHVESFNASVCPERLRDVAKSMGIDVQGLSAEQGAQAALTAIRTLSKEIGIPGGLKELGAKADDIPVLSANALKDACGLTNPRPADQQQIEEIFRNAF

SEQ ID NO: 50 - protein, 383 aa, Zymomonas mobilis
MASSTFYIPFVNEMGEGSLEKAIKDLNGSGFKNALIVSDAFMNKSGVVKQVADLLKAQGINSAVYDGVMPNPTVTAVLEGLKILKDNNSDFVISLGGGSPHDCAKAIALVATNGGEVKDYEGIDKSKKPALPLMSINTTAGTASEMTRFCIITDEVRHVKMAIVDRHVTPMVSVNDPLLMVGMPKGLTAATGMDALTHAFEAYSSTAATPITDACALKAASMIAKNLKTACDNGKDMPAREAMAYAQFLAGMAFNNASLGYVHAMAHQLGGYYNLPHGVCNAVLLPHVLAYNASVVAGRLKDVGVAMGLDIANLGDKEGAEATIQAVRDLAASIGIPANLTELGAKKEDVPLLADHALKDACALTNPRQGDQKEVEELFLSAF

SEQ ID NO: 51 - protein, 738 aa, Corynebacterium glutamicum
MAKIIWTRTDEAPLLATYSLKPVVEAFAATAGIEVETRDISLAGRILAQFPERLTEDQKVGNALAELGELAKTPEANIIKLPNISASVPQLKAAIKELQDQGYDIPELPDNATTDEEKDILARYNAVKGSAVNPVLREGNSDRRAPIAVKNFVKKFPHRMGEWSADSKTNVATMDANDFRHNEKSIILDAADEVQIKHIAADGTETILKDSLKLLEGEVLDGTVLSAKALDAFLLEQVARAKAEGILFSAHLKATMMKVSDPIIFGHVVRAYFADVFAQYGEQLLAAGLNGENGLAAILSGLESLDNGEEIKAAFEKGLEDGPDLAMVNSARGITNLHVPSDVIVDASMPAMIRTSGHMWNKDDQEQDTLAIIPDSSYAGVYQTVIEDCRKNGAFDPTTMGTVPNVGLMAQKAEEYGSHDKTFRIEADGVVQVVSSNGDVLIEHDVEANDIWRACQVKDAPIQDWVKLAVTRSRLSGMPAVFWLDPERAHDRNLASLVEKYLADHDTEGLDIQILSPVEATQLSIDRIRRGEDTISVTGNVLRDYNTDLFPILELGTSAKMLSVVPLMAGGGLFETGAGGSAPKHVQQVQEENHLRWDSLGEFLALAESFRHELNNNGNTKAGVLADALDKATEKLLNEEKSPSRKVGEIDNRGSHFWLTKFWADELAAQTEDADLAATFAPVAEALNTGAADIDAALLAVQGGATDLGGYYSPNEEKLTNIMRPVAQFNEIVDALKK

SEQ ID NO: 52 - protein, 1221 aa, Corynebacterium glutamicum
MSSASTFGQNAWLVDEMFQQFQKDPKSVDKEWRELFEAQGGPNTTPATTEAQPSAPKESAKPAPKAAPAAKAAPRVETKPADKTAPKAKESSVPQQPKLPEPGQTPIRGIFKSIAKNMDISLEIPTATSVRDMPARLMFENRAMVNDQLKRTRGGKISFTHIIGYAMVKAVMAHPDMNNSYDVIDGKPTLIVPEHINLGLAIDLPQKDGSRALVVAAIKETEKMNFSEFLAAYEDIVARSRKGKLTMDDYQGVTVSLTNPGGIGTRHSVPRLTKGQGTIIGVGSMDYPAEFQGASEDRLAELGVGKLVTITSTYDHRVIQGAVSGEFLRTMSRLLTDDSFWDEIFDAMNVPYTPMRWAQDVPNTGVDKNTRVMQLIEAYRSRGHLIADTNPLSWVQPGMPVPDHRDLDIETHNLTIWDLDRTFNVGGFGGKETMTLREVLSRLRAAYTLKVGSEYTHILDRDERTWLQDRLEAGMPKPTQAEQKYILQKLNAAEAFENFLQTKYVGQKRFSLEGAEALIPLMDSAIDTAAGQGLDEVVIGMPHRGRLNVLFNIVGKPLASIFNEFEGQMEQGQIGGSGDVKYHLGSEGQHLQMFGDGEIKVSLTANPSHLEAVNPVMEGIVRAKQDYLDKGVDGKTVVPLLLHGDAAFAGLGIVPETINLAKLRGYDVGGTIHIVVNNQIGFTTTPDSSRSMHYATDYAKAFGCPVFHVNGDDPEAVVWVGQLATEYRRRFGKDVFIDLVCYRLRGHNEADDPSMTQPKMYELITGRETVRAQYTEDLLGRGDLSNEDAEAVVRDFHDQMESVFNEVKEGGKKQAEAQTGITGSQKLPHGLETNISREELLELGQAFANTPEGFNYHPRVAPVAKKRVSSVTEGGIDWAWGELLAFGSLANSGRLVRLAGEDSRRGTFTQRHAVAIDPATAEEFNPLHELAQSKGNNGKFLVYNSALTEYAGMGFEYGYSVGNEDSIVAWEAQFGDFANGAQTIIDEYVSSGEAKWGQTSKLILLLPHGYEGQGPDHSSARIERFLQLCAEGSMTVAQPSTPANHFHLLRRHALSDLKRPLVIFTPKSMLRNKAAASAPEDFTEVTKFQSVINDPNVADAAKVKKVMLVSGKLYYELAKRKEKDGRDDIAIVRIEMLHPIPFNRISEALAGYPNAEEVLFVQDEPANQGPWPFYQEHLPELIPNMPKMRRVSRRAQSSTATGVAKVHQLEEKQLIDEAFEA

SEQ ID NO: 53 - protein, 447 aa, Corynebacterium glutamicum
MTVDEQVSNYYDMLLKRNAGEPEFHQAVAEVLESLKIVLEKDPHYADYGLIQRLCEPERQLIFRVPWVDDQGQVHVNRGFRVQFNSALGPYKGGLRFHPSVNLGIVKFLGFEQIFKNSLTGLPIGGGKGGSDFDPKGKSDLEIMRFCQSFMTELHRHIGEYRDVPAGDIGVGGREIGYLFGHYRRMANQHESGVLTGKGLTWGGSLVRTEATGYGCVYFVSEMIKAKGESISGQKIIVSGSGNVATYAIEKAQELGATVIGFSDSSGWVHTPNGVDVAKLREIKEVRRARVSVYADEVEGATYHTDGSIWDLKCDIALPCATQNELNGENAKTLADNGCRFVAEGANMPSTPEAVEVFRERDIRFGPGKAANAGGVATSALEMQQNASRDSWSFEYTDERLQVIMKNIFKTCAETAAEYGHENDYVVGANIAGFKKVADAMLAQGVI

SEQ ID NO: 54 - protein, 477 aa, Corynebacterium glutamicum
MAFETPEEIVKFIKDENVEFVDVRFTDLPGTEQHFSIPAASFDADTIEEGLAFDGSSIRGFTTIDESDMNLLPDLGTATLDPFRKAKTLNVKFFVHDPFTREAFSRDPRNVARKAEQYLASTGIADTCNFGAEAEFYLFDSVRYSTEMNSGFYEVDTEEGWWNRGKETNLDGTPNLGAKNRVKGGYFPVAPYDQTVDVRDDMVRNLAASGFALERFHHEVGGGQQEINYRFNTMLHAADDIQTFKYIIKNTARLHGKAATFMPKPLAGDNGSGMHAHQSLWKDGKPLFHDESGYAGLSDIARYYIGGILHHAGAVLAFTNATLNSYHRLVPGFEAPINLVYSQRNRSAAVRIPITGSNPKAKRIEFRAPDPSGNPYLGFAAMMMAGLDGIKNRIEPHAPVDKDLYELPPEEAASIPQAPTSLEASLKALQEDTDFLTESDVFTEDLIEAYIQYKYDNEISPVRLRPTPQEFELYFDC

SEQ ID NO: 55 - protein, 446 aa, Corynebacterium glutamicum
MNSEQEFVLSAIEERDIKFVRLWFTDILGHLKSVVVAPAELESALEEGIGFDGSAIEGYARISEADTIARPDPSTFQVLPLEAGISKLQAARLFCDVTMPDGQPSFSDPRQVLRRQVQLAADEGLTCMISPEIEFYLVQSLRTNGLPPVPTDNGGYFDQATFNEAPNFRRNAMVALEELGIPVEFSHHETAPGQQEIDLRHADALTMADNIMTFRYIMKQVARDQGVGASFMPKPFQEHAGSAMHTHMSLFEGDTNAFHDPDDSYMLSKTAKQFIAGILHHAPEFTAVTNQWVNSYKRIVYGNEAPTAATWGVSNRSALVRVPTYRLNKEESRRVEVRLPDTACNPYLAFSVMLGAGLKGIKEGYELDEPAEDDISNLSFRERRAMGYNDLPSSLDQALRQMEKSELVADILGEHVFEFFLRNKWREWRDYQEQITPWELRNNLDY

SEQ ID NO: 56 - protein, 270 aa, Corynebacterium glutamicum
MTTIAVIGGGQIGEALVSGLIAANMNPQNIRVTNRSEERGQELRDRYGILNMTDNSQAADEADVVFLCVKPKFIVEVLSEITGTLDNNSAQSVVVSMAAGISIAAMEESASAGLPVVRVMPNTPMLVGKGMSTVTKGRYVDAEQLEQVKDLLSTVGDVLEVAESDIDAVTAMSGSSPAYLFLVTEALIEAGVNLGLPRATAKKLAVASFEGAATMMKETGKEPSELRAGVSSPAGTTVAAIRELEESGIRGAFYRAAQACADRSEELGKR

SEQ ID NO: 57 - protein, 1152 aa, Corynebacterium glutamicum
MTSMNLPIELATLSDQAVDKVRSWLEYSKKESVPNADAKRLAAVLQDPNGLEFTVGFVDRVVRTEDREAAAHALYELGKIAPSTMSFLDRAQIQAGSLVGRALPQVVVPAARARIRQMVGHMIVDARDKQFAKAVAEIQSDGHRLNINLLGEAVLGRKEAAKHLDDTVRLLRRPDVEYVSIKVSSVASQISMWGFEDTVNYVVEQLTPLYIEAARAPKGTKFINLDMEEYRDLRLTMEVFKRLLSNPELHELEAGIVLQAYLPDALGAIQDLAQFGRERVNTGGAGVKVRLVKGANLPMEHVHAQITGWPVATEPSKQATDANYKRVLYWTMRKENMEGLRLGVAGHNLFDIAFAHLLSVERGVADRVEFEMLQGMASDQARAVSVDVGELLLYVPAVRPQEFDVAISYLVRRLEENAASENFMSAIFDLDADNPSFKREESRFRASISDLATLIDVPAPGPNHTQDRSKETLLDAPLVPFINEPDTNPALIQNQQWATKAVATAAEPGWLEKQTKPEVLEEGDVDKLINDVRDAAEAWAARPARERAEILYKTAEILRVRRGHLISVTAAEVGKAVEQTDPEISEAIDFARYYAHLALELDDVDNAEFTPDRVVVVTPPWNFPIAIPAGSTFAALAAGAGVIHKPSKPSQHCSAAVVEALWEAGVPREVLHCIYPANRDVGCALISHEHVDRVILTGSSETAAMFSSWRPELTINGETSGKNAIVVTPSADRDLAVADLVKSAFGHAGQKCSAASLGILVGSVYESERFRKQLVDAASSLIVDWPTNPSATVGPLTELPSDKLHHALTTLEEGESWLLKPRQLDDTGRLWSPGIKEGVKPGTFFHLTEVFGPVLGLMKATDLNEAIEFQNGNDFGLTGGLQSLDADEVRTWLDHVDVGNAYVNRGITGAIVQRQSFGGWKKSSVGLGSKAGGPNYVMLMGTWADAPSHHAPRETNPLISKLDLPGEELEWLEKANASDETAWNTEFGSPRDPSGLDVEANIFRYRPAEVVLRLDDSATPRETARALLAARRAGVTPRVLQTPGVSEQVREVLSAAGVSAETVDDSVFISNVLRGEYDENSSVRVRYLGKVSDTVRERLSVRPEVVLLDDAVTASGRVELRYWLKEQAISMTLHRFGNPVAAFHELAEELKR

SEQ ID NO: 58 - protein, 339 aa, Pseudomonas fluorescens
MGNESINWDKLGFDYIKTDKRFLQVWKNGEWQEGTLTDDNVLHISEGSTALHYGQQCFEGLKAYRCKDGSINLFRPDQNAARMQRSCARLLMPHVPTDVFIDACKQVVKANEHFIPPYGSGGALYLRPFVIGTGDNIGVRTAPEFIFSVFCIPVGAYFKGGLVPHNFQISTFDRAAPQGTGAAKVGGNYAASLMPGSEAKKAGFADAIYLDPMTHSKIEEVGSANFFGITHDNQFITPKSPSVLPGITRLSLIELAKTRLGLDVVEGEVFIDKLDQFKEAGACGTAAVISPIGGIQYNGKLHVFHSETEVGPITQKLYKELTGVQTGDVEAPQGWIVKV

SEQ ID NO: 59 - protein, 361 aa, Escherichia coli
MMKTMRIAAIPGDGIGKEVLPEGIRVLQAAAERWGFALSFEQMEWASCEYYSHHGKMMPDDWHEQLSRFDAIYFGAVGWPDTVPDHISLWGSLLKFRREFDQYVNLRPVRLFPGVPCPLAGKQPGDIDFYVVRENTEGEYSSLGGRVNEGTEHEVVIQESVFTRRGVDRILRYAFELAQSRPRKTLTSATKSNGLAISMPYWDERVEAMAENYPEIRWDKQHIDILCARFVMQPERFDVVVASNLFGDILSDLGPACTGTIGIAPSANLNPERTFPSLFEPVHGSAPDIYGKNIANPIATIWAGAMMLDFLGNGDERFQQAHNGILAAIEEVIAHGPKTPDMKGNATTPQVADAICKIILR

SEQ ID NO: 60 - protein, 572 aa, Escherichia coli
MKQTVAAYIAKTLESAGVKRIWGVTGDSLNGLSDSLNRMGTIEWMSTRHEEVAAFAAGAEAQLSGELAVCAGSCGPGNLHLINGLFDCHRNHVPVLAIAAHIPSSEIGSGYFQETHPQELFRECSHYCELVSSPEQIPQVLAIAMRKAVLNRGVSVVVLPGDVALKPAPEGATMHWYHAPQPVVTPEEEELRKLAQLLRYSSNIALMCGSGCAGAHKELVEFAGKIKAPIVHALRGKEHVEYDNPYDVGMTGLIGFSSGFHTMMNADTLVLLGTQFPYRAFYPTDAKIIQIDINPASIGAHSKVDMALVGDIKSTLRALLPLVEEKADRKFLDKALEDYRDARKGLDDLAKPSEKAIHPQYLAQQISHFAADDAIFTCDVGTPTVWAARYLKMNGKRRLLGSFNHGSMANAMPQALGAQATEPERQVVAMCGDGGFSMLMGDFLSVVQMKLPVKIVVFNNSVLGFVAMEMKAGGYLTDGTELHDTNFARIAEACGITGIRVEKASEVDEALQRAFSIDGPVLVDVVVAKEELAIPPQIKLEQAKGFSLYMLRAIISGRGDEVIELAKTNWLR

SEQ ID NO: 61 - protein, 126 aa, Escherichia coli
MIRTMLQGKLHRVKVTHADLHYEGSCAIDQDFLDAAGILENEAIDIWNVTNGKRFSTYAIAAERGSRIISVNGAAAHCASVGDIVIIASFVTMPDEEARTWRPNVAYFEGDNEMKRTAKAIPVQVA

SEQ ID NO: 62 - protein, 713 aa, Escherichia coli
MNIIAIMGPHGVFYKDEPIKELESALVAQGFQIIWPQNSVDLLKFIEHNPRICGVIFDWDEYSLDLCSDINQLNEYLPLYAFINTHSTMDVSVQDMRMALWFFEYALGQAEDIAIRMRQYTDEYLDNITPPFTKALFTYVKERKYTFCTPGHMGGTAYQKSPVGCLFYDFFGGNTLKADVSISVTELGSLLDHTGPHLEAEEYIARTFGAEQSYIVTNGTSTSNKIVGMYAAPSGSTLLIDRNCHKSLAHLLMMNDVVPVWLKPTRNALGILGGIPRREFTRDSIEEKVAATTQAQWPVHAVITNSTYDGLLYNTDWIKQTLDVPSIHFDSAWVPYTHFHPIYQGKSGMSGERVAGKVIFETQSTHKMLAALSQASLIHIKGEYDEEAFNEAFMMHTTTSPSYPIVASVETAAAMLRGNPGKRLINRSVERALHFRKEVQRLREESDGWFFDIWQPPQVDEAECWPVAPGEQWHGFNDADADHMFLDPVKVTILTPGMDEQGNMSEEGIPAALVAKFLDERGIVVEKTGPYNLLFLFSIGIDKTKAMGLLRGLTEFKRSYDLNLRIKNMLPDLYAEDPDFYRNMRIQDLAQGIHKLIRKHDLPGLMLRAFDTLPEMIMTPHQAWQRQIKGEVETIALEQLVGRVSANMILPYPPGVPLLMPGEMLTKESRTVLDFLLMLCSVGQHYPGFETDIHGAKQDEDGVYRVRVLKMAG

SEQ ID NO: 63 - protein, 732 aa, Escherichia coli
MSKLKIAVSDSCPDCFTTQRECIYINESRNIDVAAIVLSLNDVTCGKLDEIDATGYGIPVFIATENQERVPAEYLPRISGVFENCESRREFYGRQLETAASHYETQLRPPFFRALVDYVNQGNSAFDCPGHQGGEFFRRHPAGNQFVEYFGEALFRADLCNADVAMGDLLIHEGAPCIAQQHAAKVFNADKTYFVLNGTSSSNKVVLNALLTPGDLVLFDRNNHKSNHHGALLQAGATPVYLETARNPYGFIGGIDAHCFEESYLRELIAEVAPQRAKEARPFRLAVIQLGTYDGTIYNARQVVDKIGHLCDYILFDSAWVGYEQFIPMMADCSPLLLDLNENDPGILVTQSVHKQQAGFSQTSQIHKKDSHIKGQQRYVPHKRMNNAFMMHASTSPFYPLFAALNINAKMHEGVSGRNMWMDCVVNGINARKLILDNCQHIRPFVPELVDGKPWQSYETAQIAVDLRFFQFVPGEHWHSFEGYAENQYFVDPCKLLLTTPGIDARNGEYEAFGVPATILANFLRENGVVPEKCDLNSILFLLTPAEDMAKLQQLVALLVRFEKLLESDAPLAEVLPSIYKQHEERYAGYTLRQLCQEMHDLYARHNVKQLQKEMFRKEHFPRVSMNPQEANYAYLRGEVELVRLPDAEGRIAAEGALPYPPGVLCVVPGEIWGGAVLRYFSALEEGINLLPGFAPELQGVYIEEHDGRKQVWCYVIKPRDAQSTLLKGEKL

SEQ ID NO: 64 - protein, 466 aa, Escherichia coli
MDKKQVTDLRSELLDSRFGAKSISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 65 - protein, 556 aa, Escherichia coli
MSVSAFNRRWAAVILEALTRHGVRHICIAPGSRSTPLTLAAAENSAFIHHTHFDERGLGHLALGLAKVSKQPVAVIVTSGTAVANLYPALIEAGLTGEKLILLTADRPPELIDCGANQAIRQPGMFASHPTHSISLPRPTQDIPARWLVSTIDHALGTLHAGGVHINCPFAEPLYGEMDDTGLSWQQRLGDWWQDDKPWLREAPRLESEKQRDWFFWRQKRGVVVAGRMSAEEGKKVALWAQTLGWPLIGDVLSQTGQPLPCADLWLGNAKATSELQQAQIVVQLGSSLTGKRLLQWQASCEPEEYWIVDDIEGRLDPAHHRGRRLIANIADWLELHPAEKRQPWCVEIPRLAEQAMQAVIARRDAFGEAQLAHRICDYLPEQGQLFVGNSLVVRLIDALSQLPAGYPVYSNRGASGIDGLLSTAAGVQRASGKPTLAIVGDLSALYDLNALALLRQVSAPLVLIVVNNNGGQIFSLLPTPQSERERFYLMPQNVHFEHAAAMFELKYHRPQNWQELETAFADAWRTPTTTVIEMVVNDTDGAQTLQQLLAQVSHL

SEQ ID NO: 66 - protein, 420 aa, Escherichia coli
MPHSLFSTDTDLTAENLLRLPAEFGCPVWVYDAQIIRRQIAALKQFDVVRFAQKACSNIHILRLMREQGVKVDSVSLGEIERALAAGYNPQTHPDDIVFTADVIDQATLERVSELQIPVNAGSVDMLDQLGQVSPGHRVWLRVNPGFGHGHSQKTNTGGENSKHGIWYTDLPAALDVIQRHHLQLVGIHMHIGSGVDYAHLEQVCGAMVRQVIEFGQDLQAISAGGGLSVPYQQGEEAVDTEHYYGLWNAAREQIARHLGHPVKLEIEPGRFLVAQSGVLITQVRSVKQMGSRHFVLVDAGFNDLMRPAMYGSYHHISALAADGRSLEHAPTVETVVAGPLCESGDVFTQQEGGNVETRALPEVKAGDYLVLHDTGAYGASMSSNYNSRPLLPEVLFDNGQARLIRRRQTIEELLALELL

SEQ ID NO: 67 - protein, 220 aa, Escherichia coli
MSRPLLQLALDHSSLEAAQRDVTLLKDSVDIVEAGTILCLNEGLGAVKALREQCPDKIIVADWKVADAGETLAQQAFGAGANWMTIICAAPLATVEKGHAMAQRCGGEIQIELFGNWTLDDARDWHRIGVRQAIYHRGRDAQASGQQWGEADLARMKALSDIGLELSITGGITPADLPLFKDIRVKAFIAGRALAGAANPAQVAGDFHAQIDAIWGGARA

SEQ ID NO: 68 - protein, 216 aa, Escherichia coli
MSLPMLQVALDNQTMDSAYETTRLIAEEVDIIEVGTILCVGEGVRAVRDLKALYPHKIVLADAKIADAGKILSRMCFEANADWVTVICCADINTAKGALDVAKEFNGDVQIELTGYWTWEQAQQWRDAGIGQVVYHRSRDAQAAGVAWGEADITAIKRLSDMGFKVTVTGGLALEDLPLFKGIPIHVFIAGRSIRDAASPVEAARQFKRSIAELWG

SEQ ID NO: 69 - protein, 1227 aa, Mycobacterium smegmatis
MSSSPSPFGQNEWLVEEMYRKFRDDPSSVDPSWHEFLVDYSPEPTTDSASNGRTTTAAPVTPPTPAPAPAPEPKAAPKPAAKTEAKPAKPAKSATPAKGDESQILRGAAAAVVKNMNASLEVPTATSVRAIPAKLMIDNRVVINNHLKRTRGGKISFTHLLGYAIVQAVKKFPNMNRHFAVVDGKPTAITPAHTNLGLAIDLQGKDGNRSLVVAAIKRCETMRFGQFIAAYEDIVRRARDGKLTAEDFSGVTISLTNPGTLGTVHSVPRLMQGQGAIIGAGAMEYPAEFQGASEERIADLGIGKLITLTSTYDHRIIQGAESGDFLRTIHQLLLDDDFFDEIFRELGIPYEPVRWRTDNPDSIEDKNARVIELIAAYRNRGHLMADIDPLRLDNTRFRSHPDLDVNSHGLTLWDLDREFKVDGFAGVQRKKLRDILSVLRDAYCRHVGVEYTHILEPEQQRWIQERVETKHDKPTVAEQKYILSKLNAAEAFETFLQTKYVGQKRFSLEGAETVIPMMDAVIDQCAEHGLDEVVIAMPHRGRLNVLANIVGKPYSQIFSEFEGNLNPSQAHGSGDVKYHLGATGTYIQMFGDNDIEVSLTANPSHLEAVDPVLEGLVRAKQDLLDTGEEGSDNRFSVVPLMLHGDAAFAGQGVVAETLNLALLRGYRTGGTIHIVVNNQIGFTTAPTDSRSSEYCTDVAKMIGAPIFHVNGDDPEACAWVARLAVDFRQAFKKDVVIDMLCYRRRGHNEGDDPSMTQPYMYDVIDTKRGSRKAYTEALIGRGDISMKEAEDALRDYQGQLERVFNEVRELEKHEIEPSESVEADQQIPSKLATAVDKAMLQRIGDAHLALPEGFTVHPRVRPVLEKRREMAYEGRIDWAFAELLALGSLIAEGKLVRLSGQDTQRGTFTQRHAVIVDRKTGEEFTPLQLLATNPDGTPTGGKFLVYNSALSEFAAVGFEYGYSVGNPDAMVLWEAQFGDFVNGAQSIIDEFISSGEAKWGQLSDVVLLLPHGHEGQGPDHTSGRIERFLQLWAEGSMTIAMPSTPANYFHLLRRHGKDGIQRPLIVFTPKSMLRNKAAVSDIRDFTESKFRSVLEEPMYTDGEGDRNKVTRLLLTSGKIYYELAARKAKENREDVAIVRIEQLAPLPRRRLAETLDRYPNVKEKFWVQEEPANQGAWPSFGLTLPEILPDHFTGLKRISRRAMSAPSSGSSKVHAVEQQEILDTAFG

SEQ ID NO: 70 - protein, 635 aa, Saccharomyces cerevisiae
MAPVTIEKFVNQEERHLVSNRSATIPFGEYIFKRLLSIDTKSVFGVPGDFNLSLLEYLYSPSVESAGLRWVGTCNELNAAYAADGYSRYSNKIGCLITTYGVGELSALNGIAGSFAENVKVLHIVGVAKSIDSRSSNFSDRNLHHLVPQLHDSNFKGPNHKVYHDMVKDRVACSVAYLEDIETACDQVDNVIRDIYKYSKPGYIFVPADFADMSVTCDNLVNVPRISQQDCIVYPSENQLSDIINKITSWIYSSKTPAILGDVLTDRYGVSNFLNKLICKTGIWNFSTVMGKSVIDESNPTYMGQYNGKEGLKQVYEHFELCDLVLHFGVDINEINNGHYTFTYKPNAKIIQFHPNYIRLVDTRQGNEQMFKGINFAPILKELYKRIDVSKLSLQYDSNVTQYTNETMRLEDPTNGQSSIITQVHLQKTMPKFLNPGDVVVCETGSFQFSVRDFAFPSQLKYISQGFFLSIGMALPAALGVGIAMQDHSNAHINGGNVKEDYKPRLILFEGDGAAQMTIQELSTILKCNIPLEVIIWNNNGYTIERAIMGPTRSYNDVMSWKWTKLFEAFGDFDGKYTNSTLIQCPSKLALKLEELKNSNKRSGIELLEVKLGELDFPEQLKCMVEAAALKRNKK

SEQ ID NO: 71 - DNA, 48 bp, Artificial Sequence
  Description of Artificial Sequence Synthetic primer
aaagaattcgtatacaaaggaggacaatcatgaggtctgatagcgtcg

SEQ ID NO: 72 - DNA, 89 bp, Artificial Sequence
  Description of Artificial Sequence Synthetic primer
tttaagcttccgcggccgcagatccccgggtaccatataataggccttttgtttaaacgtttagtacttcaaatgagaactaagtcatc

SEQ ID NO: 73 - DNA, 47 bp, Artificial Sequence
  Description of Artificial Sequence Synthetic primer
aaagaattcgtatacaaaggaggacaatcatgcccttagccgctgtg

SEQ ID NO: 74 - DNA, 47 bp, Artificial Sequence
  Description of Artificial Sequence Synthetic primer
tttggtaccaggcctgtttaaacagtacttcaagacagattaggttg

SEQ ID NO: 75 - DNA, 1231 bp, Renibacterium salmoninarum
aaagaattcgtatacaaaggaggacaatcatgagatctgatagcgtcgtcgtcggcggtcgcgagctcctgaaaactgcctctcgcatcgtcgttaaagtcggctcttcctcactcacctccgtatccggcggcatctctgagcaggcgctttcccagcttgtcgatgtattggcggctcgcaagaagcaaggcgccgagattattctggtctcctcaggtgcgatcgcggccggcctggcccctttgggcttggcccgccgtcctaaggatctagcgacccagcagtccgctgcgagcgtgggccagagcctgttgatggcccgctacggtgccgcttttggtagccacaatgtcaccgtttcccaggtgctgctcaccgcagaagatctcacccgccgcacccaatatgccaatgtgtaccgcgcattggagcgcctacttaatctcggtgtacttcccatcgtgaatgaaaatgacaccgtggcaacccacgagattcgctttggtgacaacgatcgcctggcagctctggtggcgcacctcgtcaaggccgacgcaatggtgctgctttccgacgtcgatgcggtctacgacggcccgccaaacaccggagcccgccgcattgagcagatcagcagccctgaggatcttgccggaatcaagattggctccgtcggttccgctggagtgggtactggcggaatggcgaccaaagtccaagctgcgtccatcgcggcaggctttggcaccccagctctggttacttcagcagcgaatgccgaggcggctttggccggtgccgatgtgggcacctggttttccttgcatggcgaacgccgtccgattcgcttgctatggctggcacatctggctagcacccacggaaagctgactctggacgacggcgccgttgctgccatccgcgaacgccataaatccctgttgccagccggaatcaaagcagtgtccggcgagttcgaagcgggcgacgccgttgaactggttgacctggcgggccgcacctttgcgcacggcttggtcaattatgacgccgcggaattgccaaccatgctgggcaagaaaactaaggatctagccggagaattgggccgcgactacgtccgcgccgtcgtccatgtggatgacctggttctcatttgaagtactaaacgtttaaacaaaaggcctattatatggtacccggggatctgcggccgcggaagcttaaa

SEQ ID NO: 76 - DNA, 1444 bp, Renibacterium salmoninarum
aaagaattcgtatacaaaggaggacaatcatgacagagctcatcagttcagcgaccgcctccacggcagatgacgccaagcaggacggcgtgcaaaactcttctccggcggataacgtgccggtcgaagctgcggtctttgcgatcgcggaccgatcaaggactgcagcccgcaaacttagtcgcgctaatcgagcctggaaagaccgtgccctgttagctattgcccacgagctgttggcccgccaagacaatgtgctgtcagcgaatgcggctgatatcgcggcggccaaggaatccggcacttcggacgcattgctggatcggatgtcgctgaatcaggagcggataaagaaacttgcggtggcattggaaaaccttgccgcactgcctgatccagtgggcacagttgttcgcgggcagaccctgccaaacggacttcggctgcaacaggtcaacgtgccgatgggcgtaatagccgcgatttacgaagcgcggcccaacgtcacgatagatattgccggtttggcgctcaagagcggcaatgccgtgattttgcgtggcggtagcgcggtggcgcaaacgaacacggcattactgaatgtcatgcgggatgccttggagaaggtggggctatcggttgacgcggtccaaagcgtcgattcctttggacgcgagggtgctgctgccttgatgcgagctcgtggacgagtggatgtgctgattccacgtggtggccatgagttgattcagacggtcgttgacaattcgtcggtgccggtgatcgaaaccggtgagggtaatgtgcacattttcatcgacgaatctgccgccgtcgagtcttcggtcgaaatcttattaaactctaaaacacagcggcccagtgtttgcaacacggtagagacacttttggtgcacgaaggctccaaggcacttgggccagtcctgtccgctttggccgctgctggcgtgcgattgcacgtcgatgagcgcatcatggcaaagctcccagccggggtagtggcggtgctggctgacgaccacgactgggccactgaatatatggatcttgatctggccgtggcgatggtcgattcgcttgatcaggccatcaagcacattcgtcgctggaccacaggtcataccgaagcaatcttgtcgaataatctgctcaacaccgagaagttcgtcgctgagattgattcggcggcagtaatcgtgaatgcgtcgaccagatttaccgacggcggtgagtttggacttggcgccgaagtgggtatttctacccaaaagttgcatgcccgcgggcctatgggtttggccgaattgaccacaaccaagtggattgtgcatggcgagggccaagttcgcggctaaagtactaaacgtttaaacaaaaggcctattatatggtacccggggatctgcggccgcggaagcttaaa

SEQ ID NO: 77 - DNA, 1201 bp, Nostoc sp.
gaattcgtatacaaaggaggacaatcatgcctctggctgcagtgatgaccgcccctaaccagcctgtcgaagttcagcaactcccagacccaatcctggagaagggcggcatcatcctggaaaccctgtactccgaggtctgtggcactgacgttcaccttctacacggacgcctcgaaggtgttccttatccaatcatcccaggtcacttctccgtgggccgcgttgttgagaccggtggcgctgtttccgatgtaaacggtaagctaatccagccaggcgcaatcgcgactttccttgatgtccacgaaacttgctacaattgctggtactgtcttgttgccaaggcttccacccgctgtcctcagcgtaaggtttatggcgtgacatactccgcaaaggacggcctgttgggcggctggtccgagcttatttacctgaagccgggtgtcaaggttctgaccctgccagaagaagtctctcctaagcagttcatcgctggtggttgcgcactcccaaccgccctgcacgctatcgaccgtgcacaaatccagatcggtgacgtcgtcgtcgtccagggttgtggaccagtcggtctctccgcagcaattctcgctctgctctccggagctggcaaggttattgtgatcgataagttcgagtcccgcctggtcgtcgcaaagagcttcggcgttgacgaaacccttgcaatcaaggcagacgacccacgtcagcacatcgaacgcgtgctcgagctcaccaacggacacggtgcagacgttaccatcgaggccaccggtatcccaatcgcagttaaggaaggtcttaacatgacccgcaacggtggtcgctacgtcatcgttggtcactacaccaacaccggtgagatccttatcaacccacacctggaaatcaaccttaagcacatcgacatccgcggcacctggggtatcgacttcagccacttctaccgtatgattgagctactgaagcgtcactccgactcctccaagaacatcgcctggagctccattatcagccgttcttacaccctgaaggagatcaaccaggccctcgcagacgttgaacagggctccgtgcttaaggctgttatccagccaaacctgtcctgaagtactaaacgtttaaacaaaaggcctattatatggtacccggggatctgcggccgcggaagcttaaa

SEQ ID NO: 78 - DNA, 1258 bp, Yarrowia lipolytica
gaattcgtatacaaaggaggacaatcatgattcgcaacaacttgcgctccctttcccgcgccttcagcacctcctccatgcgtctgggcgccggaatggacgcctccaagctccagatcaccaagaccaagtccccaaaggaaaagcaggccccaaaggatctcattttcggccataccttcaccgaccacatgctgactgtcgagtggactgccaaggacggctgggctgctccacagatcaccccatacggtcctcttgagttagacccttccgccgtcgtcctgcactacgcctttgagtgtttcgagggcctcaaggcttacaaggacgagtctggaaacgtgcgtctgttccgcgttgataagaacatgcaccgcatgaacacctccgccgagcgcatctgcctgccagagtttgatggcgccgaggctgccaagctgattggccaattggccaaattagattccgctattcctgagggacgcggctactccatgtacctccgcccttctctgattggaaccaccgccgctctcggcgtcggaaccccagataaggcgctcttttacgtcattgcatccccagtcggcccatactaccctaccggattcaaggccgtcaagctggaggctactgactacgctgtccgcgcctggcctggaggagtcggaaacaagaagctgggagccaactacgctccatgtatcaagcctcagcagcaggccgcttctcgcggctaccagcagaacctgtggctgtttggcgacgagggcaacatcaccgaggtcgggaccatgaacgccttctttgtgtttgagcgcaacggcaagaaggagcttgtcactgctcctttggacggtactattttagaaggtgtcactcgcgactccattctggagctggctcgcgaacgcttgccttctgctgactggatcgtttccgagcgctactgcactattaaggaggtcgcggaggctgccgagaagggcgagcttgttgaagcctttggagctggtactgccgctgttgtctcccctatcaaggagattggatggggagagaagactattaacattcctctccagcctggcaaggaggccggtaagctgactgagactgttaatgagtggattggagatatccagtacggtaaggatgaatacaagggatggtctaaggtggtctaaagtactaaacgtttaaacaaaaggcctattatatggtacccggggatctgcggccgcggaagcttaaa

SEQ ID NO: 79 - DNA, 1738 bp, Lactococcus lactis
gaattcgtatacaaaggaggacaatcatgtacaccgttggtgattacctcctggaccgcttgcacgaactaggcatcgaggagatcttcggcgtcccaggtgactacaacctgcagttcctcgaccaaatcatctctcgcgaggacatgaagtggatcggcaacgcaaacgaacttaacgcaagctacatggctgacggctacgctcgcaccaagaaggccgctgcattcctgacaaccttcggcgtcggtgagctttcagctatcaatggccttgcgggatcctacgcagaaaacctgcctgtcgttgagattgttggttcccctacttccaaggttcagaacgatggtaagttcgtccaccacaccctcgctgacggtgacttcaagcactttatgaagatgcacgaaccagttaccgcggcccgcaccctcttgaccgcagaaaacgctacctacgaaatcgaccgcgttctttcccagctgctgaaggaacgcaagccagtttacatcaacctgcccgtcgatgttgcggcagcaaaggctgaaaagcctgctctctcactcgagaaggaatcttctaccaccaacaccaccgagcaagtcatcctgagcaagattgaagaatctctgaagaacgctcagaagccagtcgttattgcaggccacgaggtaatctccttcggtctcgaaaagaccgttactcagttcgtttctgagaccaagcttccaatcaccacactgaacttcggtaagagcgccgtcgacgaaagccttccatccttcctgggtatctacaacggcaaattgtccgaaatttccctgaagaatttcgttgagtctgccgattttatcctgatgctgggcgtgaagctgactgactcttccaccggtgctttcactcaccaccttgacgaaaacaagatgatttcccttaacatcgatgaaggtatcatcttcaacaaggtggttgaggacttcgacttccgcgctgttgtctcctccctttccgagctgaagggaattgaatacgagggccagtatatcgataagcagtacgaggagttcatcccaagctccgcccctctgagccaggatcgtctgtggcaggcggtggaatccctcacccagtccaacgagaccatcgtggcagagcagggaacctcctttttcggcgctagcaccatctttctgaagagcaactctcgcttcatcggtcagccactatggggatctatcggatacactttcccagcagcactgggctcccagatcgctgataaggaatcccgccacctcctgttcatcggcgacggttccctgcaactcaccgtgcaggagctcggcctttctatccgcgagaagctgaacccaatctgcttcatcatcaacaacgacggttacaccgttgaacgcgaaatccacggtccaacccagtcctacaacgatatccctatgtggaactactctaagctcccagaaaccttcggcgcaaccgaggatcgcgttgtttccaagatcgttcgtaccgagaacgagttcgtttctgtgatgaaggaagcacaggcagatgtgaaccgtatgtactggatcgaacttgtcctggagaaggaagacgctccaaagctcctcaagaagatgggtaagctcttcgcagagcagaataagtagagtactaaacgtttaaacaaaaggcctattatatggtacccggggatctgcggccgcgggcatgcaaa

SEQ ID NO: 80 - DNA, 1177 bp, Saccharomyces cerevisiae
gaattcgtatacaaaggaggacaatcatgagctacccagagaagttcgagggtattgccatccagtcccacgaagactggaagaacccaaagaagaccaagtacgaccctaagccattctacgaccacgacattgacatcaagatcgaagcttgcggagtatgtggttctgatatccactgcgctgccggccactggggcaatatgaagatgcctctcgtcgtcggccacgagattgttggtaaggttgttaagctcggtcctaagtctaacagcggtctgaaggttggccagcgtgtgggtgttggtgcacaggtgttttcctgccttgagtgtgaccgttgtaagaacgacaacgagccatactgcaccaagttcgtgaccacctactcccagccttacgaggatggctacgtctcccagggcggctacgccaactacgtacgcgttcacgagcacttcgtcgtaccaatccctgagaacatcccaagtcacctcgctgctcctctgctctgcggaggtttgaccgtgtactctcctctggtccgtaacggatgtggcccaggcaagaaggtcggcattgtcggtctaggcggcatcggttcaatgggaaccctcatctccaaggcaatgggcgctgaaacctacgttatcagccgttcttcccgcaagcgcgaagacgctatgaagatgggcgctgatcactacatcgccaccctggaggagggcgattggggagagaaatacttcgacaccttcgatcttatcgttgtttgcgcttcctctctgaccgatatcgatttcaacatcatgcctaaggccatgaaggttggaggccgcatcgtttccatctccatcccagagcagcacgagatgctgtccctgaagccatacggtctgaaggctgtttccatctcctactcagcactgggcagcatcaaggaacttaaccagctgctgaagctggtgtccgaaaaggacatcaagatctgggttgagaccctgccagtcggcgaagcgggcgtgcacgaagcttttgaacgtatggaaaagggtgacgtgcgctaccgcttcaccctggtcggatacgacaaggagttctccgactagagtactaaacgtttaaacaaaaggcctattatatggtacccggggatctgcggccgcgggcatgcaaa

SEQ ID NO: 81 - DNA, 1107 bp, Nostoc punctiforme
atgcccttagccgctgtgatgactgcacctaatcaaccagttgaagtgcaacaattaccagagccgattctggaaaagggtggaatcattattgaaaccttatattctgaggtttgtggaactgatgtacatttattacatgggcgtttagagggagtaccatatcctatcatccctggacacttctcagttggtcgtgtggtggaaacaggtggagcagttagtgatgtcaatggtaaattaattcagcctggagcgatcgctacttttttagatgtccacgaaacctgttacaactgctggtattgtctagtagccaaagcatccactcgctgtccaaaacgtaaagtttatggtgtcacctactcagccaaagatgggttgctgggtgggtggtctgaattaatttacctcaaaccaggggttaaagttctcactttacccgaagaagtctcaccaaagcaattcattgctggaggatgtgctttaccaactgcactacacgctattgatcgagcgcagattcaaattggtgatgtcgtcgtggtgcagggttgcggacctgtgggtttgagtgcggcaattctagctttgctctcaggtgcgggcaaagtaattgtaattgataaatttgagagccgattagtagttgcaaaatctttcggtgtagatgaaacccttgcaattagggctgatgatccacgacaacatattgagcgagttttggaattaaccaacggacacggcgctgatgtcactattgaagctacaggcattcctattgctgtcaaagagggcttaaatatgaccagaaatggaggtcgctatgttattgtcgggcattacacaaacacgggtgagattctcatcaatccacacttagagattaatctaaagcatattgatattcgcggaacttggggaattgatttcagccatttttatagaatgattgaattactaaaacgtcatagcgattccagtaaaaatattgcttggtcaagcataattagtcgttcatatacactaaaagaaattaatcaagcactcgtagatgtagagcaaggctctgtattaaaagctgtgattcaacctaatctgtcttga

SEQ ID NO: 82 - DNA, 1020 bp, Pseudomonas fluorescens
atgggtaacgaaagcatcaactgggacaagctgggttttgactacatcaagaccgacaagcggtttctccaggtctggaaaaacggcgaatggcaagaaggcaccctgaccgacgacaacgtgctgcacatcagcgagggctccaccgccctgcactacggccagcaatgctttgaaggcctcaaggcttaccgctgcaaggacggttcgatcaacctgttccgcccggaccagaacgccgcccgcatgcaacgcagctgcgcgcgcctgctgatgccgcatgtgccgaccgacgtgttcatcgacgcctgcaaacaagtggtcaaggccaacgaacatttcatcccgccgtacggcagcggcggcgcgctgtacctgcgcccgttcgtgatcggcaccggtgacaacatcggcgtgcgcaccgcgccggagttcatcttctccgtgttctgcatcccggtcggcgcctacttcaaaggcggcctggtgccacacaacttccagatctccaccttcgaccgcgccgcgccacaaggcaccggtgccgccaaggtcggtggcaactacgccgccagcctgatgccaggttccgaagcgaagaaagccggttttgccgacgcgatctacctggacccgatgacccactcgaaaatcgaagaagtcggctcggccaacttcttcgggatcacccacgacaaccagttcatcacgccgaagtcgccttcggtgctgccaggcatcacccgcctgtcgctgatcgaactggccaagacccgcctgggtctggacgtggtcgagggcgaagtgttcatcgacaaactggaccagttcaaggaagccggcgcctgcggtacggctgcggtgatctcgccgatcggcggcatccagtacaacggcaagctgcacgtgttccacagcgagaccgaagtcggcccgatcacccagaagctctacaaagagctgaccggtgtgcagactggtgacgttgaagcgccgcaaggctggatcgtcaaggtttga

SEQ ID NO: 83 - DNA, 1738 bp, Lactococcus lactis; identical to SEQ ID NO: 79

SEQ ID NO: 84 - DNA, 1177 bp, Saccharomyces cerevisiae; identical to SEQ ID NO: 80

SEQ ID NO: 85 - DNA, 34 bp, Artificial Sequence
  Description of Artificial Sequence Synthetic primer
cacacggtctccctaggacgctctcgatgaggag

SEQ ID NO: 86 - DNA, 44 bp, Artificial Sequence
  Description of Artificial Sequence Synthetic primer
cacacggtctcaagagacactagtcagacccactctagccgttg

SEQ ID NO: 87 - DNA, 45 bp, Artificial Sequence
  Description of Artificial Sequence Synthetic primer
cacaccgtctcactctacagatctgcacgcctacttaaccagcct

SEQ ID NO: 88 - DNA, 33 bp, Artificial Sequence
  Description of Artificial Sequence Synthetic primer
cacaccgtctcagatctcctgcacatgcccttt

SEQ ID NO: 89 - DNA, 33 bp, Artificial Sequence
  Description of Artificial Sequence Synthetic primer
aaaacttaagccaggatcgacaaaggactcgag

SEQ ID NO: 90 - DNA, 30 bp, Artificial Sequence
  Description of Artificial Sequence Synthetic primer
aaaagagctccaaaatcatcatgccgggcg

SEQ ID NO: 91 - DNA, 41 bp, Artificial Sequence
  Description of Artificial Sequence Synthetic primer
caacgagaccatcgtggcannncagggaacctcctttttcg

SEQ ID NO: 92 - DNA, 39 bp, Artificial Sequence
  Description of Artificial Sequence Synthetic primer
gagaccatcgtggcagagnnnggaacctcctttttcggc

SEQ ID NO: 93 - DNA, 37 bp, Artificial Sequence
  Description of Artificial Sequence Synthetic primer
ccatcgtggcagagcagnnnacctcctttttcggcgc

SEQ ID NO: 94 - DNA, 38 bp, Artificial Sequence
  Description of Artificial Sequence Synthetic primer
accatcgtggcagagcagggannntcctttttcggcgc

SEQ ID NO: 95 - DNA, 37 bp, Artificial Sequence
  Description of Artificial Sequence Synthetic primer
cagagcagggaacctccnnnttcggcgctagcaccat

SEQ ID NO: 96 - DNA, 38 bp, Artificial Sequence
  Description of Artificial Sequence Synthetic primer
tggcagagcagggaacctcctttnnnggcgctagcacc

SEQ ID NO: 97 - DNA, 45 bp, Artificial Sequence
  Description of Artificial Sequence Synthetic primer
aactctcgcttcatcggtcagnnnctatggggatctatcggatac

SEQ ID NO: 98 - DNA, 45 bp, Artificial Sequence
  Description of Artificial Sequence Synthetic primer
ttcatcggtcagccactatggnnntctatcggatacactttccca

SEQ ID NO: 99 - DNA, 41 bp, Artificial Sequence
  Description of Artificial Sequence Synthetic primer
cggtcagccactatggggannnatcggatacactttcccag

SEQ ID NO: 100 - DNA, 41 bp, Artificial Sequence
  Description of Artificial Sequence Synthetic primer
ggtcagccactatggggatctnnnggatacactttcccagc

SEQ ID NO: 101 - DNA, 41 bp, Artificial Sequence
  Description of Artificial Sequence Synthetic primer
caacaacgacggttacaccnnngaacgcgaaatccacggtc

SEQ ID NO: 102 - DNA, 42 bp, Artificial Sequence
  Description of Artificial Sequence Synthetic primer
tcaacaacgacggttacaccgttnnncgcgaaatccacggtc

SEQ ID NO: 103 - DNA, 39 bp, Artificial Sequence
  Description of Artificial Sequence Synthetic primer
acggttacaccgttgaacgcgaannncacggtccaaccc

SEQ ID NO: 104 - DNA, 45 bp, Artificial Sequence
  Description of Artificial Sequence Synthetic primer
aaggaagacgctccaaagctcnnnaagaagatgggtaagctcttc